(19) United States
(12) Patent Application Publication
(10) Pub. No.: US 2022/0306630 A1
(43) Pub. Date: Sep. 29, 2022

(54) AGONISTS OF ROR GAMMAt

(71) Applicant: BRISTOL-MYERS SQUIBB COMPANY, Princeton, NJ (US)

(72) Inventors: Lalgudi S. Harikrishnan, Skillman, NJ (US); Peter Kinam Park, New York, NY (US); Zheming Ruan, Dayton, NJ (US); Donna D. Wei, Belle Mead, NJ (US); Daniel O'Malley, New Hope, PA (US); Honghe Wan, Pennington, NJ (US); Ashok Vinayak Purandare, Pennington, NJ (US); Brian E. Fink, Yardley, PA (US)

(21) Appl. No.: 17/632,909

(22) PCT Filed: Aug. 5, 2020
(86) PCT No.: PCT/US2020/044918

§ 371 Date: Feb. 4, 2022

Related U.S. Application Data
(60) Provisional application No. 62/883,171, filed on Aug. 6, 2019.

(51) Int. Cl. C07D 471/10; C07C 317/18; C07D 213/73; C07D 213/71; C07C 255/60; C07D 213/82; C07D 231/12; C07D 231/18; C07D 263/24; C07D 213/56; C07D 401/12; C07C 311/16; C07D 213/69; C07D 233/61; C07D 211/34; C07D 235/18; C07D 213/58; C07D 211/96; C07D 295/26

(52) U.S. Cl. CPC C07D 471/10; C07C 317/18; C07D 213/73; C07D 213/71; C07C 255/60; C07D 213/82; C07D 231/12; C07D 231/18; C07D 263/24; C07D 213/56; C07D 401/12; C07C 311/16; C07D 213/69; C07D 233/61; C07D 211/34; C07D 235/18; C07D 213/58; C07D 211/96; C07D 295/26

(57) ABSTRACT

The present invention is directed to compounds of the formula (I) wherein all substituents are defined herein, as well as pharmaceutically acceptable compositions comprising compounds of the invention and methods of using said compositions in the treatment of various disorders.

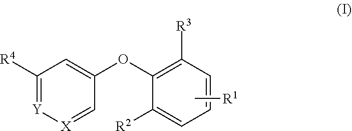

### CROSS-REFERENCE TO RELATED APPLICATIONS

This application claims the benefit of U.S. Provisional Application No. 62/883,171, filed Aug. 6, 2019, the disclosure of which is incorporated herein by reference in its entirety.

### FIELD OF THE INVENTION

The invention provides novel compounds, pharmaceutical compositions comprising the compounds, and methods of using them, for example, for the treatment or prophylaxis of certain cancers and to their use in therapy.

### BACKGROUND OF THE INVENTION

RORgt is a key lineage-defining transcription factor involved in the differentiation of naïve T cells to Th17 and Tc17 cells. IL-17 is a signature cytokine of RORgt transactivation (Ivanov et al; Cell 2006, 126, 1121).

High IL-17 levels have been associated with various autoimmune diseases. Consequently, several groups have identified RORgt inverse agonists to decrease IL-17 production aimed at suppressing immunity to treat various autoimmune diseases, most notably psoriasis (Bronner et al. Expert Opin. Ther. Pat. 2017, 27, 1, 101)

More recently RORgt agonism has been reported to increase the production of antitumor cytokines and chemokines (such as IL-17A and GM-CSF), as well as augment the expression of co-stimulatory receptors (such as CD137 and CD226) and decrease the levels of co-inhibitory receptors (such as PD1 and TIGIT) (Hu et al. Oncoimmunology, 2016, 5, 12, e1254854). High levels of Th17 cells or IL-17 has been associated with patient survival in certain cancers (Kryczek et al. Blood 2009, 114, 1141; Sfanos et al. Clin. Can. Res. 2008, 14, 3254). Therefore RORgt agonism has the potential to boost immune response to tumors and thus confer durable antitumor response. A recent review (Qiu et al J. Med. Chem. 2018, 61, 5794) summarizes the progress by various research groups towards the identification of RORgt agonists.

The present invention, therefore, provides novel cyclic dinucleotides which may be useful for the treatment of cancer.

### SUMMARY OF THE INVENTION

There is provided a compound of formula (I)

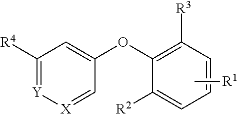

wherein all substituents are defined herein.

In another aspect, there is provided a pharmaceutical composition comprising a compound of the invention or a pharmaceutically acceptable salt thereof and one or more pharmaceutically acceptable carriers, diluents or excipients.

In another aspect, there is provided a method of treating cancer which comprises administering to a subject in need thereof a therapeutically effective amount of an agonist of RORγ.

### DETAILED DESCRIPTION OF THE INVENTION

The following are aspects and embodiments of the present invention, as well as additional aspects and embodiments that can be within the scope of those shown. The aspects of the invention are not limited to those described below.

In a first aspect, there is disclosed a compound of formula I

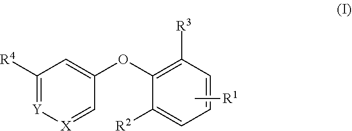

wherein

X is —N— or CR⁵, where R⁵ is hydrogen, C₁₋₃ alkyl, CN or halogen;

Y is CR⁶, where R⁶ is hydrogen, CN, halogen, O—C₁₋₃ alkyl, O—C₁₋₃ haloalkyl or C₃₋₆ cycloalkyl;

R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};

each R^{x} and R^{y} is independently hydrogen or C₁₋₃ alkyl;

R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or aryl, all of said alkyl, heterocyclyl or aryl groups substituted with 0-3 R^{1b};

R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;

R² and R³ are, independently at each occurrence, hydrogen, halogen or C₁₋₃ alkyl;

R⁴ is C₁₋₆ alkyl, C₁₋₆ alkenyl, C₁₋₆ haloalkyl, C₁₋₆ hydroxyalkyl, CO—C₁₋₃ haloalkyl or C₃₋₆ cycloalkyl, each of said groups substituted with 0-2 R^{4a};

R^{4a} is halogen or C₁₋₃ alkyl;

p is 0, 1 or 2;

r is 0, 1, 2, 3 or 4;

or a stereoisomer or pharmaceutically-acceptable salt thereof.

In a second aspect, there is disclosed a compound of formula I

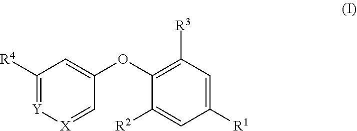

wherein

X is —N— or CR⁵, where R⁵ is hydrogen, C₁₋₃ alkyl, CN or halogen;

Y is CR⁶, where R⁶ is hydrogen, CN, halogen, O—C₁₋₃ alkyl, O—C₁₋₃ haloalkyl or C₃₋₆ cycloalkyl;

R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};

each R^{x} and R^{y} is independently hydrogen or C₁₋₃ alkyl;

R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or aryl, all of said alkyl, heterocyclyl or aryl groups substituted with 0-3 R^{1b};

R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;

R² and R³ are, independently at each occurrence, hydrogen, halogen or C₁₋₃ alkyl;

R⁴ is C₁₋₆ alkyl, C₁₋₆ alkenyl, C₁₋₆ haloalkyl, C₁₋₆ hydroxyalkyl, CO—C₁₋₃ haloalkyl or C₃₋₆ cycloalkyl, each of said groups substituted with 0-2 R^{4a};

R^{4a} is halogen or C₁₋₃ alkyl;

p is 0, 1 or 2;

r is 0, 1, 2, 3 or 4;

or a stereoisomer or pharmaceutically-acceptable salt thereof.

In a third aspect, there is disclosed a compound of the formula

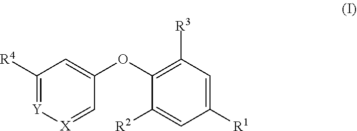

wherein

X is —N— or CR⁵, where R⁵ is hydrogen, C₁₋₃ alkyl, CN or halogen;

Y is CR⁶, where R⁶ is hydrogen, CN, halogen, O—C₁₋₃ alkyl, O—C₁₋₃ haloalkyl or C₃₋₆ cycloalkyl;

R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};

each R^{x} and R^{y} is independently hydrogen or C₁₋₃ alkyl;

R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or aryl, all of said alkyl, heterocyclyl or aryl groups substituted with 0-3 R^{1b};

R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;

R² and R³ are, independently at each occurrence, hydrogen, halogen or C₁₋₃ alkyl;

R⁴ is C₁₋₆ alkyl, C₁₋₆ haloalkyl, C₁₋₆ hydroxyalkyl, CO—C₁₋₃ haloalkyl or C₃₋₆ cycloalkyl, each of said groups substituted with 0-2 R^{4a};

R^{4a} is halogen or C₁₋₃ alkyl;

p is 0 or 1;

r is 0, 1, 2 or 3;

or a stereoisomer or pharmaceutically-acceptable salt thereof.

In a fourth aspect, there is disclosed a compound of the formula

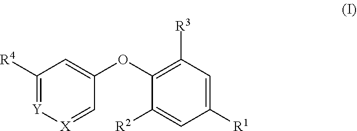

wherein

X is —N— or CR⁵, where R⁵ is hydrogen, C₁₋₃ alkyl, CN or halogen;

Y is CR⁶, where R⁶ is hydrogen, CN, halogen, O—C₁₋₃ alkyl, O—C₁₋₃ haloalkyl or C₃₋₆ cycloalkyl;

R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};

each R^{x} and R^{y} is independently hydrogen or C₁₋₃ alkyl;

R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or phenyl, all of said alkyl, heterocyclyl or phenyl groups substituted with 0-3 R^{1b};

R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;

R² and R³ are, independently at each occurrence, CH₃, Cl or F;

R⁴ is C₁₋₆ alkyl, C₁₋₆ haloalkyl, C₁₋₆ hydroxyalkyl, CO—C₁₋₃ haloalkyl or C₃₋₆ cycloalkyl, each of said groups substituted with 0-2 R^{4a};

R^{4a} is halogen or C₁₋₃ alkyl;

p is 0 or 1;

r is 0, 1, 2 or 3;

or a stereoisomer or pharmaceutically-acceptable salt thereof.

In a fifth aspect, there is disclosed a compound of the formula

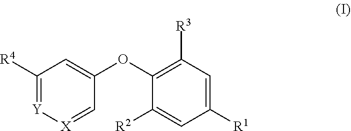

wherein

X is —N— or CR⁵, where R⁵ is hydrogen, C₁₋₃ alkyl, CN or halogen;

Y is CR⁶, where R⁶ is hydrogen, CN, halogen, O—C₁₋₃ alkyl, O—C₁₋₃ haloalkyl or C₃₋₆ cycloalkyl;

R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};

each R^{x} and R^{y} is independently hydrogen or C₁₋₃ alkyl;

R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or phenyl, all of said alkyl, heterocyclyl or phenyl groups substituted with 0-3 R^{1b};

R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;

R² and R³ are, independently at each occurrence, Cl or F;

R⁴ is C₁₋₆ alkyl, C₁₋₆ haloalkyl, C₁₋₆ hydroxyalkyl, CO—C₁₋₃ haloalkyl or C₃₋₆ cycloalkyl, each of said groups substituted with 0-2 R^{4a};

R^{4a} is halogen or C₁₋₃ alkyl;

p is 0 or 1;

r is 0, 1, 2 or 3;

or a stereoisomer or pharmaceutically-acceptable salt thereof.

In a sixth aspect, there is disclosed a compound of the formula

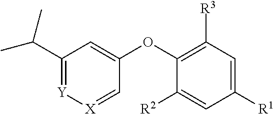

wherein

X is —N— or CR⁵, where R⁵ is hydrogen, C₁₋₃ alkyl, CN or halogen;

Y is CR⁶, where R⁶ is hydrogen, CN, halogen, O—C₁₋₃ alkyl, O—C₁₋₃ haloalkyl or C₃₋₆ cycloalkyl;

R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};

each R^{x} and R^{y} is independently hydrogen or C₁₋₃ alkyl;

R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or phenyl, all of said alkyl, heterocyclyl or phenyl groups substituted with 0-3 R^{1b};

R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;

R² and R³ are, independently at each occurrence, Cl or F;

p is 0 or 1;

r is 0, 1, 2 or 3;

or a stereoisomer or pharmaceutically-acceptable salt thereof.

In a 7th aspect, there is disclosed a compound of the formula

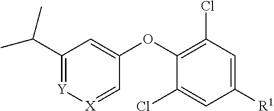

wherein

X is —N— or CR⁵, where R⁵ is hydrogen, C₁₋₃ alkyl, CN or halogen;

Y is CR⁶, where R⁶ is hydrogen, CN, halogen, O—C₁₋₃ alkyl, O—C₁₋₃ haloalkyl or C₃₋₆ cycloalkyl;

R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};

each R^{x} and R^{y} is independently hydrogen or C₁₋₃ alkyl;

R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or phenyl, all of said alkyl, heterocyclyl or phenyl groups substituted with 0-3 R^{1b};

R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;

p is 0 or 1;

r is 0, 1, 2 or 3;

or a stereoisomer or pharmaceutically-acceptable salt thereof.

In an 8th aspect, there is disclosed a compound of the formula

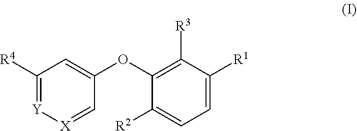

wherein

X is —N— or CR⁵, where R⁵ is hydrogen, C₁₋₃ alkyl, CN or halogen;

Y is CR⁶, where R⁶ is hydrogen, CN, halogen, O—C₁₋₃ alkyl, O—C₁₋₃ haloalkyl or C₃₋₆ cycloalkyl;

R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};

each R^{x} and R^{y} is independently hydrogen or C₁₋₃ alkyl;

R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or aryl, all of said alkyl, heterocyclyl or aryl groups substituted with 0-3 R^{1b};

R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;

R² and R³ are, independently at each occurrence, hydrogen, halogen or C₁₋₃ alkyl;

R⁴ is C₁₋₆ alkyl, C₁₋₆ alkenyl, C₁₋₆ haloalkyl, C₁₋₆ hydroxyalkyl, CO—C₁₋₃ haloalkyl or C₃₋₆ cycloalkyl, each of said groups substituted with 0-2 R^{4a};

R^{4a} is halogen or C₁₋₃ alkyl;

p is 0, 1 or 2;

r is 0, 1, 2, 3 or 4;

or a stereoisomer or pharmaceutically-acceptable salt thereof.

In a 9th aspect, there is disclosed a compound of the formula

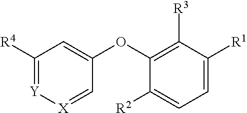

wherein

X is —N— or CR⁵, where R⁵ is hydrogen, C₁₋₃ alkyl, CN or halogen;

Y is CR⁶, where R⁶ is hydrogen, CN, halogen, O—C₁₋₃ alkyl, O—C₁₋₃ haloalkyl or C₃₋₆ cycloalkyl;

R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};

each R^{x} and R^{y} is independently hydrogen or C₁₋₃ alkyl;

R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or aryl, all of said alkyl, heterocyclyl or aryl groups substituted with 0-3 R^{1b};

R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;

R² and R³ are, independently at each occurrence, hydrogen, halogen or C₁₋₃ alkyl;

R⁴ is C₁₋₆ alkyl, C₁₋₆ haloalkyl, C₁₋₆ hydroxyalkyl, CO—C₁₋₃ haloalkyl or C₃₋₆ cycloalkyl, each of said groups substituted with 0-2 R^{4a};

R^{4a} is halogen or C₁₋₃ alkyl;

p is 0 or 1;

r is 0, 1, 2 or 3;

or a stereoisomer or pharmaceutically-acceptable salt thereof.

In a 10th aspect, there is disclosed a compound of the formula

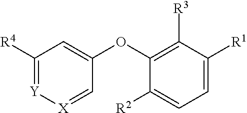

wherein

X is —N— or CR⁵, where R⁵ is hydrogen, C₁₋₃ alkyl, CN or halogen;

Y is CR⁶, where R⁶ is hydrogen, CN, halogen, O—C₁₋₃ alkyl, O—C₁₋₃ haloalkyl or C₃₋₆ cycloalkyl;

R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};

each R^{x} and R^{y} is independently hydrogen or C₁₋₃ alkyl;

R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or phenyl, all of said alkyl, heterocyclyl or phenyl groups substituted with 0-3 R^{1b};

R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;

R² and R³ are, independently at each occurrence, CH₃, Cl or F;

R⁴ is C₁₋₆ alkyl, C₁₋₆ haloalkyl, C₁₋₆ hydroxyalkyl, CO—C₁₋₃ haloalkyl or C₃₋₆ cycloalkyl, each of said groups substituted with 0-2 R^{4a};

R^{4a} is halogen or C₁₋₃ alkyl;

p is 0 or 1;

r is 0, 1, 2 or 3;

or a stereoisomer or pharmaceutically-acceptable salt thereof.

In an 11th aspect, there is disclosed a compound of the formula

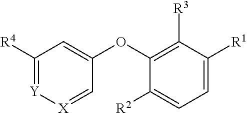

wherein

X is —N— or CR⁵, where R⁵ is hydrogen, C₁₋₃ alkyl, CN or halogen;

Y is CR⁶, where R⁶ is hydrogen, CN, halogen, O—C₁₋₃ alkyl, O—C₁₋₃ haloalkyl or C₃₋₆ cycloalkyl;

R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};

each R^{x} and R^{y} is independently hydrogen or C₁₋₃ alkyl;

R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or phenyl, all of said alkyl, heterocyclyl or phenyl groups substituted with 0-3 R^{1b};

R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;

R² and R³ are, independently at each occurrence, Cl or F;

R⁴ is C₁₋₆ alkyl, C₁₋₆ haloalkyl, C₁₋₆ hydroxyalkyl, CO—C₁₋₃ haloalkyl or C₃₋₆ cycloalkyl, each of said groups substituted with 0-2 R^{4a};

R^{4a} is halogen or C₁₋₃ alkyl;

p is 0 or 1;

r is 0, 1, 2 or 3;

or a stereoisomer or pharmaceutically-acceptable salt thereof.

In a 12th aspect, there is disclosed a compound of the formula

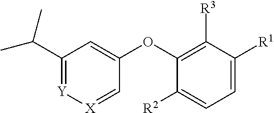

wherein

X is —N— or CR⁵, where R⁵ is hydrogen, C₁₋₃ alkyl, CN or halogen;

Y is CR⁶, where R⁶ is hydrogen, CN, halogen, O—C₁₋₃ alkyl, O—C₁₋₃ haloalkyl or C₃₋₆ cycloalkyl;

R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};

each R^{x} and R^{y} is independently hydrogen or C₁₋₃ alkyl;

R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or phenyl, all of said alkyl, heterocyclyl or phenyl groups substituted with 0-3 R^{1b};

R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;

R² and R³ are, independently at each occurrence, Cl or F;

p is 0 or 1;

r is 0, 1, 2 or 3;

or a stereoisomer or pharmaceutically-acceptable salt thereof.

In a 13th aspect, there is disclosed a compound of the formula

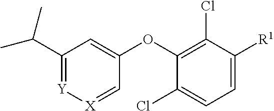

wherein

X is —N— or CR⁵, where R⁵ is hydrogen, C₁₋₃ alkyl, CN or halogen;

Y is CR⁶, where R⁶ is hydrogen, CN, halogen, O—C₁₋₃ alkyl, O—C₁₋₃ haloalkyl or C₃₋₆ cycloalkyl;

R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};

each R^{x} and R^{y} is independently hydrogen or C₁₋₃ alkyl;

R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or phenyl, all of said alkyl, heterocyclyl or phenyl groups substituted with 0-3 R^{1b};

R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;

p is 0 or 1;

r is 0, 1, 2 or 3;

or a stereoisomer or pharmaceutically-acceptable salt thereof.

In another aspect, there are disclosed the following compounds of the invention:
2,4-dichloro-3-(3-isopropyl-4-methoxyphenoxy)benzonitrile,
4,6-dichloro-5-(3-isopropyl-4-methoxyphenoxy)-2-phenyl-1H-benzo[d]imidazole,
N-({2,4-dichloro-3-[4-methoxy-3-(propan-2-yl)phenoxy]phenyl}methyl)-2-[(1-methanesulfonylpiperidin-4-yl)oxy]acetamide,
N-({2,4-dichloro-3-[4-methoxy-3-(propan-2-yl)phenoxy]phenyl}methyl)-2-{[1-(ethanesulfonyl)piperidin-4-yl]oxy}acetamide,
2-benzyl-4,6-dichloro-5-[4-methoxy-3-(propan-2-yl)phenoxy]-1H-1,3-benzodiazole,
4,6-dichloro-5-[4-methoxy-3-(propan-2-yl)phenoxy]-2-[(pyridin-3-yl)methyl]-1H-1,3-benzodiazole,
3,5-dichloro-4-[4-methoxy-3-(propan-2-yl)phenoxy]aniline,
{3,5-dichloro-4-[4-methoxy-3-(propan-2-yl)phenoxy]phenyl}methanol

or a pharmaceutically acceptable salt thereof.

In another aspect, there is provided a compound selected from any subset list of compounds within the scope of any of the above aspects.

### OTHER EMBODIMENTS OF THE INVENTION

In another embodiment, the invention provides a pharmaceutical composition, comprising a pharmaceutically acceptable carrier and a therapeutically effective amount of at least one of the compounds of the invention or a stereoisomer, a tautomer, a pharmaceutically acceptable salt, or a solvate thereof.

In another embodiment, the invention provides a process for making a compound of the invention or a stereoisomer, a tautomer, a pharmaceutically acceptable salt, or a solvate thereof.

In another embodiment, the invention provides a method for the treatment and/or prophylaxis of various types of cancer, comprising administering to a patient in need of such treatment and/or prophylaxis a therapeutically effective amount of one or more compounds of the invention, alone, or, optionally, in combination with another compound of the invention and/or at least one other type of therapeutic agent.

In another embodiment, the invention provides a method for the treatment and/or prophylaxis of various types of cancer, including small cell lung cancer, non-small cell lung cancer, colorectal cancer, melanoma, renal cell carcinoma, head and neck cancer, Hodgkin's lymphoma, bladder cancer, esophageal carcinoma, gastric carcinoma, ovarian carcinoma, cervical carcinoma, pancreatic carcinoma, prostate carcinoma, breast cancers, urinary carcinoma, brain tumors such as glioblastoma, non-Hodgkin's lymphoma, acute lymphatic leukemia (ALL), chronic lymphatic leukemia (CLL), acute myeloid leukemia (AML), chronic myeloid leukemia (CML), hepatocellular carcinoma, multiple myeloma, gastrointestinal stromal tumors, mesothelioma, and other solid tumors or other hematological cancers

In another embodiment, the invention provides a method for the treatment and/or prophylaxis of various types of cancer, including without limitation, small cell lung cancer, non-small cell lung cancer, colorectal cancer, melanoma, renal cell carcinoma, head and neck cancer, Hodgkin's lymphoma or bladder cancer.

In another embodiment, the invention provides a compound of the present invention for use in therapy.

In another embodiment, the invention provides a combined preparation of a compound of the present invention and additional therapeutic agent(s) for simultaneous, separate or sequential use in therapy.

### Therapeutic Applications

The compounds of the invention induce the expression of pro-inflammatory cytokines such as IL17 in vitro in human cells, animal cells and human blood.

The compounds of the invention are agonists of RORgt.

The term “agonist” refers to any substance that activates a biologic receptor in vitro or in vivo to provoke a physiological response.

“RORgt” is an abbreviation of “Retinoic acid receptor related Orphan Receptor Gamma t”. RORgt is a transcription factor that in humans is encoded by the gene RORC.

Since RORgt and RORg have identical ligand binding domains, in the context of small molecule modulators, RORgt and RORg can be used interchangeably. RORgt and RORg are two isoforms produced from the same RORC gene. Activation of RORgt by agonists leads to induction of pro-inflammatory cytokines, including IL-17.

Another object of the present invention is the compounds of Formula (I), for use in a therapeutic treatment in humans or animals. In particular, the compounds of the present invention may be used for therapeutic or diagnostic applications in human or animal health.

The term “therapeutic agent” refers to one or more substances that are administered to a human or animal in order to achieve some kind of therapeutic effect in that human or animal, including to prevent, cure, or mitigate the effects of, infection or disease, and/or to otherwise improve the health of that human or animal.

The term “monotherapy” refers to the use of a single substance and/or strategy to treat a human or animal in any clinical or medical context, as opposed to the use of multiple substances and/or strategies to treat a human or animal in the same clinical or medical context, regardless of whether the multiple substances and/or strategies are used sequentially in any order or concurrently.

The term “chemotherapeutic agent” herein refers to one or more chemical substances that are administered to a human or animal in order to kill tumors, or slow or stop the growth of tumors, and/or slow or stop the division of cancerous cells and/or prevent or slow metastasis. Chemotherapeutic agents are often administered to treat cancer, but are also indicated for other diseases.

The term “chemotherapy” refers to medical treatment of a human or animal with one or more chemotherapeutic agents (see definition above).

The term “chemoimmunotherapy” refers to the combined use, whether sequentially in any order or concurrently, of chemotherapy substances and/or strategies, and immunotherapy substances and/or strategies. Chemoimmunotherapy is often employed to treat cancer, but can also be employed to treat other diseases.

The term “immune system” refers to the ensemble, or to any one or more components, of the molecules, substances (e.g. bodily fluids), anatomic structures (e.g. cells, tissue and organs) and physiologic processes involved in preventing infection in the body, in protecting the body during infection or during disease, and/or in helping the body to recuperate after infection or disease. A complete definition of “immune system” is beyond the scope of this patent; however, this term should be understood by any ordinary practitioner in the field.

The term “immune agent” refers to any endogenous or exogenous substance that can interact with any one or more components of the immune system. The term “immune agent” includes antibodies, antigens, vaccines and their constituent components, nucleic acids, synthetic drugs, natural or synthetic organic compounds, cytokines, natural or modified cells, synthetic analogs thereof, and/or fragments thereof.

The term “antagonist” refers to any substance that inhibits, counteracts, downregulates, and/or desensitizes a biologic receptor in vitro or in vivo to provoke a physiological response.

The term “immunotherapy” refers to any medical treatment in which one or more components of a human's or animal's immune system is deliberately modulated in order to directly or indirectly achieve some therapeutic benefit, including systemic and/or local effects, and preventative and/or curative effects. Immunotherapy can involve administering one or more immune agents (see definition above), either alone or in any combination, to a human or animal subject by any route (e.g. orally, intravenously, dermally, by injection, by inhalation, etc.), whether systemically, locally or both.

“Immunotherapy” can involve provoking, increasing, decreasing, halting, preventing, blocking or otherwise modulating the production of cytokines, and/or activating or deactivating cytokines or immune cells, and/or modulating the levels of immune cells, and/or delivering one or more therapeutic or diagnostic substances to a particular location in the body or to a particular type of cell or tissue, and/or destroying particular cells or tissue. Immunotherapy can be used to achieve local effects, systemic effects or a combination of both.

The term “immunosuppressed” describes the state of any human or animal subject whose immune system is functionally diminished, deactivated or otherwise compromised, or in whom one or more immune components is functionally diminished, deactivated or otherwise compromised.

“Immunosuppression” can be the cause, consequence or byproduct of disease, infection, exhaustion, malnutrition, medical treatment or some other physiologic or clinical state.

The terms “immunomodulating substance”, “immunomodulatory substance”, “immunomodulatory agent” and “immunomodulator”, used here synonymously, refer to any substance that, upon administration to a human or animal, directly influences the functioning of the immune system of that human or animal. Examples of common immunomodulators include, but are not limited to, antigens, antibodies and small-molecule drugs.

The term “vaccine” refers to a biological preparation administered to a human or animal in order to elicit or enhance a specific immune system response and/or protection against one or more antigens in that human or animal.

The term “vaccination” refers to treatment of a human or animal with a vaccine or to the act of administering a vaccine to a human or animal.

The term “adjuvant” refers to a secondary therapeutic substance that is administered together (either sequentially in any order, or concurrently) with a primary therapeutic substance to achieve some kind of complimentary, synergic or otherwise beneficial effect that could not be achieved through use of the primary therapeutic substance alone. An adjuvant can be used together with a vaccine, chemotherapy, or some other therapeutic substance. Adjuvants can enhance the efficacy of the primary therapeutic substance, reduce the toxicity or side effects of the primary therapeutic substance, or provide some kind of protection to the subject that receives the primary therapeutic substance, such as, but not limited to, improved functioning of the immune system.

In one embodiment, the compounds of Formula (I) can increase the amount of IL-17 in a subject. This includes but is not limited to IL-17 produced by TH17 cells.

In one embodiment, the compounds of Formula (I) can be administered as immunotherapy to a human or an animal to induce in vivo production of one or more cytokines that are therapeutically beneficial to that human or animal. This type of immunotherapy could be used alone or in combination with other treatment strategies, whether sequentially in any order, or concurrently. It could be used to prevent, cure, and/or mitigate the effects of infection or disease in that human or animal, and/or to modulate the immune system of that human or animal to achieve some other therapeutic benefit.

In one particular embodiment, the compounds of the present invention can be used for cytokine induction immunotherapy of immunosuppressed individuals.

In this example, a compound of Formula (I) would be administered to an immunosuppressed human or animal subject to induce in vivo production of one or more cytokines that directly or indirectly enhance the immune system of that human or animal. Subjects that might benefit from such treatment include those suffering from autoimmune disorders, immune system deficiencies or defects, microbial or viral infections, infectious diseases, or cancer.

The present invention thus discloses a method for inducing cytokine in immunosuppressed individuals, said method comprising administering to a patient in need thereof a compound of Formula (I) or a pharmaceutically acceptable salt or prodrug thereof.

In another embodiment, the compounds of the present invention can be used for cytokine induction immunotherapy in combination with chemotherapy. In this example, a compound of Formula (I) would be administered together with one or more chemotherapeutic agents, sequentially in any order or concomitantly, to a cancer patient to stop the growth of, shrink and/or destroy tumors in that patient. The chemoimmunotherapy resulting from the combination of cytokine induction, provided by the compound(s) of the present invention, and cytotoxicity, provided by the chemotherapeutic agent(s), might be less toxic to the patient, cause fewer side effects in the patient and/or exhibit greater anti-tumor efficacy than would the chemotherapeutic agent(s) when used as monotherapy.

The present invention thus discloses a method for treating cancer, said method comprising administering to a patient in need thereof: a chemotherapeutic agent; and a compound of Formula (I) or a pharmaceutically acceptable salt or prodrug thereof.

Another object of the present invention is the compound of Formula (I) for use in the treatment of a bacterial infection, a viral infection or a cancer.

As used herein, “cancer” refers to the physiological condition in subjects that is characterized by unregulated or dysregulated cell growth or death. The term “cancer” includes solid tumors and blood-born tumors, whether malignant or benign.

In a preferred embodiment, the cancer is from the following group: small cell lung cancer, non-small cell lung cancer, colorectal cancer, melanoma, renal cell carcinoma, head and neck cancer, Hodgkin's lymphoma or bladder cancer.

The present invention thus discloses a method for treating a bacterial infection, a viral infection or a cancer, said method comprising administering to a patient in need thereof a compound of Formula (I) or a pharmaceutically acceptable salt or prodrug thereof.

Another object of the present invention is the compound of Formula (I) for use in the treatment of a pathology that may be alleviated by the induction of an immune response via the RORg or RORgt pathway.

While it is possible that for use in therapy, a compound of formula (I) as well as pharmaceutically acceptable salts thereof may be administered as the compound itself, it is more commonly presented as a pharmaceutical composition.

Pharmaceutical compositions may be presented in unit dose forms containing a predetermined amount of active ingredient pep unit dose. Preferred unit dosage compositions are those containing a daily dose or sub-dose, or an appropriate fraction thereof, of an active ingredient. Such unit doses may therefore be administered more than once a day. Preferred unit dosage compositions are those containing a daily dose or sub-dose (for administration more than once a day), as herein above recited, or an appropriate fraction thereof, of an active ingredient.

Types of cancers that may be treated with the compounds of this invention include, but are not limited to, brain cancers, skin cancers, bladder cancers, ovarian cancers, breast cancers, gastric cancers, pancreatic cancers, prostate cancers, colorectal cancers, blood cancers, lung cancers and bone cancers. Examples of such cancer types include neuroblastoma, intestinal carcinoma such as rectal carcinoma, colon carcinomas, familiar adenomatous polyposis carcinoma and hereditary non-polyposis colorectal cancer, esophageal carcinoma, labial carcinoma, larynx carcinoma, nasopharyngeal cancers, oral cavity cancers, salivary gland carcinoma, peritoneal cancers, soft tissue sarcoma, urothelial cancers, sweat gland carcinoma, gastric carcinoma, adenocarcinoma, medullary thyroid carcinoma, papillary thyroid carcinoma, renal carcinoma, kidney parenchymal carcinoma, ovarian carcinoma, cervical carcinoma, uterine corpus carcinoma, endometrial carcinoma, pancreatic carcinoma, prostate carcinoma, testis carcinoma, breast cancers including HER2 Negative, urinary carcinoma, melanoma, brain tumors such as glioblastoma, astrocytoma, meningioma, medulloblastoma and peripheral neuroectodermal tumors, Hodgkin's lymphoma, non-Hodgkin's lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, diffuse large B-cell lymphoma (DLBCL), hepatocellular carcinoma, multiple myeloma, seminoma, osteosarcoma, chondrosarcoma, anal canal cancers, adrenal cortex carcinoma, chordoma, fallopian tube cancer, gastrointestinal stromal tumors, myeloproliferative diseases, mesothelioma, biliary tract cancers, Ewing sarcoma and other rare tumor types.

Compounds of the invention are useful for the treatment of certain types of cancer by themselves or in combination or co-administration with other therapeutic agents or radiation therapy. Thus, in one embodiment, the compounds of the invention are co-administered with radiation therapy or a second therapeutic agent with cytostatic or antineoplastic activity. Suitable cytostatic chemotherapy compounds include, but are not limited to (i) antimetabolites; (ii) DNA-fragmenting agents, (iii) DNA-crosslinking agents, (iv) intercalating agents (v) protein synthesis inhibitors, (vi) topoisomerase I poisons, such as camptothecin or topotecan; (vii) topoisomerase II poisons, (viii) microtubule-directed agents, (ix) kinase inhibitors (x) miscellaneous investigational agents (xi) hormones and (xii) hormone antagonists. It is contemplated that compounds of the invention may be useful in combination with any known agents falling into the above 12 classes as well as any future agents that are currently in development. In particular, it is contemplated that compounds of the invention may be useful in combination with current Standards of Care as well as any that evolve over the foreseeable future. Specific dosages and dosing regimens would be based on physicians' evolving knowledge and the general skill in the art.

Further provided herein are methods of treatment wherein compounds of the invention are administered with one or more immuno-oncology agents. The immuno-oncology agents used herein, also known as cancer immunotherapies, are effective to enhance, stimulate, and/or up-regulate immune responses in a subject. In one aspect, the administration of a compound of the invention with an immuno-oncology agent has a synergistic effect in inhibiting tumor growth.

In one aspect, the compound(s) of the invention are sequentially administered prior to administration of the immuno-oncology agent. In another aspect, compound(s) of the invention are administered concurrently with the immunology-oncology agent. In yet another aspect, compound(s) of the invention are sequentially administered after administration of the immuno-oncology agent.

In another aspect, compounds of the invention may be co-formulated with an immuno-oncology agent.

Immuno-oncology agents include, for example, a small molecule drug, antibody, or other biologic molecule. Examples of biologic immuno-oncology agents include, but are not limited to, cancer vaccines, antibodies, and cytokines. In one aspect, the antibody is a monoclonal antibody. In another aspect, the monoclonal antibody is humanized or human.

In one aspect, the immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses (often referred to as immune checkpoint regulators).

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTOR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α 1β2, FAS, FASL, RELT, DR6, TROY, NGFR.

In one aspect, T cell responses can be stimulated by a combination of a compound of the invention and one or more of (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM4-4, and (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

Other agents that can be combined with compounds of the invention for the treatment of cancer include antagonists of inhibitory receptors on NK cells or agonists of activating receptors on NK cells. For example, compounds of the invention can be combined with antagonists of KIR, such as lirilumab.

Yet other agents for combination therapies include agents that inhibit or deplete macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

In another aspect, compounds of the invention can be used with one or more of agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell anergy or exhaustion) and agents that trigger innate immune activation and/or inflammation at tumor sites.

In one aspect, the immuno-oncology agent is a CTLA-4 antagonist, such as an antagonistic CTLA-4 antibody. Suitable CTLA-4 antibodies include, for example, YERVOY (ipilimumab) or tremelimumab.

In another aspect, the immuno-oncology agent is a PD-1 antagonist, such as an antagonistic PD-1 antibody. The PD-1 antibody can be selected from Opdivo (nivolumab), Keytruda (pembrolizumab), PDR001 (Novartis; see WO2015/112900), MEDI-0680 (AMP-514) (AstraZeneca; see WO2012/145493), REGN-2810 (Sanofi/Regeneron; see WO2015/112800), JS001 (Taizhou Junshi), BGB-A317 (Beigene; see WO2015/35606), INCSHR1210 (SHR-1210) (Incyte/Jiangsu Hengrui Medicine; see WO2015/085847), TSR-042 (ANB001) (Tesara/AnaptysBio; see WO2014/179664), GLS-010 (Wuxi/Harbin Gloria Pharmaceuticals), AM-0001 (Armo/Ligand), or STI-1110 (Sorrento; see WO2014/194302). The immuno-oncology agent may also include pidilizumab (CT-011), though its specificity for PD-1 binding has been questioned. Another approach to target the PD-1 receptor is the recombinant protein composed of the extracellular domain of PD-L2 (B7-DC) fused to the Fc portion of IgG1, called AMP-224 In one aspect,

In another aspect, the immuno-oncology agent is a PD-L1 antagonist, such as an antagonistic PD-L1 antibody. The PD-L1 antibody can be selected from Tecentriq (atezolizumab), durvalumab, avelumab, STI-1014 (Sorrento; see WO2013/181634), or CX-072 (CytomX; see WO2016/149201).

In another aspect, the immuno-oncology agent is a LAG-3 antagonist, such as an antagonistic LAG-3 antibody. Suitable LAG3 antibodies include, for example, BMS-986016 (WO10/19570, WO14/08218), or IMP-731 or IMP-321 (WO08/132601, WO09/44273).

In another aspect, the immuno-oncology agent is a CD137 (4-1BB) agonist, such as an agonistic CD137 antibody. Suitable CD137 antibodies include, for example, urelumab and PF-05082566 (WO12/32433).

In another aspect, the immuno-oncology agent is a GITR agonist, such as an agonistic GITR antibody. Suitable GITR antibodies include, for example, BMS-986153, BMS-986156, TRX-518 (WO06/105021, WO09/009116) and MK-4166 (WO11/028683).

In another aspect, the immuno-oncology agent is an IDO antagonist. Suitable IDO antagonists include, for example, INCB-024360 (WO2006/122150, WO07/75598, WO08/36653, WO08/36642), indoximod, or NLG-919 (WO09/73620, WO09/1156652, WO11/56652, WO12/142237).

In another aspect, the immuno-oncology agent is an OX40 agonist, such as an agonistic OX40 antibody. Suitable OX40 antibodies include, for example, MEDI-6383 or MEDI-6469.

In another aspect, the immuno-oncology agent is an OX40L antagonist, such as an antagonistic OX40 antibody. Suitable OX40L antagonists include, for example, RG-7888 (WO06/029879).

In another aspect, the immuno-oncology agent is a CD40 agonist, such as an agonistic CD40 antibody. In yet another embodiment, the immuno-oncology agent is a CD40 antagonist, such as an antagonistic CD40 antibody. Suitable CD40 antibodies include, for example, lucatumumab or dacetuzumab.

In another aspect, the immuno-oncology agent is a CD27 agonist, such as an agonistic CD27 antibody. Suitable CD27 antibodies include, for example, varlilumab.

In another aspect, the immuno-oncology agent is MGA271 (to B7H3) (WO11/109400).

The combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single dosage form having a fixed ratio of each therapeutic agent or in multiple, single dosage forms for each of the therapeutic agents. Sequential or substantially simultaneous administration of each therapeutic agent can be effected by any appropriate route including, but not limited to, oral routes, intravenous routes, intratumoral routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. Combination therapy also can embrace the administration of the therapeutic agents as described above in further combination with other biologically active ingredients and non-drug therapies (e.g., surgery or radiation treatment.) Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

Another object of the present invention is the compounds of Formula (I) for use in adoptive cellular therapy to treat cancer, immune disorders and infections.

The present invention may be embodied in other specific forms without departing from the spirit or essential attributes thereof. This invention encompasses all combinations of preferred aspects of the invention noted herein. It is understood that any and all embodiments of the present invention may be taken in conjunction with any other embodiment or embodiments to describe additional embodiments. It is also understood that each individual element of the embodiments is its own independent embodiment. Furthermore, any element of an embodiment is meant to be combined with any and all other elements from any embodiment to describe an additional embodiment.

### Pharmaceutical Compositions and Dosing

The invention also provides pharmaceutically acceptable compositions which comprise a therapeutically effective amount of one or more of the compounds of Formula I, formulated together with one or more pharmaceutically acceptable carriers (additives) and/or diluents, and optionally, one or more additional therapeutic agents described above. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intratumoral, intravenous or epidural injection as, for example, a sterile solution or suspension, or sustained release formulation; (3) topical application, for example, as a cream, ointment, or a controlled release patch or spray applied to the skin; or intratumorally.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient.

Formulations of the present invention include those suitable for oral, intratumoral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the patient being treated and the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

Pharmaceutical compositions of this invention suitable for parenteral administration comprise one or more compounds of the invention in combination with one or more pharmaceutically acceptable sterile isotonic aqueous or non-aqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

In some cases, in order to prolong the effect of a drug, it is desirable to slow the absorption of the drug from subcutaneous, intratumoral or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsuled matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, oral, intravenous, intracerebroventricular and subcutaneous doses of the compounds of this invention for a patient will range from about 0.01 to about 50 mg per kilogram of body weight per day.

While it is possible for a compound of the present invention to be administered alone, it is preferable to administer the compound as a pharmaceutical formulation (composition).

### Definitions

Unless specifically stated otherwise herein, references made in the singular may also include the plural. For example, “a” and “an” may refer to either one, or one or more.

Unless otherwise indicated, any heteroatom with unsatisfied valences is assumed to have hydrogen atoms sufficient to satisfy the valences.

Throughout the specification and the appended claims, a given chemical formula or name shall encompass all stereo and optical isomers and racemates thereof where such isomers exist. Unless otherwise indicated, all chiral (enantiomeric and diastereomeric) and racemic forms are within the scope of the invention. Many geometric isomers of C═C double bonds, C═N double bonds, ring systems, and the like can also be present in the compounds, and all such stable isomers are contemplated in the present invention. Cis- and trans- (or E- and Z-) geometric isomers of the compounds of the present invention are described and may be isolated as a mixture of isomers or as separated isomeric forms. The present compounds can be isolated in optically active or racemic forms. Optically active forms may be prepared by resolution of racemic forms or by synthesis from optically active starting materials. All processes used to prepare compounds of the present invention and intermediates made therein are considered to be part of the present invention. When enantiomeric or diastereomeric products are prepared, they may be separated by conventional methods, for example, by chromatography or fractional crystallization. Depending on the process conditions the end products of the present invention are obtained either in free (neutral) or salt form. Both the free form and the salts of these end products are within the scope of the invention. If so desired, one form of a compound may be converted into another form. A free base or acid may be converted into a salt; a salt may be converted into the free compound or another salt; a mixture of isomeric compounds of the present invention may be separated into the individual isomers. Compounds of the present invention, free form and salts thereof, may exist in multiple tautomeric forms, in which hydrogen atoms are transposed to other parts of the molecules and the chemical bonds between the atoms of the molecules are consequently rearranged. It should be understood that all tautomeric forms, insofar as they may exist, are included within the invention.

For purposes of clarity and in accordance with standard convention in the art, the symbol

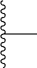

is used in formulas and tables to show the bond that is the point of attachment of the moiety or substituent to the core/nucleus of the structure.

Additionally, for purposes of clarity, where a substituent has a dash (-) that is not between two letters or symbols; this is used to indicate a point of attachment for a substituent. For example, —CONH₂ is attached through the carbon atom.

Additionally, for purposes of clarity, when there is no substituent shown at the end of a solid line, this indicates that there is a methyl (CH₃) group connected to the bond.

The term “counter ion” is used to represent a negatively charged species such as chloride, bromide, hydroxide, acetate, and sulfate or a positively charged species such as sodium (Na+), potassium (K+), ammonium (RₙNHₘ+ where n=0-4 and m=0-4) and the like.

The term “electron withdrawing group” (EWG) refers to a substituent which polarizes a bond, drawing electron density towards itself and away from other bonded atoms. Examples of EWGs include, but are not limited to, CF₃, CF₂CF₃, CN, halogen, haloalkyl, NO₂, sulfone, sulfoxide, ester, sulfonamide, carboxamide, alkoxy, alkoxyether, alkenyl, alkynyl, OH, C(O)alkyl, CO₂H, phenyl, heteroaryl, —O-phenyl, and —O— heteroaryl. Preferred examples of EWG include, but are not limited to, CF₃, CF₂CF₃, CN, halogen, SO₂(C₁₋₄ alkyl), CONH(C₁₋₄ alkyl), CON(C₁₋₄ alkyl)₂, and heteroaryl. More preferred examples of EWG include, but are not limited to, CF₃ and CN.

As used herein, the term “amine protecting group” means any group known in the art of organic synthesis for the protection of amine groups which is stable to an ester reducing agent, a disubstituted hydrazine, R4-M and R7-M, a nucleophile, a hydrazine reducing agent, an activator, a strong base, a hindered amine base and a cyclizing agent. Such amine protecting groups fitting these criteria include those listed in Wuts, P. G. M. and Greene, T. W. Protecting Groups in Organic Synthesis, 4th Edition, Wiley (2007) and The Peptides: Analysis, Synthesis, Biology, Vol. 3, Academic Press, New York (1981), the disclosure of which is hereby incorporated by reference. Examples of amine protecting groups include, but are not limited to, the following: (1) acyl types such as formyl, trifluoroacetyl, phthalyl, and p-toluenesulfonyl; (2) aromatic carbamate types such as benzyloxycarbonyl (Cbz) and substituted benzyloxycarbonyls, 1-(p-biphenyl)-1-methylethoxycarbonyl, and 9-fluorenylmethyloxycarbonyl (Fmoc); (3) aliphatic carbamate types such as tert-butyloxycarbonyl (Boc), ethoxycarbonyl, diisopropylmethoxycarbonyl, and allyloxycarbonyl; (4) cyclic alkyl carbamate types such as cyclopentyloxycarbonyl and adamantyloxycarbonyl; (5) alkyl types such as triphenylmethyl and benzyl; (6) trialkylsilane such as trimethylsilane; (7) thiol containing types such as phenylthiocarbonyl and dithiasuccinoyl; and (8) alkyl types such as triphenylmethyl, methyl, and benzyl; and substituted alkyl types such as 2,2,2-trichloroethyl, 2-phenylethyl, and t-butyl; and trialkylsilane types such as trimethylsilane.

In cases wherein there are nitrogen atoms (e.g., amines) on compounds of the present invention, these may be converted to N-oxides by treatment with an oxidizing agent (e.g., mCPBA and/or hydrogen peroxides) to afford other compounds of this invention. Thus, shown and claimed nitrogen atoms are considered to cover both the shown nitrogen and its N-oxide (N→O) derivative.

When any variable occurs more than one time in any constituent or formula for a compound, its definition at each occurrence is independent of its definition at every other occurrence. Thus, for example, if a group is shown to be substituted with 0-3 R, then said group may optionally be substituted with up to three R groups, and at each occurrence R is selected independently from the definition of R. Also, combinations of substituents and/or variables are permissible only if such combinations result in stable compounds.

When a bond to a substituent is shown to cross a bond connecting two atoms in a ring, then such substituent may be bonded to any atom on the ring. When a substituent is listed without indicating the atom in which such substituent is bonded to the rest of the compound of a given formula, then such substituent may be bonded via any atom in such substituent. Combinations of substituents and/or variables are permissible only if such combinations result in stable compounds.

As used herein, the term “alkyl” or “alkylene” is intended to include both branched and straight-chain saturated aliphatic hydrocarbon groups having the specified number of carbon atoms. For example, “C₁₋₁₀ alkyl” (or alkylene), is intended to include C₁, C₂, C₃, C₄, C₅, C₆, C₇, C₈, C₉, and C₁₀ alkyl groups. Additionally, for example, “C₁-C₆ alkyl” denotes alkyl having 1 to 6 carbon atoms. Alkyl groups can be unsubstituted or substituted so that one or more of its hydrogens are replaced by another chemical group, for example, aryl or heteroaryl groups which are optionally substituted for example with alkyl, halo or haloalkyl. Example alkyl groups include, but are not limited to, methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like.

The term “cycloalkyl” refers to cyclized alkyl groups, including mono-, bi- or poly-cyclic ring systems. C₃₋₇ cycloalkyl is intended to include C₃, C₄, C₅, C₆, and C₇ cycloalkyl groups. Example cycloalkyl groups include, but are not limited to, cyclopropyl, cyclobutyl, cyclopentyl, cyclohexyl, norbornyl, and the like. As used herein, “carbocycle” or “carbocyclic residue” is intended to mean any stable 3, 4, 5, 6, or 7-membered monocyclic or bicyclic or 7-, 8-, 9-, 10-, 11-, 12-, or 13-membered bicyclic or tricyclic ring, any of which may be saturated, partially unsaturated, unsaturated or aromatic. Examples of such carbocycles include, but are not limited to, cyclopropyl, cyclobutyl, cyclobutenyl, cyclopentyl, cyclopentenyl, cyclohexyl, cycloheptyl, cycloheptenyl, adamantyl, cyclooctyl, cyclooctenyl, cyclooctadienyl, [3.3.0]bicyclooctane, [4.3.0]bicyclononane, [4.4.0]bicyclodecane, [2.2.2]bicyclooctane, fluorenyl, phenyl, naphthyl, indanyl, adamantyl, anthracenyl, and tetrahydronaphthyl (tetralin). As shown above, bridged rings are also included in the definition of carbocycle (e.g., [2.2.2]bicyclooctane). Preferred carbocycles, unless otherwise specified, are cyclopropyl, cyclobutyl, cyclopentyl, cyclohexyl, and phenyl. When the term “carbocycle” is used, it is intended to include “aryl”. A bridged ring occurs when one or more carbon atoms link two non-adjacent carbon atoms. Preferred bridges are one or two carbon atoms. It is noted that a bridge always converts a monocyclic ring into a bicyclic ring. When a ring is bridged, the substituents recited for the ring may also be present on the bridge.

The terms “halo” and “halogen,” as used herein, refer to F, Cl, Br, and I.

The term “heteroatom” refers to oxygen (O), sulfur (S), and nitrogen (N).

The terms “heterocycle”, “heterocycloalkyl”, “heterocyclo”, “heterocyclic”, or “heterocyclyl” may be used interchangeably and refer to substituted and unsubstituted 3- to 7-membered monocyclic groups, 7- to 11-membered bicyclic groups, and 10- to 15-membered tricyclic groups, in which at least one of the rings has at least one heteroatom (O, S or N), said heteroatom containing ring preferably having 1, 2, or 3 heteroatoms selected from O, S, and N. Each ring of such a group containing a heteroatom can contain one or two oxygen or sulfur atoms and/or from one to four nitrogen atoms provided that the total number of heteroatoms in each ring is four or less, and further provided that the ring contains at least one carbon atom. The nitrogen and sulfur atoms may optionally be oxidized and the nitrogen atoms may optionally be quaternized. The fused rings completing the bicyclic and tricyclic groups may contain only carbon atoms and may be saturated, partially saturated, or fully unsaturated. The heterocyclo group may be attached at any available nitrogen or carbon atom. As used herein the terms “heterocycle”, “heterocycloalkyl”, “heterocyclo”, “heterocyclic”, and “heterocyclyl” include “heteroaryl” groups and “spiroheterocyclic” groups, as defined below.

Exemplary monocyclic heterocycle groups include azetidinyl, pyrrolidinyl, oxetanyl, imidazolinyl, oxazolidinyl, isoxazolinyl, thiazolidinyl, isothiazolidinyl, triazolyl, tetrahydrofuranyl, piperidyl, pyridyl, pyrazolyl, piperazinyl, 2-oxopiperazinyl, 2-oxopiperidyl, 2-oxopyrrolidinyl, 2-oxoazepinyl, 2-oxooxazolidinyl, azepinyl, 1,1-dioxo-thianyl, 1-pyridonyl, 4-piperidonyl, 6-oxo-1,6-dihydropyridin-3-yl, tetrahydropyranyl or oxanyl, morpholinyl, thiamorpholinyl, thiamorpholinyl sulfoxide, thiamorpholinyl sulfone, 1,3-dioxolane and tetrahydro-1,1-dioxothienyl and the like.

Exemplary bicyclic heterocyclo groups include benzothiazolyl, quinuclidinyl, tetrahydroisoquinoline (THIQ) and isoquinoline.

The term “spiroheterocyclo” “spiroheterocyclic”, or “spiroheterocyclyl” refers to a heterocyclyl ring attached to the molecular moiety by a carbon atom in the heterocyclyl ring that is shared with the molecular moiety. Exemplary spiroheterocycles of the invention include diazaspiro[3.5]nonane and diazaspiro[3.3]heptane.

Additional heterocyclyl groups include

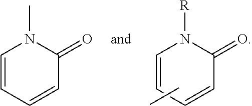

The term “heteroaryl” refers to substituted and unsubstituted aromatic 5- or 6-membered monocyclic groups and 9- or 10-membered bicyclic groups that have at least one heteroatom (O, S or N) in at least one of the rings, said heteroatom-containing ring preferably having 1, 2, or 3 heteroatoms independently selected from O, S, and/or N. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic group are aromatic and may contain only carbon atoms. The nitrogen and sulfur atoms may optionally be oxidized and the nitrogen atoms may optionally be quaternized. Bicyclic heteroaryl groups must include only aromatic rings. The heteroaryl group may be attached at any available nitrogen or carbon atom of any ring. The heteroaryl ring system may be unsubstituted or may contain one or more substituents.

Exemplary monocyclic heteroaryl groups include pyrrolyl, pyrazolyl, pyrazolinyl, imidazolyl, oxazolyl, isoxazolyl, thiazolyl, thiadiazolyl, isothiazolyl, furanyl, thiophenyl, oxadiazolyl, pyridinyl, pyrazinyl, pyrimidinyl, pyridazinyl, and triazinyl.

Exemplary bicyclic heteroaryl groups include indolyl, benzothiazolyl, benzodioxolyl, benzoxazolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, and pyrrolopyridyl.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington: The Science and Practice of Pharmacy, 22^{nd} Edition, Allen, L. V. Jr., Ed.; Pharmaceutical Press, London, UK (2012), the disclosure of which is hereby incorporated by reference.

In addition, compounds of formula I may have prodrug forms. Any compound that will be converted in vivo to provide the bioactive agent (i.e., a compound of formula I) is a prodrug within the scope and spirit of the invention. Various forms of prodrugs are well known in the art. For examples of such prodrug derivatives, see:

a) Bundgaard, H., ed., Design of Prodrugs, Elsevier (1985), and Widder, K. et al., eds., Methods in Enzymology, 112:309-396, Academic Press (1985);

b) Bundgaard, H., Chapter 5, “Design and Application of Prodrugs,” A Textbook of Drug Design and Development, pp. 113-191, Krosgaard-Larsen, P. et al., eds., Harwood Academic Publishers (1991);

c) Bundgaard, H., Adv. Drug Deliv. Rev., 8:1-38 (1992);

d) Bundgaard, H. et al., J. Pharm. Sci., 77:285 (1988);

e) Kakeya, N. et al., Chem. Pharm. Bull., 32:692 (1984); and

f) Rautio, J (Editor). Prodrugs and Targeted Delivery (Methods and Principles in Medicinal Chemistry), Vol 47, Wiley-VCH, 2011.

Compounds containing a carboxy group can form physiologically hydrolyzable esters that serve as prodrugs by being hydrolyzed in the body to yield formula I compounds per se. Such prodrugs are preferably administered orally since hydrolysis in many instances occurs principally under the influence of the digestive enzymes. Parenteral administration may be used where the ester per se is active, or in those instances where hydrolysis occurs in the blood. Examples of physiologically hydrolyzable esters of compounds of formula I include C₁₋₆alkyl, C₁₋₆alkylbenzyl, 4-methoxybenzyl, indanyl, phthalyl, methoxymethyl, C₁₋₆ alkanoyloxy-C₁₋₆alkyl (e.g., acetoxymethyl, pivaloyloxymethyl or propionyloxymethyl), C₁₋₆alkoxycarbonyloxy-C₁₋₆alkyl (e.g., methoxycarbonyl-oxymethyl or ethoxycarbonyloxymethyl, glycyloxymethyl, phenylglycyloxymethyl, (5-methyl-2-oxo-1,3-dioxolen-4-yl)-methyl), and other well known physiologically hydrolyzable esters used, for example, in the penicillin and cephalosporin arts. Such esters may be prepared by conventional techniques known in the art.

Preparation of prodrugs is well known in the art and described in, for example, King, F. D., ed., Medicinal Chemistry: Principles and Practice, The Royal Society of Chemistry, Cambridge, UK (2^{nd} edition, reproduced, 2006); Testa, B. et al., Hydrolysis in Drug and Prodrug Metabolism. Chemistry, Biochemistry and Enzymology, VCHA and Wiley-VCH, Zurich, Switzerland (2003); Wermuth, C. G., ed., The Practice of Medicinal Chemistry, 3^{rd} edition, Academic Press, San Diego, Calif. (2008).

The term “solvate” means a physical association of a compound of this invention with one or more solvent molecules, whether organic or inorganic. This physical association includes hydrogen bonding. In certain instances the solvate will be capable of isolation, for example when one or more solvent molecules are incorporated in the crystal lattice of the crystalline solid. The solvent molecules in the solvate may be present in a regular arrangement and/or a non-ordered arrangement. The solvate may comprise either a stoichiometric or nonstoichiometric amount of the solvent molecules. “Solvate” encompasses both solution-phase and isolable solvates. Exemplary solvates include, but are not limited to, hydrates, ethanolates, methanolates, and isopropanolates. Methods of solvation are generally known in the art.

As used herein, the term “patient” refers to organisms to be treated by the methods of the present invention. Such organisms preferably include, but are not limited to, mammals (e.g., murines, simians, equines, bovines, porcines, canines, felines, and the like), and most preferably refers to humans.

As used herein, the term “effective amount” means that amount of a drug or pharmaceutical agent, i.e., a compound of the invention, that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. An effective amount can be administered in one or more administrations, applications or dosages and is not intended to be limited to a particular formulation or administration route. The term also includes within its scope amounts effective to enhance normal physiological function

As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

Examples of bases include, but are not limited to, alkali metals (e.g., sodium) hydroxides, alkaline earth metals (e.g., magnesium), hydroxides, ammonia, and compounds of formula NW₄⁺, wherein W is C₁₋₄ alkyl, and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

### Methods of Preparation

The compounds of the present invention can be prepared in a number of ways well known to one skilled in the art of organic synthesis. The compounds of the present invention can be synthesized using the methods described below, together with synthetic methods known in the art of synthetic organic chemistry, or variations thereon as appreciated by those skilled in the art. Preferred methods include, but are not limited to, those described below. All references cited herein are hereby incorporated by reference in their entirety.

The compounds of this invention may be prepared using the reactions and techniques described in this section. The reactions are performed in solvents appropriate to the reagents and materials employed and are suitable for the transformations being effected. Also, in the description of the synthetic methods described below, it is to be understood that all proposed reaction conditions, including choice of solvent, reaction atmosphere, reaction temperature, duration of the experiment and work up procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents that are compatible with the reaction conditions will be readily apparent to one skilled in the art and alternate methods must then be used. This will sometimes require a judgment to modify the order of the synthetic steps or to select one particular process scheme over another in order to obtain a desired compound of the invention. It will also be recognized that another major consideration in the planning of any synthetic route in this field is the judicious choice of the protecting group used for protection of the reactive functional groups present in the compounds described in this invention. An authoritative account describing the many alternatives to the trained practitioner is Greene and Wuts (Protective Groups In Organic Synthesis, Fourth Edition, Wiley and Sons, 2007).

Compounds of Formula (I) may be prepared by reference to the methods illustrated in the following Scheme. As shown therein, the end product is a compound having the same structural formula as Formula (I). It will be understood that any compound of Formula (I) may be produced by the schemes by the suitable selection of reagents with appropriate substitution. Solvents, temperatures, pressures, and other reaction conditions may readily be selected by one of ordinary skill in the art. Starting materials are commercially available or readily prepared by one of ordinary skill in the art. Constituents of compounds are as defined herein or elsewhere in the specification.

Compounds of general formula i can be prepared according to the method outlined in Scheme i. Substituted phenol iA can be reacted with aryl fluoride iB to provide biaryl ether iC. Reduction of the nitro group in iC followed by aclylation can yield compounds of general formula i. It should be noted and obvious to those skilled in the art that intermediates such as aniline iD can be reductively aminated with various aldehydes or reacted with various electrophiles such as sulfonyl chlorides, isocyanates or isothiocyanates to yield the corresponding N-substituted compounds.

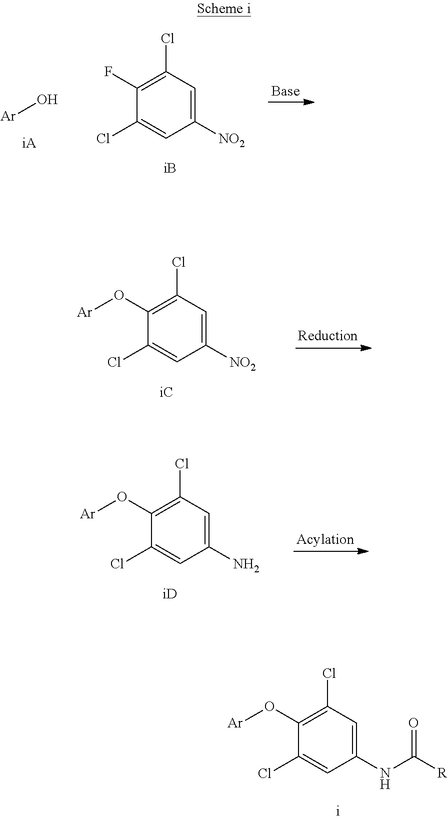

Alternatively, substituted phenol iA can be reacted with aryl fluoride iiA to afford biaryl ether iiB. Metal mediated coupling of bromo compound iiB with various amides can provide compounds of general formula i according to the method outlined in Scheme ii.

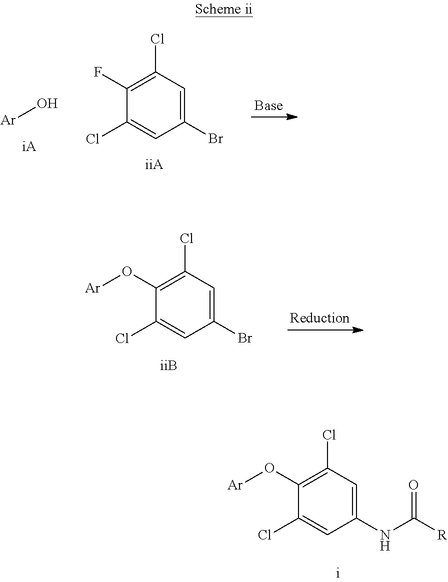

In another variation, substituted phenol iA can be reacted meta nitroaryl fluoride iiA to obtain biaryl ether iiiB (Scheme iii). Reduction of the nitro group and acylation of the resulting aniline iiiC can afford compounds of general formula iii.

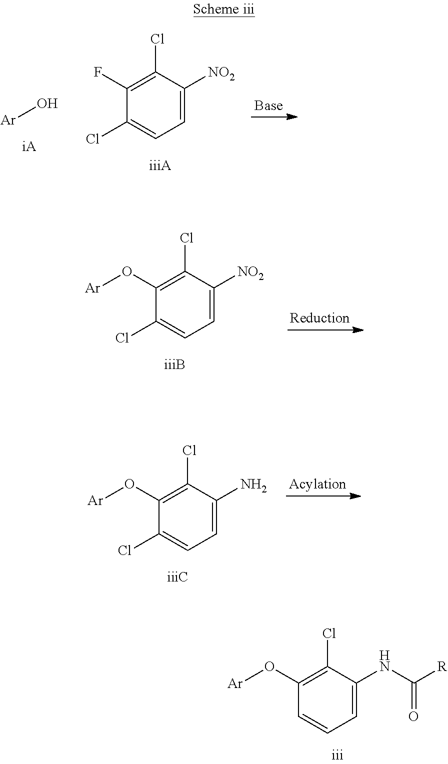

In yet another variation, substituted phenol iA can be reacted with cyanoaryl fluoride ivA to obtain biaryl ether ivB (Scheme iv). Hydrolysis of the cyano group can afford the corresponding carboxylic acid ivC that can be coupled to amines to afford amides of general formula iv.

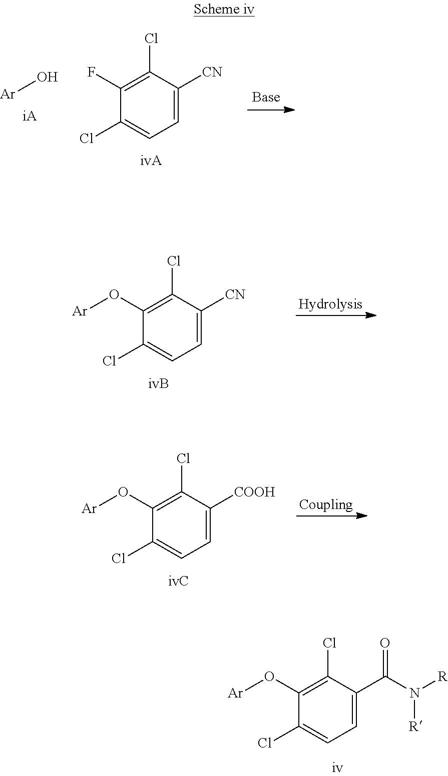

Alternatively cyano compound ivB can be reduced to obtain the corresponding substituted benzylic amine vA (Scheme v). Amine vA can be acylated to get compounds of general formula v.

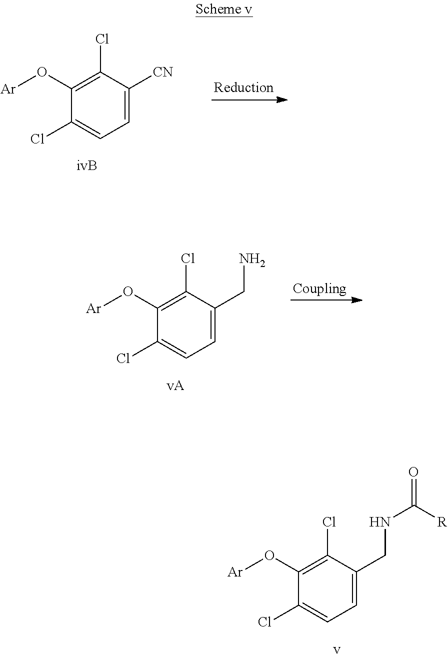

Variously substituted phenols vi (alternatives to phenol iA) can be prepared from the corresponding aryl bromide viA via palladium mediated coupling (Scheme vi).

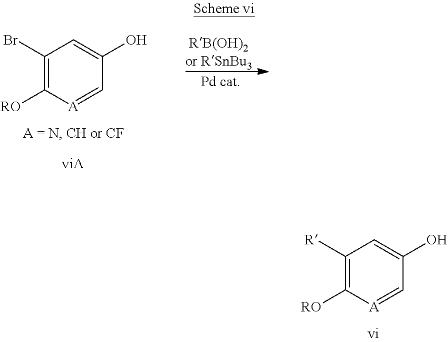

### EXAMPLES

Preparation of compounds of Formula (I), and intermediates used in the preparation of compounds of Formula (I), can be prepared using procedures shown in the following Examples and related procedures. The methods and conditions used in these examples, and the actual compounds prepared in these Examples, are not meant to be limiting, but are meant to demonstrate how the compounds of Formula (I) can be prepared. Starting materials and reagents used in these examples, when not prepared by a procedure described herein, are generally either commercially available, or are reported in the chemical literature, or may be prepared by using procedures described in the chemical literature.

Abbreviations

| | |
|---|---|
| Ac | acetyl |
| ACN | acetonitrile |
| AcOH | acetic acid |
| anhyd. | anhydrous |
| aq. | aqueous |
| Bn | benzyl |
| Bu | butyl |
| Boc | tert-butoxycarbonyl |
| BOP | benzotriazol-1-yloxytris-(dimethylamino)-phosphonium |
| | hexafluorophosphate |
| DAST | (diethylamino)sulfur trifluoride |
| DCE | dichloroethane |
| DCM | dichloromethane |
| DMAP | dimethylaminopyridine |
| DEA | diethylamine |
| DIPEA | diisopropylethylamine |
| DMF | dimethylformamide |
| DMSO | dimethylsulfoxide |
| EDC | 1-(3-dimethylaminopropyl)-3-ethylcarbodiimide |
| | hydrochloride |
| EtOAc | ethyl acetate |
| Et | ethyl |
| EtOH | ethanol |
| H or H₂ | hydrogen |
| h, hr or hrs | hour(s) |
| HATU | O-(7-azabenzotriazol-1-yl)-N, N, N′, N′- |
| | tetramethyluronium hexafluorophosphate |
| HCTU | O-(6-Chlorobenzotriazol-1-yl)-N,N,N′,N′- |
| | tetramethyluronium hexafluorophosphate |
| hex | hexane |
| i | iso |
| IPA | isopropyl alcohol |
| HOAc | acetic acid |
| HCl | hydrochloric acid |
| HPLC | high pressure liquid chromatography |
| LC | liquid chromatography |
| LCMS | liquid chromatography mass spectrometry |
| M | molar |
| mL or ml | milliliter |
| mM | millimolar |
| Me | methyl |
| MeOH | methanol |
| MHz | megahertz |
| min. | minute(s) |
| mins | minute(s) |
| M⁺¹ | (M + H)⁺ |
| MS | mass spectrometry |
| n or N | normal |
| NBS | n-bromosuccinimide |
| nm | nanometer |
| nM | nanomolar |
| NCS | N-chlorosuccinimide |
| NMP | N-methylpyrrolidine |
| Pd/C | palladium on carbon |
| PdCl₂(dppf)₂ | [1,1′-bis(diphenylphosphino)ferrocene]dichloropalladium(II) |
| Pd(PPh₃)₄ | tetrakis(triphenylphosphine)palladium |
| Ph | phenyl |
| PPh₃ | triphenylphosphine |
| Pr | propyl |
| PSI | pounds per square inch |
| PyBOP | bromotripyrrolidinophosphonium hexafluorophosphate |
| Ret Time | retention time |
| sat. | saturated |
| SFC | supercritical fluid chromatography |
| TEA | triethylamine |
| TFA | trifluoroacetic acid |
| THF | tetrahydrofuran |
| TsCl | 4-toluenesulfonyl chloride |

### Analytical LCMS Methods:

Method A: Waters Acquity UPLC BEH C18 (2.1×50 mm), 1.7 micron; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm.

Method B: Waters Acquity UPLC BEH C18 (2.1×50 mm), 1.7 micron; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0-100% B over 3 minutes, then a 0.75-minute hold at 100% B; Flow: 1.0 mL/min; Detection: UV at 220 nm.

Method C: Waters Acquity UPLC BEH C18 (2.1×50 mm), 1.7 micron; Mobile Phase A=100% water with 0.05% TFA; Mobile Phase B=100% acetonitrile with 0.05% TFA; Gradient=2-98% B over 1 minute, then a 0.5-minute hold at 98% B; Flow rate: 0.8 mL/min; Detection: UV at 220 nm.

Method D: Waters Acquity Xbridge C18 (4.6×50 mm), 5 micron; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 4 minutes; Flow: 4.0 mL/min; Detection: UV at 220 nm.

Method E: Shimadzu Xterra C18 (4.6×50 mm), 5 micron; Mobile Phase A: 5:95 MeOH:water with 0.1% trifluoroacetic acid; Mobile Phase B: 95:5 MeOH:water with 0.1% trifluoroacetic acid; Temperature: 50° C.; Gradient: 0-100% B over 4 minutes; then 1 minute hold at 100% B; Flow: 4.0 mL/min; Detection: UV at 220 nm.

Method F: Waters Acquity UPLC BEH C18 (2.1×50 mm), 1.7 micron; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 1 minute, then a 0.70-minute hold at 100% B; Flow: 0.8 mL/min; Detection: UV at 220 nm.

Method G: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.75 min hold at 100% B; Flow: 1 mL/min; Detection: MS and UV (220 nm).

Method H: ACE Ucore SuperC18, 30 mm×125 mm, 2.5 μm particles; Mobile Phase A: 5:95 acetonitrile:water with 0.05% TFA; Mobile Phase B: 95:5 acetonitrile:water with 0.05% TFA; Gradient: 10% B to 100% B over 12 min, then a 3 min hold at 100% B; Flow: 0.5 mL/min; Detection: MS and UV (220 nm).

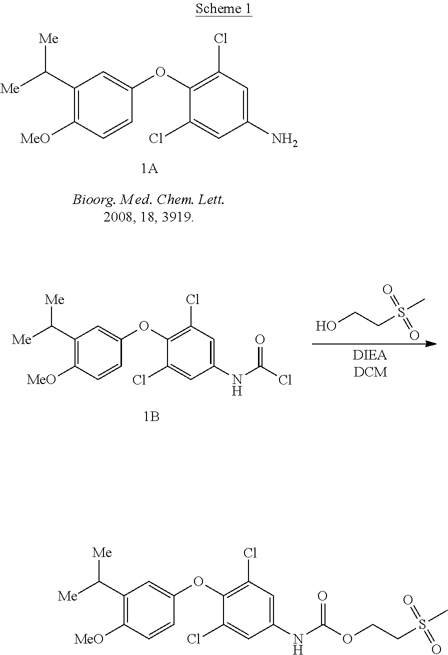

### Example 1

### 2-(methylsulfonyl)ethyl (3,5-dichloro-4-(3-isopropyl-4-methoxyphenoxy)phenyl)carbamate

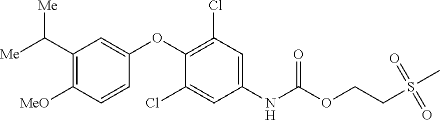 [SMILES: COc1ccc(Oc2c(Cl)cc(NC(=O)OCCS(C)(=O)=O)cc2Cl)cc1C(C)C]

### Intermediate 1B: (3,5-dichloro-4-(3-isopropyl-4-methoxyphenoxy)phenyl)carbamic Chloride

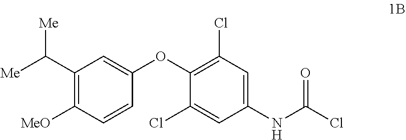 [SMILES: COc1ccc(Oc2c(Cl)cc(NC(=O)Cl)cc2Cl)cc1C(C)C]

To a solution of phosgene (1.094 mL, 1.533 mmol) in DCM (1 mL) was added 3,5-dichloro-4-(3-isopropyl-4-methoxyphenoxy)aniline (100 mg, 0.307 mmol) and then DIEA (0.049 mL, 0.353 mmol) in 1 mL of DCM dropwise. The resulting solution was stirred at room temperature for 30 min. The reaction mixture was concentrated. The residue was used as such next step.

### Example 1: 2-(methylsulfonyl)ethyl (3,5-dichloro-4-(3-isopropyl-4-methoxyphenoxy)phenyl)carbamate

To a solution of (3,5-dichloro-4-(3-isopropyl-4-methoxyphenoxy)phenyl)carbamic chloride 1B (15 mg, 0.039 mmol) in DCM (2 mL) was added 2-(methylsulfonyl)ethanol (23.96 mg, 0.193 mmol) and then DIEA (0.013 mL, 0.077 mmol). The mixture was stirred for 2 h. The solvent was removed and the residue was purified via reverse phase preparative LC/MS to obtain 2-(methylsulfonyl)ethyl (3,5-dichloro-4-(3-isopropyl-4 methoxyphenoxy)phenyl) carbamate Example 1 (7.2 mg, 0.015 mmol, 39.2% yield). LCMS m/z 476.2 (M+H); rt 2.27 min; Method B; ¹H NMR (500 MHz, DMSO-d₆) δ 7.68 (s, 2H), 6.84 (d, J=8.9 Hz, 1H), 6.81-6.69 (m, 1H), 6.40 (dd, J=8.9, 3.0 Hz, 1H), 4.48 (t, J=5.7 Hz, 2H), 3.73 (s, 3H), 3.56 (t, J=5.6 Hz, 1H), 3.27-3.13 (m, 1H), 3.08 (s, 3H), 1.23 (s, 3H), 1.11 (d, J=6.8 Hz, 6H).

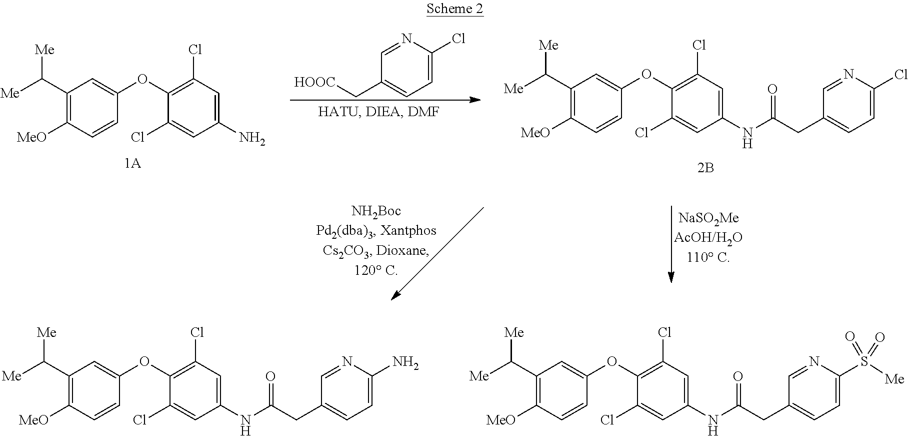 [SMILES: COc1ccc(Oc2c(Cl)cc(N)cc2Cl)cc1C(C)C ; O=C(O)Cc1ccc(Cl)nc1 ; COc1ccc(Oc2c(Cl)cc(NC(=O)Cc3ccc(Cl)nc3)cc2Cl)cc1C(C)C ; COc1ccc(Oc2c(Cl)cc(NC(=O)Cc3ccc(N)nc3)cc2Cl)cc1C(C)C ; COc1ccc(Oc2c(Cl)cc(NC(=O)Cc3ccc(S(C)(=O)=O)nc3)cc2Cl)cc1C(C)C]

### Example 2

### 2-(6-aminopyridin-3-yl)-N-(3,5-dichloro-4-(3-isopropyl-4-methoxyphenoxy) phenyl)acetamide

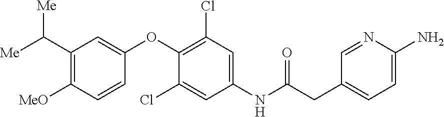 [SMILES: COc1ccc(Oc2c(Cl)cc(NC(=O)Cc3ccc(N)nc3)cc2Cl)cc1C(C)C]

### Intermediate 2B: 2-(6-chloropyridin-3-yl)-N-(3,5-dichloro-4-(3-isopropyl-4-methoxyphenoxy) Phenyl)acetamide

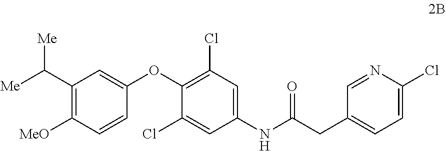 [SMILES: COc1ccc(Oc2c(Cl)cc(NC(=O)Cc3ccc(Cl)nc3)cc2Cl)cc1C(C)C]

To a solution of 2-(2-chloropyridin-3-yl)acetic acid (57.9 mg, 0.337 mmol) in DMF (5 mL) was added HATU (128 mg, 0.337 mmol) and then stirred for 5 min. To this solution was added 3,5-dichloro-4-(3-isopropyl-4-methoxyphenoxy)aniline 1A (100 mg, 0.307 mmol) and DIEA (0.134 mL, 0.766 mmol). The reaction mixture was stirred for 14 h at room temperature. The reaction mixture was quenched with water and the product was extracted with EtOAc (3×10 mL). The combined organic layers were washed with brine (1×10 mL), dried over magnesium sulfate and concentrated. The residue was purified by silica gel chromatography using 0-50% EtOAc in hexanes to afford 2B (45 mg, 0.94 mmol, 31% yield). LCMS m/z 478.7 (M+H); rt 3.22 min; Method D.

### Example 2: 2-(6-aminopyridin-3-yl)-N-(3,5-dichloro-4-(3-isopropyl-4-methoxyphenoxy) Phenyl)acetamide

To a reaction vial charged with 2-(6-chloropyridin-3-yl)-N-(3,5-dichloro-4-(3-isopropyl-4-methoxyphenoxy)phenyl)acetamide 2B (20 mg, 0.042 mmol), tert-butyl carbamate (9.77 mg, 0.083 mmol), Pd₂(dba)₃ (7.63 mg, 8.34 μmol), cesium carbonate (27.2 mg, 0.083 mmol), and Xantphos (4.82 mg, 8.34 μmol), was added dioxane (1 mL). The suspension was purged with nitrogen for 5 minutes, sealed, and heated to 120° C. for 30 min under microwave irradiation. The reaction mixture was cooled to room temperature, diluted with MeOH, and then filtered. The filtrate was concentrated under reduced pressure and the residue was treated with 20% TFA in DCM (2 mL) for 1 hour. The reaction mixture was concentrated and the crude product was purified via reverse phase preparative LC/MS to obtain Example 2, (4.9 mg, 0.011 mmol, 25% yield). LCMS m/z 460.3 (M+H); rt 3.22 min; Method D. ¹H NMR (500 MHz, DMSO-d₆) δ 10.65-10.48 (m, 1H), 7.86 (br. s., 1H), 7.83 (s, 2H), 7.62 (d, J=8.7 Hz, 1H), 6.84 (d, J=9.0 Hz, 1H), 6.78 (d, J=2.9 Hz, 1H), 6.71 (d, J=8.7 Hz, 1H), 6.41 (dd, J=8.9, 2.9 Hz, 1H), 3.90 (s, 1H), 3.73 (s, 3H), 3.57 (s, 1H), 3.25-3.13 (m, 1H), 2.89 (s, 1H), 2.73 (s, 1H), 1.11 (d, J=6.9 Hz, 6H).

### Example 3

### N-(3,5-dichloro-4-(3-isopropyl-4-methoxyphenoxy)phenyl)-2-(6-(methylsulfonyl) Pyridin-3-yl)acetamide

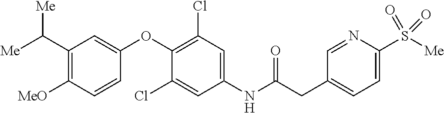 [SMILES: COc1ccc(Oc2c(Cl)cc(NC(=O)Cc3ccc(S(C)(=O)=O)nc3)cc2Cl)cc1C(C)C]

To a solution of 2-(6-chloropyridin-3-yl)-N-(3,5-dichloro-4-(3-isopropyl-4-methoxyphenoxy) phenyl)acetamide 2B (20 mg, 0.042 mmol) in water (2 mL) and acetic acid (0.5 mL) was added sodium methanesulfinate (8.51 mg, 0.083 mmol). The mixture was heated to 110° C. for 10 h. The reaction mixture was quenched with saturated sodium bicarbonate (5 mL) and then extracted with DCM (3×3 mL). The combined organic layers were washed with brine (1×10 mL) and then dried over magnesium sulfate. The solvent was removed and the crude material was purified via reverse phase preparative LC/MS to obtain Example 3, (8.2 mg, 0.016 mmol, 38% yield). LCMS m/z 523.1 (M+H); rt 2.22 min; Method B. ¹H NMR (500 MHz, DMSO-d₆) □ 10.72 (s, 1H), 8.74 (s, 1H), 8.14-8.00 (m, 2H), 7.86-7.77 (m, 2H), 6.84 (d, J=8.9 Hz, 1H), 6.78 (d, J=2.6 Hz, 1H), 6.41 (dd, J=8.9, 2.8 Hz, 1H), 3.97-3.89 (m, 2H), 3.73 (s, 3H), 3.29 (s, 2H), 3.23-3.09 (m, 2H), 1.11 (d, J=6.8 Hz, 6H).

### Scheme 3

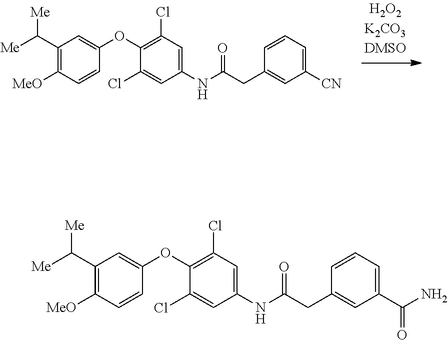 [SMILES: COc1ccc(Oc2c(Cl)cc(NC(=O)Cc3cccc(C#N)c3)cc2Cl)cc1C(C)C ; COc1ccc(Oc2c(Cl)cc(NC(=O)Cc3cccc(C(N)=O)c3)cc2Cl)cc1C(C)C]

### Example 4

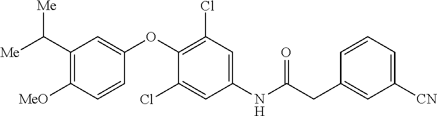 [SMILES: COc1ccc(Oc2c(Cl)cc(NC(=O)Cc3cccc(C#N)c3)cc2Cl)cc1C(C)C]

Example 4 was synthesized using the procedure described for intermediate 2B. LCMS m/z 469.29 (M+H); rt 2.46 min; Method A.

### Example 5

### 3-(2-((3,5-dichloro-4-(3-isopropyl-4-methoxyphenoxy)phenyl)amino)-2-oxoethyl)benzamide

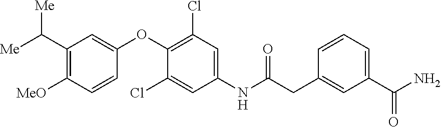 [SMILES: COc1ccc(Oc2c(Cl)cc(NC(=O)Cc3cccc(C(N)=O)c3)cc2Cl)cc1C(C)C]

A mixture of 2-(3-cyanophenyl)-N-(3,5-dichloro-4-(3-isopropyl-4-methoxyphenoxy)phenyl) acetamide Example 4 (32 mg, 0.068 mmol) and potassium carbonate (18.85 mg, 0.136 mmol) in DMSO (0.5 mL) was cooled in water bath. To the mixture was added hydrogen peroxide (0.125 mL, 2.045 mmol, 50%) and the resulting mixture was stirred at room temperature for 2 h. The reaction mixture was diluted with water, followed by addition of sodium sulfite solution. The white solid was filtered and washed with DCM. The filtrate was washed with brine (1×10 mL) and dried over magnesium sulfate. The solvent was removed and the crude material was purified by reverse phase preparative LC/MS to obtain Example 5 (19.9 mg, 0.04 mmol, 60% yield). LCMS m/z 487.3 (M+H); rt 2.16 min; Method B. ¹H NMR (500 MHz, DMSO-d₆) δ 10.61 (s, 1H), 8.00 (br. s., 1H), 7.83 (s, 3H), 7.77 (d, J=7.7 Hz, 1H), 7.51-7.30 (m, 3H), 6.88-6.72 (m, 2H), 6.41 (dd, J=8.8, 2.9 Hz, 1H), 3.73 (s, 4H), 3.42 (br. s., 1H), 3.26-3.10 (m, 1H), 1.11 (d, J=6.8 Hz, 6H).

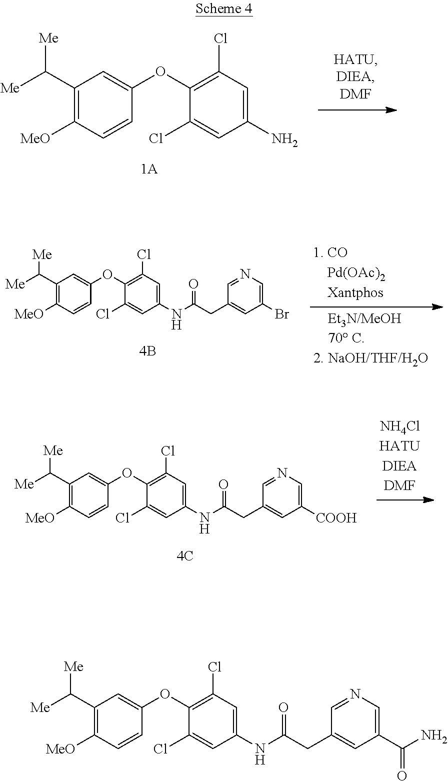 [SMILES: COc1ccc(Oc2c(Cl)cc(N)cc2Cl)cc1C(C)C ; COc1ccc(Oc2c(Cl)cc(NC(=O)Cc3cncc(Br)c3)cc2Cl)cc1C(C)C ; COc1ccc(Oc2c(Cl)cc(NC(=O)Cc3cncc(C(=O)O)c3)cc2Cl)cc1C(C)C ; COc1ccc(Oc2c(Cl)cc(NC(=O)Cc3cncc(C(N)=O)c3)cc2Cl)cc1C(C)C]

### Example 6

### 5-(2-((3,5-dichloro-4-(3-isopropyl-4-methoxyphenoxy)phenyl)amino)-2-oxoethyl)nicotinamide

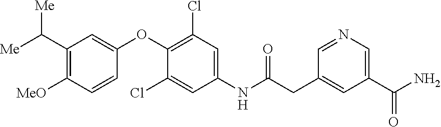 [SMILES: COc1ccc(Oc2c(Cl)cc(NC(=O)Cc3cncc(C(N)=O)c3)cc2Cl)cc1C(C)C]

### Intermediate 4B: 2-(5-bromopyridin-3-yl)-N-(3,5-dichloro-4-(3-isopropyl-4-methoxyphenoxy) Phenyl)acetamide

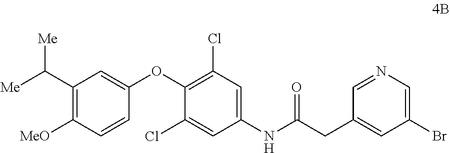 [SMILES: COc1ccc(Oc2c(Cl)cc(NC(=O)Cc3cncc(Br)c3)cc2Cl)cc1C(C)C]

To a solution of 2-(5-bromopyridin-3-yl)acetic acid (116 mg, 0.460 mmol) in DMF (5 mL) was added HATU (175 mg, 0.46 mmol) and then stirred for a while. To this solution was added 3,5-dichloro-4-(3-isopropyl-4-methoxyphenoxy)aniline 1A (100 mg, 0.307 mmol) and DIEA (0.214 mL, 1.23 mmol). The mixture was stirred for 10 h at room temperature. The reaction mixture was quenched with water and the product was extracted with EtOAc (3×10 mL). The combined organic layers were washed with brine (1×10 mL) and dried over magnesium sulfate. The crude product was purified by silica gel chromatography using 0-30% EtOAc in hexanes to afford 4B (61.6 mg, 0.118 mmol, 38% yield). LCMS m/z 524.7 (M+H); rt 4.18 min; Method E.

### Intermediate 4C: 5-(2-((3,5-dichloro-4-(3-isopropyl-4-methoxyphenoxy)phenyl)amino)-2-oxoethyl)nicotinic Acid

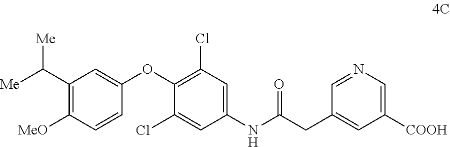 [SMILES: COc1ccc(Oc2c(Cl)cc(NC(=O)Cc3cncc(C(=O)O)c3)cc2Cl)cc1C(C)C]

A mixture of 2-(5-bromopyridin-3-yl)-N-(3,5-dichloro-4-(3-isopropyl-4-methoxyphenoxy) phenyl)acetamide 4B (61.6 mg, 0.118 mmol), Pd(OAc)₂ (1.319 mg, 5.88 μmol), XANTPHOS (6.80 mg, 0.012 mmol), MeOH (2 mL) and Et₃N (2 mL, 14.35 mmol) was stirred under atmosphere of carbon monoxide at 70° C. overnight. The reaction mixture was then cooled to room temperature, diluted with EtOAc, filtrated through Celite and concentrated under reduced pressure. The resulting residue was dissolved 1:1 (2/2 mL THF/MeOH) and then 1N NaOH (2 mL) was added. The mixture was stirred for 1 h. The mixture was concentrated to remove THE and then adjusted pH to 3 with 1N HCl. The resulting suspension was filtered. The residue was washed with water (2×) and then air-dried to obtain Intermediate 4C which was used as such in the next step. LCMS m/z 489.1 (M+H); rt 1.97 min; Method B. ¹H NMR (500 MHz, DMSO-d₆) δ 10.76 (br. s., 1H), 8.26 (br. s., 1H), 7.83 (s, 3H), 6.87-6.70 (m, 3H), 6.46-6.26 (m, 1H), 3.94-3.78 (m, 2H), 3.73 (s, 3H), 3.24-3.12 (m, 1H), 1.11 (d, J=6.8 Hz, 6H).

### Example 6: 5-(2-((3,5-dichloro-4-(3-isopropyl-4-methoxyphenoxy)phenyl)amino)-2-oxoethyl)nicotinamide

To a solution of 5-(2-((3,5-dichloro-4-(3-isopropyl-4-methoxyphenoxy) phenyl)amino)-2-oxoethyl)nicotinic acid 4C (20 mg, 0.041 mmol) in DMF (2 mL) was added ammonium chloride (10.93 mg, 0.204 mmol), HATU (15.54 mg, 0.041 mmol) and DIEA (7.14 μl, 0.041 mmol). The reaction mixture was stirred for 10 h. The crude material was purified by reverse phase preparative LC/MS to obtain Example 6, (10.5 mg, 0.022 mmol, 53% yield). LCMS m/z 488.0.1 (M+H); rt 1.96 min; Method B. ¹H NMR (500 MHz, DMSO-d₆) δ 10.63 (s, 1H), 8.96 (br. s., 1H), 8.67 (br. s., 1H), 8.17 (br. s., 2H), 7.83 (s, 2H), 7.59 (br. s., 1H), 6.90-6.71 (m, 2H), 6.42 (dd, J=8.9, 3.1 Hz, 1H), 3.82 (s, 2H), 3.74 (s, 3H), 3.27-3.09 (m, 1H), 1.12 (d, J=7.0 Hz, 6H).

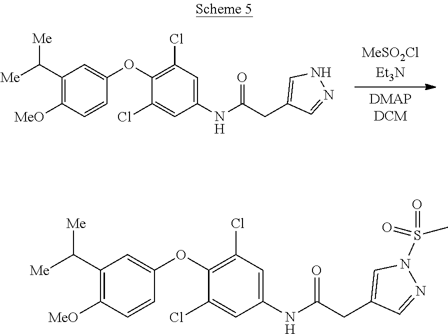 [SMILES: COc1ccc(Oc2c(Cl)cc(NC(=O)Cc3cn[nH]c3)cc2Cl)cc1C(C)C ; COc1ccc(Oc2c(Cl)cc(NC(=O)Cc3cnn(S(C)(=O)=O)c3)cc2Cl)cc1C(C)C]

### Example 7

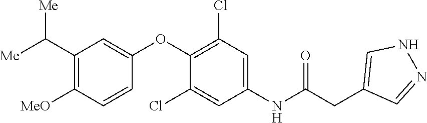 [SMILES: COc1ccc(Oc2c(Cl)cc(NC(=O)Cc3cn[nH]c3)cc2Cl)cc1C(C)C]

Example 7 was synthesized using the method described for intermediate 2B. LCMS m/z 434.2 (M+H); rt 2.15 min; Method A.

### Example 8

### N-(3,5-dichloro-4-(3-isopropyl-4-methoxyphenoxy)phenyl)-2-(1-(methylsulfonyl)-1H-pyrazol-4-yl)acetamide

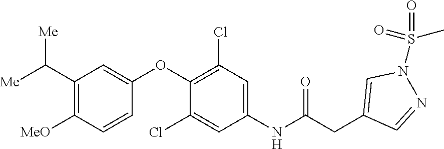 [SMILES: COc1ccc(Oc2c(Cl)cc(NC(=O)Cc3cnn(S(C)(=O)=O)c3)cc2Cl)cc1C(C)C]

A solution of N-(3,5-dichloro-4-(3-isopropyl-4-methoxyphenoxy)phenyl)-3-(1H-pyrazol-4-yl)propanamide (18.2 mg, 0.042 mmol), triethylamine (0.018 mL, 0.126 mmol) and DMAP (0.513 mg, 4.20 μmol) in DCM (2 mL) was treated with methanesulfonyl chloride (6.50 μl, 0.084 mmol) and stirred at rt for 2 h. The reaction mixture was concentrated and then the crude material was purified by reverse phase preparative LC/MS to obtain Example 8 (15.2 mg, 0.03 mmol, 73% yield). LCMS m/z 512.2 (M+H); rt 2.3 min; Method B. ¹H NMR (500 MHz, DMSO-d₆) δ 10.53 (s, 1H), 8.21 (s, 1H), 8.00-7.89 (m, 1H), 7.83 (s, 2H), 6.88-6.75 (m, 2H), 6.41 (dd, J=8.8, 2.9 Hz, 1H), 3.74 (s, 3H), 3.65 (s, 2H), 3.54 (s, 3H), 3.26-3.06 (m, 1H), 1.17-0.98 (m, 6H).

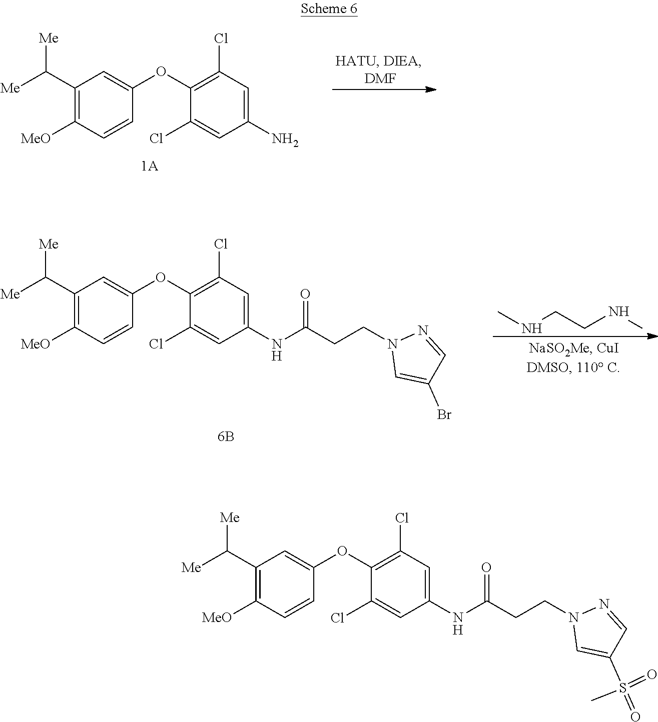

### Example 9

### N-(3,5-dichloro-4-(3-isopropyl-4-methoxyphenoxy)phenyl)-3-(4-(methylsulfonyl)-1H-pyrazol-1-yl)propanamide

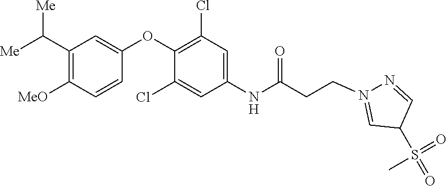

### Intermediate 6B: 3-(4-bromo-1H-pyrazol-1-yl)-N-(3,5-dichloro-4-(3-isopropyl-4-methoxyphenoxy)phenyl)propanamide

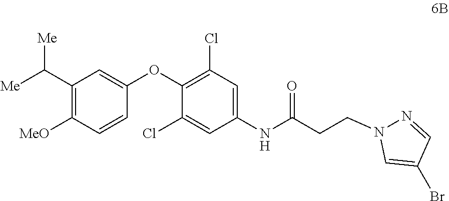 [SMILES: COc1ccc(Oc2c(Cl)cc(NC(=O)CCn3cc(Br)cn3)cc2Cl)cc1C(C)C]

To a solution of 3-(4-bromo-1H-pyrazol-1-yl)propanoic acid (43 mg, 0.196 mmol) in DMF (5 mL) was added HATU (82 mg, 0.216 mmol) and then stirred for 5 min. To this solution was added 3,5-dichloro-4-(3-isopropyl-4-methoxyphenoxy)aniline 1A (64.0 mg, 0.196 mmol) and DIEA (0.103 mL, 0.589 mmol). The mixture was stirred for 3 h at room temperature. It was quenched with water and the product was extracted with EtOAc (3×10 mL). The combined organic layers were washed with brine (1×10 mL) and dried over magnesium sulfate. The resulting crude 6B was used as such next step. LCMS m/z 527.6 (M+H); rt 4.06 min; Method E.

### Example 9: N-(3,5-dichloro-4-(3-isopropyl-4-methoxyphenoxy)phenyl)-3-(4-(methylsulfonyl)-1H-pyrazol-1-yl)propanamide

A pressure vessel was charged with 3-(4-bromo-1H-pyrazol-1-yl)-N-(3,5-dichloro-4-(3-isopropyl-4-methoxyphenoxy)phenyl)propanamide 6B (52.7 mg, 0.1 mmol) and DMSO (2 mL). To this solution was added sodium methanesulfinate (30.6 mg, 0.300 mmol), N,N′-dimethylethylenediamine (2.155 μl, 0.020 mmol) and copper(I)iodide (1.905 mg, 10.00 μmol). The vessel was sealed and vented into a balloon partially filled with nitrogen then placed in an oil bath preheated to 110° C. The reaction mixture was stirred for 10 h. The mixture was quenched with water and extracted with EtOAc (2×10 mL). The combined organic layers were washed with brine (1×10 mL) and dried over magnesium sulfate. The crude material was purified via reverse phase preparative LC/MS to obtain Example 9 (9.7 mg, 0.02 mmol, 18% yield in two steps). LCMS m/z 526.2 (M+H); rt 2.18 min; Method A. ¹H NMR (500 MHz, DMSO-d₆) δ 10.67-10.20 (m, 1H), 8.42 (s, 1H), 7.93 (s, 1H), 7.78 (d, J=11.9 Hz, 2H), 6.84 (dd, J=8.8, 3.1 Hz, 1H), 6.77 (br. s., 1H), 6.46-6.34 (m, 1H), 4.49 (t, J=6.3 Hz, 1H), 3.73 (s, 2H), 3.57-3.39 (m, 3H), 3.24-3.11 (m, 3H), 3.00-2.74 (m, 2H), 1.11 (d, J=6.7 Hz, 6H).

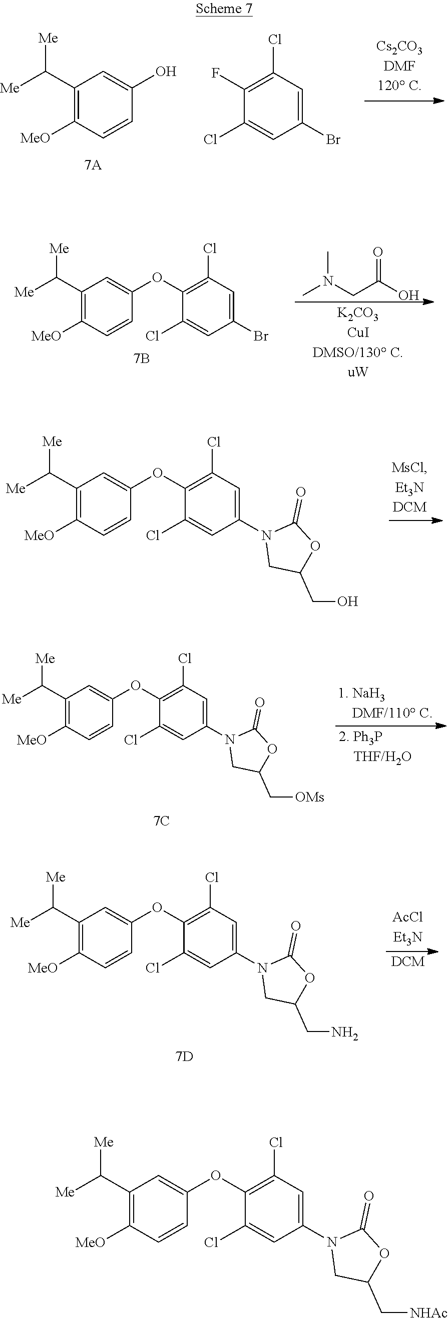

### Example 10

### 3-(3,5-dichloro-4-(3-isopropyl-4-methoxyphenoxy)phenyl)-5-(hydroxymethyl) Oxazolidin-2-one

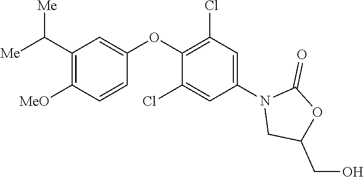 [SMILES: COc1ccc(Oc2c(Cl)cc(N3CC(CO)OC3=O)cc2Cl)cc1C(C)C]

### Intermediate 7B: 5-bromo-1,3-dichloro-2-(3-isopropyl-4-methoxyphenoxy)benzene

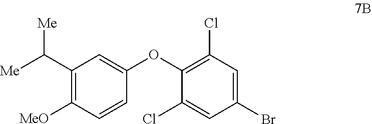 [SMILES: COc1ccc(Oc2c(Cl)cc(Br)cc2Cl)cc1C(C)C]

A pressure vessel containing a suspension of 3-isopropyl-4-methoxyphenol 7A (1000 mg, 6.02 mmol), 5-bromo-1,3-dichloro-2-fluorobenzene (1614 mg, 6.62 mmol), and cesium carbonate (2940 mg, 9.02 mmol) in DMF (15 mL) was heated at 120° C. for 10 h. The reaction mixture was cooled to room temperature and quenched with water. The resulting mixture was extracted with EtOAc (3×25 mL). The combined organic layers were dried (magnesium sulfate), filtered, and concentrated. The crude product was dissolved in a small amount of dichloromethane adsorbed onto a plug of silica gel, and purified by flash chromatography (Silica, 0% to 25% EtOAc/hexanes, 24 g column, 15 min gradient) to afford 5-bromo-1,3-dichloro-2-(3-isopropyl-4-methoxyphenoxy)benzene 7B (1200 mg, 3.08 mmol, 51.1% yield) as a clear film. ¹H NMR (400 MHz, CHLOROFORM-d) δ 7.59-7.53 (m, 2H), 6.91-6.82 (m, 1H), 6.76-6.69 (m, 1H), 6.51-6.44 (m, 1H), 3.87-3.71 (m, 3H), 3.31 (spt, J=6.9 Hz, 1H), 1.27-1.16 (m, 6H).

### Example 10: 3-(3,5-dichloro-4-(3-isopropyl-4-methoxyphenoxy)phenyl)-5-(hydroxymethyl) oxazolidin-2-one

A mixture of 5-bromo-1,3-dichloro-2-(3-isopropyl-4-methoxyphenoxy)benzene 7B (200 mg, 0.513 mmol), 5-(hydroxymethyl)oxazolidin-2-one (90 mg, 0.769 mmol), copper(I)iodide (29.3 mg, 0.154 mmol), K₂CO₃ (354 mg, 2.56 mmol), and N,N-dimethylglycine hydrochloride (50.1 mg, 0.359 mmol) in DMSO (5 mL) was stirred under microwave irradiation at 130° C. for 120 min. The reaction mixture was cooled to room temperature, quenched with water, and adjusted pH to 5. The precipitate formed was filtered, washed with water and air-dried. The crude residue was purified via reverse phase preparative LC/MS to obtain Example 10 (57 mg, 0.134 mmol, 26% yield). LCMS m/z 425.9 (M+H); rt 2.18 min; Method B. ¹H NMR (500 MHz, DMSO-d₆) δ 7.82 (s, 2H), 6.88-6.75 (m, 2H), 6.41 (dd, J=8.8, 3.0 Hz, 1H), 5.33 (t, J=5.6 Hz, 1H), 4.74 (d, J=3.5 Hz, 1H), 4.12 (t, J=9.0 Hz, 1H), 3.92-3.83 (m, 1H), 3.77-3.66 (m, 2H), 3.59-3.51 (m, 2H), 3.24-3.10 (m, 2H), 1.11 (d, J=6.9 Hz, 6H).

### Example 11

### N-((3-(3,5-dichloro-4-(3-isopropyl-4-methoxyphenoxy)phenyl)-2-oxooxazolidin-5-yl)methyl)acetamide

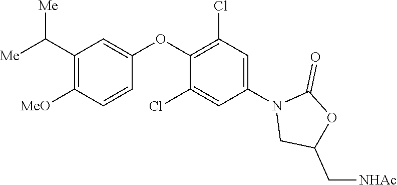 [SMILES: COc1ccc(Oc2c(Cl)cc(N3CC(CNC(C)=O)OC3=O)cc2Cl)cc1C(C)C]

### Intermediate 7C: (3-(3,5-dichloro-4-(3-isopropyl-4-methoxyphenoxy)phenyl)-2-oxooxazolidin-5-yl)methyl Methanesulfonate

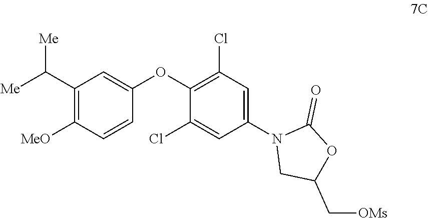 [SMILES: COc1ccc(Oc2c(Cl)cc(N3CC(COS(C)(=O)=O)OC3=O)cc2Cl)cc1C(C)C]

To an ice-cold solution of 3-(3,5-dichloro-4-(3-isopropyl-4-methoxyphenoxy)phenyl)-5-(hydroxymethyl)oxazolidin-2-one (171 mg, 0.4 mmol) in DCM (10 mL) was added TEA (0.167 mL, 1.200 mmol), followed by MsCl (0.041 mL, 0.520 mmol). The reaction mixture was stirred for 1 h. The resulting mixture was diluted with DCM (10 mL), washed with water, brine, dried over magnesium sulfate and concentrated. Obtained crude (3-(3,5-dichloro-4-(3-isopropyl-4-methoxyphenoxy)phenyl)-2-oxooxazolidin-5-yl)methyl methanesulfonate 7C (190 mg, 0.377 mmol, 94% yield) that was used as such in the next step. LCMS: rt 3.67 min; Method E.

### Intermediate 7D: 5-(aminomethyl)-3-(3,5-dichloro-4-(3-isopropyl-4-methoxyphenoxy) phenyl)oxazolidin-2-one

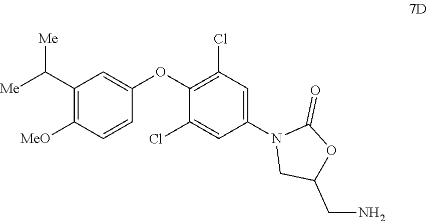 [SMILES: COc1ccc(Oc2c(Cl)cc(N3CC(CN)OC3=O)cc2Cl)cc1C(C)C]

To a solution of (3-(3,5-dichloro-4-(3-isopropyl-4-methoxyphenoxy)phenyl)-2-oxooxazolidin-5-yl)methyl methanesulfonate 7C (220 mg, 0.436 mmol) in DMF (3 mL) was added sodium azide (56.7 mg, 0.872 mmol). The mixture was stirred at 70° C. for 2 h. The mixture was quenched with water and extracted with DCM (3×10 mL). Combined organic layer was washed with brine (1×30 mL), dried over magnesium sulfate and concentrated. To the residue was added 3:1 of THF/water (4 mL) and Ph₃P (172 mg, 0.654 mmol). The resulting mixture was stirred at 50° C. for 10 h. To the reaction mixture was added water and extracted with EtOAc (2×15 mL). The combined organic layers were dried over magnesium sulfate and concentrated. The residue was purified by silica gel chromatography (12 g Column, 0-50% EtOAc/Hexane, 25 min) to obtain 5-(aminomethyl)-3-(3,5-dichloro-4-(3-isopropyl-4-methoxyphenoxy)phenyl)oxazolidin-2-one 7D (80 mg, 0.188 mmol, 43.1% yield). LCMS m/z 456.9 (M+Na); rt 3.23 min; Method E.

### Example 11: N-((3-(3,5-dichloro-4-(3-isopropyl-4-methoxyphenoxy)phenyl)-2-oxooxazolidin-5-yl)methyl)acetamide

To a solution of 5-(aminomethyl)-3-(3,5-dichloro-4-(3-isopropyl-4-methoxyphenoxy) phenyl)oxazolidin-2-one 7D (15 mg, 0.035 mmol) in DCM (2 mL) at 0° C. was added Et₃N (0.015 mL, 0.106 mmol) and acetyl chloride (2.77 mg, 0.035 mmol). The mixture was stirred for 1 h at room temperature. Solvent was removed and the crude material was purified by reverse phase preparative LC/MS to obtain Example 11, (6.1 mg, 0.013 mmol, 37% yield). LCMS m/z 467.2 (M+H); rt 2.10 min; Method B. ¹H NMR (500 MHz, DMSO-d₆) δ 8.25 (t, J=5.3 Hz, 1H), 7.80 (s, 2H), 6.86 (d, J=8.9 Hz, 1H), 6.80 (d, J=2.7 Hz, 1H), 6.43 (dd, J=8.9, 3.1 Hz, 1H), 4.77 (br. s., 1H), 4.29-4.11 (m, 1H), 3.84-3.77 (m, 1H), 3.75 (s, 3H), 3.48-3.32 (m, 2H), 3.27-3.10 (m, 1H), 1.86 (s, 3H), 1.13 (d, J=7.0 Hz, 6H).

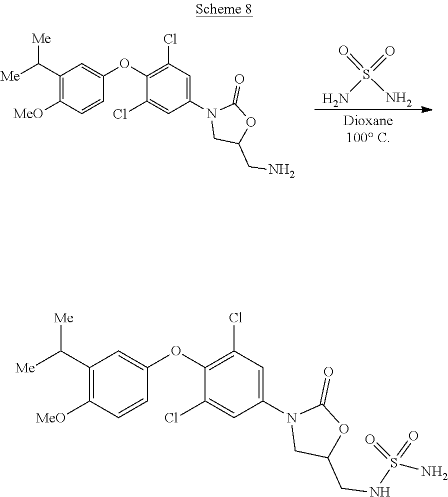 [SMILES: COc1ccc(Oc2c(Cl)cc(N3CC(CN)OC3=O)cc2Cl)cc1C(C)C ; NS(N)(=O)=O ; COc1ccc(Oc2c(Cl)cc(N3CC(CNS(N)(=O)=O)OC3=O)cc2Cl)cc1C(C)C]

### Example 12

### N-[(3-{3,5-dichloro-4-[4-methoxy-3-(propan-2-yl)phenoxy]phenyl}-2-oxo-1,3-oxazolidin-5-yl)methyl]aminosulfonamide

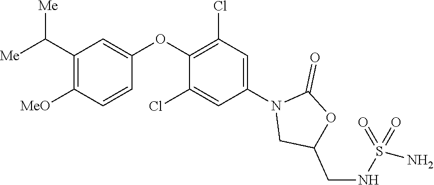 [SMILES: COc1ccc(Oc2c(Cl)cc(N3CC(CNS(N)(=O)=O)OC3=O)cc2Cl)cc1C(C)C]

To a solution of 5-(aminomethyl)-3-(3,5-dichloro-4-(3-isopropyl-4-methoxyphenoxy) phenyl)oxazolidin-2-one (15 mg, 0.035 mmol) in dioxane (2 mL) was added sulfuric diamide (3.39 mg, 0.035 mmol). The mixture was heated to 100° C. for 1 h. Solvent was removed and the crude material was purified by reverse phase preparative LC/MS to obtain Example 12, (6.5 mg, 0.013 mmol, 36% yield). LCMS m/z 503.9 (M+H); rt 2.12 min; Method B. ¹H NMR (500 MHz, DMSO-d₆) δ 7.81 (s, 2H), 7.03 (t, J=6.4 Hz, 1H), 6.86 (d, J=9.0 Hz, 1H), 6.81 (d, J=2.8 Hz, 1H), 6.72 (s, 1H), 6.41 (dd, J=8.8, 2.9 Hz, 1H), 4.88-4.72 (m, 1H), 4.18 (t, J=8.9 Hz, 1H), 3.98-3.85 (m, 1H), 3.74 (s, 3H), 3.46-3.11 (m, 4H), 1.12 (d, J=6.8 Hz, 6H).

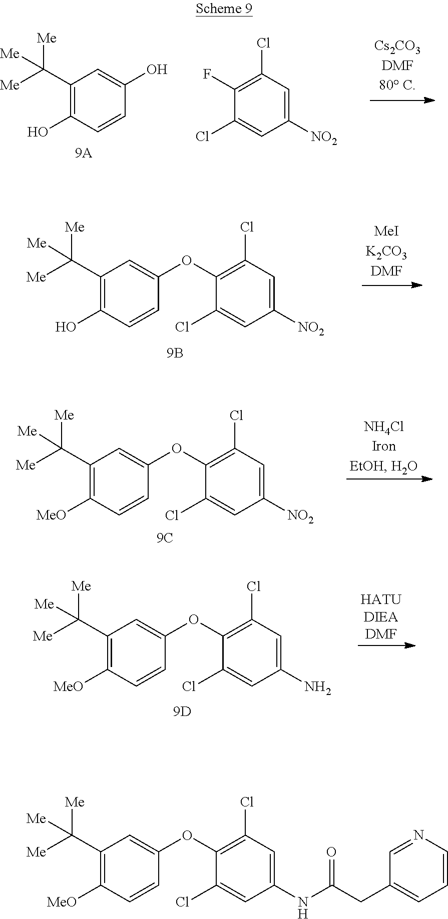 [SMILES: CC(C)(C)c1cc(O)ccc1O ; O=[N+]([O-])c1cc(Cl)c(F)c(Cl)c1 ; CC(C)(C)c1cc(Oc2c(Cl)cc([N+](=O)[O-])cc2Cl)ccc1O ; COc1ccc(Oc2c(Cl)cc([N+](=O)[O-])cc2Cl)cc1C(C)(C)C ; COc1ccc(Oc2c(Cl)cc(N)cc2Cl)cc1C(C)(C)C ; COc1ccc(Oc2c(Cl)cc(NC(=O)Cc3cccnc3)cc2Cl)cc1C(C)(C)C]

### Example 13

### N-(4-(3-(tert-butyl)-4-methoxyphenoxy)-3,5-dichlorophenyl)-2-(pyridin-3-yl)acetamide

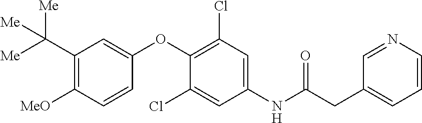 [SMILES: COc1ccc(Oc2c(Cl)cc(NC(=O)Cc3cccnc3)cc2Cl)cc1C(C)(C)C]

### Intermediate 9B: 2-(tert-butyl)-4-(2,6-dichloro-4-nitrophenoxy)phenol

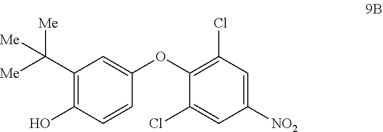 [SMILES: CC(C)(C)c1cc(Oc2c(Cl)cc([N+](=O)[O-])cc2Cl)ccc1O]

A pressure vessel containing a suspension of 2-(tert-butyl)benzene-1,4-diol 9A (500 mg, 3.01 mmol), 1,3-dichloro-2-fluoro-5-nitrobenzene (695 mg, 3.31 mmol), and cesium carbonate (1470 mg, 4.51 mmol) in DMF (10 mL) was heated at 80° C. for 10 h. The reaction was then allowed to cool to room temperature. The mixture was quenched with water, and the aqueous layer was extracted with EtOAc (3×25 mL). The combined organic layers were dried over magnesium sulfate and concentrated. The residue was dissolved in minimal DCM, adsorbed onto a plug of SiO2, and purified by flash chromatography (Silica, 0% to 20% EtOAc/hexanes, 24 g column, 25 min gradient) to afford 2-(tert-butyl)-4-(2,6-dichloro-4-nitrophenoxy)phenol 9B (367 mg, 1.03 mmol, 34.3% yield) and 3-(tert-butyl)-4-(2,6-dichloro-4-nitrophenoxy)phenol (380 mg, 1.07 mmol, 35.5% yield). ¹H NMR (400 MHz, CHLOROFORM-d) δ 8.34 (s, 2H), 6.96 (d, J=3.1 Hz, 1H), 6.52 (dd, J=8.7, 3.0 Hz, 1H), 6.10 (d, J=8.6 Hz, 1H), 4.57 (br s, 1H), 1.55-1.51 (m, 9H), 0.07-0.03 (m, 1H), 0.02-−0.02 (m, 1H).

### Intermediate 9D: 4-(3-(tert-butyl)-4-methoxyphenoxy)-3,5-dichloroaniline

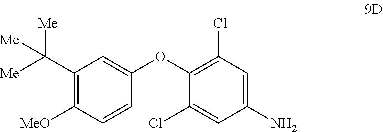 [SMILES: COc1ccc(Oc2c(Cl)cc(N)cc2Cl)cc1C(C)(C)C]

Potassium carbonate (276 mg, 2.000 mmol) and Mel (0.125 mL, 2.000 mmol) were added to a solution of 2-(tert-butyl)-4-(2,6-dichloro-4-nitrophenoxy)phenol 9B (356 mg, 1 mmol) in DMF (6 mL). The reaction was stirred at room temperature for 20 h. The reaction mixture was quenched with water. The precipitate formed was filtered, washed with water and air-dried. The crude residue 9C was treated with ammonium chloride (214 mg, 4.00 mmol) and iron (335 mg, 6.00 mmol) in EtOH/water (10/3) at 80° C. for 10 h. The reaction mixture was cooled to room temperature, filtered and the residue was washed with EtOAc (3×). The filtrate was concentrated and purified by silica gel chromatography (24 g column, 0-50%, EtOAc/Hex, 24 min) to obtain 4-(3-(tert-butyl)-4-methoxyphenoxy)-3,5-dichloroaniline 9D (359 mg, 1.055 mmol, 100% yield). LCMS m/z 340.9 (M+H); rt 1.12 min; Method C.

### Example 13: N-(4-(3-(tert-butyl)-4-methoxyphenoxy)-3,5-dichlorophenyl)-2-(pyridin-3-yl)acetamide

To a solution of 2-(pyridin-3-yl)acetic acid.HCl (12.91 mg, 0.074 mmol) in DMF (2 mL) was added HATU (28.3 mg, 0.074 mmol) and stirred for 5 min. To the resulting solution was added 4-(3-(tert-butyl)-4-methoxyphenoxy)-3,5-dichloroaniline 9D (23 mg, 0.068 mmol) and DIEA (0.047 mL, 0.270 mmol). The mixture was stirred for 36 h at room temperature. The crude material was purified by reverse phase preparative LC/MS to obtain Example 13 (2.9 mg, 0.006 mmol, 9% yield). LCMS m/z 459.2 (M+H); rt 2.07 min; Method B. ¹H NMR (500 MHz, DMSO-d₆) δ 10.66 (s, 1H), 8.56 (br s, 1H), 8.51 (br s, 1H), 7.84-7.79 (m, 3H), 7.45 (t, J=6.8 Hz, 1H), 6.87 (d, J=8.9 Hz, 1H), 6.80 (br d, J=2.4 Hz, 1H), 6.43 (dd, J=8.7, 2.9 Hz, 1H), 3.78-3.75 (m, 2H), 1.28 (s, 9H).

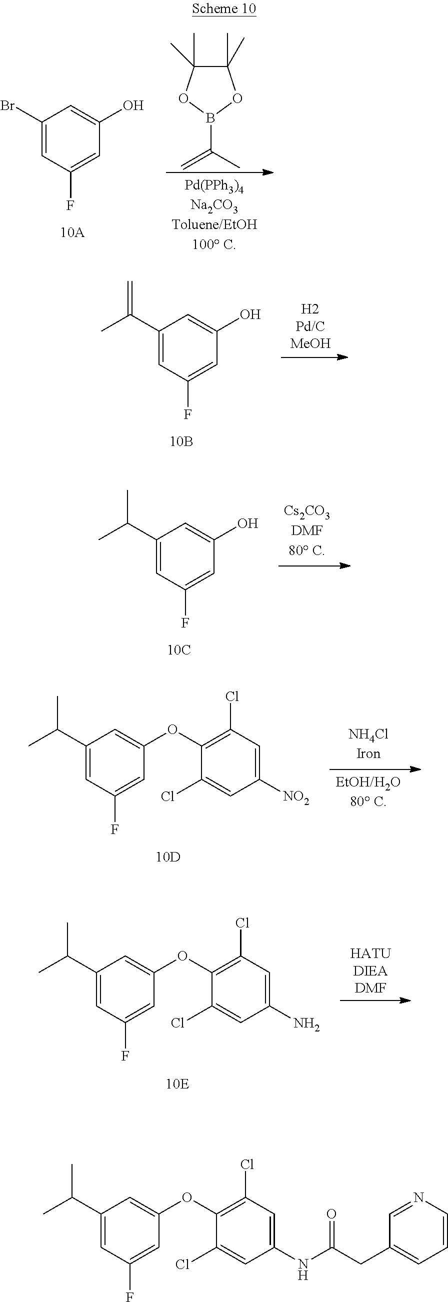

### Example 14

### N-(3,5-dichloro-4-(3-fluoro-5-isopropylphenoxy)phenyl)-2-(pyridin-3-yl)acetamide

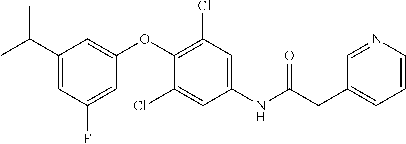 [SMILES: CC(C)c1cc(F)cc(Oc2c(Cl)cc(NC(=O)Cc3cccnc3)cc2Cl)c1]

### Intermediate 10B: 3-fluoro-5-(prop-1-en-2-yl)phenol

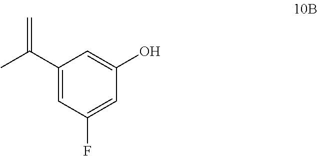

To a 20 mL microwave vessel was added 3-bromo-5-fluorophenol 10A (1000 mg, 5.24 mmol), 4,4,5,5-tetramethyl-2-(prop-1-en-2-yl)-1,3,2-dioxaborolane (1320 mg, 7.85 mmol), ethanol (10 mL), toluene (10 mL) and 2 M aqueous sodium carbonate (1.650 mL, 7.85 mmol). The mixture was sonicated and degassed with nitrogen for 5 min. The mixture was then treated with tetrakis(triphenylphosphine)palladium (45.5 mg, 0.039 mmol). The vessel was sealed and heated under microwave irradiation to 100° C. for 120 min. The reaction mixture was concentrated. The residue was partitioned between EtOAc and saturated aqueous ammonium chloride. The aqueous layer was extracted two more times with EtOAc. The combined organic layers were washed with brine, dried over sodium sulfate and concentrated. The crude residue was purified by silica gel chromatography (24 g column, 0-50% EtOAc/Hexane, 20 min) to afford 3-fluoro-5-(prop-1-en-2-yl)phenol 10B (797 mg, ˜100% yield), which was used as such in the next step.

### Intermediate 10C: 3-fluoro-5-isopropylphenol

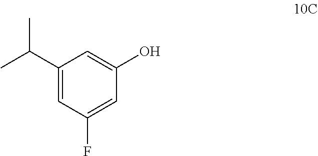

To a solution of 3-fluoro-5-(prop-1-en-2-yl)phenol 10B (797 mg, 5.24 mmol)) in MeOH (15 mL) was added Pd/C (0.5 mmol, 0.1%). The pressure vessel was evacuated and back-filled with nitrogen three times. The reaction mixture was then hydrogenated under 50 PSI hydrogen pressure for 5 h. the pressure vessel was then evacuated and back-filled with nitrogen for three times. The reaction mixture was filtered and the filtrate was concentrated to obtain 3-fluoro-5-isopropylphenol 10C (543 mg, 67.3% yield in two steps). LCMS m/z 153.2 (M−H); rt 0.90 min; Method F.

### Intermediate 10D: 1,3-dichloro-2-(3-fluoro-5-isopropylphenoxy)-5-nitrobenzene

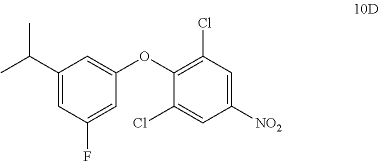 [SMILES: CC(C)c1cc(F)cc(Oc2c(Cl)cc([N+](=O)[O-])cc2Cl)c1]

A pressure vessel containing a suspension of 3-fluoro-5-isopropylphenol 10C (53 mg, 0.344 mmol), 1,3-dichloro-2-fluoro-5-nitrobenzene (83 mg, 0.395 mmol), and cesium carbonate (224 mg, 0.688 mmol) in DMF (5 mL) was heated at 80° C. for 2 h. The reaction mixture was cooled to room temperature and quenched with water. The resulting mixture was extracted with EtOAc (3×25 mL). The combined organic layers were dried over magnesium sulfate and concentrated to obtain crude 1,3-dichloro-2-(3-fluoro-5-isopropylphenoxy)-5-nitrobenzene 10D that was used as such in the next step.

### Intermediate 10E: 3,5-dichloro-4-(3-fluoro-5-isopropylphenoxy)aniline

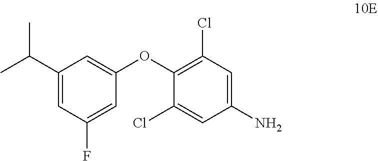 [SMILES: CC(C)c1cc(F)cc(Oc2c(Cl)cc(N)cc2Cl)c1]

To a vial containing a suspension of 1,3-dichloro-2-(3-fluoro-5-(prop-1-en-2-yl)phenoxy)-5-nitrobenzene 10D in ethanol (15 mL) was added a solution of ammonium chloride (110 mg, 2.06 mmol) in water (5 mL), followed by iron (154 mg, 2.75 mmol). The resulting mixture was heated at 80° C. for 2 h. The reaction was then allowed to cool to room temperature. The mixture was filtered and washed with EtOAc (120 mL). The organic phase was then washed with 1:1 mixture of brine and 1.5 M aqueous K₂HPO₄ (60 mL). The aqueous layer was back-extracted with EtOAc (3×30 mL). The combined organic layers were dried (magnesium sulfate), filtered through a pad of Celite, and concentrated. The residue was purified by silica gel chromatography (12 g column, 0-50% EtOAc/Hexane, 25 min) to obtain 3,5-dichloro-4-(3-fluoro-5-isopropylphenoxy)aniline 10E (94.3 mg, 0.30 mmol, 87% yield for two steps). LCMS m/z 314.0 (M+H); rt 1.13 min; Method C. ¹H NMR (400 MHz, CHLOROFORM-d) δ 6.74-6.69 (m, 2H), 6.67-6.57 (m, 2H), 6.39-6.16 (m, 1H), 3.82-3.36 (m, 2H), 2.95-2.75 (m, 1H), 1.28-1.16 (m, 6H).

### Example 14: N-(3,5-dichloro-4-(3-fluoro-5-isopropylphenoxy)phenyl)-2-(pyridin-3-yl)acetamide

To a solution of 3,5-dichloro-4-(3-fluoro-5-isopropylphenoxy)aniline (55 mg, 0.175 mmol) in DMF (2 mL) was added 2-(pyridin-3-yl)acetic acid.HCl (60.8 mg, 0.350 mmol), HATU (100 mg, 0.263 mmol) and then DIEA (0.122 mL, 0.700 mmol). The mixture was stirred overnight. The crude material was purified by reverse phase preparative LC/MS to obtain Example 14 (13.4 mg, 0.031 mmol, 18% yield). LCMS m/z 443.2 (M+H); rt 2.42 min; Method B. ¹H NMR (500 MHz, DMSO-d₆) δ 10.71 (s, 1H), 8.56-8.39 (m, 2H), 7.84 (s, 2H), 7.75 (d, J=7.7 Hz, 1H), 7.44-7.28 (m, 1H), 6.81 (d, J=9.5 Hz, 1H), 6.57 (s, 1H), 6.42 (d, J=10.0 Hz, 1H), 3.30-3.07 (m, 2H), 2.85 (dt, J=13.7, 6.9 Hz, 1H), 1.13 (d, J=6.8 Hz, 6H).

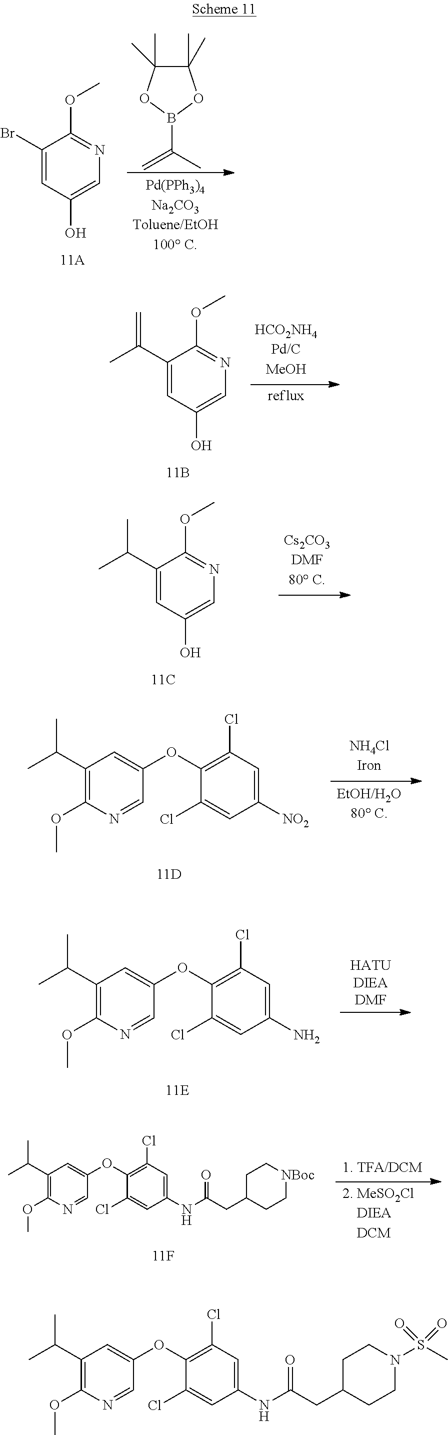

### Example 15

### N-(3,5-dichloro-4-((5-isopropyl-6-methoxypyridin-3-yl)oxy)phenyl)-2-(1-(methylsulfonyl)piperidin-4-yl)acetamide

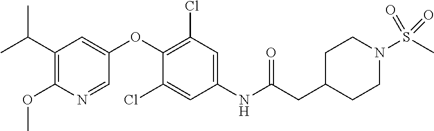 [SMILES: COc1ncc(Oc2c(Cl)cc(NC(=O)CC3CCN(S(C)(=O)=O)CC3)cc2Cl)cc1C(C)C]

### Intermediate 11B: 6-methoxy-5-(prop-1-en-2-yl)pyridin-3-ol

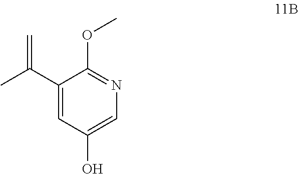

To a 20 mL microwave vessel was added 5-bromo-6-methoxypyridin-3-ol 11A (250 mg, 1.23 mmol), 4,4,5,5-tetramethyl-2-(prop-1-en-2-yl)-1,3,2-dioxaborolane (412 mg, 2.45 mmol), ethanol (6 mL), toluene (6 mL) and 2 M aqueous sodium carbonate (1.225 mL, 2.45 mmol). The mixture was sonicated and degassed with nitrogen for 5 min. The mixture was then treated with tetrakis(triphenylphosphine)palladium (70.8 mg, 0.061 mmol). The vessel was sealed and heated under microwave irradiation to 100° C. for 120 min. The reaction mixture was concentrated. The residue was partitioned between EtOAc and saturated ammonium chloride. The aqueous layer was extracted two more times with EtOAc. The combined organic layers were washed with brine, dried over sodium sulfate and concentrated. The crude residue was purified by silica gel chromatography (12 g column, 0-70% EtOAc/Hexane, 16 min) to afford 6-methoxy-5-(prop-1-en-2-yl)pyridin-3-ol 11B (100 mg, 50% yield), LCMS m/z 166.2 (M+H); rt 0.73 min; Method C. which was directly used for next step.

### Intermediate 11C: 5-isopropyl-6-methoxypyridin-3-ol

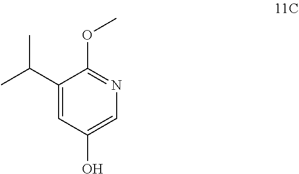

A 10 mL vessel containing 6-methoxy-5-(prop-1-en-2-yl)pyridin-3-ol 11B (100 mg, 0.605 mmol) was outfitted with a reflux condenser and evacuated and backfilled with nitrogen three times. The substrate was dissolved in MeOH (15 mL), then 10% palladium on carbon (32.2 mg, 0.030 mmol) and ammonium formate (191 mg, 3.03 mmol) were added. The mixture was stirred at reflux under nitrogen atmosphere for 2 h. The mixture was filtered, washed with EtOAc and the filtrate was concentrated. The crude product was dissolved into EtOAc again and filtered to get rid of ammonium formate. The filtrate was concentrated to obtain 5-isopropyl-6-methoxypyridin-3-ol 11C (100 mg, 0.598 mmol, 99% yield). LCMS m/z 168.1 (M+H); rt 1.88 min; Method E.

### Intermediate 11F: tert-butyl 4-(2-((3,5-dichloro-4-((5-isopropyl-6-methoxypyridin-3-yl)oxy)phenyl)amino)-2-oxoethyl)piperidine-1-carboxylate

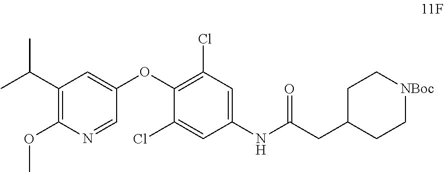 [SMILES: COc1ncc(Oc2c(Cl)cc(NC(=O)CC3CCN(C(=O)OC(C)(C)C)CC3)cc2Cl)cc1C(C)C]

Intermediate 11F was prepared according to methods described for Example 13 from Intermediate 11C, through intermediate 11D and 11E. LCMS m/z 551.9 (M+H); rt 4.23 min; Method E.

### Example 15: N-(3,5-dichloro-4-((5-isopropyl-6-methoxypyridin-3-yl)oxy)phenyl)-2-(1-(methylsulfonyl)piperidin-4-yl)acetamide

To tert-butyl 4-(2-((3,5-dichloro-4-((5-isopropyl-6-methoxypyridin-3-yl)oxy)phenyl)amino)-2-oxoethyl)piperidine-1-carboxylate 11F (83 mg, 0.15 mmol) was added 30% TFA in DCM (3 mL). The mixture was stirred for 1 h. The reaction mixture was concentrated. To the residue was added DCM (3 mL) and DIEA (0.105 mL, 0.600 mmol). The mixture was cooled to 0° C. and methanesulfonyl chloride (51.5 mg, 0.450 mmol) was added. The mixture was stirred for 1 h and then concentrated. The crude material was purified by reverse phase preparative LC/MS to obtain Example 15 (8.3 mg, 0.015 mmol, 10% yield). LCMS m/z 530.0 (M+H); rt 2.31 min; Method B. ¹H NMR (500 MHz, DMSO-d₆) δ 10.35 (s, 1H), 7.87-7.72 (m, 2H), 7.52-7.35 (m, 1H), 7.29-7.09 (m, 1H), 3.90-3.76 (m, 3H), 3.54 (d, J=11.6 Hz, 1H), 3.07 (dt, J=13.4, 6.7 Hz, 1H), 2.84 (s, 3H), 2.76-2.63 (m, 3H), 2.31 (d, J=7.0 Hz, 2H), 1.95-1.70 (m, 3H), 1.34-1.21 (m, 2H), 1.14 (d, J=7.0 Hz, 6H).

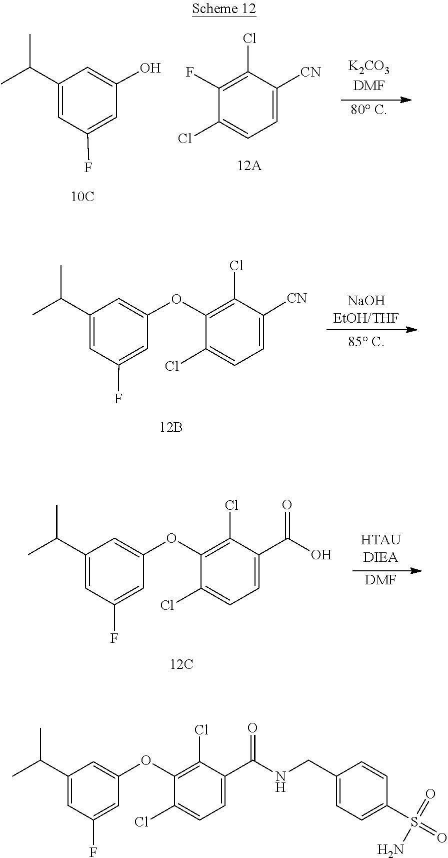

### Example 16

### 2,4-dichloro-3-(3-fluoro-5-isopropylphenoxy)-N-(4-sulfamoylbenzyl)benzamide

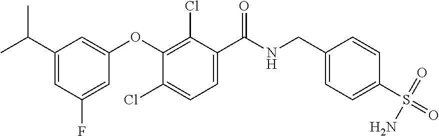 [SMILES: CC(C)c1cc(F)cc(Oc2c(Cl)ccc(C(=O)NCc3ccc(S(N)(=O)=O)cc3)c2Cl)c1]

### Intermediate 12B: 2,4-dichloro-3-(3-fluoro-5-isopropylphenoxy)benzonitrile

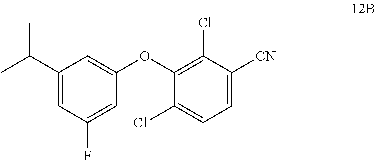 [SMILES: CC(C)c1cc(F)cc(Oc2c(Cl)ccc(C#N)c2Cl)c1]

To a solution of 3-fluoro-5-isopropylphenol 10C (150 mg, 0.973 mmol) and 2,4-dichloro-3-fluorobenzonitrile 12A (185 mg, 0.973 mmol) in DMF (4 mL) was added potassium carbonate (269 mg, 1.946 mmol). The reaction mixture was stirred at 80° C. for 2 h. The reaction mixture was diluted with cold water and extracted with EtOAc (3×10 mL). The combined organic layers were washed with brine (1×15 mL), dried over sodium sulfate and concentrated to obtain crude 2,4-dichloro-3-(3-fluoro-5-isopropylphenoxy)benzonitrile

12B (300 mg, 0.925 mmol, 95% yield) that was used as such in next step.

### Intermediate 12C: 2,4-dichloro-3-(3-fluoro-5-isopropylphenoxy)benzoic Acid

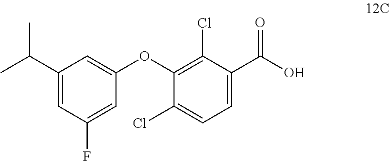 [SMILES: CC(C)c1cc(F)cc(Oc2c(Cl)ccc(C(=O)O)c2Cl)c1]

To a suspension of 2,4-dichloro-3-(3-fluoro-5-isopropylphenoxy)benzonitrile 12B (300 mg, 0.925 mmol) in EtOH (2 mL) and THE (1 mL) was added aqueous 3 M sodium hydroxide (2.468 mL, 7.40 mmol). The reaction mixture was stirred at 85° C. for 10 h. The reaction mixture was neutralized with 1 M HCl to pH 3. The precipitate formed was filtered, washed with water and air-dried to obtain 2,4-dichloro-3-(3-fluoro-5-isopropylphenoxy)benzoic acid 12C (235 mg, 0.685 mmol, 74.0% yield). LCMS m/z 341.2 (M−H); rt 0.77 min; Method F.

### Example 16: 2,4-dichloro-3-(3-fluoro-5-isopropylphenoxy)-N-(4-sulfamoylbenzyl)benzamide

To a solution of 2,4-dichloro-3-(3-fluoro-5-isopropylphenoxy)benzoic acid 12C (15 mg, 0.044 mmol) in DMF (2 mL) was added HATU (18.28 mg, 0.048 mmol). The mixture was stirred for 5 min and then DIEA (0.023 mL, 0.131 mmol) and (1-(methylsulfonyl)piperidin-4-yl)methanamine (10.93 mg, 0.057 mmol) were added. The reaction mixture was stirred for 2 h. The crude material was purified by reverse phase preparative LC/MS to obtain Example 16 (15.3 mg, 0.030 mmol, 68% yield). LCMS m/z 511.2 (M+H); rt 2.06 min; Method B. ¹H NMR (500 MHz, DMSO-d₆) δ 9.23 (br. s., 1H), 7.79 (d, J=8.0 Hz, 2H), 7.72 (d, J=8.2 Hz, 1H), 7.53 (d, J=7.9 Hz, 2H), 7.50 (d, J=8.2 Hz, 1H), 7.35 (br. s., 2H), 6.85 (d, J=9.8 Hz, 1H), 6.67 (br. s., 1H), 6.40 (d, J=10.0 Hz, 1H), 4.53 (d, J=5.6 Hz, 2H), 2.97-2.78 (m, 1H), 1.16 (d, J=6.6 Hz, 6H).

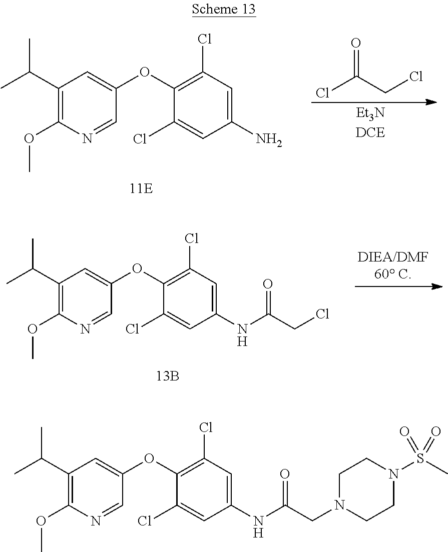 [SMILES: COc1ncc(Oc2c(Cl)cc(N)cc2Cl)cc1C(C)C ; O=C(Cl)CCl ; COc1ncc(Oc2c(Cl)cc(NC(=O)CCl)cc2Cl)cc1C(C)C ; COc1ncc(Oc2c(Cl)cc(NC(=O)CN3CCN(S(C)(=O)=O)CC3)cc2Cl)cc1C(C)C]

### Example 17

### N-(3,5-dichloro-4-((5-isopropyl-6-methoxypyridin-3-yl)oxy)phenyl)-2-(4-(methylsulfonyl)piperazin-1-yl)acetamide

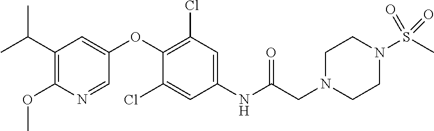 [SMILES: COc1ncc(Oc2c(Cl)cc(NC(=O)CN3CCN(S(C)(=O)=O)CC3)cc2Cl)cc1C(C)C]

### Intermediate 13B: 2-chloro-N-(3,5-dichloro-4-((5-isopropyl-6-methoxypyridin-3-yl)oxy) Phenyl)acetamide

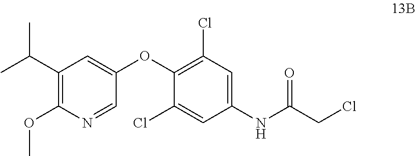 [SMILES: COc1ncc(Oc2c(Cl)cc(NC(=O)CCl)cc2Cl)cc1C(C)C]

To a solution of 3,5-dichloro-4-((5-isopropyl-6-methoxypyridin-3-yl)oxy)aniline 11E (50 mg, 0.153 mmol) in DCM (3 mL) at 0° C. was added triethylamine (0.032 mL, 0.229 mmol) and 2-chloroacetyl chloride (25.9 mg, 0.229 mmol). The reaction mixture was stirred at room temperature for 3 h. The reaction mixture was concentrated to obtain 2-chloro-N-(3,5-dichloro-4-((5-isopropyl-6-methoxypyridin-3-yl)oxy)phenyl)acetamide 13B (60 mg, 0.149 mmol, 97% yield) that was used as such in the next step.

### Example 17: N-(3,5-dichloro-4-((5-isopropyl-6-methoxypyridin-3-yl)oxy)phenyl)-2-(4-(methylsulfonyl)piperazin-1-yl)acetamide

To a solution of 2-chloro-N-(3,5-dichloro-4-((5-isopropyl-6-methoxypyridin-3-yl)oxy)phenyl)acetamide 13B (40 mg, 0.099 mmol) in DMF (2 mL) was added DIEA (0.069 mL, 0.396 mmol) and then 1-(methylsulfonyl)piperazine (32.5 mg, 0.198 mmol). The mixture was heated to 60° C. for 2 h. The crude material was purified by reverse phase preparative LC/MS to obtain Example 17 (18.1 mg, 0.034 mmol, 34% yield). LCMS m/z 531.0 (M+H); rt 1.92 min; Method B. ¹H NMR (500 MHz, DMSO-d₆) δ 10.12 (s, 1H), 7.94 (s, 2H), 7.43 (d, J=2.7 Hz, 1H), 7.22 (d, J=2.7 Hz, 1H), 3.83 (s, 3H), 3.28-3.13 (m, 6H), 3.11-2.98 (m, 1H), 2.89 (s, 3H), 2.62 (br. s., 4H), 1.14 (d, J=6.7 Hz, 6H).

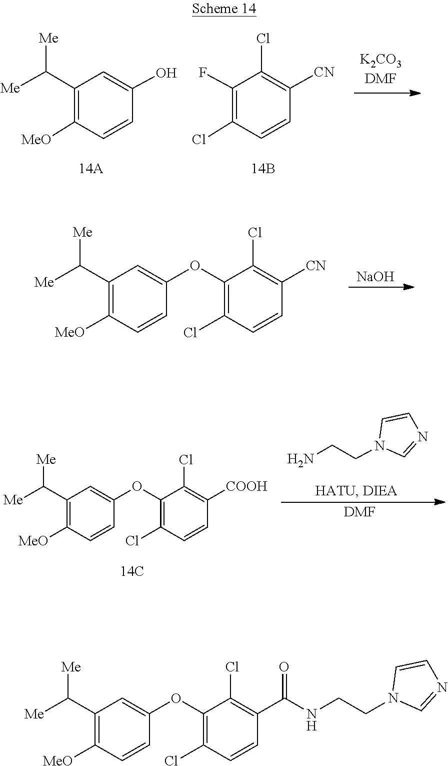

### Example 18

### 2,4-dichloro-3-(3-isopropyl-4-methoxyphenoxy)benzonitrile

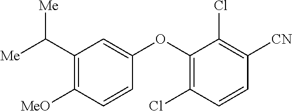 [SMILES: COc1ccc(Oc2c(Cl)ccc(C#N)c2Cl)cc1C(C)C]

To a solution of 3-isopropyl-4-methoxyphenol 14A (17.50 mg, 0.105 mmol) and 2,4-dichloro-3-fluorobenzonitrile 14B (20 mg, 0.105 mmol) in DMF (1 mL) was added potassium carbonate (29.1 mg, 0.211 mmol). The reaction mixture was stirred at 80° C. for 2 h. The reaction mixture was diluted with cold water. The precipitate formed was collected by filtration. The compound was purified by reverse phase preparative LC/MS to give Example 18 (10.1 mg, 29% yield). LCMS m/z 336.0 (M+H); rt 2.57 min; Method A.

### Example 19

### N-(2-(1H-imidazol-1-yl)ethyl)-2,4-dichloro-3-(3-isomethoxyphenoxy)benzamide

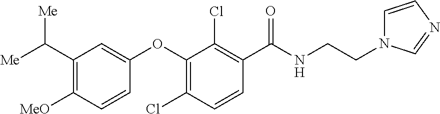 [SMILES: COc1ccc(Oc2c(Cl)ccc(C(=O)NCCn3ccnc3)c2Cl)cc1C(C)C]

### Intermediate 14C: 2,4-dichloro-3-(3-isopropyl-4-methoxyphenoxy)benzoic Acid

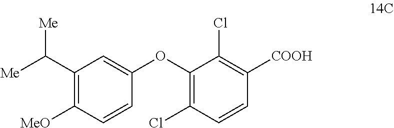 [SMILES: COc1ccc(Oc2c(Cl)ccc(C(=O)O)c2Cl)cc1C(C)C]

To a suspension of 2,4-dichloro-3-(3-isopropyl-4-methoxyphenoxy)benzonitrile Example 18 (860 mg, 2.56 mmol) in EtOH (20 mL) and THE (10 mL) was added 3 M aqueous sodium hydroxide (8.53 mL, 25.6 mmol). The reaction mixture was stirred at 90° C. for 3 h. The solvent was removed in vacuo and the residue was purified by silica gel chromatography using 0-100% EtOAc in hexanes followed by 0-10% MeOH in DCM to give 14C (540 mg, 59% yield). LCMS m/z 355.0 (M+H); rt 1.06 min; Method C.

### Example 19: N-(2-(1H-imidazol-1-yl)ethyl)-2,4-dichloro-3-(3-isomethoxyphenoxy)benzamide

To a solution of 2,4-dichloro-3-(3-isopropyl-4-methoxyphenoxy)benzoic acid 14C (30 mg, 0.084 mmol) and 2-(1H-imidazol-1-yl)ethanamine (9.39 mg, 0.084 mmol) in DMF (1 mL) were added HATU (48.2 mg, 0.127 mmol) and DIEA (0.044 mL, 0.253 mmol). The reaction mixture was stirred at room temperature for 16 h and purified by reverse phase preparative HPLC to obtain Example 19 (12.5 mg, 33.0% yield). ¹H NMR (500 MHz, DMSO-d₆) δ 8.73 (br t, J=5.4 Hz, 1H), 7.73-7.60 (m, 2H), 7.28 (d, J=8.3 Hz, 1H), 7.21 (s, 1H), 6.90 (s, 1H), 6.88-6.81 (m, 2H), 6.36 (dd, J=8.9, 3.1 Hz, 1H), 4.15 (br t, J=5.8 Hz, 2H), 3.74 (s, 3H), 3.57 (m, 2H), 3.27-3.18 (m, 1H), 1.13 (d, J=6.9 Hz, 6H); LCMS m/z 448.0 (M+H); rt 1.92 min; Method A.

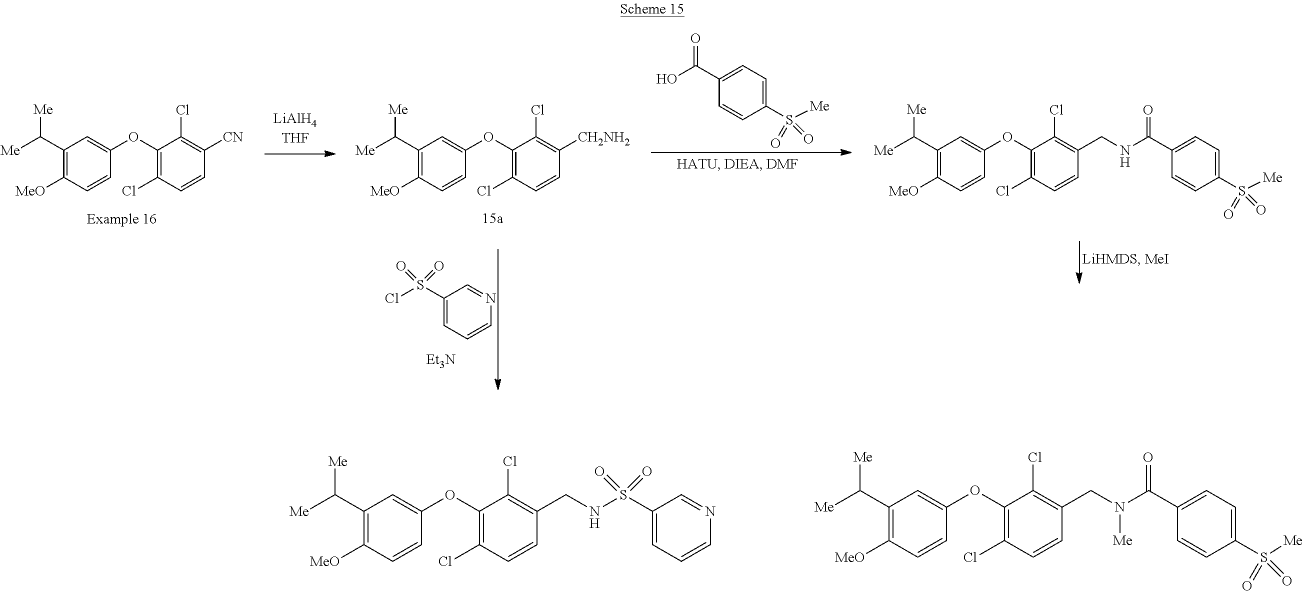

### Example 20

### N-(2,4-dichloro-3-(3-isopropyl-4-methoxyphenoxy)benzyl)-4-(methylsulfonyl)benzamide

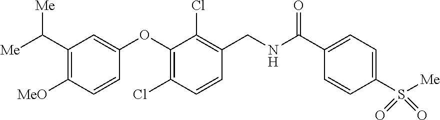 [SMILES: COc1ccc(Oc2c(Cl)ccc(CNC(=O)c3ccc(S(C)(=O)=O)cc3)c2Cl)cc1C(C)C]

### Intermediate 15A: (2,4-dichloro-3-((5-isopropyl-6-methoxypyridin-3-yl)oxy)phenyl)methanamine

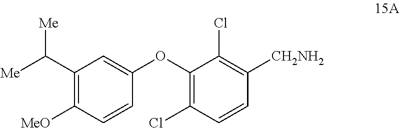

To a solution of 2,4-dichloro-3-(3-isopropyl-4-methoxyphenoxy)benzonitrile Example 16 (1500 mg, 4.46 mmol) in THE (15 mL) at 0° C. was added lithium aluminum hydride (5.58 mL, 11.15 mmol, 2 M in THF) dropwise. The reaction mixture was stirred at 0° C. for 1 h and then at room temperature for 2 h. The reaction mixture was quenched with a small amount of wet sodium sulfate and stirred at room temperature for 1 h. The resulting mixture was partitioned between EtOAc and sat. NaHCO₃. The organic layer was separated and concentrated. The residue was purified by silica gel chromatography using 0-5% MeOH in DCM to afford 15A (830 mg, 55% yield). LCMS m/z 339.8 (M+H); rt 0.81 min; Method C.

### Example 20: N-(2,4-dichloro-3-(3-isopropyl-4-methoxyphenoxy)benzyl)-4-(methylsulfonyl)benzamide

To a solution of 4-(methylsulfonyl)benzoic acid (11.77 mg, 0.059 mmol) and (2,4-dichloro-3-(3-isopropyl-4-methoxyphenoxy)phenyl)methanamine 15A (20 mg, 0.059 mmol) in DMF (1 mL) were added HATU (33.5 mg, 0.088 mmol) and DIEA (0.031 mL, 0.176 mmol). The reaction mixture was stirred at room temperature for 16 h. The reaction mixture was purified by reverse phase preparative LC/MS to obtain Example 20 (22.7 mg, 72.1% yield). LCMS m/z 521.8 (M+H); rt 1.05 min; Method C; ¹H NMR (400 MHz, METHANOL-d₄) δ 9.25 (br t, J=5.6 Hz, 1H), 8.13-8.05 (m, 4H), 7.48 (d, J=8.4 Hz, 1H), 7.35 (d, J=8.4 Hz, 1H), 6.82 (s, 1H), 6.77 (d, J=3.1 Hz, 1H), 6.47 (dd, J=8.9, 3.1 Hz, 1H), 4.75-4.67 (m, 2H), 3.79 (s, 3H), 3.31-3.23 (m, 1H), 3.18 (s, 3H), 1.15 (d, J=6.8 Hz, 6H).

### Example 21

### N-(2,4-dichloro-3-(3-isopropyl-4-methoxyphenoxy)benzyl)pyridine-3-sulfonamide

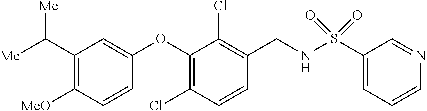 [SMILES: COc1ccc(Oc2c(Cl)ccc(CNS(=O)(=O)c3cccnc3)c2Cl)cc1C(C)C]

To a solution of (2,4-dichloro-3-(3-isopropyl-4-methoxyphenoxy) phenyl)methanamine 15A (20 mg, 0.059 mmol) in DCM (0.5 mL) at room temperature were added pyridine-3-sulfonyl chloride.HCl (13.84 mg, 0.065 mmol) and triethylamine (0.025 mL, 0.176 mmol). The reaction mixture was stirred at room temperature for 16 h and purified by reverse phase preparative LC/MS to obtain Example 21 (6.9 mg, 25% yield). LCMS m/z 481.1 (M+H); rt 2.38 min; Method A; ¹H NMR (500 MHz, DMSO-d₆) δ 8.90 (br s, 1H), 8.81 (br d, J=4.0 Hz, 1H), 8.16 (br d, J=7.9 Hz, 1H), 7.61 (dd, J=7.6, 4.9 Hz, 1H), 7.54 (d, J=8.2 Hz, 1H), 7.34 (d, J=8.2 Hz, 1H), 6.83 (d, J=8.9 Hz, 1H), 6.80 (d, J=2.7 Hz, 1H), 6.26 (dd, J=8.7, 2.9 Hz, 1H), 4.20 (s, 2H), 3.73 (s, 3H), 3.25-3.12 (m, 1H), 1.12 (br d, J=6.7 Hz, 6H).

### Example 22

### N-(2,4-dichloro-3-(3-isopropyl-4-methoxyphenoxy)benzyl)-N-methyl-4-(methylsulfonyl)benzamide

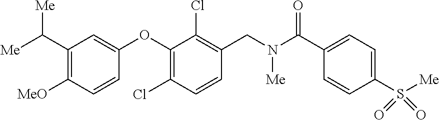 [SMILES: COc1ccc(Oc2c(Cl)ccc(CN(C)C(=O)c3ccc(S(C)(=O)=O)cc3)c2Cl)cc1C(C)C]

To a solution of N-(2,4-dichloro-3-(3-isopropyl-4-methoxyphenoxy)benzyl)-4-(methylsulfonyl)benzamide Example 20 (20 mg, 0.038 mmol) in THE (1 mL) at 0° C. was added a 1 M solution of lithium bis(trimethylsilyl)amide (0.077 mL, 0.077 mmol) in toluene. The reaction mixture was stirred at 0° C. for 30 min. To the resulting mixture was added iodomethane (13.58 mg, 0.096 mmol). The reaction mixture was stirred at 0° C. for 1 h and another portion of 1 M solution of lithium bis(trimethylsilyl)amide (0.077 mL, 0.077 mmol) in toluene and iodomethane (13.58 mg, 0.096 mmol) were added. The reaction mixture was stirred at 0° C. for another 1 h. The reaction mixture was diluted with MeOH and purified by reverse phase preparative LC/MS to obtain Example 22 (4.3 mg, 21% yield). LCMS m/z 535.9 (M+H); rt 1.08 min; Method C; ¹H NMR (400 MHz, CHLOROFORM-d) δ 8.05-7.94 (m, 4H), 7.45-7.40 (m, 1H), 7.39-7.34 (m, 1H), 6.88 (d, J=3.1 Hz, 1H), 6.79-6.73 (m, 1H), 6.72 (d, J=8.9 Hz, 1H), 6.45 (dd, J=8.8, 3.1 Hz, 1H), 4.79 (d, J=6.1 Hz, 2H), 3.80 (s, 3H), 3.38-3.25 (m, 1H), 2.09 (br s, 6H), 1.20 (d, J=6.8 Hz, 6H).

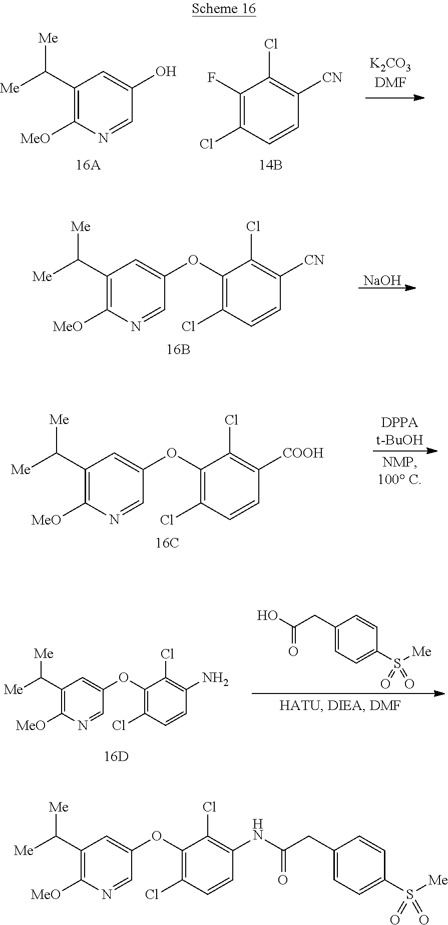

### Example 23

### N-(2,4-dichloro-3-((5-isopropyl-6-methoxypyridin-3-yl)oxy)phenyl)-2-(4-(methylsulfonyl)phenyl)acetamide

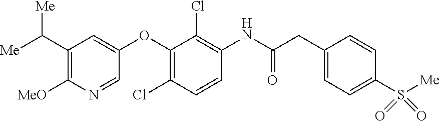 [SMILES: COc1ncc(Oc2c(Cl)ccc(NC(=O)Cc3ccc(S(C)(=O)=O)cc3)c2Cl)cc1C(C)C]

### Intermediate 16B: 2,4-dichloro-3-((5-isopropyl-6-methoxypyridin-3-yl)oxy)benzonitrile

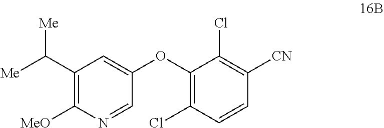 [SMILES: COc1ncc(Oc2c(Cl)ccc(C#N)c2Cl)cc1C(C)C]

To a solution of 5-isopropyl-6-methoxypyridin-3-ol 16A (2.0 g, 11.96 mmol) and 2,4-dichloro-3-fluorobenzonitrile 14B (2.273 g, 11.96 mmol) in DMF (10 mL) was added potassium carbonate (2.480 g, 17.94 mmol). The reaction mixture was stirred at 80° C. for 1 h. The reaction mixture was diluted with cold water and extracted with EtOAc (3×). The combined organics was dried over magnesium sulfate and concentrated to give a viscous oil, which was purified by silica gel chromatography eluting with 0-30% EtOAc in hexanes to ether 16B (3.14 g, 82% yield). LCMS m/z 339.2 (M+H); rt 1.14 min; Method C; ¹H NMR (400 MHz, CHLOROFORM-d) δ 7.54 (d, J=2.0 Hz, 2H), 7.40 (d, J=3.1 Hz, 1H), 7.18 (d, J=2.9 Hz, 1H), 3.93 (s, 3H), 3.18 (dt, J=13.8, 6.9 Hz, 1H), 1.23 (d, J=6.8 Hz, 6H).

### Intermediate 16C: 2,4-dichloro-3-((5-isopropyl-6-methoxypyridin-3-yl)oxy)benzoic Acid

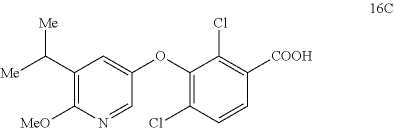 [SMILES: COc1ncc(Oc2c(Cl)ccc(C(=O)O)c2Cl)cc1C(C)C]

To a solution of 2,4-dichloro-3-((5-isopropyl-6-methoxypyridin-3-yl)oxy)benzonitrile 16B (2.14 g, 6.35 mmol) in THE (10 mL) and MeOH (10 mL) was added 3 M sodium hydroxide (6.35 mL, 19.04 mmol). The reaction mixture was stirred at 80° C. for 16 h. The reaction mixture was neutralized with 1 N HCl to pH 3-4, and extracted with EtOAc (3×). The organics was dried over magnesium sulfate and concentrated to give acid 16C (2.10 g, 93% yield) as a pale yellow solid. LCMS m/z 356.3 (M+H); rt 1.04 min; Method C.

### Intermediate 16D: 2,4-dichloro-3-((5-isopropyl-6-methoxypyridin-3-yl)oxy)aniline

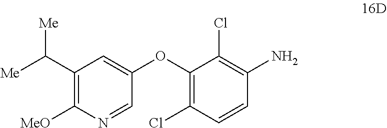 [SMILES: COc1ncc(Oc2c(Cl)ccc(N)c2Cl)cc1C(C)C]

To a solution of 2,4-dichloro-3-((5-isopropyl-6-methoxypyridin-3-yl)oxy)benzoic acid 16C (250 mg, 0.702 mmol) in NMP (3 mL) were added diphenyl phosphorazidate, DPPA (193 mg, 0.702 mmol) and TEA (0.147 mL, 1.053 mmol). The reaction mixture was heated at 100° C. for 3 h. The reaction mixture was concentrated and purified by reverse phase preparative LC/MS to give the desired product 16D (163 mg, 71% yield). LCMS m/z 327.2 (M+H); rt 1.10 min; Method C; ¹H NMR (400 MHz, CHLOROFORM-d) δ 7.51 (d, J=2.9 Hz, 1H), 7.31 (d, J=2.8 Hz, 1H), 7.17 (d, J=8.8 Hz, 1H), 6.65 (d, J=8.8 Hz, 1H), 6.40 (br s, 2H), 4.01 (s, 3H), 3.18 (dt, J=13.8, 6.9 Hz, 1H), 1.23 (d, J=6.8 Hz, 6H).

### Example 23: N-(2,4-dichloro-3-((5-isopropyl-6-methoxypyridin-3-yl)oxy)phenyl)-2-(4-(methylsulfonyl)phenyl)acetamide

To a solution of 2-(4-(methylsulfonyl)phenyl)acetic acid (19.64 mg, 0.092 mmol) and 2,4-dichloro-3-((5-isopropyl-6-methoxypyridin-3-yl)oxy)aniline 16D (20 mg, 0.061 mmol) in DMF (0.5 mL) were added HATU (46.5 mg, 0.122 mmol) and N-ethyl-N-isopropylpropan-2-amine (23.70 mg, 0.183 mmol). The reaction mixture was stirred at 65° C. for 6 h and purified by reverse phase preparative LC/MS to obtain Example 23 (7.0 mg, 20% yield). LCMS m/z 523.2 (M+H); rt 1.06 min; Method C; ¹H NMR (400 MHz, METHANOL-d₄) δ 7.92 (m, 3H), 7.61 (br d, J=8.2 Hz, 2H), 7.44 (s, 1H), 7.38 (d, J=9.0 Hz, 1H), 7.34 (d, J=2.9 Hz, 1H), 7.14 (d, J=2.9 Hz, 1H), 3.90 (s, 2H), 3.88 (s, 3H), 3.18-3.09 (m, 1H), 3.07 (s, 3H), 1.17 (d, J=6.8 Hz, 6H).

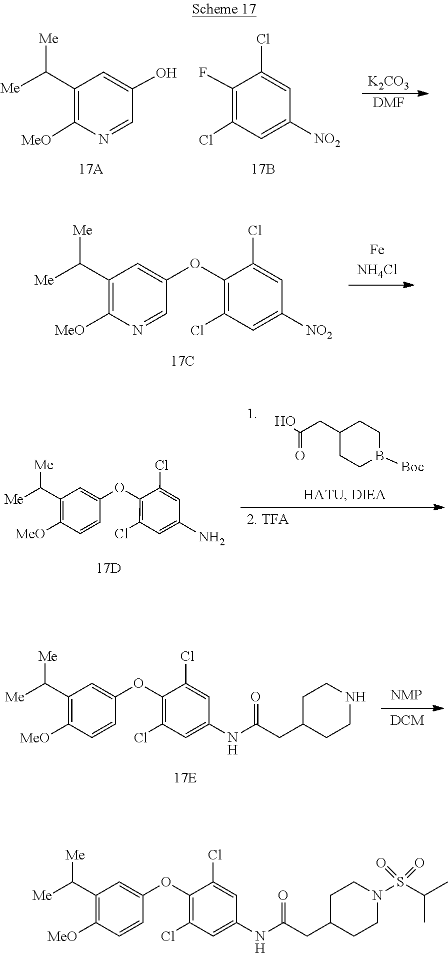

### Example 24

### N-(3,5-dichloro-4-((5-isopropyl-6-methoxypyridin-3-yl)oxy)phenyl)-2-(1-(isopropylsulfonyl)piperidin-4-yl)acetamide

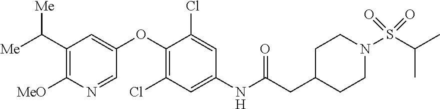 [SMILES: COc1ncc(Oc2c(Cl)cc(NC(=O)CC3CCN(S(=O)(=O)C(C)C)CC3)cc2Cl)cc1C(C)C]

### Intermediate 17C: 5-(2,6-dichloro-4-nitrophenoxy)-3-isopropyl-2-methoxypyridine

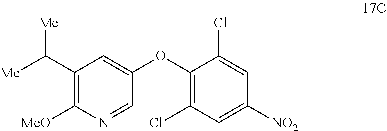 [SMILES: COc1ncc(Oc2c(Cl)cc([N+](=O)[O-])cc2Cl)cc1C(C)C]

To a solution of 5-isopropyl-6-methoxypyridin-3-ol 17A (2.5 g, 14.95 mmol) and 1,3-dichloro-2-fluoro-5-nitrobenzene 17B (3.14 g, 14.95 mmol) in DMF (10 mL) was added potassium carbonate (3.10 g, 22.43 mmol). The reaction mixture was stirred at 60° C. for 1 h. The reaction mixture was diluted with cold water and extracted with EtOAc (3×). The combined organic extracts were concentrated to give desired product 17C (5.1 g, 95% yield). LCMS m/z 359.2 (M+H); rt 1.20 min; Method C; ¹H NMR (400 MHz, CHLOROFORM-d) δ 8.32 (s, 2H), 7.49-7.43 (m, 1H), 7.20-7.16 (m, 1H), 3.92 (s, 3H), 3.24-3.12 (m, 1H), 1.23 (d, J=7.0 Hz, 6H).

### Intermediate 17D: 3,5-dichloro-4-((5-isopropyl-6-methoxypyridin-3-yl)oxy)aniline

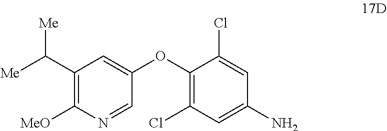 [SMILES: COc1ncc(Oc2c(Cl)cc(N)cc2Cl)cc1C(C)C]

To a suspension of 5-(2,6-dichloro-4-nitrophenoxy)-3-isopropyl-2-methoxypyridine 17C (5.10 g, 14.28 mmol) in EtOH (100 mL) and water (25 mL) was added iron (7.97 g, 143 mmol) and ammonium chloride (7.64 g, 143 mmol). The reaction mixture was stirred at 80° C. for 1.5 h. The mixture was diluted with EtOAc (100 mL) and filtered through Celite. The filtrate was washed with water, brine, dried over magnesium sulfate and concentrated to give the desired aniline 17D (4.4 g, 94% yield) as a tan colored solid. LCMS m/z 327.4 (M+H); rt 1.08 min; Method C; ¹H NMR (400 MHz, CHLOROFORM-d) δ 7.76 (d, J=2.8 Hz, 1H), 7.42 (d, J=2.7 Hz, 1H), 7.29 (s, 2H), 6.70 (s, 2H), 3.99 (s, 3H), 3.81 (br s, 2H).

### Intermediate 17E: N-(3,5-dichloro-4-((5-isopropyl-6-methoxypyridin-3-yl)oxy)phenyl)-2-(piperidin-4-yl)acetamide

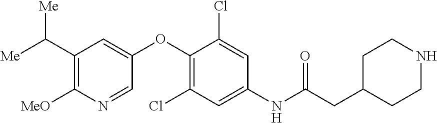 [SMILES: COc1ncc(Oc2c(Cl)cc(NC(=O)CC3CCNCC3)cc2Cl)cc1C(C)C]

To a solution of 2-(1-(tert-butoxycarbonyl)piperidin-4-yl)acetic acid (1.78 g, 7.33 mmol) and 3,5-dichloro-4-((5-isopropyl-6-methoxypyridin-3-yl)oxy)aniline 17D (2.40 g, 7.33 mmol) in DMF (20 mL) were added HATU (4.18 g, 11.00 mmol) and DIEA (3.84 mL, 22.00 mmol). The reaction mixture was stirred at 65° C. for 18 h. The reaction mixture was diluted with water and extracted with EtOAc (3×). The combined organic extracts were dried over magnesium sulfate and concentrated. The residue was purified by silica gel chromatography using 0-50% EtOAc in hexanes to obtain desired amide (2.84 g, 70% yield) as a light yellow solid. LCMS m/z 552.5 (M+H); rt 1.23 min; Method C.

To the solution of the above compound tert-butyl 4-(2-((3,5-dichloro-4-((5-isopropyl-6-methoxypyridin-3-yl)oxy)phenyl)amino)-2-oxoethyl)piperidine-1-carboxylate (2.84 g, 5.14 mmol) in DCM (100 mL) at 0° C. was added hydrogen chloride, 4 M in 1,4-dioxane (12.85 mL, 51.4 mmol). The reaction mixture was stirred at rt for 2 h. The reaction mixture was concentrated in vacuo to give an off-white solid. The residue was dissolved in EtOAc and washed with sat. sodium bicarbonate and brine. The organic layer was dried over magnesium sulfate and concentrated in vacuo to give the desired product 17E as an off white solid (2.38 g, 95% yield). LCMS m/z 452.3 (M+H); rt 0.95 min; Method C.

### Example 24: N-(3,5-dichloro-4-((5-isopropyl-6-methoxypyridin-3-yl)oxy)phenyl)-2-(1-(isopropylsulfonyl)piperidin-4-yl)acetamide

To a solution of N-(3,5-dichloro-4-((5-isopropyl-6-methoxypyridin-3-yl)oxy)phenyl)-2-(piperidin-4-yl)acetamide 17E (20 mg, 0.044 mmol) and 4-methylmorpholine (13.4 mg, 0.133 mmol) in DCM (1 mL) was added propane-2-sulfonyl chloride (7.57 mg, 0.053 mmol). The reaction mixture was stirred at rt overnight and purified by reverse phase preparative LC/MS to obtain Example 24 (9.8 mg, 40% yield). ¹H NMR (500 MHz, DMSO-d₆) δ 7.83 (s, 2H), 7.42 (d, J=2.7 Hz, 1H), 7.21 (d, J=2.7 Hz, 1H), 3.83 (s, 3H), 3.63 (br d, J=12.5 Hz, 2H), 3.51 (br s, 2H), 3.33-3.24 (m, 1H), 3.12-3.01 (m, 1H), 2.93-2.83 (m, 2H), 2.30 (d, J=7.0 Hz, 2H), 2.01-1.88 (m, 1H), 1.73 (br d, J=11.3 Hz, 2H), 1.21 (d, J=6.7 Hz, 6H), 1.14 (d, J=6.7 Hz, 6H); LCMS m/z 558.2 (M+H); rt 2.50 min; Method A.

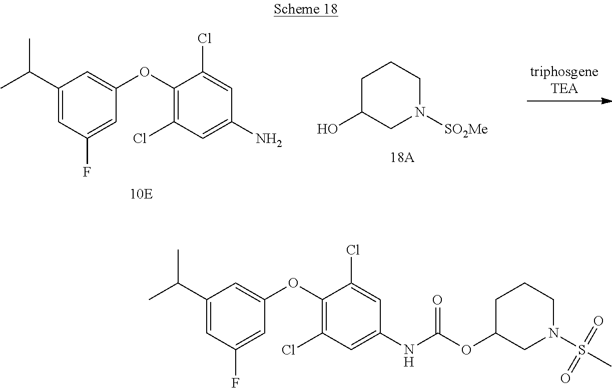 [SMILES: CC(C)c1cc(F)cc(Oc2c(Cl)cc(N)cc2Cl)c1 ; CS(=O)(=O)N1CCCC(O)C1 ; CC(C)c1cc(F)cc(Oc2c(Cl)cc(NC(=O)OC3CCCN(S(C)(=O)=O)C3)cc2Cl)c1]

### Example 25

### 1-(methylsulfonyl)piperidin-3-yl (3,5-dichloro-4-(3-fluoro-5-isopropylphenoxy)phenyl)carbamate

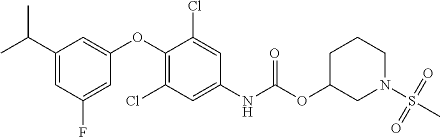 [SMILES: CC(C)c1cc(F)cc(Oc2c(Cl)cc(NC(=O)OC3CCCN(S(C)(=O)=O)C3)cc2Cl)c1]

Triphosgene (54.4 mg, 0.183 mmol) was added to a solution of 3,5-dichloro-4-(3-fluoro-5-isopropylphenoxy)aniline 10E (48 mg, 0.153 mmol) and triethylamine (85 μl, 0.611 mmol) in dichloromethane (1.5 mL) and the reaction mixture was stirred at room temperature for 0.5 h. 1-(methylsulfonyl)piperidin-3-ol 18A (41.1 mg, 0.229 mmol) was then added and the reaction mixture was stirred at room temperature for 1 h. The reaction was quenched with methanol and the reaction mixture was evaporated in vacuo. The crude product was purified by reverse phase preparative LC/MS to obtain Example 25 (35.1 mg, 44% yield). LCMS m/z 519.2 (M+H); rt 2.48 min; Method G.

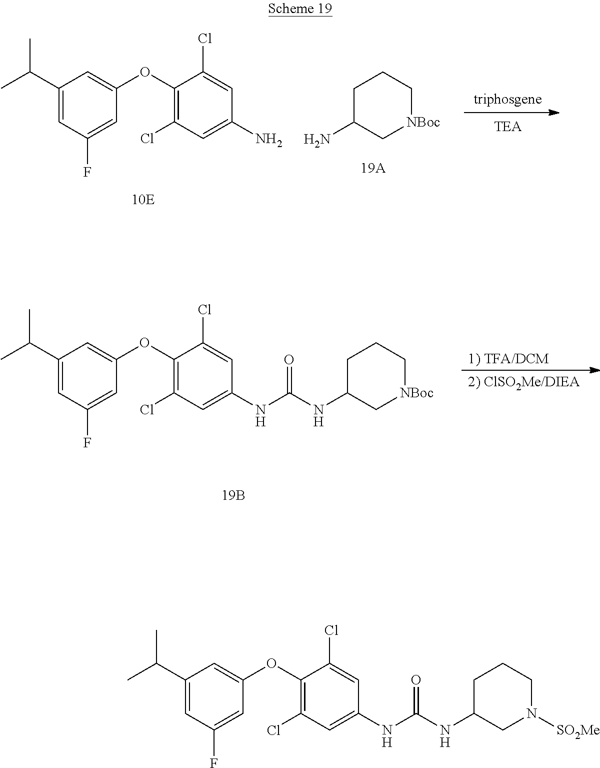 [SMILES: CC(C)c1cc(F)cc(Oc2c(Cl)cc(N)cc2Cl)c1 ; CC(C)(C)OC(=O)N1CCCC(N)C1 ; CC(C)c1cc(F)cc(Oc2c(Cl)cc(NC(=O)NC3CCCN(C(=O)OC(C)(C)C)C3)cc2Cl)c1 ; CC(C)c1cc(F)cc(Oc2c(Cl)cc(NC(=O)NC3CCCN(S(C)(=O)=O)C3)cc2Cl)c1]

### Example 26

### 1-(3,5-dichloro-4-(3-fluoro-5-isopropylphenoxy)phenyl)-3-(1-(methylsulfonyl)piperidin-3-yl)urea

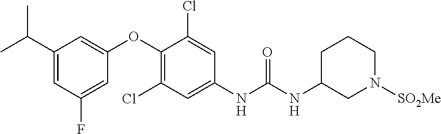 [SMILES: CC(C)c1cc(F)cc(Oc2c(Cl)cc(NC(=O)NC3CCCN(S(C)(=O)=O)C3)cc2Cl)c1]

### Intermediate 19B: tert-butyl 3-(3-(3,5-dichloro-4-(3-fluoro-5-isopropyl Phenoxy)phenyl)ureido)piperidine-1-carboxylate

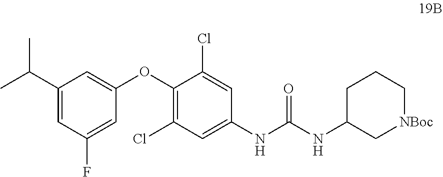 [SMILES: CC(C)c1cc(F)cc(Oc2c(Cl)cc(NC(=O)NC3CCCN(C(=O)OC(C)(C)C)C3)cc2Cl)c1]

Triphosgene (54.4 mg, 0.183 mmol) was added to a solution of 3,5-dichloro-4-(3-fluoro-5-isopropylphenoxy)aniline 10E (48 mg, 0.153 mmol) and triethylamine (85 μl, 0.611 mmol) in dichloromethane (1.5 mL) and the reaction mixture was stirred at room temperature for 0.5 hour. tert-butyl 3-aminopiperidine-1-carboxylate 19A (45.9 mg, 0.229 mmol) was then added and the reaction mixture was stirred at room temperature for 2 h. Solvent was evaporated in vacuo and the crude product was purified by flash chromatography on silica gel using an automated ISCO system (24 g column, eluting with 0-80% ethyl acetate/hexanes). Obtained tert-butyl 3-(3-(3,5-dichloro-4-(3-fluoro-5-isopropylphenoxy)phenyl)ureido)piperidine-1-carboxylate 19B (71 mg, 0.131 mmol, 86% yield) as a foam. LCMS m/z 539.8 (M+H); rt 1.2 min; Method C.

### Example 26: 1-(3,5-dichloro-4-(3-fluoro-5-isopropylphenoxy)phenyl)-3-(1-(methylsulfonyl)piperidin-3-yl)urea

tert-butyl 3-(3-(3,5-dichloro-4-(3-fluoro-5-isopropylphenoxy)phenyl)ureido)piperidine-1-carboxylate 19B (71 mg, 0.131 mmol) was treated with 25% TFA in DCE (1 mL) at room temperature for 1 h. Solvent was evaporated in vacuo and the residue was redissolved in dichloromethane and evaporated (repeated once). The crude intermediate was dissolved in dichloromethane and triethylamine (0.2 mL) was added to neutralize the acid, concentrated and dried under vacuum for 1 h.

The crude intermediate was dissolved in dichloromethane (5 mL) and DIEA (92 μl, 0.525 mmol) was added. The mixture was cooled to 0° C. and methanesulfonyl chloride (20.47 μl, 0.263 mmol) was added. The resulting mixture stirred at room temperature for 1 h. Solvent was evaporated in vacuo. The residue was purified by reverse phase preparative LC/MS to obtain Example 26 (61.3 mg, 88% yield). LCMS m/z 518.3 (M+H); rt 2.29 min; Method G.

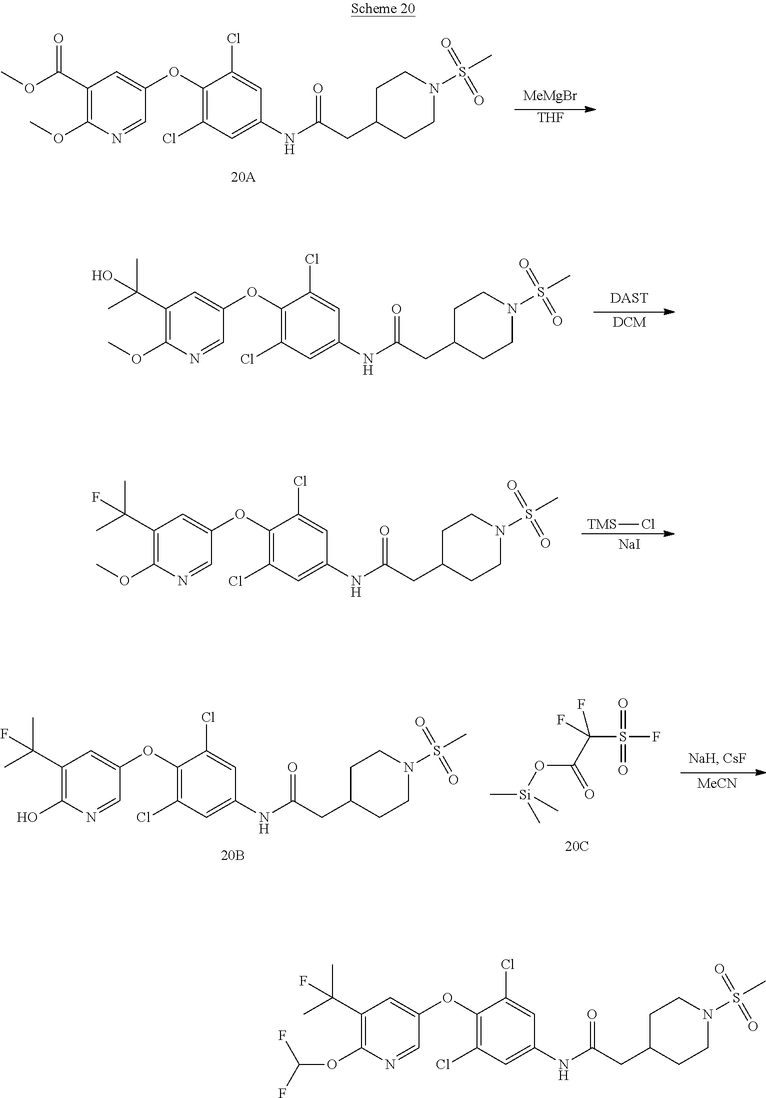

### Example 27

### N-(3,5-dichloro-4-((5-(2-hydroxypropan-2-yl)-6-methoxypyridin-3-yl)oxy)phenyl)-2-(1-(methylsulfonyl)piperidin-4-yl)acetamide

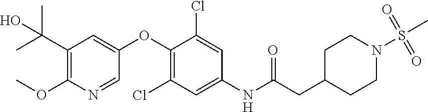 [SMILES: COc1ncc(Oc2c(Cl)cc(NC(=O)CC3CCN(S(C)(=O)=O)CC3)cc2Cl)cc1C(C)(C)O]

3 M methylmagnesium bromide in ether (0.329 mL, 0.988 mmol) was added to a solution of methyl 5-(2,6-dichloro-4-(2-(1-(methylsulfonyl)piperidin-4-yl)acetamido)phenoxy)-2-methoxynicotinate (0.090 g, 0.165 mmol, (prepared using the procedure described for Example 24) in THE (5.49 mL) at −78° C. and the reaction mixture was stirred at room temperature for 1 h. The reaction was quenched with saturated ammonium chloride and extracted with ethyl acetate (3×). The organic layer was dried over magnesium sulfate and concentrated in vacuo. The crude product was purified by flash chromatography on silica gel using an automated ISCO system (24 g gold column, eluting with 5-100% ethyl acetate/hexanes). Obtained N-(3,5-dichloro-4-((5-(2-hydroxypropan-2-yl)-6-methoxypyridin-3-yl)oxy)phenyl)-2-(1-(methylsulfonyl)piperidin-4-yl)acetamide Example 27 (77 mg, 0.138 mmol, 84% yield) as a foam. LCMS m/z 546.2 (M+H); rt 0.94 min; Method C.

### Example 28

### N-(3,5-dichloro-4-((5-(2-fluoropropan-2-yl)-6-methoxypyridin-3-yl)oxy)phenyl)-2-(1-(methylsulfonyl)piperidin-4-yl)acetamide

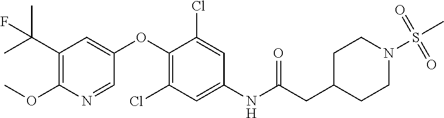 [SMILES: COc1ncc(Oc2c(Cl)cc(NC(=O)CC3CCN(S(C)(=O)=O)CC3)cc2Cl)cc1C(C)(C)F]

DAST (0.020 mL, 0.150 mmol) was added to a solution of N-(3,5-dichloro-4-((5-(2-hydroxypropan-2-yl)-6-methoxypyridin-3-yl)oxy)phenyl)-2-(1-(methylsulfonyl)piperidin-4-yl)acetamide (41 mg, 0.075 mmol, Example 27) in dichloromethane (1 mL) at −78° C. and the reaction mixture was stirred at −78° C. for 10 min, slowly warmed up to room temperature and was stirred at room temperature for 0.5 h. The reaction mixture was quenched with methanol and saturated sodium bicarbonate solution (2 mL) was added. The layers were separated and aqueous layer was extracted with dichloromethane two more times. The combined organic layers were dried over magnesium sulfate and concentrated in vacuo. The crude product was purified by reverse phase preparative LC/MS to obtain Example 28 (25.8 mg, 63% yield). LCMS m/z 548.1 (M+H); rt 2.25 min; Method G.

### Example 29

### N-(3,5-dichloro-4-((6-(difluoromethoxy)-5-(2-fluoropropan-2-yl)pyridin-3-yl)oxy)phenyl)-2-(1-(methylsulfonyl)piperidin-4-yl)acetamide

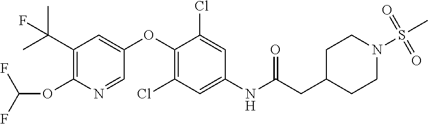 [SMILES: CC(C)(F)c1cc(Oc2c(Cl)cc(NC(=O)CC3CCN(S(C)(=O)=O)CC3)cc2Cl)cnc1OC(F)F]

### Intermediate 20B: N-(3,5-dichloro-4-((5-(2-fluoropropan-2-yl)-6-hydroxypyridin-3-yl)oxy)phenyl)-2-(1-(methylsulfonyl)piperidin-4-yl)acetamide

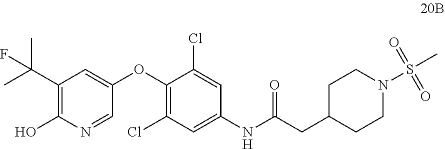 [SMILES: CC(C)(F)c1cc(Oc2c(Cl)cc(NC(=O)CC3CCN(S(C)(=O)=O)CC3)cc2Cl)cnc1O]

TMS-Cl (608 μl, 4.76 mmol) was added to a solution of N-(3,5-dichloro-4-((5-(2-fluoropropan-2-yl)-6-methoxypyridin-3-yl)oxy)phenyl)-2-(1-(methylsulfonyl)piperidin-4-yl)acetamide Example 28 (261 mg, 0.476 mmol) and sodium iodide (713 mg, 4.76 mmol) in acetonitrile (9518 μl) at room temperature and the reaction mixture was stirred at room temperature overnight. The reaction was quenched with methanol and saturated sodium bicarbonate was added. The mixture was extracted with 4/1 dichloromethane/methanol three times. The organic layer was dried over magnesium sulfate and concentrated in vacuo, The crude was dissolved in methanol/dichloromethane (1/4) and adsorbed onto silica (24 g) and purified by flash chromatography on silica gel using an automated ISCO system (40 g column, eluting with 0-100% ethyl acetate/hexanes) to give N-(3,5-dichloro-4-((5-(2-fluoropropan-2-yl)-6-hydroxypyridin-3-yl)oxy)phenyl)-2-(1-(methylsulfonyl)piperidin-4-yl)acetamide 20B (241 mg, 0.451 mmol, 95% yield) as a brown oil. LCMS m/z 534.2 (M+H); rt 0.88 min; Method C.

### Example 29: N-(3,5-dichloro-4-((6-(difluoromethoxy)-5-(2-fluoropropan-2-yl)pyridin-3-yl)oxy)phenyl)-2-(1-(methylsulfonyl)piperidin-4-yl)acetamide

A mixture of N-(3,5-dichloro-4-((5-(2-fluoropropan-2-yl)-6-hydroxypyridin-3-yl)oxy)phenyl)-2-(1-(methylsulfonyl)piperidin-4-yl)acetamide 20B (40 mg, 0.075 mmol) and NaH (3.89 mg, 0.097 mmol) in acetonitrile (748 μL) was stirred at room temperature for 0.5 h and CsF (1.137 mg, 7.48 μmol) was added followed by slow addition of trimethylsilyl 2,2-difluoro-2-(fluorosulfonyl)acetate (19.17 μl, 0.097 mmol). The reaction mixture was stirred at room temperature for 15 min. The reaction was quenched with water, partitioned between ethyl acetate and water. The layers were separated and aqueous layer was extracted with ethyl acetate two more times. The combined organic layers were dried over magnesium sulfate and concentrated in vacuo. The crude residue was purified by reverse phase preparative LC/MS to obtain Example 29 (61.3 mg, 88% yield). LCMS m/z 584.2 (M+H); rt 2.30 min; Method G.

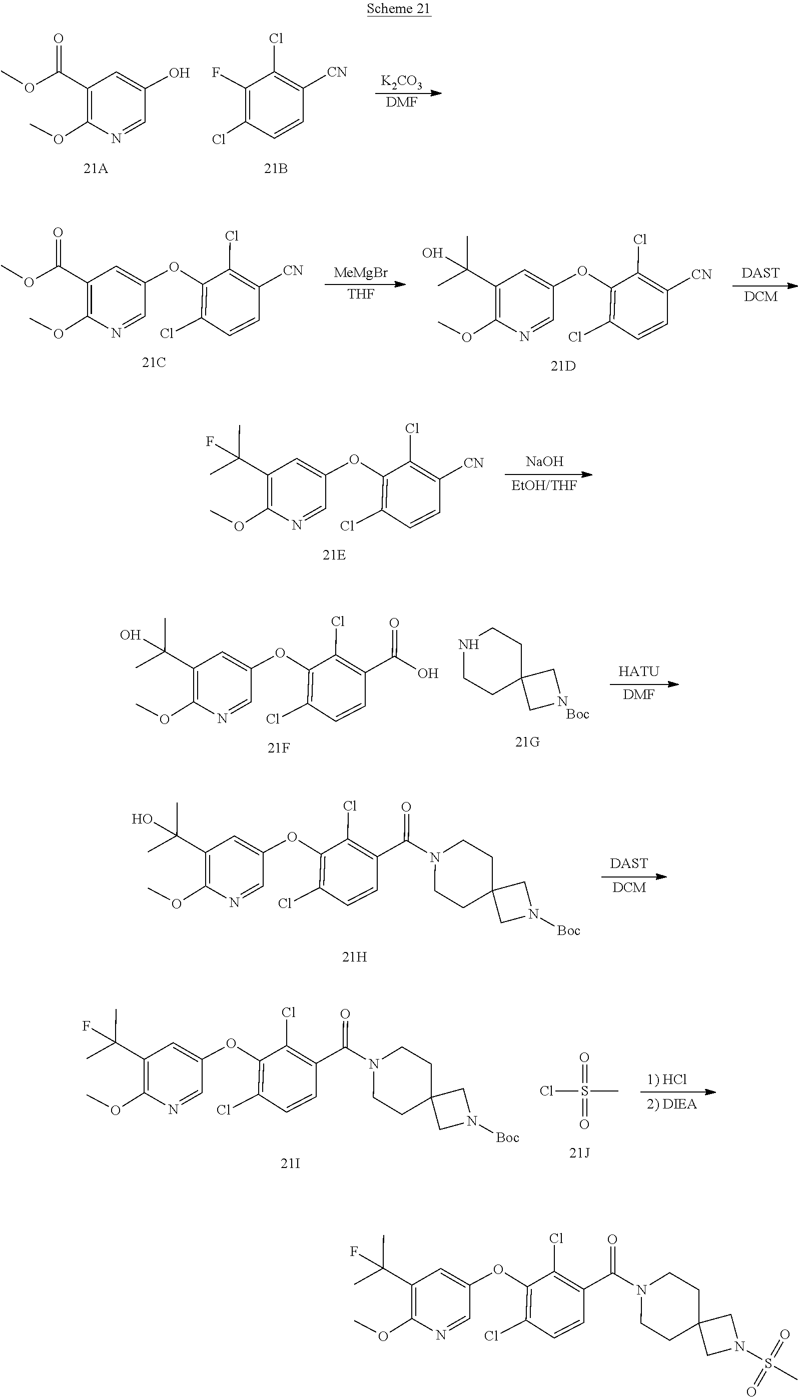

### Example 30

### (2,4-dichloro-3-((5-(2-fluoropropan-2-yl)-6-methoxypyridin-3-yl)oxy)phenyl)(2-(methylsulfonyl)-2,7-diazaspiro[3.5]nonan-7-yl)methanone

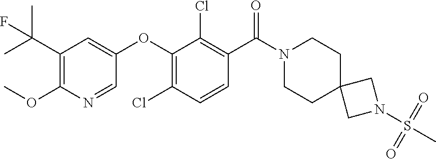 [SMILES: COc1ncc(Oc2c(Cl)ccc(C(=O)N3CCC4(CC3)CN(S(C)(=O)=O)C4)c2Cl)cc1C(C)(C)F]

### Intermediate 21C: methyl 5-(2,6-dichloro-3-cyanophenoxy)-2-methoxynicotinate

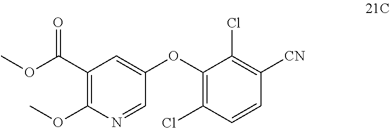 [SMILES: COC(=O)c1cc(Oc2c(Cl)ccc(C#N)c2Cl)cnc1OC]

A mixture of methyl 5-hydroxy-2-methoxynicotinate 21A (0.56 g, 3.06 mmol), 2,4-dichloro-3-fluorobenzonitrile 21B (0.697 g, 3.67 mmol) and potassium carbonate (0.845 g, 6.11 mmol) in DMF (10.19 mL) was heated to 80° C. for 2 h. The reaction mixture was diluted with ethyl acetate and washed with water and brine. The organic layer was dried over magnesium sulfate and concentrated in vacuo. The crude product was purified by flash chromatography on silica gel using an automated ISCO system (80 g column, eluting with 0-100% ethyl acetate/hexanes) to give methyl 5-(2,6-dichloro-3-cyanophenoxy)-2-methoxynicotinate 21C (0.597 g, 1.690 mmol, 55.3% yield) as a white solid. LCMS m/z 353.1 (M+H); rt 0.97 min; Method C.

### Intermediate 21D: 2,4-dichloro-3-((5-(2-hydroxypropan-2-yl)-6-methoxypyridin-3-yl)oxy)benzonitrile

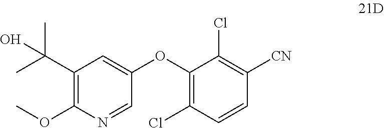 [SMILES: COc1ncc(Oc2c(Cl)ccc(C#N)c2Cl)cc1C(C)(C)O]

3 M methylmagnesium bromide in ether (1.963 mL, 5.89 mmol) was added to a solution of methyl 5-(2,6-dichloro-3-cyanophenoxy)-2-methoxynicotinate 21C (0.52 g, 1.472 mmol) in THE (14.72 mL) dropwise at 0° C. and the reaction mixture was stirred at 0° C. for 2 h. The reaction was quenched with saturated ammonium chloride and extracted with ethyl acetate (3×). The organic layer was dried over magnesium sulfate and concentrated in vacuo. The crude ether 21D was used as such in the next step. LCMS m/z 353.1 (M+H); rt 0.98 min; Method C.

### Intermediate 21E: 2,4-dichloro-3-((5-(2-fluoropropan-2-yl)-6-methoxypyridin-3-yl)oxy)benzonitrile

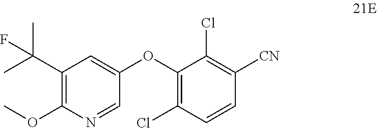 [SMILES: COc1ncc(Oc2c(Cl)ccc(C#N)c2Cl)cc1C(C)(C)F]

DAST (0.389 mL, 2.94 mmol) was added to a suspension of 2,4-dichloro-3-((5-(2-hydroxypropan-2-yl)-6-methoxypyridin-3-yl)oxy)benzonitrile 21D (0.52 g, 1.472 mmol) in dichloromethane (14.72 mL) at −78° C., the reaction mixture was stirred at −78° C. for 10 min and then warmed up to room temperature and stirred for 1 h. The reaction mixture was quenched with methanol and saturated sodium bicarbonate was added. The resulting mixture was extracted with dichloromethane (3×). The organic layer was dried over magnesium sulfate and concentrated in vacuo. The crude product was purified by flash chromatography on silica gel using an automated ISCO system (80 g column, eluting with 0-30% ethyl acetate/hexanes) to obtain 2,4-dichloro-3-((5-(2-fluoropropan-2-yl)-6-methoxypyridin-3-yl)oxy)benzonitrile 21E (0.375 g, 1.056 mmol, 71.7% yield). LCMS m/z 355.0 (M+H); rt 1.13 min; Method C.

### Intermediate 21F: 2,4-dichloro-3-((5-(2-hydroxypropan-2-yl)-6-methoxypyridin-3-yl)oxy)benzoic Acid

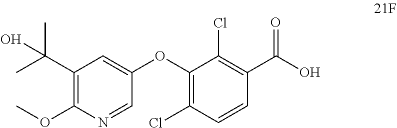 [SMILES: COc1ncc(Oc2c(Cl)ccc(C(=O)O)c2Cl)cc1C(C)(C)O]

A mixture of 2,4-dichloro-3-((5-(2-fluoropropan-2-yl)-6-methoxypyridin-3-yl)oxy)benzonitrile 21E (0.375 g, 1.056 mmol) and 3 M aqueous NaOH (2.82 mL, 8.45 mmol) in EtOH (3.52 mL)/THF (1.76 mL) was stirred at 85° C. overnight. The reaction mixture was cooled to room temperature and neutralized with concentrated HCl to pH 5. The organic solvents were evaporated in vacuo and the aqueous solution was lyophilized to give the crude 21F which was used without purification. LCMS m/z 372.0 (M+H); rt 0.88 min; Method C.

### Intermediate 21H: tert-butyl 7-(2,4-dichloro-3-((5-(2-hydroxypropan-2-yl)-6-methoxypyridin-3-yl)oxy)benzoyl)-2,7-diazaspiro[3.5]nonane-2-carboxylate

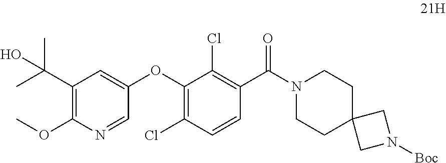 [SMILES: COc1ncc(Oc2c(Cl)ccc(C(=O)N3CCC4(CC3)CN(C(=O)OC(C)(C)C)C4)c2Cl)cc1C(C)(C)O]

HATU (73.6 mg, 0.193 mmol) was added to a solution of 2,4-dichloro-3-((5-(2-hydroxypropan-2-yl)-6-methoxypyridin-3-yl)oxy)benzoic acid 21F (80 mg, 0.097 mmol), tert-butyl 2,7-diazaspiro[3.5]nonane-2-carboxylate (43.8 mg, 0.193 mmol) and DIEA (84 μL, 0.484 mmol) in DMF (967 μL) at room temperature and the reaction mixture was stirred at room temperature overnight. The reaction mixture was diluted with ethyl acetate and washed with water and brine. The organic layer was dried over magnesium sulfate and concentrated in vacuo. The crude 21H was purified by flash chromatography on silica gel using an automated ISCO system (24 g column, eluting with 0-6% 2 N ammonia in methanol/dichloromethane) to obtain tert-butyl 7-(2,4-dichloro-3-((5-(2-hydroxypropan-2-yl)-6-methoxypyridin-3-yl)oxy)benzoyl)-2,7-diazaspiro[3.5]nonane-2-carboxylate 21H (37 mg, 0.064 mmol, 65.9% yield). LCMS m/z 580.2 (M+H); rt 1.05 min; Method C.

### Intermediate 21I: tert-butyl 7-(2,4-dichloro-3-((5-(2-fluoropropan-2-yl)-6-methoxypyridin-3-yl)oxy)benzoyl)-2,7-diazaspiro[3.5]nonane-2-carboxylate

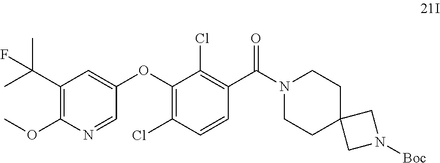 [SMILES: COc1ncc(Oc2c(Cl)ccc(C(=O)N3CCC4(CC3)CN(C(=O)OC(C)(C)C)C4)c2Cl)cc1C(C)(C)F]

DAST (16.84 μl, 0.127 mmol) was added to a solution of tert-butyl 7-(2,4-dichloro-3-((5-(2-hydroxypropan-2-yl)-6-methoxypyridin-3-yl)oxy)benzoyl)-2,7-diazaspiro[3.5]nonane-2-carboxylate 21H (37 mg, 0.064 mmol) in dichloromethane (1275 μl) at −78° C. and the resulting mixture was stirred for 10 min and then warmed to room temperature and stirred for another 1 h. The reaction was quenched with methanol and water. The reaction mixture was partitioned between dichloromethane and water. The layers were separated and aqueous layer was extracted with dichloromethane two more times. The combined organic layers were dried over magnesium sulfate and concentrated in vacuo. The resulting crude 21I was used without purification in the next step. LCMS m/z 582.2 (M+H); rt 1.16 min; Method C.

### Example 30: (2,4-dichloro-3-((5-(2-fluoropropan-2-yl)-6-methoxypyridin-3-yl)oxy)phenyl)(2-(methylsulfonyl)-2,7-diazaspiro[3.5]nonan-7-yl)methanone

tert-butyl 7-(2,4-dichloro-3-((5-(2-fluoropropan-2-yl)-6-methoxypyridin-3-yl)oxy)benzoyl)-2,7-diazaspiro[3.5]nonane-2-carboxylate 21I (37.3 mg, 0.064 mmol) was treated with 4 M HCl in dioxane (320 μl, 1.280 mmol) at room temperature for 1 h. Solvent was evaporated and the crude was dried under high vacuum for 0.5 h.

To the crude intermediate in dichloromethane (1280 μl), DIEA (44.7 μl, 0.256 mmol) and methanesulfonyl chloride (9.97 μl, 0.128 mmol) were added. The reaction mixture was stirred at room temperature for 0.5 h. The reaction mixture was partitioned between dichloromethane and water. The layers were separated and aqueous layer was extracted with dichloromethane/methanol (3/1) two more times. The combined organic layers were dried over magnesium sulfate and concentrated in vacuo. The crude product was purified by reverse phase preparative LC/MS to obtain Example 30 (16.8 mg, 47% yield). LCMS m/z 560.2 (M+H); rt 2.08 min; Method G.

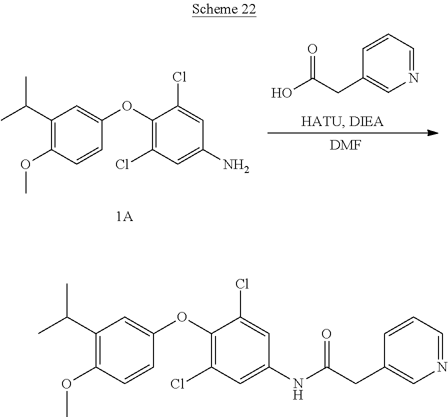

### Example 31

### N-(3,5-dichloro-4-(3-isopropyl-4-methoxyphenoxy)phenyl)-2-(pyridin-3-yl)acetamide

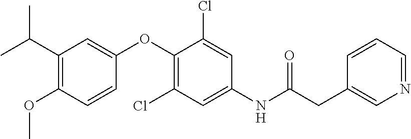 [SMILES: COc1ccc(Oc2c(Cl)cc(NC(=O)Cc3cccnc3)cc2Cl)cc1C(C)C]

### Example 31: N-(3,5-dichloro-4-(3-isopropyl-4-methoxyphenoxy)phenyl)-2-(pyridin-3-yl)acetamide

To a 1 dram vial containing 2-(pyridin-3-yl)acetic acid hydrochloride (42.6 mg, 0.245 mmol) was added HATU (69.9 mg, 0.184 mmol) in DMF (0.30 mL), followed by 3,5-dichloro-4-(3-isopropyl-4-methoxyphenoxy)aniline 1A (40.0 mg, 0.123 mmol) in DMF (0.30 mL). DIEA (0.11 mL, 0.613 mmol) was then added, and the mixture was allowed to stir at room temperature for 3 days. The reaction was then quenched with a drop of water, diluted with DMF, and purified by reversed phase HPLC to afford N-(3,5-dichloro-4-(3-isopropyl-4-methoxyphenoxy)phenyl)-2-(pyridin-3-yl)acetamide Example 31 (29.7 mg, 0.067 mmol, 54% yield) as the trifluoroacetic acid salt. LCMS m/z 445.3 (M+H); rt 1.97 min; conditions B; ¹H NMR (500 MHz, DMSO-d₆) δ 10.68 (s, 1H), 8.61 (br. s., 2H), 7.94 (d, J=7.9 Hz, 1H), 7.81 (s, 2H), 7.56 (br. s., 1H), 6.83 (d, J=9.0 Hz, 1H), 6.76 (d, J=2.9 Hz, 1H), 6.40 (dd, J=8.8, 3.1 Hz, 1H), 3.81 (s, 2H), 3.72 (s, 3H), 3.22-3.14 (m, 1H), 1.10 (d, J=6.9 Hz, 6H).

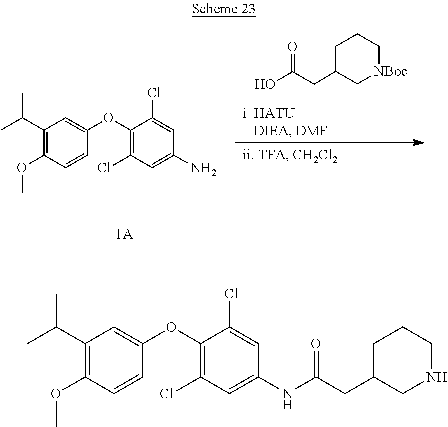 [SMILES: CC(C)(C)OC(=O)N1CCCC(CC(=O)O)C1 ; COc1ccc(Oc2c(Cl)cc(N)cc2Cl)cc1C(C)C ; COc1ccc(Oc2c(Cl)cc(NC(=O)CC3CCCNC3)cc2Cl)cc1C(C)C]

### Example 32

### N-(3,5-dichloro-4-(3-isopropyl-4-methoxyphenoxy)phenyl)-2-(piperidin-3-yl)acetamide

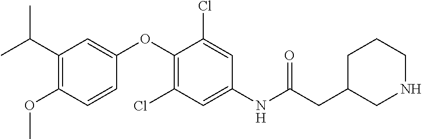 [SMILES: COc1ccc(Oc2c(Cl)cc(NC(=O)CC3CCCNC3)cc2Cl)cc1C(C)C]

To a 1 dram vial containing 2-(1-(tert-butoxycarbonyl)piperidin-3-yl)acetic acid (40.6 mg, 0.167 mmol) was added HATU (47.6 mg, 0.125 mmol) in DMF (0.25 mL), followed by 3,5-dichloro-4-(3-isopropyl-4-methoxyphenoxy)aniline 1A (27.2 mg, 0.083 mmol) in DMF (0.25 mL). DIEA (44 μL, 0.250 mmol) was then added, and the mixture was allowed to stir at room temperature over the weekend for 3 days. The reaction was then quenched with a drop of water, and concentrated in vacuo. The residue was partitioned between CH₂Cl₂ (5 mL) and 1.5 M aqueous K₂HPO₄ (10 mL). The aqueous phase was then back-extracted once with CH₂Cl₂ (5 mL), and the combined organic layers were dried (MgSO₄), filtered, and concentrated in vacuo.

The residue was dissolved in CH₂Cl₂ (1.0 mL), then trifluoroacetic acid (50 μL, 0.649 mmol) was added, and the resulting mixture was stirred at room temperature. After 1 hour, another portion of trifluoroacetic acid (50 μL, 0.649 mmol) was added. After stirring at room temperature for another 16 hours, the reaction was concentrated in vacuo, diluted with DMF, and purified by reversed phase HPLC to afford racemic N-(3,5-dichloro-4-(3-isopropyl-4-methoxyphenoxy)phenyl)-2-(piperidin-3-yl)acetamide Example 32 (34.2 mg, 0.076 mmol, 91% yield) as the acetic acid salt. LCMS m/z 451.3 (M+H); rt 1.92 min; conditions B; ¹H NMR (500 MHz, DMSO-d₆) δ 7.82 (s, 2H), 6.83 (d, J=9.2 Hz, 1H), 6.77 (d, J=3.1 Hz, 1H), 6.41 (dd, J=8.9, 3.1 Hz, 1H), 3.73 (s, 3H), 3.25-3.12 (m, 1H), 3.07-3.00 (m, 1H), 3.00-2.91 (m, 1H), 2.57-2.52 (m, 1H; obscured by DMSO solvent peak), 2.37 (t, J=11.3 Hz, 1H), 2.31-2.19 (m, 2H), 2.05-1.95 (m, 1H), 1.85 (s, 3H), 1.79-1.71 (m, 1H), 1.68-1.59 (m, 1H), 1.53-1.40 (m, 1H), 1.20-1.13 (m, 1H), 1.11 (d, J=7.0 Hz, 6H).

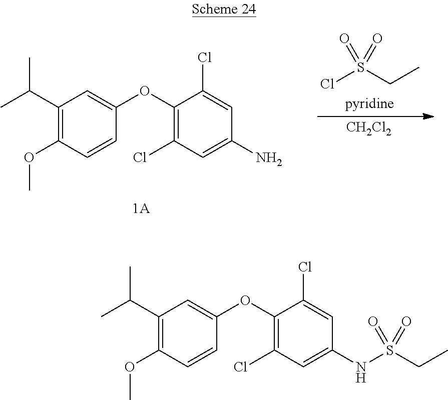

### Example 33

### N-(3,5-dichloro-4-(3-isopropyl-4-methoxyphenoxy)phenyl)ethanesulfonamide

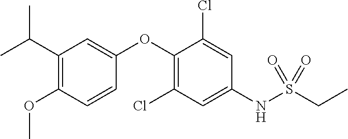 [SMILES: CCS(=O)(=O)Nc1cc(Cl)c(Oc2ccc(OC)c(C(C)C)c2)c(Cl)c1]

To a vial containing a solution of 3,5-dichloro-4-(3-isopropyl-4-methoxyphenoxy)aniline 1A (20 mg, 0.061 mmol) and pyridine (20 μL, 0.247 mmol) in CH₂Cl₂ (0.30 mL) was added ethanesulfonyl chloride (10 μL, 0.106 mmol). The mixture was stirred at room temperature. After 20 hours, the reaction was quenched with a drop of water, diluted with DMF, and purified by reversed phase HPLC to afford N-(3,5-dichloro-4-(3-isopropyl-4-methoxyphenoxy)phenyl)ethanesulfonamide Example 33 (22.0 mg, 0.052 mmol, 85% yield). LCMS m/z 418.1 (M+H); rt 2.27 min; conditions B; ¹H NMR (500 MHz, DMSO-d₆) δ 7.35 (s, 2H), 6.88-6.74 (m, 2H), 6.40 (dd, J=8.9, 3.0 Hz, 1H), 3.72 (s, 3H), 3.28-3.12 (m, 3H), 1.22 (t, J=7.3 Hz, 3H), 1.11 (d, J=6.9 Hz, 6H).

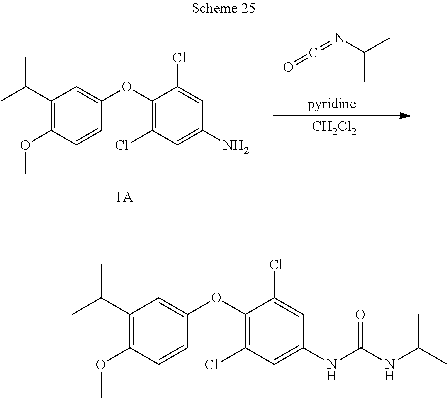

### Example 34

### 1-(3,5-dichloro-4-(3-isopropyl-4-methoxyphenoxy)phenyl)-3-isopropylurea

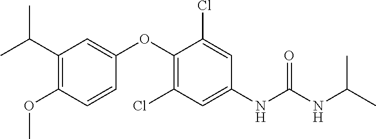 [SMILES: COc1ccc(Oc2c(Cl)cc(NC(=O)NC(C)C)cc2Cl)cc1C(C)C]

To a vial containing a suspension of 3,5-dichloro-4-(3-isopropyl-4-methoxyphenoxy)aniline 1A (20 mg, 0.061 mmol) in CH₂Cl₂ (0.30 mL) was added 2-isocyanatopropane (40 μL, 0.408 mmol) and pyridine (50 μL, 0.618 mmol). After 21 hours, the reaction was quenched with a drop of water, diluted with DMF, and purified by reversed phase HPLC to afford 1-(3,5-dichloro-4-(3-isopropyl-4-methoxyphenoxy)phenyl)-3-isopropylurea Example 34 (24.9 mg, 0.061 mmol, 99% yield). LCMS m/z 411.1 (M+H); rt 2.33 min; conditions B; ¹H NMR (500 MHz, DMSO-d₆) δ 8.70 (s, 1H), 7.59 (s, 2H), 6.83 (d, J=8.9 Hz, 1H), 6.72 (d, J=3.1 Hz, 1H), 6.39 (dd, J=8.9, 2.9 Hz, 1H), 6.22 (d, J=7.6 Hz, 1H), 3.23-3.10 (m, 1H), 1.08 (d, J=5.9 Hz, 12H). Note: some proton resonances obscured by water peak.

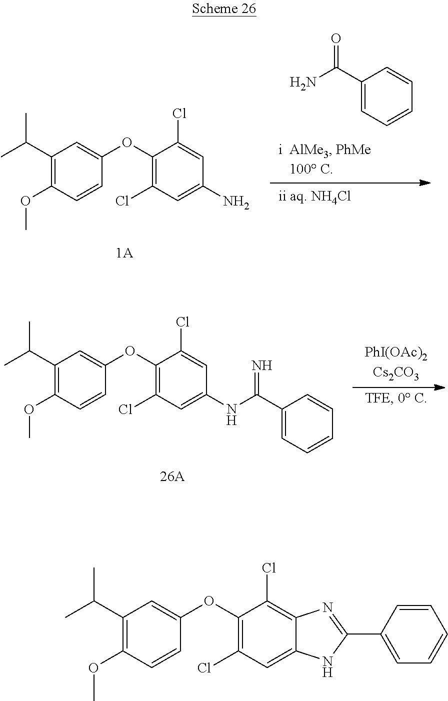 [SMILES: COc1ccc(Oc2c(Cl)cc(N)cc2Cl)cc1C(C)C ; NC(=O)c1ccccc1 ; COc1ccc(Oc2c(Cl)cc(NC(=N)c3ccccc3)cc2Cl)cc1C(C)C ; COc1ccc(Oc2c(Cl)cc3[nH]c(-c4ccccc4)nc3c2Cl)cc1C(C)C]

### Example 35

### 4,6-dichloro-5-(3-isopropyl-4-methoxyphenoxy)-2-phenyl-1H-benzo[d]imidazole

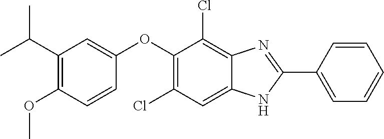 [SMILES: COc1ccc(Oc2c(Cl)cc3[nH]c(-c4ccccc4)nc3c2Cl)cc1C(C)C]

### Intermediate 26A: N-(3,5-dichloro-4-(3-isopropyl-4-methoxyphenoxy) phenyl)benzimidamide

To an oven-dried 1 dram pressure relief vial containing a cooled (0° C.) suspension of 3,5-dichloro-4-(3-isopropyl-4-methoxyphenoxy)aniline 1A (50 mg, 0.153 mmol) and benzamide (18.6 mg, 0.153 mmol) in toluene (0.23 mL) was added trimethylaluminum, 2.0 M in toluene (0.22 mL, 0.429 mmol) dropwise via syringe under nitrogen atmosphere. The resulting mixture was stirred at room temperature for 5 min, then heated to 100° C. After 22 hours, the mixture was cooled to 0° C., diluted with CH₂Cl₂ (4.6 mL), then slowly poured into ice-cold saturated aqueous NH₄Cl (0.30 mL). THE (3.5 mL) was added and the mixture was stirred for 30 min at room temperature, dried (Na₂SO₄), filtered through a Celite plug, and concentrated in vacuo. The crude product was dissolved in a small amount of CH₂Cl₂, adsorbed onto a plug of SiO₂, and purified by flash chromatography (SiO₂, 4 g column, 0-5% MeOH/CH₂Cl₂, 10.6 min gradient, 18 mL/min) to afford N-(3,5-dichloro-4-(3-isopropyl-4-methoxyphenoxy)phenyl)benzimidamide 26A (17.6 mg, 0.041 mmol, 27% yield). LCMS m/z 429.2 (M+H); rt 0.87 min; conditions C.

### Example 35: 4,6-dichloro-5-(3-isopropyl-4-methoxyphenoxy)-2-phenyl-1H-benzo[d]imidazole

To a 1 dram vial containing a cooled (0° C.) suspension of N-(3,5-dichloro-4-(3-isopropyl-4-methoxyphenoxy)phenyl)benzimidamide 26A (17.6 mg, 0.041 mmol) and cesium carbonate (20 mg, 0.061 mmol) in trifluoroethanol (0.30 mL) was added iodobenzene diacetate (18 mg, 0.056 mmol). The resulting mixture was stirred at 0° C. under ambient atmosphere for 45 min. The reaction was then diluted with EtOAc and washed with brine. The aqueous phase was back-extracted with EtOAc three times, and then the combined organic layers were concentrated in vacuo. The residue was taken up in MeOH and purified by reversed phase HPLC to afford 4,6-dichloro-5-(3-isopropyl-4-methoxyphenoxy)-2-phenyl-1H-benzo[d]imidazole Example 35 (5.3 mg, 0.012 mmol, 30% yield). LCMS m/z 427.2 (M+H); rt 2.34 min; conditions B; ¹H NMR (500 MHz, DMSO-d₆) δ 8.19 (d, J=6.7 Hz, 2H), 7.77 (s, 1H), 7.65-7.50 (m, 3H), 6.88-6.74 (m, 2H), 6.41 (dd, J=8.9, 3.1 Hz, 1H), 3.71 (s, 3H), 3.22-3.15 (m, 1H), 1.10 (d, J=7.0 Hz, 6H).

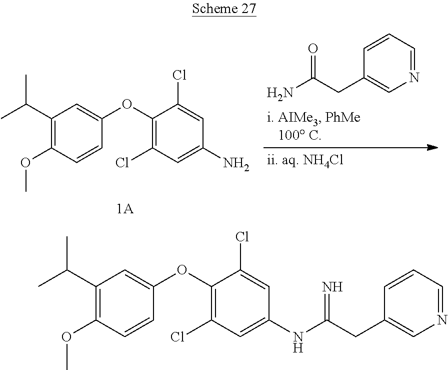 [SMILES: COc1ccc(Oc2c(Cl)cc(N)cc2Cl)cc1C(C)C ; NC(=O)Cc1cccnc1 ; COc1ccc(Oc2c(Cl)cc(NC(=N)Cc3cccnc3)cc2Cl)cc1C(C)C]

### Example 36

### N-(3,5-dichloro-4-(3-isopropyl-4-methoxyphenoxy)phenyl)-2-(pyridin-3-yl)acetimidamide

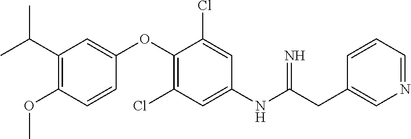 [SMILES: COc1ccc(Oc2c(Cl)cc(NC(=N)Cc3cccnc3)cc2Cl)cc1C(C)C]

To an oven-dried 1 dram pressure relief vial containing a cooled (0° C.) suspension of 3,5-dichloro-4-(3-isopropyl-4-methoxyphenoxy)aniline 1A (50 mg, 0.153 mmol) and 2-(pyridin-3-yl)acetamide (20.9 mg, 0.153 mmol) in toluene (0.23 mL) was added trimethylaluminum, 2.0 M in toluene (0.43 mL, 0.858 mmol) dropwise via syringe under nitrogen atmosphere. The resulting mixture was stirred at room temperature for 30 min, then heated to 100° C. After 20 hours, the reaction was cooled to 0° C., and additional 2-(pyridin-3-yl)acetamide (20.9 mg, 0.153 mmol) was added. The mixture was stirred at room temperature for 5 min, then heated to 100° C. At 40 hours, the mixture was cooled to 0° C., diluted with CH₂Cl₂ (4.6 mL), then slowly poured into ice-cold saturated aqueous NH₄Cl (0.60 mL). THE (3.5 mL) was added and the mixture was stirred for 30 min at room temperature, dried (Na₂SO₄), filtered through a Celite plug, and concentrated in vacuo. The crude product was dissolved in DMF and purified by reversed phase HPLC to afford N-(3,5-dichloro-4-(3-isopropyl-4-methoxyphenoxy)phenyl)-2-(pyridin-3-yl)acetimidamide Example 36 (12.9 mg, 0.028 mmol, 18% yield). LCMS m/z 444.3 (M+H); rt 1.62 min; conditions B.

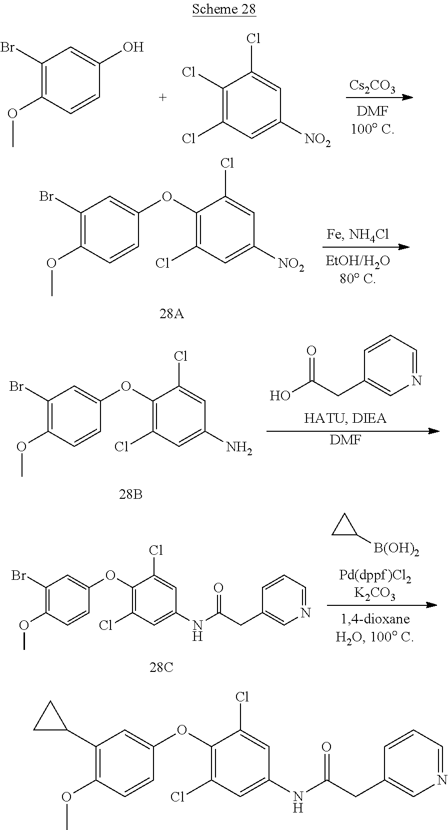

### Example 37

### N-(3,5-dichloro-4-(3-cyclopropyl-4-methoxyphenoxy)phenyl)-2-(pyridin-3-yl)acetamide

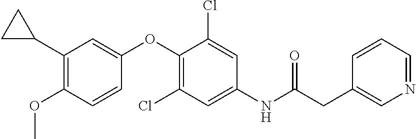 [SMILES: COc1ccc(Oc2c(Cl)cc(NC(=O)Cc3cccnc3)cc2Cl)cc1C1CC1]

### Intermediate 28A: 2-(3-bromo-4-methoxyphenoxy)-1,3-dichloro-5-nitrobenzene

A 2 dram pressure relief vial containing a suspension of 3-bromo-4-methoxyphenol (102 mg, 0.502 mmol), 1,2,3-trichloro-5-nitrobenzene (114 mg, 0.502 mmol), and cesium carbonate (246 mg, 0.754 mmol) in DMF (2.5 mL) was stirred at 100° C. for 2 hours. The reaction was then allowed to cool to room temperature and diluted with water (25 mL). The pH was adjusted to ˜4-5 with 2 N aqueous HCl, and then the aqueous layer was extracted with EtOAc (3×25 mL). The combined organic layers were dried (MgSO4), filtered, and concentrated in vacuo. The crude product was dissolved in a small amount of CH₂Cl₂, adsorbed onto a plug of SiO₂, and purified by flash chromatography (SiO₂, 24 g column, 0% EtOAc/hexanes to 25% EtOAc/hexanes, 11.5 min gradient, 35 mL/min) to afford 2-(3-bromo-4-methoxyphenoxy)-1,3-dichloro-5-nitrobenzene 28A (169 mg, 0.431 mmol, 86% yield) as a clear yellow film. ¹H NMR (400 MHz, CHLOROFORM-d) δ 8.34-8.26 (m, 2H), 7.08 (d, J=2.9 Hz, 1H), 6.84 (d, J=9.0 Hz, 1H), 6.76 (dd, J=8.9, 3.0 Hz, 1H), 3.87 (s, 3H).

### Intermediate 28B: 4-(3-bromo-4-methoxyphenoxy)-3,5-dichloroaniline

To a vial containing a suspension of 2-(3-bromo-4-methoxyphenoxy)-1,3-dichloro-5-nitrobenzene 28A (169 mg, 0.431 mmol) in ethanol (1.2 mL) was added a solution of ammonium chloride (115 mg, 2.16 mmol) in water (0.40 mL), followed by iron powder (241 mg, 4.31 mmol). The resulting mixture was stirred at 80° C. for 30 min. After cooling to room temperature, the reaction was diluted with EtOAc (16 mL), and washed with 1:1 mixture of brine and 1.5 M aqueous K₂HPO₄ (8 mL). The aqueous layer was back-extracted with EtOAc (3×8 mL). The combined organic layers were dried (Na₂SO₄), filtered through a pad of Celite, and concentrated in vacuo to afford crude 4-(3-bromo-4-methoxyphenoxy)-3,5-dichloroaniline 28B. The crude material was carried directly into the next step without further purification.

### Intermediate 28C: N-(4-(3-bromo-4-methoxyphenoxy)-3,5-dichlorophenyl)-2-(pyridin-3-yl)acetamide

To a round bottom flask containing 2-(pyridin-3-yl)acetic acid hydrochloride (150 mg, 0.862 mmol) was added HATU (246 mg, 0.647 mmol) in DMF (1.1 mL), followed by 4-(3-bromo-4-methoxyphenoxy)-3,5-dichloroaniline 28B in DMF (1.1 mL). DIEA (0.38 mL, 2.16 mmol) was then added, and the mixture was allowed to stir at room temperature. After 23 hours, the reaction was diluted with water (20 mL) and extracted with EtOAc (3×20 mL). The combined organic layers were dried (Na₂SO₄), filtered, and concentrated in vacuo. The crude product was dissolved in a small amount of CH₂Cl₂, adsorbed onto a plug of SiO₂, and purified by flash chromatography (SiO₂, 24 g RediSep Rf Gold column, 0-100% EtOAc/hexanes, 23 min gradient, 35 mL/min) to afford N-(4-(3-bromo-4-methoxyphenoxy)-3,5-dichlorophenyl)-2-(pyridin-3-yl)acetamide 28C (265 mg, ˜70% purity, 0.385 mmol, 89% yield over 2 steps), contaminated with residual DIEA and DMF.

### Example 37: N-(3,5-dichloro-4-(3-cyclopropyl-4-methoxyphenoxy)phenyl)-2-(pyridin-3-yl)acetamide

To a 1 dram pressure release vial was added N-(4-(3-bromo-4-methoxyphenoxy)-3,5-dichlorophenyl)-2-(pyridin-3-yl)acetamide 28C (26.5 mg, ˜70% purity, 38.5 μmol), cyclopropylboronic acid (7.4 mg, 86 μmol), 1,4-dioxane (0.25 mL), and a solution of potassium carbonate (11.9 mg, 86 μmol) in H₂O (0.050 mL). Pd(dppf)Cl₂ (1.6 mg, 2.16 μmol) was then added, and nitrogen was bubbled through the resulting suspension for 5 min. The reaction was then stirred at 100° C. for 20 hours. After cooling to room temperature, the reaction mixture was diluted with DMF and purified by reversed phase HPLC to afford N-(3,5-dichloro-4-(3-cyclopropyl-4-methoxyphenoxy)phenyl)-2-(pyridin-3-yl)acetamide Example 37 (7.0 mg, 15.2 μmol, 39% yield). LCMS m/z 443.1 (M+H); rt 1.76 min; conditions B; ¹H NMR (500 MHz, DMSO-d₆) δ 10.63 (s, 1H), 8.53 (br. s., 1H), 8.48 (br. s., 1H), 7.81 (s, 2H), 7.75 (d, J=7.9 Hz, 1H), 7.38 (dd, J=7.7, 5.0 Hz, 1H), 6.81 (d, J=8.9 Hz, 1H), 6.41 (d, J=3.1 Hz, 1H), 6.36 (dd, J=8.9, 3.1 Hz, 1H), 3.80-3.69 (m, 5H), 2.08 (tt, J=8.4, 5.3 Hz, 1H), 0.92-0.83 (m, 2H), 0.60-0.51 (m, 2H).

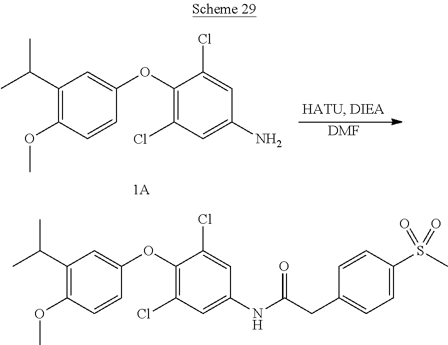

### Example 38

### N-(3,5-dichloro-4-(3-isopropyl-4-methoxyphenoxy)phenyl)-2-(4-(methylsulfonyl)phenyl)acetamide

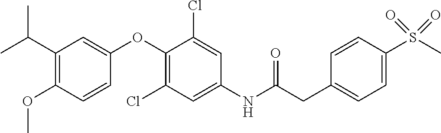 [SMILES: COc1ccc(Oc2c(Cl)cc(NC(=O)Cc3ccc(S(C)(=O)=O)cc3)cc2Cl)cc1C(C)C]

To a 1 dram vial containing 2-(4-(methylsulfonyl)phenyl)acetic acid (19.7 mg, 0.092 mmol) was added HATU (26.2 mg, 0.069 mmol) in DMF (0.23 mL), followed by 3,5-dichloro-4-(3-isopropyl-4-methoxyphenoxy)aniline 1A (15.0 mg, 0.046 mmol) in DMF (0.23 mL). DIEA (24 μL, 0.138 mmol) was then added, and the mixture was allowed to stir at room temperature. After 4 days, added additional portions of 2-(4-(methylsulfonyl)phenyl)acetic acid (19.7 mg, 0.092 mmol) and HATU (26.2 mg, 0.069 mmol). After 4 more hours, the reaction was quenched with a drop of water, diluted with DMF and purified by reversed phase HPLC to afford N-(3,5-dichloro-4-(3-isopropyl-4-methoxyphenoxy)phenyl)-2-(4-(methylsulfonyl)phenyl)acetamide Example 38 (19.0 mg, 0.035 mmol, 77% yield). LCMS m/z 522.2 (M+H); rt 2.42 min; conditions B; ¹H NMR (500 MHz, DMSO-d₆) δ 10.67 (s, 1H), 7.94-7.85 (m, J=8.2 Hz, 2H), 7.81 (s, 2H), 7.64-7.54 (m, J=8.2 Hz, 2H), 6.83 (d, J=8.9 Hz, 1H), 6.77 (d, J=3.1 Hz, 1H), 6.40 (dd, J=8.9, 3.1 Hz, 1H), 3.82 (s, 2H), 3.72 (s, 3H), 3.22-3.14 (m, 4H), 1.10 (d, J=6.9 Hz, 6H).

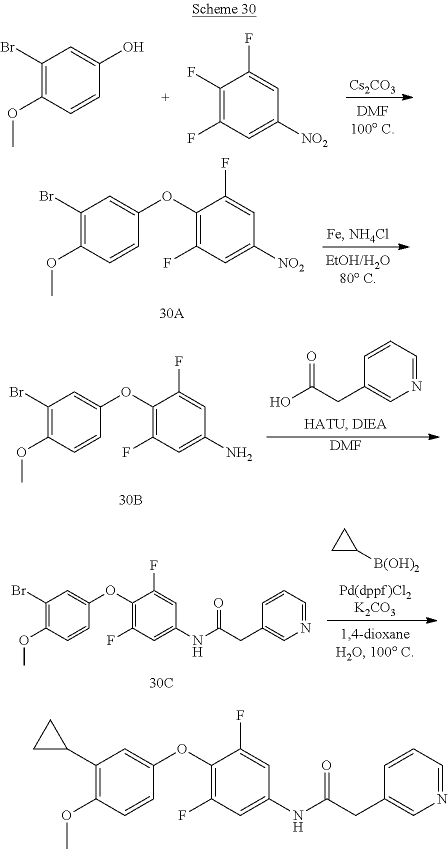

### Example 39

### N-(4-(3-cyclopropyl-4-methoxyphenoxy)-3,5-difluorophenyl)-2-(pyridin-3-yl)acetamide

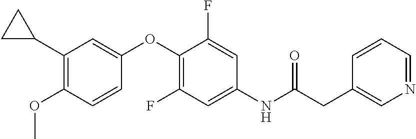 [SMILES: COc1ccc(Oc2c(F)cc(NC(=O)Cc3cccnc3)cc2F)cc1C1CC1]

### Intermediate 30A: 2-(3-bromo-4-methoxyphenoxy)-1,3-difluoro-5-nitrobenzene

A 2 dram pressure relief vial containing a suspension of 3-bromo-4-methoxyphenol (203 mg, 1.00 mmol), 1,2,3-trifluoro-5-nitrobenzene (177 mg, 1.00 mmol), and cesium carbonate (489 mg, 1.50 mmol) in DMF (4.0 mL) was stirred at 100° C. After 1 hour, the reaction was allowed to cool to room temperature and partitioned between EtOAc (40 mL) and water (40 mL). The aqueous layer was extracted with EtOAc (2×40 mL), and then the combined organic layers were dried (Na₂SO₄), filtered, and concentrated in vacuo. The crude product was dissolved in a small amount of CH₂Cl₂, adsorbed onto a plug of SiO₂, and purified by flash chromatography (SiO₂, 40 g column, 0-25% EtOAc/hexanes, 28.8 min gradient, 40 mL/min) to afford 2-(3-bromo-4-methoxyphenoxy)-1,3-difluoro-5-nitrobenzene (353 mg, 0.980 mmol, 98% yield) 30A as a clear, pale yellow film. ¹H NMR (400 MHz, CHLOROFORM-d) δ 7.99-7.91 (m, 2H), 7.21 (d, J=3.1 Hz, 1H), 6.94 (dd, J=9.0, 3.1 Hz, 1H), 6.85 (d, J=9.0 Hz, 1H), 3.88 (s, 3H).

### Intermediate 30B: 4-(3-bromo-4-methoxyphenoxy)-3,5-difluoroaniline

To a vial containing a suspension of 2-(3-bromo-4-methoxyphenoxy)-1,3-difluoro-5-nitrobenzene 30A (353 mg, 0.980 mmol) in ethanol (3.0 mL) was added a solution of ammonium chloride (262 mg, 4.90 mmol) in water (1.0 mL), followed by iron powder (547 mg, 9.80 mmol). The resulting mixture was stirred at 80° C. for 45 min. After cooling to room temperature, the reaction was diluted with EtOAc (40 mL), and washed with 1:1 mixture of brine and 1.5 M aqueous K₂HPO₄ (20 mL). The aqueous layer was back-extracted with EtOAc (3×20 mL). The combined organic layers were dried (Na₂SO₄), filtered through a pad of Celite, and concentrated in vacuo to afford 4-(3-bromo-4-methoxyphenoxy)-3,5-difluoroaniline 30B (309.5 mg, 0.938 mmol, 96% yield) as an off-white solid. LCMS m/z 330.0 (M+H); rt 0.96 min; conditions C; ¹H NMR (400 MHz, CHLOROFORM-d) δ 7.14 (d, J=2.9 Hz, 1H), 6.89 (dd, J=9.0, 2.9 Hz, 1H), 6.81 (d, J=9.0 Hz, 1H), 6.33-6.23 (m, 2H), 3.85 (s, 3H), 3.80 (br. s., 2H).

### Intermediate 30C: N-(4-(3-bromo-4-methoxyphenoxy)-3,5-difluorophenyl)-2-(pyridin-3-yl)acetamide

To a round bottom flask containing 2-(pyridin-3-yl)acetic acid hydrochloride (105 mg, 0.606 mmol) was added HATU (173 mg, 0.454 mmol) in DMF (0.75 mL), followed by 4-(3-bromo-4-methoxyphenoxy)-3,5-difluoroaniline 30B (100 mg, 0.303 mmol) in DMF (0.750 mL). DIEA (0.265 mL, 1.52 mmol) was then added, and the mixture was allowed to stir at room temperature. After 2 days, the reaction was diluted with water (15 mL) and extracted with EtOAc (3×15 mL). The combined organic layers were dried (Na₂SO₄), filtered, and concentrated in vacuo. The crude product was dissolved in a small amount of CH₂Cl₂, adsorbed onto a plug of SiO₂, and purified by flash chromatography (SiO₂, 24 g column, 0-8% MeOH/CH₂Cl₂, 24 g column, 11.5 min gradient, 35 mL/min) to afford N-(4-(3-bromo-4-methoxyphenoxy)-3,5-difluorophenyl)-2-(pyridin-3-yl)acetamide 30C (192.5 mg, ˜70% purity, 0.300 mmol, 99% yield), contaminated with residual DIEA and DMF.

### Example 39: N-(4-(3-cyclopropyl-4-methoxyphenoxy)-3,5-difluorophenyl)-2-(pyridin-3-yl)acetamide

To a 1 dram pressure release vial was added N-(4-(3-bromo-4-methoxyphenoxy)-3,5-difluorophenyl)-2-(pyridin-3-yl)acetamide 30C (32.3 mg, 0.050 mmol), cyclopropylboronic acid (12.97 mg, 0.151 mmol), 1,4-dioxane (0.25 mL), and a solution of potassium carbonate (13.9 mg, 0.101 mmol) in H₂O (0.050 mL). Pd(dppf)Cl₂ (1.8 mg, 2.52 μmol) was then added, and nitrogen was bubbled through the resulting suspension for 5 min. The reaction was then stirred at 100° C. for 20 hours. After cooling to room temperature, the reaction mixture was diluted with DMF purified by reversed phase HPLC to afford N-(4-(3-cyclopropyl-4-methoxyphenoxy)-3,5-difluorophenyl)-2-(pyridin-3-yl)acetamide Example 39 (9.3 mg, 0.022 mmol, 44% yield). LCMS m/z 410.8 (M+H); rt 1.69 min; conditions B; ¹H NMR (600 MHz, DMSO-d₆) δ 10.67 (s, 1H), 8.63-8.43 (m, 2H), 7.74 (d, J=7.7 Hz, 1H), 7.47 (d, J=10.3 Hz, 2H), 7.38 (br. s., 1H), 6.84 (d, J=8.8 Hz, 1H), 6.55 (dd, J=8.8, 2.9 Hz, 1H), 6.48 (d, J=2.9 Hz, 1H), 3.75 (s, 3H), 3.73 (s, 2H), 2.11-2.04 (m, 1H), 0.90-0.84 (m, 2H), 0.58-0.53 (m, 2H).

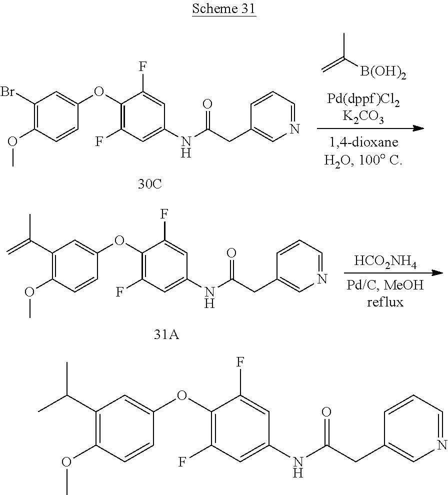

### Example 40

### N-(3,5-difluoro-4-(3-isopropyl-4-methoxyphenoxy)phenyl)-2-(pyridin-3-yl)acetamide

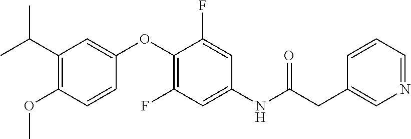 [SMILES: COc1ccc(Oc2c(F)cc(NC(=O)Cc3cccnc3)cc2F)cc1C(C)C]

### Intermediate 31A: N-(3,5-difluoro-4-(4-methoxy-3-(prop-1-en-2-yl)phenoxy)phenyl)-2-(pyridin-3-yl)acetamide

To a 1 dram pressure release vial was added N-(4-(3-bromo-4-methoxyphenoxy)-3,5-difluorophenyl)-2-(pyridin-3-yl)acetamide 30C (40.7 mg, 0.063 mmol), 4,4,5,5-tetramethyl-2-(prop-1-en-2-yl)-1,3,2-dioxaborolane (0.036 mL, 0.190 mmol), 1,4-dioxane (0.25 mL), and a solution of potassium carbonate (17.5 mg, 0.127 mmol) in H₂O (0.050 mL). Pd(dppf)Cl₂ (2.3 mg, 3.17 μmol) was then added, and nitrogen was bubbled through the resulting suspension for 5 min. The reaction was then stirred at 100° C. for 18 hours and concentrated in vacuo. The crude product was dissolved in a small amount of CH₂Cl₂, adsorbed onto a plug of Celite, and purified by flash chromatography (SiO₂, 4 g column, 0-10% MeOH/CH₂Cl₂, 10.6 min gradient, 18 mL/min) to afford N-(3,5-difluoro-4-(4-methoxy-3-(prop-1-en-2-yl)phenoxy)phenyl)-2-(pyridin-3-yl)acetamide 31A as a brown film. The partially purified material was carried into the next step. LCMS m/z 411.2 (M+H); rt 0.83 min; conditions C.

### Example 40: N-(3,5-difluoro-4-(3-isopropyl-4-methoxyphenoxy)phenyl)-2-(pyridin-3-yl)acetamide

A 10 mL round bottom flask containing partially purified N-(3,5-difluoro-4-(4-methoxy-3-(prop-1-en-2-yl)phenoxy)phenyl)-2-(pyridin-3-yl)acetamide 31A was outfitted with a reflux condenser and evacuated and backfilled with nitrogen three times. The substrate was dissolved in MeOH (0.63 mL), then palladium on carbon (6.8 mg, 3.17 μmol) and ammonium formate (40.0 mg, 634 μmol) were added. The mixture was stirred at reflux under nitrogen atmosphere. After 1.5 hours, added a second portion of ammonium formate (80 mg, 1270 μmol) and stirred again at reflux. After another 3.5 hours, additional MeOH (0.63 mL), ammonium formate (80 mg, 1270 μmol), and palladium on carbon (26.0 mg, 12.2 μmol) were added. After refluxing for another 1 hour, the reaction was allowed to cool to room temperature, diluted with CH₂Cl₂, and filtered through a Celite plug. The filtrate was concentrated in vacuo. The residue was then dissolved in DMF and purified by reversed phase HPLC to afford N-(3,5-difluoro-4-(3-isopropyl-4-methoxyphenoxy)phenyl)-2-(pyridin-3-yl)acetamide Example 40 (2.6 mg, 5.99 μmol, 9% yield over 2 steps). LCMS m/z 413.0 (M+H); rt 1.85 min; conditions B; ¹H NMR (500 MHz, DMSO-d₆) δ 10.68 (s, 1H), 8.64-8.40 (m, 2H), 7.73 (br. d, J=7.7 Hz, 1H), 7.47 (d, J=10.2 Hz, 2H), 7.42-7.34 (m, 1H), 6.90-6.80 (m, 2H), 6.59 (dd, J=8.8, 3.0 Hz, 1H), 3.72 (overlapping s, 5H), 3.24-3.17 (m, 1H), 1.10 (d, J=6.9 Hz, 6H).

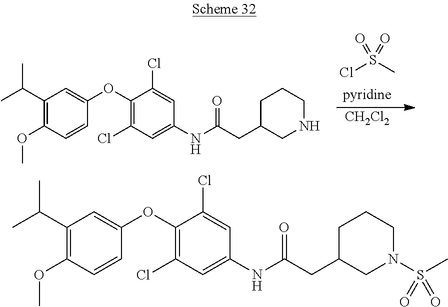

### Example 41

### N-(3,5-dichloro-4-(3-isopropyl-4-methoxyphenoxy)phenyl)-2-(1-(methylsulfonyl)piperidin-3-yl)acetamide

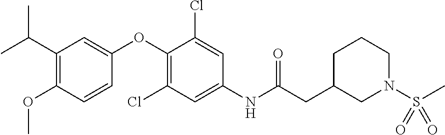 [SMILES: COc1ccc(Oc2c(Cl)cc(NC(=O)CC3CCCN(S(C)(=O)=O)C3)cc2Cl)cc1C(C)C]

To a vial containing a solution of N-(3,5-dichloro-4-(3-isopropyl-4-methoxyphenoxy)phenyl)-2-(piperidin-3-yl)acetamide Example 32 (5.2 mg, 0.011 mmol) and pyridine (20 μL, 0.247 mmol) in CH₂Cl₂ (115 μL) was added methanesulfonyl chloride (10 μL, 0.129 mmol). The mixture was stirred at room temperature for 2.5 hours. The reaction was then quenched with a drop of water, diluted with DMF and purified by reversed phase HPLC to afford racemic N-(3,5-dichloro-4-(3-isopropyl-4-methoxyphenoxy)phenyl)-2-(1-(methylsulfonyl)piperidin-3-yl)acetamide Example 41 (2.3 mg, 4.17 μmol, 36% yield). LCMS m/z 529.3 (M+H); rt 2.37 min; conditions B; ¹H NMR (500 MHz, DMSO-d₆) δ 10.36 (s, 1H), 7.81 (s, 2H), 6.83 (d, J=9.0 Hz, 1H), 6.78 (d, J=3.1 Hz, 1H), 6.40 (dd, J=8.8, 3.2 Hz, 1H), 3.72 (s, 3H), 3.24-3.14 (m, 1H), 2.84 (s, 3H), 2.78-2.71 (m, J=10.0, 10.0 Hz, 1H), 2.38-2.28 (m, 2H), 2.12-2.01 (m, J=5.5, 5.5 Hz, 1H), 1.79-1.69 (m, J=8.3, 4.0 Hz, 2H), 1.57-1.44 (m, J=9.9, 3.7 Hz, 1H), 1.20-1.13 (m, J=7.6 Hz, 1H), 1.11 (d, J=6.9 Hz, 6H). Note: some proton resonances obscured by water/solvent peaks.

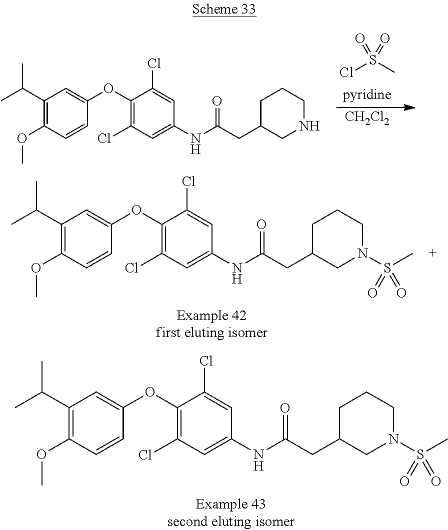

### Examples 42 and 43

### N-(3,5-dichloro-4-(3-isopropyl-4-methoxyphenoxy)phenyl)-2-(1-(methylsulfonyl)piperidin-3-yl)acetamide

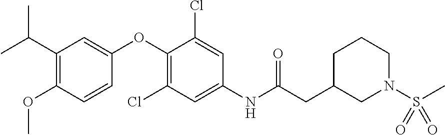 [SMILES: COc1ccc(Oc2c(Cl)cc(NC(=O)CC3CCCN(S(C)(=O)=O)C3)cc2Cl)cc1C(C)C]

### Example 42

### First Eluting Isomer

### Example 43

### Second Eluting Isomer

To a vial containing a solution of N-(3,5-dichloro-4-(3-isopropyl-4-methoxyphenoxy)phenyl)-2-(piperidin-3-yl)acetamide hydrochloride Example 32 (47.3 mg, 97.0 μmol) and pyridine (160 μL, 1940 μmol) in DCM (0.49 mL) was added methanesulfonyl chloride (75 μL, 970 μmol). The mixture was stirred at room temperature for 1 h. The reaction was then quenched with a drop of water, diluted with DMF and purified by reversed phase HPLC. The purified racemic material was then separated by chiral SFC to afford Example 42 (first eluting isomer) (3.4 mg, 6.29 μmol, 13% yield) and Example 43 (second eluting isomer) (2.8 mg, 5.29 μmol, 11% yield).

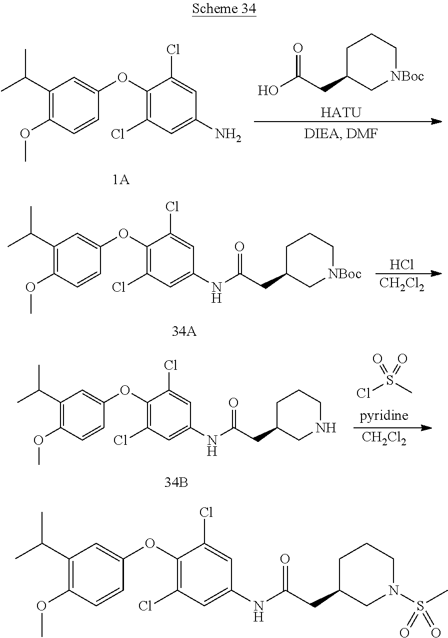

### Example 44

### (R)—N-(3,5-dichloro-4-(3-isopropyl-4-methoxyphenoxy)phenyl)-2-(1-(methylsulfonyl)piperidin-3-yl)acetamide

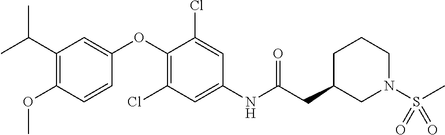 [SMILES: COc1ccc(Oc2c(Cl)cc(NC(=O)C[C@H]3CCCN(S(C)(=O)=O)C3)cc2Cl)cc1C(C)C]

### Intermediate 34A: (R)-tert-butyl 3-(2-((3,5-dichloro-4-(3-isopropyl-4-methoxyphenoxy)phenyl)amino)-2-oxoethyl)piperidine-1-carboxylate

To a 2 dram vial containing (R)-2-(1-(tert-butoxycarbonyl)piperidin-3-yl)acetic acid (149 mg, 0.613 mmol) was added HATU (175 mg, 0.460 mmol) in DMF (0.75 mL), followed by 3,5-dichloro-4-(3-isopropyl-4-methoxyphenoxy)aniline 1A (100 mg, 0.307 mmol) in DMF (0.750 mL). DIEA (0.161 mL, 0.920 mmol) was then added, and the mixture was allowed to stir at room temperature. After 20 hours, added another portion of (R)-2-(1-(tert-butoxycarbonyl)piperidin-3-yl)acetic acid (37.3 mg, 0.153 mmol) and HATU (58.3 mg, 0.153 mmol) and continued stirring at room temperature. After another 4 days, the reaction mixture was partitioned between CH₂Cl₂ (15 mL) and 1.5 M aqueous K₂HPO₄ (30 mL). The aqueous phase was then back-extracted once with CH₂Cl₂ (15 mL), and the combined organic layers were dried (Na₂SO₄), filtered, and concentrated in vacuo. The crude product was adsorbed onto Celite, and purified by flash chromatography (SiO₂, 12 g column, 0-50% EtOAc/hexanes, 20 min gradient, 30 mL/min) to afford (R)-tert-butyl 3-(2-((3,5-dichloro-4-(3-isopropyl-4-methoxyphenoxy)phenyl)amino)-2-oxoethyl)piperidine-1-carboxylate 34A (155 mg, 0.281 mmol, 92% yield). LCMS m/z 551.1 (M+H); rt 3.63 min; conditions Z.

### Intermediate 34B: (R)—N-(3,5-dichloro-4-(3-isopropyl-4-methoxyphenoxy)phenyl)-2-(piperidin-3-yl)acetamide Hydrochloride

To a solution of (R)-tert-butyl 3-(2-((3,5-dichloro-4-(3-isopropyl-4-methoxyphenoxy)phenyl)amino)-2-oxoethyl)piperidine-1-carboxylate 34A (155 mg, 0.281 mmol) in CH2Cl2 (1.4 mL) was added hydrochloric acid, 4.0 M in 1,4-dioxane (1.4 mL, 5.63 mmol). The reaction was stirred at room temperature. After 1.5 hours, the reaction was concentrated in vacuo to afford crude (R)—N-(3,5-dichloro-4-(3-isopropyl-4-methoxyphenoxy)phenyl)-2-(piperidin-3-yl)acetamide hydrochloride 34B as a light brown residue. The crude material was carried forward without further purification. LCMS m/z 451.1 (M+H); rt 0.88 min; conditions C.

### Example 44: (R)—N-(3,5-dichloro-4-(3-isopropyl-4-methoxyphenoxy)phenyl)-2-(1-(methylsulfonyl)piperidin-3-yl)acetamide

To a vial containing a solution of crude (R)—N-(3,5-dichloro-4-(3-isopropyl-4-methoxyphenoxy)phenyl)-2-(piperidin-3-yl)acetamide hydrochloride 34B (34.1 mg, 70.0 μmol) and pyridine (0.11 mL, 1.40 mmol) in CH₂Cl₂ (0.35 mL) was added methanesulfonyl chloride (54 μL, 700 μmol). The mixture was stirred at room temperature for 1 hour. The reaction was then quenched with a drop of water, diluted with DMF and purified by reversed phase HPLC to afford (R)—N-(3,5-dichloro-4-(3-isopropyl-4-methoxyphenoxy)phenyl)-2-(1-(methylsulfonyl)piperidin-3-yl)acetamide Example 44 (5.0 mg, 9.07 μmol, 13% yield). LCMS m/z 529.0 (M+H); rt 2.52 min; conditions B.

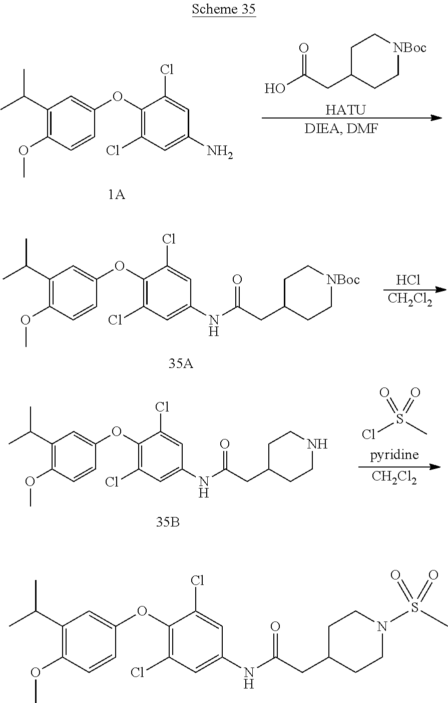

### Example 45

### N-(3,5-dichloro-4-(3-isopropyl-4-methoxyphenoxy)phenyl)-2-(1-(methylsulfonyl)piperidin-4-yl)acetamide

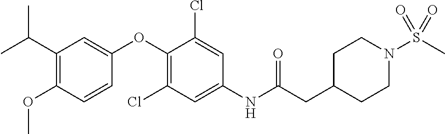 [SMILES: COc1ccc(Oc2c(Cl)cc(NC(=O)CC3CCN(S(C)(=O)=O)CC3)cc2Cl)cc1C(C)C]

### Intermediate 35A: tert-butyl 4-(2-((3,5-dichloro-4-(3-isopropyl-4-methoxyphenoxy)phenyl)amino)-2-oxoethyl)piperidine-1-carboxylate

To a 2 dram vial containing 2-(1-(tert-butoxycarbonyl)piperidin-4-yl)acetic acid (149 mg, 0.613 mmol) was added HATU (175 mg, 0.460 mmol) in DMF (0.75 mL), followed by 3,5-dichloro-4-(3-isopropyl-4-methoxyphenoxy)aniline 1A (100 mg, 0.307 mmol) in DMF (0.75 mL). DIEA (0.161 mL, 0.920 mmol) was then added, and the mixture was allowed to stir at room temperature. After 20 hours, added another portion of 2-(1-(tert-butoxycarbonyl)piperidin-4-yl)acetic acid (37.3 mg, 0.153 mmol) and HATU (58.3 mg, 0.153 mmol) and continued stirring at room temperature. After another 4 days, the reaction mixture was partitioned between CH₂Cl₂ (15 mL) and 1.5 M aqueous K₂HPO₄ (30 mL). The aqueous phase was then back-extracted once with CH₂Cl₂ (15 mL), and the combined organic layers were dried (Na₂SO₄), filtered, and concentrated in vacuo. The crude product was adsorbed onto Celite, and purified by flash chromatography (SiO₂, 12 g column, 0-50% EtOAc/hexanes, 13 min gradient, 30 mL/min) to afford tert-butyl 4-(2-((3,5-dichloro-4-(3-isopropyl-4-methoxyphenoxy)phenyl)amino)-2-oxoethyl)piperidine-1-carboxylate 35A (83.4 mg, 0.151 mmol, 49% yield). LCMS m/z 551.0 (M+H); rt 3.58 min; conditions Z.

### Intermediate 35B: N-(3,5-dichloro-4-(3-isopropyl-4-methoxyphenoxy)phenyl)-2-(piperidin-4-yl)acetamide Hydrochloride

To a solution of tert-butyl 4-(2-((3,5-dichloro-4-(3-isopropyl-4-methoxyphenoxy)phenyl)amino)-2-oxoethyl)piperidine-1-carboxylate 35A (83.4 mg, 0.151 mmol) in CH₂Cl₂ (0.76 mL) was added hydrochloric acid, 4.0 M in 1,4-dioxane (0.76 mL, 3.02 mmol). The reaction was stirred at room temperature. After 2 hours, the reaction was concentrated in vacuo to afford crude N-(3,5-dichloro-4-(3-isopropyl-4-methoxyphenoxy)phenyl)-2-(piperidin-4-yl)acetamide hydrochloride 35B as an off-white solid. The crude material was carried forward without further purification. LCMS m/z 451.1 (M+H); rt 0.87 min; conditions C.

### Example 45: N-(3,5-dichloro-4-(3-isopropyl-4-methoxyphenoxy)phenyl)-2-(1-(methylsulfonyl)piperidin-4-yl)acetamide

To a vial containing a solution of crude N-(3,5-dichloro-4-(3-isopropyl-4-methoxyphenoxy)phenyl)-2-(piperidin-4-yl)acetamide hydrochloride 35B (18.3 mg, 37.5 μmol) and pyridine (61 μL, 750 μmol) in CH₂Cl₂ (190 μL) was added methanesulfonyl chloride (29 μL, 375 μmol). The mixture was stirred at room temperature for 1 hour. The reaction was then quenched with a drop of water, diluted with DMF and purified by reversed phase HPLC to afford N-(3,5-dichloro-4-(3-isopropyl-4-methoxyphenoxy)phenyl)-2-(1-(methylsulfonyl)piperidin-4-yl)acetamide Example 45 (6.5 mg, 12.3 μmol, 33% yield). LCMS m/z 529.2 (M+H); rt 2.34 min; conditions B; ¹H NMR (500 MHz, DMSO-d₆) δ 10.38 (s, 1H), 7.83 (s, 2H), 6.84 (d, J=9.0 Hz, 1H), 6.78 (d, J=3.1 Hz, 1H), 6.41 (dd, J=8.8, 3.1 Hz, 1H), 3.73 (s, 3H), 3.57-3.52 (m, 2H), 3.24-3.18 (m, 1H), 2.85 (s, 3H), 2.71 (t, J=11.3 Hz, 2H), 2.31 (d, J=7.1 Hz, 2H), 1.94-1.83 (m, 1H), 1.82-1.71 (m, 2H), 1.33-1.19 (m, 2H), 1.12 (d, J=6.9 Hz, 6H).

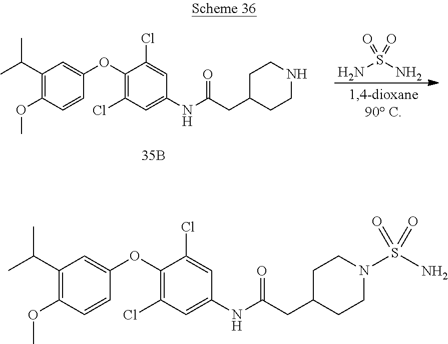 [SMILES: COc1ccc(Oc2c(Cl)cc(NC(=O)CC3CCNCC3)cc2Cl)cc1C(C)C ; NS(N)(=O)=O ; COc1ccc(Oc2c(Cl)cc(NC(=O)CC3CCN(S(N)(=O)=O)CC3)cc2Cl)cc1C(C)C]

### Example 46

### N-(3,5-dichloro-4-(3-isopropyl-4-methoxyphenoxy)phenyl)-2-(1-sulfamoylpiperidin-4-yl)acetamide

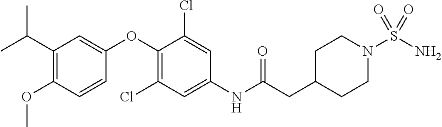 [SMILES: COc1ccc(Oc2c(Cl)cc(NC(=O)CC3CCN(S(N)(=O)=O)CC3)cc2Cl)cc1C(C)C]

A 1 dram pressure relief vial containing crude N-(3,5-dichloro-4-(3-isopropyl-4-methoxyphenoxy)phenyl)-2-(piperidin-4-yl)acetamide 35B (6.91 mg, 15.3 μmol) and sulfuric diamide (29.4 mg, 306 μmol) in 1,4-dioxane (0.30 mL) was stirred at 90° C. After 20 hours, a second portion of sulfuric diamide (29.4 mg, 306 μmol) was added, and the reaction was stirred again at 90° C. At 25 hours, the reaction was diluted with DMF and purified by reversed phase HPLC to afford N-(3,5-dichloro-4-(3-isopropyl-4-methoxyphenoxy)phenyl)-2-(1-sulfamoylpiperidin-4-yl)acetamide Example 46 (0.8 mg, 1.42 μmol, 9% yield). LCMS m/z 530.1 (M+H); rt 2.25 min; conditions B; ¹H NMR (500 MHz, DMSO-d₆) δ 7.84 (s, 2H), 6.83 (d, J=8.9 Hz, 1H), 6.79 (d, J=2.9 Hz, 1H), 6.40 (dd, J=9.1, 2.8 Hz, 1H), 3.73 (s, 3H), 3.24-3.14 (m, 1H), 2.30 (d, J=6.5 Hz, 2H), 1.87-1.80 (m, 1H), 1.80-1.73 (m, 2H), 1.30-1.23 (m, 2H), 1.12 (d, J=6.9 Hz, 6H). Note: some proton resonances obscured by water/solvent peaks.

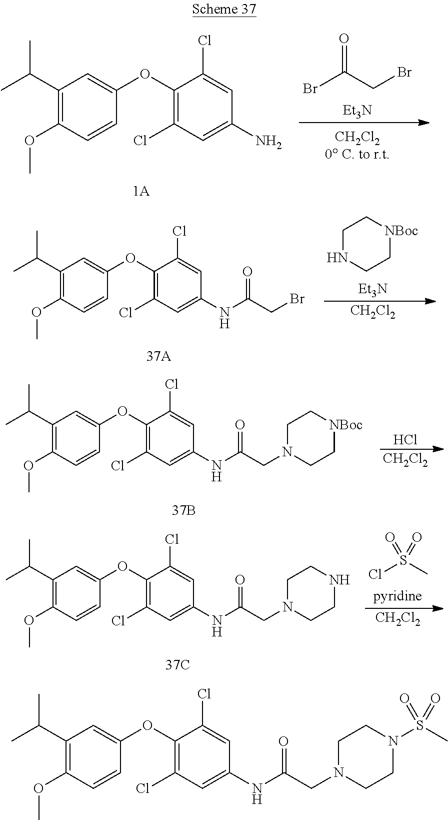

### Example 47

### N-(3,5-dichloro-4-(3-isopropyl-4-methoxyphenoxy)phenyl)-2-(4-(methylsulfonyl)piperazin-1-yl)acetamide

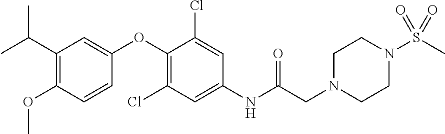 [SMILES: COc1ccc(Oc2c(Cl)cc(NC(=O)CN3CCN(S(C)(=O)=O)CC3)cc2Cl)cc1C(C)C]

### Intermediate 37A: 2-bromo-N-(3,5-dichloro-4-(3-isopropyl-4-methoxyphenoxy) phenyl)acetamide

To a cooled (0° C.) solution of 3,5-dichloro-4-(3-isopropyl-4-methoxyphenoxy)aniline 1A (178 mg, 0.545 mmol) and triethylamine (90 μL, 0.646 mmol) in CH₂Cl₂ (2.7 mL) was added 2-bromoacetyl bromide (50 μL, 0.574 mmol) dropwise via syringe. The resulting mixture was stirred overnight under nitrogen atmosphere while allowing the cooling bath to warm slowly to room temperature. After 18 hours, the reaction mixture was passed through a short pad of silica and rinsed with CH₂Cl₂. The filtrate was concentrated in vacuo to afford crude 2-bromo-N-(3,5-dichloro-4-(3-isopropyl-4-methoxyphenoxy)phenyl)acetamide 37A (210 mg, 0.470 mmol, 86% yield), which was used without further purification. LCMS m/z 446.0, 448.0, 450.0 (M+H); rt 1.12 min; conditions C.

### Intermediate 37B: tert-butyl 4-(2-((3,5-dichloro-4-(3-isopropyl-4-methoxyphenoxy) phenyl)amino)-2-oxoethyl)piperazine-1-carboxylate

To a 1 dram vial containing a solution of 2-bromo-N-(3,5-dichloro-4-(3-isopropyl-4-methoxyphenoxy)phenyl)acetamide 37A (100 mg, 0.224 mmol) and tert-butyl piperazine-1-carboxylate (62.5 mg, 0.335 mmol) in CH₂Cl₂ (1.0 mL) was added triethylamine (0.062 mL, 0.447 mmol). The resulting mixture was stirred at room temperature. The crude product was dissolved in a small amount of CH₂Cl₂, adsorbed onto a plug of SiO₂, and purified by flash chromatography (SiO₂, 12 g column, 0-75% EtOAc/hexanes, 12 g column, 11.2 min gradient, 30 mL/min) to afford tert-butyl 4-(2-((3,5-dichloro-4-(3-isopropyl-4-methoxyphenoxy)phenyl)amino)-2-oxoethyl)piperazine-1-carboxylate 37B (96.9 mg, 0.175 mmol, 78% yield) as a clear colorless film. LCMS m/z 552.3 (M+H); rt 0.95 min; conditions C.

### Intermediate 37C: N-(3,5-dichloro-4-(3-isopropyl-4-methoxyphenoxy)phenyl)-2-(piperazin-1-yl)acetamide Hydrochloride

To a solution of tert-butyl 4-(2-((3,5-dichloro-4-(3-isopropyl-4-methoxyphenoxy)phenyl)amino)-2-oxoethyl)piperazine-1-carboxylate 37B (96.9 mg, 0.175 mmol) in CH₂Cl₂ (3.5 mL) was added hydrochloric acid, 4.0 M in 1,4-dioxane (0.88 mL, 3.51 mmol). The reaction was stirred at room temperature. After 20 hours, the reaction was concentrated in vacuo to afford N-(3,5-dichloro-4-(3-isopropyl-4-methoxyphenoxy)phenyl)-2-(piperazin-1-yl)acetamide hydrochloride 37C as a white solid. The crude material was carried forward without further purification. LCMS m/z 452.2 (M+H); rt 0.87 min; conditions C.

### Example 47: N-(3,5-dichloro-4-(3-isopropyl-4-methoxyphenoxy)phenyl)-2-(4-(methylsulfonyl)piperazin-1-yl)acetamide

To a vial containing a solution of N-(3,5-dichloro-4-(3-isopropyl-4-methoxyphenoxy)phenyl)-2-(piperazin-1-yl)acetamide hydrochloride 37C (25 mg, 0.043 mmol) and pyridine (0.070 mL, 0.869 mmol) in CH₂Cl₂ (0.20 mL) was added methanesulfonyl chloride (0.034 mL, 0.435 mmol). The mixture was stirred at room temperature for 1 hour. The reaction was then quenched with a drop of water, diluted with DMF and purified by reversed phase HPLC to afford N-(3,5-dichloro-4-(3-isopropyl-4-methoxyphenoxy)phenyl)-2-(4-(methylsulfonyl)piperazin-1-yl)acetamide Example 47 (17.1 mg, 0.032 mmol, 74% yield). LCMS m/z 430.2 (M+H); rt 1.89 min; conditions B; ¹H NMR (500 MHz, DMSO-d₆) δ 10.10 (s, 1H), 7.93 (s, 2H), 6.84 (d, J=9.1 Hz, 1H), 6.78 (d, J=2.8 Hz, 1H), 6.42 (dd, J=8.9, 2.6 Hz, 1H), 3.73 (s, 3H), 3.24 (s, 2H), 3.22-3.14 (m, 5H), 2.90 (s, 3H), 2.68-2.59 (m, 4H), 1.12 (d, J=6.9 Hz, 6H).

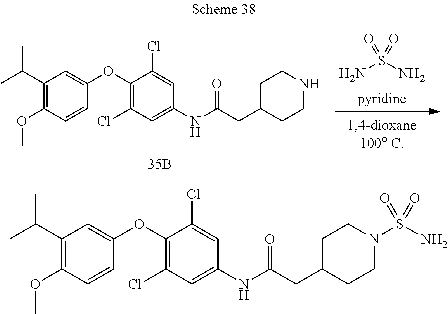 [SMILES: COc1ccc(Oc2c(Cl)cc(NC(=O)CC3CCNCC3)cc2Cl)cc1C(C)C ; NS(N)(=O)=O ; COc1ccc(Oc2c(Cl)cc(NC(=O)CC3CCN(S(N)(=O)=O)CC3)cc2Cl)cc1C(C)C]

### Example 48

### N-(3,5-dichloro-4-(3-isopropyl-4-methoxyphenoxy)phenyl)-2-(4-sulfamoylpiperazin-1-yl)acetamide

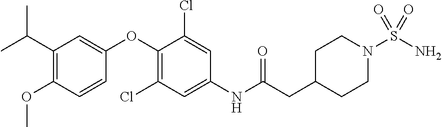 [SMILES: COc1ccc(Oc2c(Cl)cc(NC(=O)CC3CCN(S(N)(=O)=O)CC3)cc2Cl)cc1C(C)C]

A 1 dram pressure relief vial containing N-(3,5-dichloro-4-(3-isopropyl-4-methoxyphenoxy)phenyl)-2-(piperazin-1-yl)acetamide hydrochloride 35B (25 mg, 0.043 mmol), sulfuric diamide (125 mg, 1.30 mmol), and pyridine (20 μL, 0.247 mmol) in 1,4-dioxane (0.30 mL) was stirred at 100° C. After 3 days, the reaction was diluted with DMF and purified by reversed phase HPLC to afford N-(3,5-dichloro-4-(3-isopropyl-4-methoxyphenoxy)phenyl)-2-(4-sulfamoylpiperazin-1-yl)acetamide Example 48 (15.3 mg, 0.029 mmol, 66% yield). LCMS m/z 531.1 (M+H); rt 1.81 min; conditions B; ¹H NMR (500 MHz, DMSO-d₆) δ 10.10 (s, 1H), 7.92 (s, 2H), 6.84 (d, J=9.1 Hz, 1H), 6.80 (s, 2H), 6.77 (d, J=2.8 Hz, 1H), 6.41 (dd, J=8.8, 3.0 Hz, 1H), 3.73 (s, 3H), 3.25-3.14 (m, 3H), 3.08-3.00 (m, 4H), 2.65-2.56 (m, 4H), 1.11 (d, J=6.9 Hz, 6H).

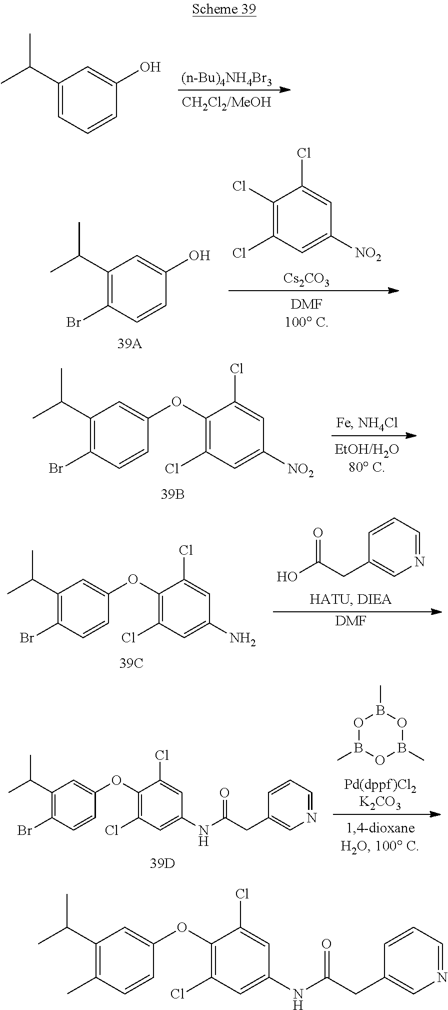

### Example 49

### N-(3,5-dichloro-4-(3-isopropyl-4-methylphenoxy)phenyl)-2-(pyridin-3-yl)acetamide

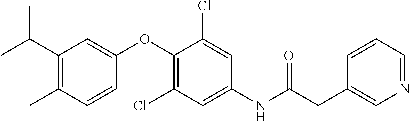 [SMILES: Cc1ccc(Oc2c(Cl)cc(NC(=O)Cc3cccnc3)cc2Cl)cc1C(C)C]

### Intermediate 39A: 4-bromo-3-isopropylphenol

To a 250 mL round bottom flask containing 3-isopropylphenol (0.817 g, 6.00 mmol) in CH₂Cl₂ (45.0 ml) and MeOH (30 mL) was added tetrabutylammonium tribromide (3.18 g, 6.60 mmol) as a solid, portion wise. The resulting clear yellow-orange solution was stirred at room temperature under nitrogen atmosphere. The solution gradually became a pale yellow color, and after 45 min the reaction mixture was concentrated in vacuo. Water (45 mL) was then added, and the mixture was extracted with ether (4×60 mL). The combined organic layers were dried (MgSO₄), filtered, and concentrated in vacuo to afford a 76:13:11 mixture of 4-bromo-3-isopropylphenol 39A/2-bromo-5-isopropylphenol/2,4-dibromo-5-isopropylphenol as a clear, brown oil (1.33 g, 99% overall mass recovery). ¹H NMR (400 MHz, CHLOROFORM-d, peaks for major product) 6 7.36 (d, J=8.6 Hz, 1H), 6.77 (d, J=2.9 Hz, 1H), 6.55 (dd, J=8.5, 3.0 Hz, 1H), 4.79 (s, 1H), 3.36-3.24 (m, 1H), 1.21 (d, J=6.8 Hz, 6H). The crude product mixture was carried into the next step without further purification.

### Intermediate 39B: 2-(4-bromo-3-isopropylphenoxy)-1,3-dichloro-5-nitrobenzene

A 20 mL pressure relief vial containing a suspension of crude 4-bromo-3-isopropylphenol 39A (593 mg total mass of the 76:13:11 mixture), 1,2,3-trichloro-5-nitrobenzene (600 mg, 2.65 mmol), and cesium carbonate (1.30 g, 3.97 mmol) in DMF (10 mL) was stirred at 100° C. After 1.5 hours, the reaction was allowed to cool to room temperature and partitioned between EtOAc (100 mL) and water (100 mL). The aqueous layer was extracted with EtOAc (2×100 mL), and then the combined organic layers were washed with 10% aqueous LiCl (2×50 mL), dried (Na₂SO₄), filtered, and concentrated in vacuo. The crude product was dissolved in a small amount of CH₂Cl₂, adsorbed onto a plug of SiO₂, and purified by flash chromatography (SiO₂, 80 g column, 0-5% EtOAc/hexanes, 25 min gradient, 60 mL/min) to afford ˜85% pure 2-(4-bromo-3-isopropylphenoxy)-1,3-dichloro-5-nitrobenzene 39B as a light yellow-orange solid, contaminated with mono-bromo and di-bromo impurities (929 mg, 85% overall mass recovery). ¹H NMR (500 MHz, CHLOROFORM-d, peaks for major product) 6 8.31 (s, 2H), 7.43 (d, J=8.7 Hz, 1H), 6.87 (d, J=3.1 Hz, 1H), 6.41 (dd, J=8.7, 3.1 Hz, 1H), 3.33 (spt, J=6.8 Hz, 1H), 1.22 (d, J=6.9 Hz, 6H).

### Intermediate 39C: 4-(4-bromo-3-isopropylphenoxy)-3,5-dichloroaniline

To a vial containing a suspension of 2-(4-bromo-3-isopropylphenoxy)-1,3-dichloro-5-nitrobenzene 39B (929 mg, ˜85% purity) in ethanol (6.6 mL) was added a solution of ammonium chloride (0.610 g, 11.4 mmol) in water (2.2 mL), followed by iron (1.27 g, 22.8 mmol). The resulting mixture was stirred at 80° C. for 1.5 hours. After cooling to room temperature, the reaction was diluted with EtOAc (80 mL), and washed with 1:1 mixture of brine and 1.5 M aqueous K₂HPO₄ (40 mL). The aqueous layer was back-extracted with EtOAc (3×40 mL). The combined organic layers were dried (Na₂SO₄), filtered through a pad of Celite, and concentrated in vacuo to afford ˜82% pure 4-(4-bromo-3-isopropylphenoxy)-3,5-dichloroaniline 39C as a clear, pale orange oil, contaminated with mono-bromo and di-bromo impurities (914 mg). The crude material was carried directly into the next step without further purification. ¹H NMR (400 MHz, CHLOROFORM-d, peaks for major product) 6 7.37 (d, J=8.8 Hz, 1H), 6.87 (d, J=3.1 Hz, 1H), 6.69 (s, 2H), 6.42 (dd, J=8.6, 3.1 Hz, 1H), 3.76 (br s, 2H), 3.30 (spt, J=6.8 Hz, 1H), 1.21 (d, J=6.8 Hz, 6H).

### Intermediate 39D: N-(4-(4-bromo-3-isopropylphenoxy)-3,5-dichlorophenyl)-2-(pyridin-3-yl)acetamide

To a round bottom flask containing 2-(pyridin-3-yl)acetic acid hydrochloride (0.439 g, 2.53 mmol) was added HATU (0.721 g, 1.898 mmol) in DMF (3.16 ml), followed by 4-(4-bromo-3-isopropylphenoxy)-3,5-dichloroaniline 39C (479.5 mg, ˜82% pure) in DMF (3.2 mL). DIEA (1.1 mL, 6.33 mmol) was then added, and the mixture was allowed to stir at room temperature. After 15 hours, the reaction was diluted with 1.5 M aqueous K₂HPO₄ (60 mL) and extracted with CH₂Cl₂ (3×60 mL). The combined organic layers were washed with 10% aqueous LiCl (60 mL), dried (Na₂SO₄), filtered, and concentrated in vacuo. The crude product was dissolved in a small amount of CH₂Cl₂, adsorbed onto a plug of SiO₂, and purified by flash chromatography (SiO₂, 40 g column, 0-5% MeOH/CH₂Cl₂, 14.4 min gradient, 40 mL/min) to afford ˜85% pure N-(4-(4-bromo-3-isopropylphenoxy)-3,5-dichlorophenyl)-2-(pyridin-3-yl)acetamide 39D as a yellow foam, contaminated with mono-bromo and di-bromo impurities (702 mg). LCMS m/z 492.8, 494.8, 496.9 (M+H); rt 0.96 min; conditions C.

### Example 49: N-(3,5-dichloro-4-(3-isopropyl-4-methylphenoxy)phenyl)-2-(pyridin-3-yl)acetamide

To a 1 dram pressure release vial was added N-(4-(4-bromo-3-isopropylphenoxy)-3,5-dichlorophenyl)-2-(pyridin-3-yl)acetamide 39D (40 mg, ˜85% purity), 2,4,6-trimethyl-1,3,5,2,4,6-trioxatriborinane (34 μL, 0.243 mmol), 1,4-dioxane (450 μL), and a solution of potassium carbonate (22.4 mg, 0.162 mmol) in H₂O (90 μL). Pd(dppf)Cl₂ (3.0 mg, 4.05 μmol) was then added, and nitrogen was bubbled through the resulting suspension for 5 min. The reaction was then stirred at 100° C. for 20 hours. After cooling to room temperature, the reaction mixture was diluted with DMF and purified by reversed phase HPLC to afford N-(3,5-dichloro-4-(3-isopropyl-4-methylphenoxy)phenyl)-2-(pyridin-3-yl)acetamide Example 49 (11.4 mg, 0.027 mmol, 33% yield). LCMS m/z 429.2 (M+H); rt 1.96 min; conditions B. ¹H NMR (500 MHz, DMSO-d₆) δ 10.69 (s, 1H), 8.62-8.40 (m, 2H), 7.80 (s, 2H), 7.75 (d, J=7.9 Hz, 1H), 7.44-7.32 (m, 1H), 7.01 (d, J=8.4 Hz, 1H), 6.73 (d, J=2.8 Hz, 1H), 6.33 (dd, J=8.4, 2.8 Hz, 1H), 3.73 (s, 2H), 3.09-2.96 (m, 1H), 2.19 (s, 3H), 1.09 (d, J=6.8 Hz, 6H).

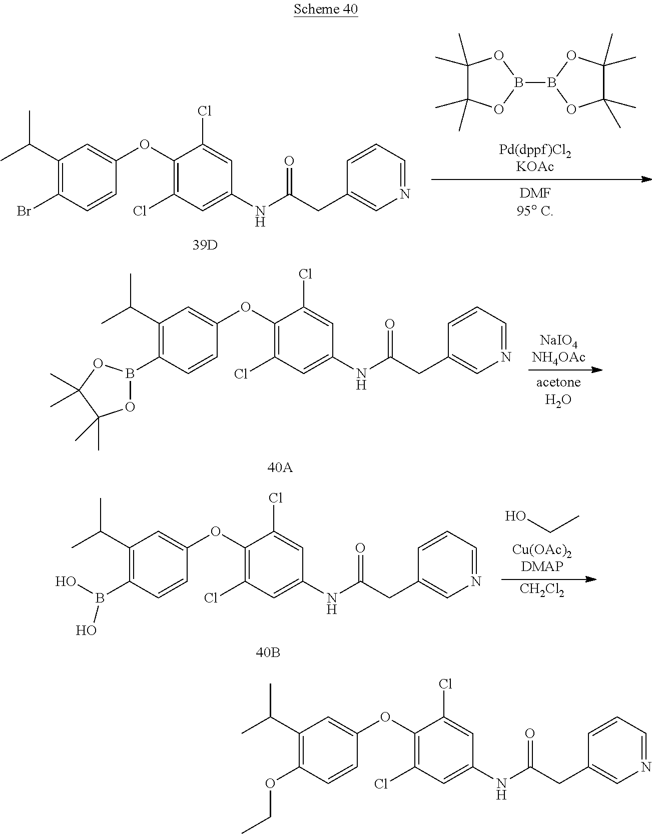

### Example 50

### N-(3,5-dichloro-4-(4-ethoxy-3-isopropylphenoxy)phenyl)-2-(pyridin-3-yl)acetamide

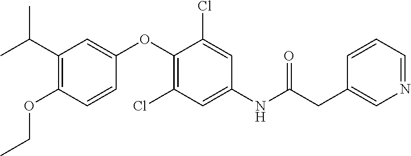 [SMILES: CCOc1ccc(Oc2c(Cl)cc(NC(=O)Cc3cccnc3)cc2Cl)cc1C(C)C]

To a 40 mL pressure release vial was added N-(4-(4-bromo-3-isopropylphenoxy)-3,5-dichlorophenyl)-2-(pyridin-3-yl)acetamide 39D (340 mg, 85% purity), 4,4,4′,4′,5,5,5′,5′-octamethyl-2,2′-bi(1,3,2-dioxaborolane) (0.262 g, 1.03 mmol), potassium acetate (0.236 g, 2.41 mmol), and DMF (4.0 mL). Pd(dppf)Cl₂ (0.050 g, 0.069 mmol) was then added, and nitrogen was bubbled through the resulting suspension for 5 min. The reaction was then stirred at 95° C. for 16 hours. The mixture was allowed to cool to room temperature, and then 1.0 M aqueous HCl was added (10 mL). The reaction was stirred at room temperature for 8 hours. Cleavage of the pinacol boronate ester was not observed by LCMS. The pH of the mixture was then adjusted to ˜7 with 1.0 M aqueous NaOH, then EtOAc (50 mL) was added and the layers were separated. The aqueous phase was extracted with EtOAc (2×25 mL), then organic layers were combined, washed with brine (20 mL), dried (Na₂SO₄), filtered through a Celite pad, and concentrated in vacuo. The crude product was dissolved in a small amount of CH₂Cl₂, adsorbed onto a plug of SiO₂, and purified by flash chromatography (SiO₂, 24 g column, 0-10% MeOH/CH₂Cl₂, 24 g column, 11.5 min gradient, 35 mL/min) to afford a ˜2:1 mixture of N-(3,5-dichloro-4-(3-isopropyl-4-(4,4,5,5-tetramethyl-1,3,2-dioxaborolan-2-yl)phenoxy)phenyl)-2-(pyridin-3-yl)acetamide 40A and the des-bromo reduction side product.

To a vial containing a solution of partially purified N-(3,5-dichloro-4-(3-isopropyl-4-(4,4,5,5-tetramethyl-1,3,2-dioxaborolan-2-yl)phenoxy)phenyl)-2-(pyridin-3-yl)acetamide 40A in acetone (6.0 mL) was added ammonium acetate (97 mg, 1.25 mmol), water (6.0 mL), and sodium periodate (402 mg, 1.88 mmol). The resulting mixture was stirred at room temperature. After 3 days, LCMS analysis of the reaction mixture showed partial conversion to the boronic acid. The reaction was diluted with 1.0 M aqueous NaOH (20 mL) and washed with CH₂Cl₂ (2×10 mL). The aqueous layer was then acidified to ˜pH 4-5 with 1.0 M aqueous HCl, resulting in the formation of a precipitate. The solid was filtered, washed with water, and dried under high vacuum to afford crude (4-(2,6-dichloro-4-(2-(pyridin-3-yl)acetamido)phenoxy)-2-isopropylphenyl)boronic acid 40B (26.4 mg), which was carried forward without further purification.

To a vial containing a suspension of crude (4-(2,6-dichloro-4-(2-(pyridin-3-yl)acetamido)phenoxy)-2-isopropylphenyl)boronic acid 40B (13.4 mg, 0.029 mmol), DMAP (3.57 mg, 0.029 mmol), and copper(II) acetate (2.65 mg, 0.015 mmol) in CH₂Cl₂ (0.20 mL) was added ethanol (10 μL, 0.171 mmol). The resulting mixture was stirred at room temperature under ambient atmosphere for 2 days. The reaction was then diluted with DMF and purified by reversed phase HPLC to afford N-(3,5-dichloro-4-(4-ethoxy-3-isopropylphenoxy)phenyl)-2-(pyridin-3-yl)acetamide Example 50 (0.6 mg, 1.25 μmol, 4% yield). LCMS m/z 459.0 (M+H); rt 2.07 min; conditions B; ¹H NMR (500 MHz, DMSO-d₆) δ 10.66 (br. s., 1H), 8.63-8.43 (m, 2H), 7.83 (s, 2H), 7.75 (d, J=8.8 Hz, 1H), 7.38 (d, J=5.5 Hz, 1H), 6.81 (d, J=9.1 Hz, 1H), 6.76 (d, J=3.0 Hz, 1H), 6.39 (dd, J=9.1, 3.0 Hz, 1H), 3.96 (q, J=6.9 Hz, 2H), 3.74 (s, 2H), 3.24-3.17 (m, 1H), 1.31 (t, J=6.9 Hz, 3H), 1.12 (d, J=6.9 Hz, 6H).

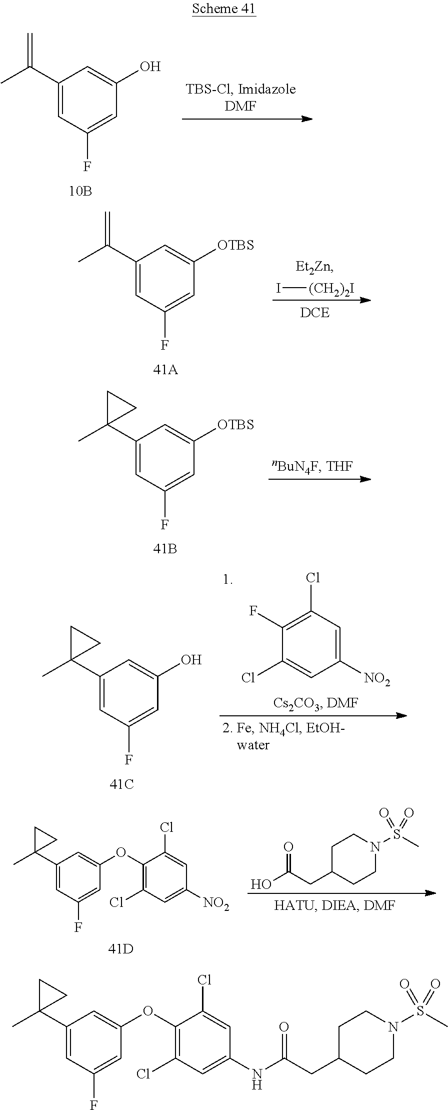

### Example 51

### N-(3,5-dichloro-4-(3-fluoro-5-(1-methylcyclopropyl)phenoxy)phenyl)-2-(1-(methylsulfonyl)piperidin-4-yl)acetamide

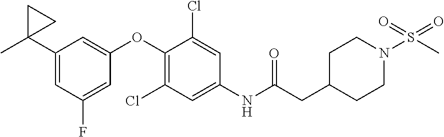 [SMILES: CC1(c2cc(F)cc(Oc3c(Cl)cc(NC(=O)CC4CCN(S(C)(=O)=O)CC4)cc3Cl)c2)CC1]

### Intermediate 41A: tert-butyl(3-fluoro-5-(prop-1-en-2-yl)phenoxy)dimethylsilane

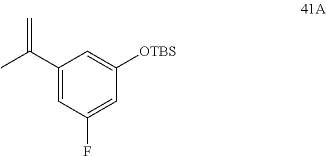 [SMILES: C=C(C)c1cc(F)cc(O[Si](C)(C)C(C)(C)C)c1]

A vial was charged with 3-fluoro-5-(prop-1-en-2-yl)phenol (1.2 g, 7.89 mmol) and imidazole (1.074 g, 15.77 mmol) in DMF (9.86 ml) and cooled to 0° C. tert-butylchlorodimethylsilane (1.426 g, 9.46 mmol) was added to this solution portion wise and the reaction was allowed to warm to room temperature for 16 hours. The reaction was diluted with ethyl acetate-water (25:10 ml). The organic layer was separated, washed with cold aqueous 1N HCl followed by water, dried (over Na₂SO₄) and concentrated. The residue was purified via ISCO silica gel chromatography (40 gm column; eluting with hexane/EtOAc; 0 to 40% gradient). tert-butyl(3-fluoro-5-(prop-1-en-2-yl)phenoxy)dimethylsilane 41A (1.8 g, 6.35 mmol) was isolated. LCMS m/z 267.1 (M+H); Retention time: 1.47 min (Method C); ¹H NMR (400 MHz, CDCl₃) δ 0.22 (s, 6H), 1.1 (s, 9H), 2.21 (dd, J=0.8, 1.5 Hz 3H), 5.07 (dd, J=1.5, 2.2 Hz, 1H), 5.40 (dd, J=0.8, 2.2 Hz, 1H), 6.46 (dt, J=2.5, 1.9, 1.9 Hz, 2H), 6.57 (dt, J=11.4, 2.5, 2.5 Hz, 1H), 6.8 (dt, J=11.4, 2.5, 1.9 Hz, 1H).

### Intermediate 41B: tert-butyl(3-fluoro-5-(1-methylcyclopropyl)phenoxy) dimethylsilane

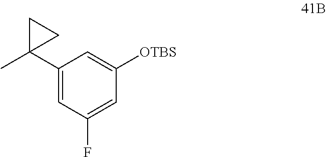 [SMILES: CC1(c2cc(F)cc(O[Si](C)(C)C(C)(C)C)c2)CC1]

Diethylzine (113 ml, 113 mmol) was added dropwise to a stirred, ice-cooled solution of tert-butyl(3-fluoro-5-(prop-1-en-2-yl)phenoxy)dimethylsilane 41A (1.5 g, 5.63 mmol) in 1,2-dichloroethane (28.2 ml). The solution was stirred at 0° C. for 30 min and then diiodomethane (7.54 g, 28.2 mmol) was added. The solution was allowed to warm to room temperature and was stirred overnight. The reaction was quenched by pouring into ice cold aqueous saturated solution of ammonium chloride. The mixture was allowed to stir for 30 min and then filtered over a bed of Celite. The organic layer was separated, washed with water, dried (Na₂SO₄) and concentrated. The crude residue was purified directly by ISCO silica gel chromatography (24 g, eluting with 0-50% EtOAc-hexanes gradient). tert-butyl(3-fluoro-5-(1-methylcyclopropyl)phenoxy) dimethylsilane 41B (1.1 g, 1.98 mmol) was obtained. LCMS m/z 281.2 (M+H); Retention time: 1.50 min (Method C); ¹H NMR (400 MHz, CDCl₃) δ 0.23 (s, 6H), 0.96 (dd, J=9.4, 5.9 Hz, 1H), 1.1 (dd, J=9.4, 5.9 Hz, 1H) 1.2 (s, 9H), 1.53 (s, 3H), 6.38 (dt, J=11.1, 2.7, 2.3 Hz, 2H), 6.46 (dd, J=2.10, 1.9 Hz, 1H), 6.55 (dt, J=11.4, 2.5, 1.9 Hz, 1H).

### Intermediate 41C: 3-fluoro-5-(1-methylcyclopropyl)phenol

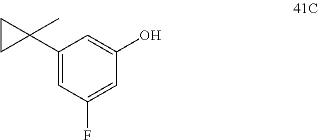

Tetra-n-butylammonium (3209 μl, 3.21 mmol) was added to a stirred, solution of tert-butyl(3-fluoro-5-(1-methylcyclopropyl)phenoxy)dimethylsilane 41B (450 mg, 1.605 mmol) in THE (8 ml). The solution was stirred overnight at room temperature. The solvent was evaporated and the crude residue was purified directly by ISCO silica gel chromatography (12 G, 0-50% EtOAc-hexanes gradient). 3-fluoro-5-(1-methylcyclopropyl)phenol 41C (160 mg, 0.9 mmol) was obtained. LCMS m/z 165.2 (M−H); Retention time: 0.92 min (Method C).

### Intermediate 41D: 3,5-dichloro-4-(3-fluoro-5-(1-methylcyclopropyl) Phenoxy)aniline

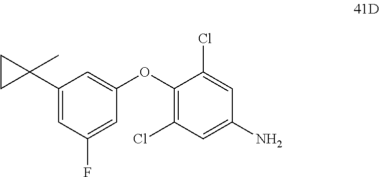 [SMILES: CC1(c2cc(F)cc(Oc3c(Cl)cc(N)cc3Cl)c2)CC1]

To a solution of 3-fluoro-5-(1-methylcyclopropyl)phenol 41C (150 mg, 0.903 mmol) and 1,3-dichloro-2-fluoro-5-nitrobenzene (227 mg, 1.083 mmol) in DMF (2.5 ml) was added Cs₂CO₃ (588 mg, 1.805 mmol). The reaction was heated to 80° C. overnight. The starting material had disappeared on LCMS: a new peak was formed but did not ionize in either positive or negative mode. The reaction was cooled, poured into water, and extracted with EtOAc (3×10 ml). The organic layers were washed with 10% LiCl solution, dried and concentrated.

The material was suspended in 12 mL EtOH and 4 mL water. Iron (403 mg, 7.22 mmol) and ammonium chloride (290 mg, 5.42 mmol) were added, and the reaction was heated to 80° C. After 2.5 hours, the reaction was cooled and filtered through Celite, rinsing with MeOH and EtOAc. The filtrate was concentrated. The residue was purified via ISCO (40 g column; Hex/EtOAc; 0 to 100% gradient) to give 3,5-dichloro-4-(3-fluoro-5-(1-methylcyclopropyl)phenoxy)aniline 41D (185 mg, 0.5 mmol) as brown solid. LCMS m/z 326.0 (M+H); Retention time: 1.1 min (Method C).

### Example 51: N-(3,5-dichloro-4-(3-fluoro-5-(1-methylcyclopropyl)phenoxy)phenyl)-2-(1-(methylsulfonyl)piperidin-4-yl)acetamide

A mixture of 3,5-dichloro-4-(3-fluoro-5-(1-methylcyclopropyl)phenoxy)aniline (0.04 g, 0.123 mmol), 2-(1-(methylsulfonyl)piperidin-4-yl)acetic acid (0.027 g, 0.123 mmol), DIEA (0.064 ml, 0.368 mmol) and HATU (0.056 g, 0.147 mmol) in DMF (0.613 ml) was stirred at room temperature overnight.

The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 50-100% B over 20 minutes, then a 7-minute hold at 100% B; Flow: 20 mL/min. Fractions containing the desired product were combined and dried via centrifugal evaporation. N-(3,5-dichloro-4-(3-fluoro-5-(1-methylcyclopropyl)phenoxy)phenyl)-2-(1-(methylsulfonyl)piperidin-4-yl)acetamide Example 51 (29 mg, 0.056 mmol) was obtained. LCMS m/z 529.1 (M+H); Retention time: 1.1 min (Method C).

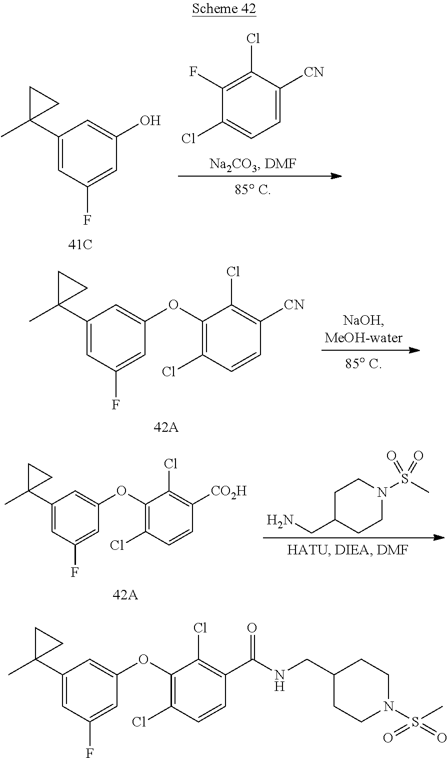 [SMILES: CC1(c2cc(O)cc(F)c2)CC1 ; N#Cc1ccc(Cl)c(F)c1Cl ; CC1(c2cc(F)cc(Oc3c(Cl)ccc(C#N)c3Cl)c2)CC1 ; CC1(c2cc(F)cc(Oc3c(Cl)ccc(C(=O)O)c3Cl)c2)CC1 ; CS(=O)(=O)N1CCC(CN)CC1 ; CC1(c2cc(F)cc(Oc3c(Cl)ccc(C(=O)NCC4CCN(S(C)(=O)=O)CC4)c3Cl)c2)CC1]

### Example 52

### 2,4-dichloro-3-(3-fluoro-5-(1-methylcyclopropyl)phenoxy)-N-((1-(methylsulfonyl)piperidin-4-yl)methyl)benzamide

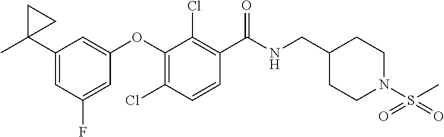 [SMILES: CC1(c2cc(F)cc(Oc3c(Cl)ccc(C(=O)NCC4CCN(S(C)(=O)=O)CC4)c3Cl)c2)CC1]

### Intermediate 42A: 2,4-dichloro-3-(3-fluoro-5-(1-methylcyclopropyl) Phenoxy)benzonitrile

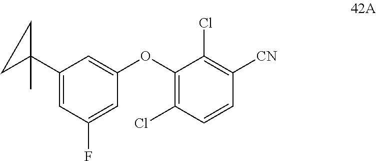 [SMILES: CC1(c2cc(F)cc(Oc3c(Cl)ccc(C#N)c3Cl)c2)CC1]

To a solution of 3-fluoro-5-(1-methylcyclopropyl)phenol 41C (300 mg, 1.805 mmol) and 2,4-dichloro-3-fluorobenzonitrile (412 mg, 2.166 mmol) in DMF (4.5 ml) was added potassium carbonate (374 mg, 2.71 mmol). The reaction mixture was stirred at 80° C. for 1 h. The reaction mixture was diluted with cold water and extracted with EtOAc (3×20 ml). The combined organic extracts were dried, concentrated to give a thick oil, which was purified by ISCO (80 g column, eluted with 0-30% EtOAc in hexanes) to give 2,4-dichloro-3-(3-fluoro-5-(1 methylcyclopropyl) phenoxy)benzonitrile 42A (420 mg, 1.1 mmol) as a thick oil, later solidified as a white solid. LCMS m/z 336.1 (M+H); Retention time: 1.12 min (Method C).

### Intermediate 42B: 2,4-dichloro-3-(3-fluoro-5-(1-methylcyclopropyl)phenoxy) Benzoic Acid

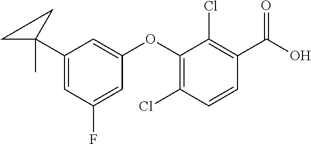 [SMILES: CC1(c2cc(F)cc(Oc3c(Cl)ccc(C(=O)O)c3Cl)c2)CC1]

3 M aqueous NaOH (4.36 ml, 13.09 mmol) was added to a solution of 2,4-dichloro-3-(3-fluoro-5-(1-methylcyclopropyl)phenoxy)benzonitrile 42A (0.55 g, 1.636 mmol) in EtOH (5.45 ml)/THF (2.73 ml) and the reaction mixture was heated at 85° C. overnight. The reaction was cooled to RT, acidified with 1 N aq. HCl and concentrated. The residue was taken up in a mixture of ethyl acetate and water. The organic layer was washed with water, dried and concentrated. The crude product, 2,4-dichloro-3-(3-fluoro-5-(1-methylcyclopropyl)phenoxy) benzoic acid 42B (390 mg, 0.98 mmol) was taken forward as such to the next step. LCMS m/z 355.2 (M+H); Retention time: 1.12 min (Method C).

### Example 52: 2,4-dichloro-3-(3-fluoro-5-(1-methylcyclopropyl)phenoxy)-N-((1-(methylsulfonyl)piperidin-4-yl)methyl)benzamide

A mixture of 2,4-dichloro-3-(3-fluoro-5-(1-methylcyclopropyl)phenoxy) benzoic acid 42B (0.05 g, 0.14 mmol), (1-(methylsulfonyl)piperidin-4-yl)methanamine hydrochloride (0.05 g, 0.21 mmol), DIEA (0.064 ml, 0.368 mmol) and HATU (0.07 g, 0.17 mmol) in DMF (0.613 ml) was stirred at room temperature overnight. The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 37-77% B over 20 minutes, then a 4-minute hold at 100% B; Flow: 20 mL/min. Fractions containing the desired product were combined and dried via centrifugal evaporation. 2,4-dichloro-3-(3-fluoro-5-(1-methylcyclopropyl)phenoxy)-N-((1-(methylsulfonyl)piperidin-4-yl)methyl)benzamide Example 52 (11.4 mg, 0.02 mmol) was obtained. LCMS m/z 528.9 (M+H); Retention time: 2.2 min (Method B).

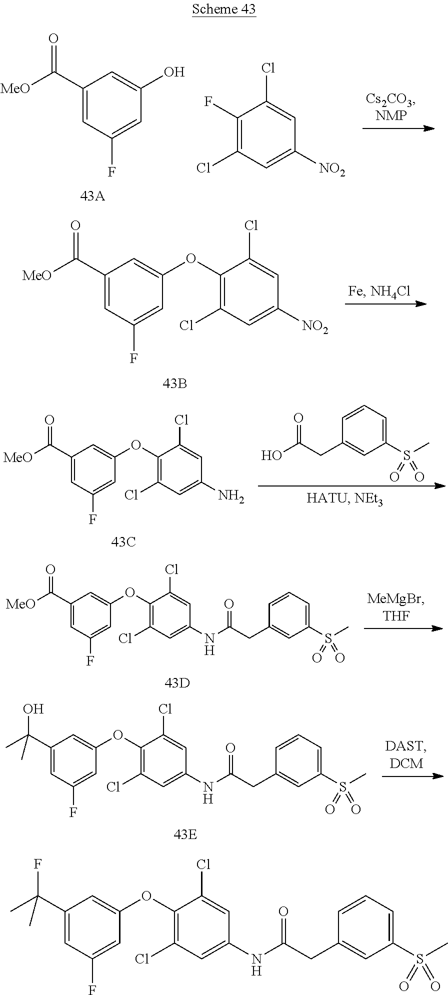

### Example 53

### N-(3,5-dichloro-4-(3-fluoro-5-(2-fluoropropan-2-yl)phenoxy)phenyl)-2-(3-(methylsulfonyl)phenyl)acetamide

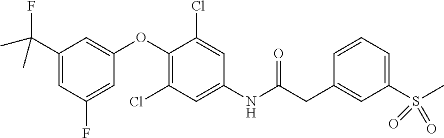 [SMILES: CC(C)(F)c1cc(F)cc(Oc2c(Cl)cc(NC(=O)Cc3cccc(S(C)(=O)=O)c3)cc2Cl)c1]

### Intermediate 43B: methyl 3-(2,6-dichloro-4-nitrophenoxy)-5-fluorobenzoate

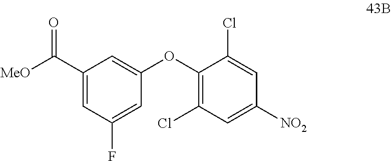 [SMILES: COC(=O)c1cc(F)cc(Oc2c(Cl)cc([N+](=O)[O-])cc2Cl)c1]

To a solution of methyl 3-fluoro-5-hydroxybenzoate (0.200 g, 1.176 mmol) and 1,3-dichloro-2-fluoro-5-nitrobenzene (0.370 g, 1.763 mmol) in NMP (4.70 ml) was added Cs₂CO₃ (0.766 g, 2.351 mmol). The reaction was heated to 120° C. After 1 hour, the reaction was cooled. Water was added; a precipitate formed, but most material could not be collected by filtration. The filtrate was extracted three times with EtOAc. The organic layers were washed with 10% LiCl solution, then combined with solid material from filtration. The material was absorbed onto silica gel. The residue was purified via ISCO (12 g column; Hex/EtOAc; 0 to 30% gradient) to give methyl 3-(2,6-dichloro-4-nitrophenoxy)-5-fluorobenzoate (0.375 g, 1.041 mmol, 89% yield). ¹H NMR (400 MHz, CHLOROFORM-d) δ 8.35 (s, 2H), 7.54-7.49 (m, 1H), 7.22 (s, 1H), 6.85 (dt, J=9.0, 2.4 Hz, 1H), 3.91 (s, 3H)

### Intermediate 43C: Methyl 3-(4-amino-2,6-dichlorophenoxy)-5-fluorobenzoate

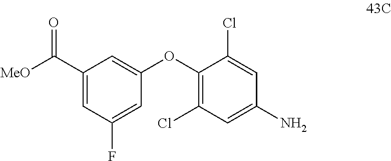 [SMILES: COC(=O)c1cc(F)cc(Oc2c(Cl)cc(N)cc2Cl)c1]

To a suspension of methyl 3-(2,6-dichloro-4-nitrophenoxy)-5-fluorobenzoate (0.200 g, 0.555 mmol) in EtOH (4.17 ml) and water (1.388 ml) was added iron (0.248 g, 4.44 mmol) and ammonium chloride (0.178 g, 3.33 mmol). The reaction was heated to 80° C. After 2.5 hours, the reaction was cooled, then filtered through Celite, washing with MeOH. The filtrate was concentrated, dissolved in DCM/MeOH, and filtered. The filtrate was concentrated, dissolved in DCM, filtered, and concentrated to give methyl 3-(4-amino-2,6-dichlorophenoxy)-5-fluorobenzoate (0.190 g, 0.576 mmol, 104% yield). ¹H NMR (400 MHz, CHLOROFORM-d) δ 7.48-7.37 (m, 1H), 7.33-7.27 (m, 1H), 6.78 (dt, J=9.5, 2.3 Hz, 1H), 6.68 (s, 2H)

### Intermediate 43D: Methyl 3-(2,6-dichloro-4-(2-(3-(methylsulfonyl)phenyl) Acetamido)phenoxy)-5-fluorobenzoate

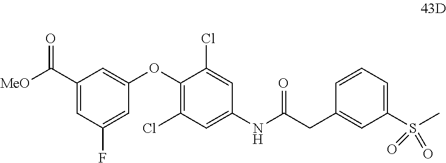 [SMILES: COC(=O)c1cc(F)cc(Oc2c(Cl)cc(NC(=O)Cc3cccc(S(C)(=O)=O)c3)cc2Cl)c1]

To a solution of 2-(3-(methylsulfonyl)phenyl)acetic acid (0.039 g, 0.182 mmol) and methyl 3-(4-amino-2,6-dichlorophenoxy)-5-fluorobenzoate (0.030 g, 0.091 mmol) in DMF (0.606 ml) was added HATU (0.073 g, 0.191 mmol) and triethylamine (0.051 ml, 0.363 mmol). After 16 hours, the reaction was diluted with water and extracted three times with EtOAc. The organic layers were concentrated. The residue was purified via ISCO (24 g column; Hex/EtOAc; 0 to 100% gradient) to give methyl 3-(2,6-dichloro-4-(2-(3-(methylsulfonyl)phenyl)acetamido)phenoxy)-5-fluorobenzoate (0.033 g, 0.063 mmol, 69.0% yield). LCMS m/z 526.1 (M+H); rt 1.01 min; Condition C.

### Intermediate 43E: N-(3,5-dichloro-4-(3-fluoro-5-(2-hydroxypropan-2-yl)phenoxy)phenyl)-2-(3-(methylsulfonyl)phenyl)acetamide

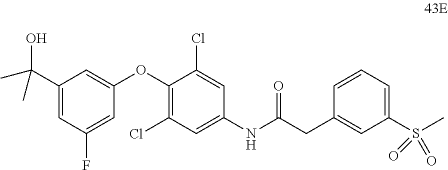 [SMILES: CC(C)(O)c1cc(F)cc(Oc2c(Cl)cc(NC(=O)Cc3cccc(S(C)(=O)=O)c3)cc2Cl)c1]

A solution of methyl 3-(2,6-dichloro-4-(2-(3-(methylsulfonyl)phenyl) acetamido)phenoxy)-5-fluorobenzoate (0.033 g, 0.063 mmol) was cooled in an ice bath. methylmagnesium bromide (3M in Et₂O) (0.104 mL, 0.313 mmol) was added. After 45 minutes, the reaction was quenched with sat. NH₄Cl solution and extracted three times with EtOAc. The organic layers were concentrated. The residue was purified via ISCO (12 g column; Hex/EtOAc; 0 to 100% gradient;) to give N-(3,5-dichloro-4-(3-fluoro-5-(2-hydroxypropan-2-yl)phenoxy)phenyl)-2-(3-(methylsulfonyl)phenyl)acetamide (24.7 mg, 73%). ¹H NMR (400 MHz, METHANOL-d4) δ 7.96 (s, 1H), 7.89 (d, J=7.8 Hz, 1H), 7.80 (s, 2H), 7.72 (d, J=7.7 Hz, 1H), 7.66-7.59 (m, 1H), 6.90 (dt, J=9.8, 2.0 Hz, 1H), 6.80 (t, J=1.6 Hz, 1H), 6.36 (dt, J=9.8, 2.3 Hz, 1H), 3.86 (s, 2H), 3.14 (s, 3H), 1.46 (s, 6H).

### Example 53: N-(3,5-dichloro-4-(3-fluoro-5-(2-fluoropropan-2-yl)phenoxy)phenyl)-2-(3-(methylsulfonyl)phenyl)acetamide

A solution of N-(3,5-dichloro-4-(3-fluoro-5-(2-hydroxypropan-2-yl)phenoxy)phenyl)-2-(3-(methylsulfonyl)phenyl)acetamide (0.017 g, 0.032 mmol) in DCM (0.323 ml) was cooled in a dry ice/acetone bath. DAST (1M in DCM) (0.040 ml, 0.040 mmol) was added. After 1.5 hours, the reaction was quenched with MeOH. After 10 minutes, the reaction was warmed to room temperature. The reaction was diluted with sat. NaHCO₃ solution and extracted twice with DCM. The organic layers were concentrated. The residue was purified via ISCO (12 g column; Hex/EtOAc; 0 to 100% gradient;). The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 51-76% B over 25 minutes, then a 2-minute hold at 100% B; Flow: 20 mL/min. Fractions containing the desired product were combined and dried via centrifugal evaporation to give N-(3,5-dichloro-4-(3-fluoro-5-(2-fluoropropan-2-yl)phenoxy)phenyl)-2-(3-(methylsulfonyl)phenyl)acetamide (7.1 mg. 42%). LCMS m/z 545.1 (M+NH₄); rt 2.11 min; Conditions A. ¹H NMR (500 MHz, DMSO-d₆) δ 10.72 (br s, 1H), 7.93-7.88 (m, 1H), 7.86-7.79 (m, 3H), 7.73-7.66 (m, 1H), 7.66-7.58 (m, 1H), 6.96 (br d, J=9.6 Hz, 1H), 6.71 (s, 1H), 6.60 (br d, J=10.0 Hz, 1H), 3.89-3.78 (m, 2H), 3.25-3.17 (m, 3H), 1.67-1.47 (m, 6H).

The following examples were synthesized according to the procedures described above.

| | | | Procedure |
|---|---|---|---|
| | | | Analogous |
| | | | to Example |
| Example | Structure & Name | Analytical Data | No. |
| 54 | 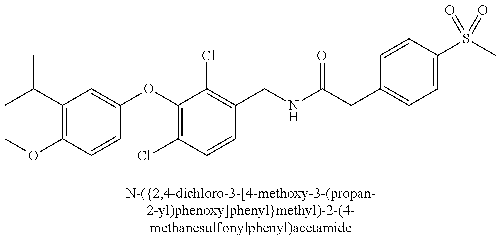 [SMILES: COc1ccc(Oc2c(Cl)ccc(CNC(=O)Cc3ccc(S(C)(=O)=O)cc3)c2Cl)cc1C(C)C] | Method C: rt = 1.03 min; Obs. Adducts: [M + H]; Obs. Mass: 535.8 | 20 |
| 55 | 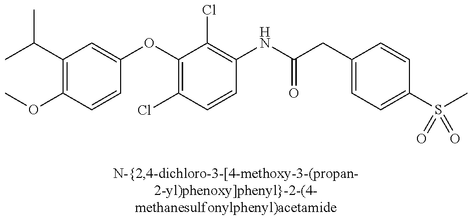 [SMILES: COc1ccc(Oc2c(Cl)ccc(NC(=O)Cc3ccc(S(C)(=O)=O)cc3)c2Cl)cc1C(C)C] | Method C: rt = 1.06 min; Obs. Adducts: [M + H]; Obs. Mass: 521.8 | 23 |
| 56 | 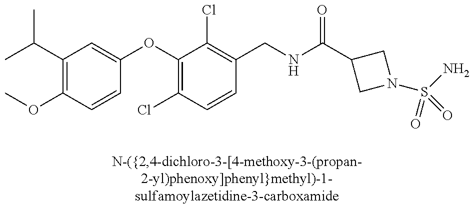 [SMILES: COc1ccc(Oc2c(Cl)ccc(CNC(=O)C3CN(S(N)(=O)=O)C3)c2Cl)cc1C(C)C] | Method C: rt = 0.97 min; Obs. Adducts: [M + H]; Obs. Mass: 501.9 | 20 |
| 57 | 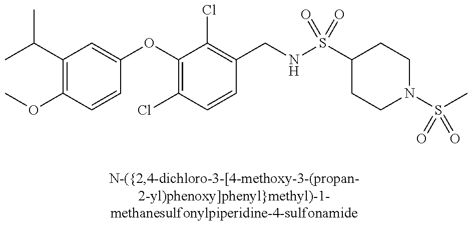 [SMILES: COc1ccc(Oc2c(Cl)ccc(CNS(=O)(=O)C3CCN(S(C)(=O)=O)CC3)c2Cl)cc1C(C)C] | Method C: rt = 1.04 min; Obs. Adducts: [M + H]; Obs. Mass: 564.7 | 20 |
| 58 | 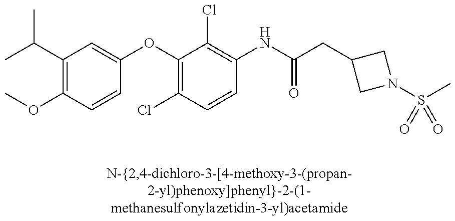 [SMILES: COc1ccc(Oc2c(Cl)ccc(NC(=O)CC3CN(S(C)(=O)=O)C3)c2Cl)cc1C(C)C] | Method C: rt = 0.99 min; Obs. Adducts: [M + H]; Obs. Mass: 500.8 | 23 |
| 59 | 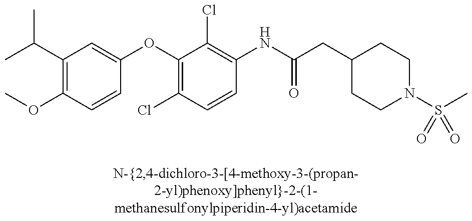 [SMILES: COc1ccc(Oc2c(Cl)ccc(NC(=O)CC3CCN(S(C)(=O)=O)CC3)c2Cl)cc1C(C)C] | Method C: rt = 1.06 min; Obs. Adducts: [M + H]; Obs. Mass: 528.9 | 23 |
| 60 | 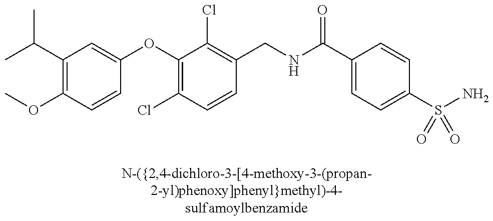 [SMILES: COc1ccc(Oc2c(Cl)ccc(CNC(=O)c3ccc(S(N)(=O)=O)cc3)c2Cl)cc1C(C)C] | Method C: rt = 1.02 min; Obs. Adducts: [M + H]; Obs. Mass: 522.9 | 20 |
| 61 | 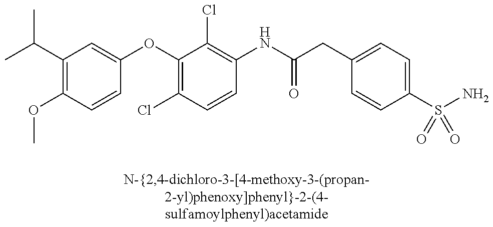 [SMILES: COc1ccc(Oc2c(Cl)ccc(NC(=O)Cc3ccc(S(N)(=O)=O)cc3)c2Cl)cc1C(C)C] | Method C: rt = 1.03 min; Obs. Adducts: [M + H]; Obs. Mass: 522.8 | 23 |
| 62 | 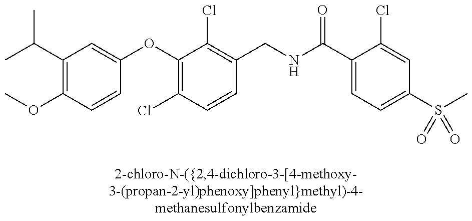 [SMILES: COc1ccc(Oc2c(Cl)ccc(CNC(=O)c3ccc(S(C)(=O)=O)cc3Cl)c2Cl)cc1C(C)C] | Method C: rt = 1.08 min; Obs. Adducts: [M + H]; Obs. Mass: 555.8 | 20 |
| 63 | 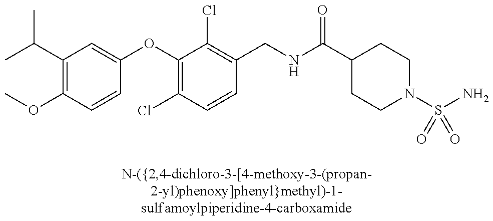 [SMILES: COc1ccc(Oc2c(Cl)ccc(CNC(=O)C3CCN(S(N)(=O)=O)CC3)c2Cl)cc1C(C)C] | Method C: rt = 1.00 min; Obs. Adducts: [M + H]; Obs. Mass: 530.1 | 20 |
| 64 | 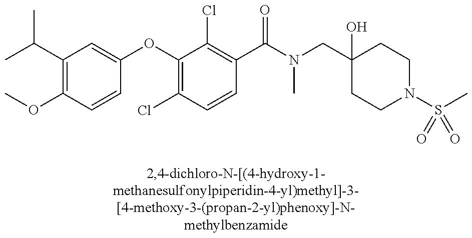 [SMILES: COc1ccc(Oc2c(Cl)ccc(C(=O)N(C)CC3(O)CCN(S(C)(=O)=O)CC3)c2Cl)cc1C(C)C] | Method C: rt = 1.00 min; Obs. Adducts: [M + H]; Obs. Mass: 558.9 | 19 |
| 65 | 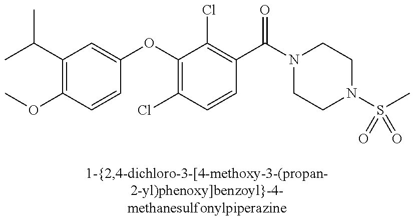 [SMILES: COc1ccc(Oc2c(Cl)ccc(C(=O)N3CCN(S(C)(=O)=O)CC3)c2Cl)cc1C(C)C] | Method C: rt = 1.01 min; Obs. Adducts: [M + H]; Obs. Mass: 500.9 | 19 |
| 66 | 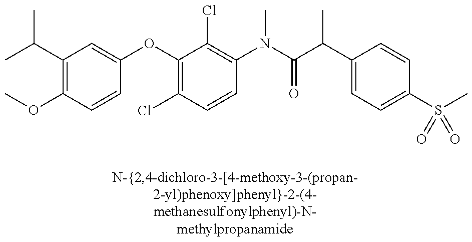 [SMILES: COc1ccc(Oc2c(Cl)ccc(N(C)C(=O)C(C)c3ccc(S(C)(=O)=O)cc3)c2Cl)cc1C(C)C] | Method C: rt = 1.09 min; Obs. Adducts: [M + H]; Obs. Mass: 549.8 | 20 |
| 67 | 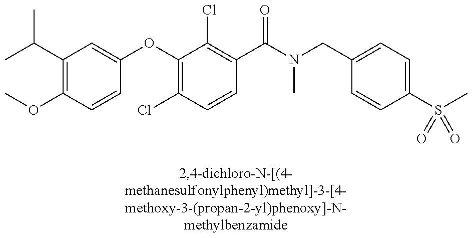 [SMILES: COc1ccc(Oc2c(Cl)ccc(C(=O)N(C)Cc3ccc(S(C)(=O)=O)cc3)c2Cl)cc1C(C)C] | Method C: rt = 1.05 min; Obs. Adducts: [M + H]: Obs. Mass: 535.8 | 19 |
| 68 | 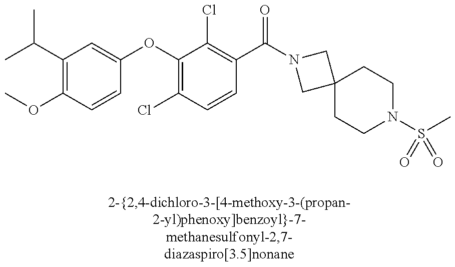 [SMILES: COc1ccc(Oc2c(Cl)ccc(C(=O)N3CC4(CCN(S(C)(=O)=O)CC4)C3)c2Cl)cc1C(C)C] | Method C: rt = 1.01 min; Obs. Adducts: [M + H]; Obs. Mass: 540.7 | 19 |
| 69 | 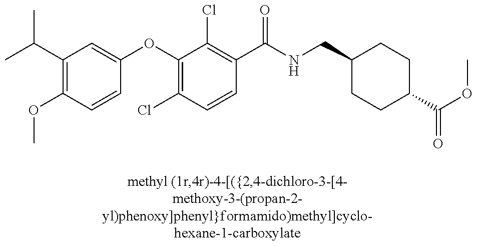 [SMILES: COc1ccc(Oc2c(Cl)ccc(C(=O)NC[C@H]3CC[C@H](C(=O)OC)CC3)c2Cl)cc1C(C)C] | Method C: rt = 1.10 min; Obs. Adducts: [M + H]; Obs. Mass: 507.8 | 19 |
| 70 | 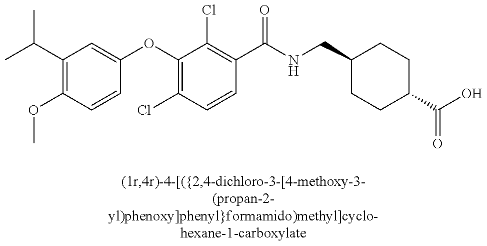 [SMILES: COc1ccc(Oc2c(Cl)ccc(C(=O)NC[C@H]3CC[C@H](C(=O)O)CC3)c2Cl)cc1C(C)C] | Method C: rt = 1.01 min; Obs. Adducts: [M + H]; Obs. Mass: 493.8 | 19 |
| 71 | 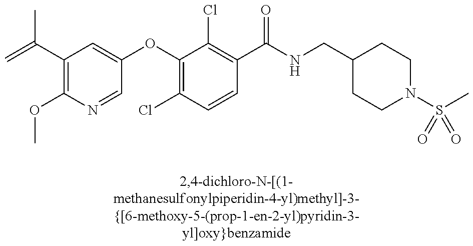 [SMILES: C=C(C)c1cc(Oc2c(Cl)ccc(C(=O)NCC3CCN(S(C)(=O)=O)CC3)c2Cl)cnc1OC] | Method C: rt = 0.98 min; Obs. Adducts: [M + H]; Obs. Mass: 527.7 | 19 |
| 72 | 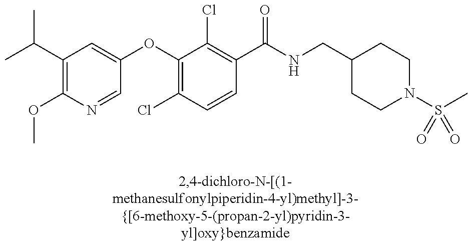 [SMILES: COc1ncc(Oc2c(Cl)ccc(C(=O)NCC3CCN(S(C)(=O)=O)CC3)c2Cl)cc1C(C)C] | Method C: rt = 0.99 min; Obs. Adducts: [M + H]; Obs. Mass: 529.8 | 19 |
| 73 | 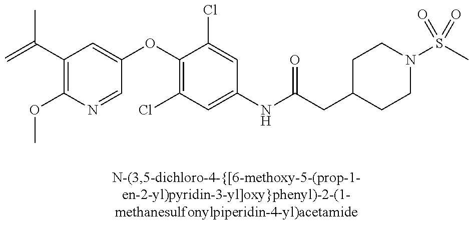 [SMILES: C=C(C)c1cc(Oc2c(Cl)cc(NC(=O)CC3CCN(S(C)(=O)=O)CC3)cc2Cl)cnc1OC] | Method C: rt = 1.11 min; Obs. Adducts: [M + H]; Obs. Mass: 528.1 | 15 |
| 74 | 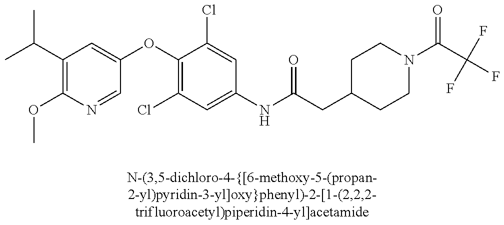 [SMILES: COc1ncc(Oc2c(Cl)cc(NC(=O)CC3CCN(C(=O)C(F)(F)F)CC3)cc2Cl)cc1C(C)C] | Method C: rt = 1.17 min; Obs. Adducts: [M + H]; Obs. Mass: 548.4 | 15 |
| 75 | 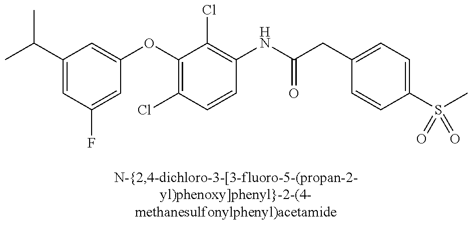 [SMILES: CC(C)c1cc(F)cc(Oc2c(Cl)ccc(NC(=O)Cc3ccc(S(C)(=O)=O)cc3)c2Cl)c1] | Method C: rt = 1.09 min; Obs. Adducts: [M + H]; Obs. Mass: 510.2 | 23 |
| 76 | 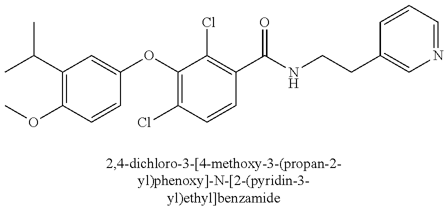 [SMILES: COc1ccc(Oc2c(Cl)ccc(C(=O)NCCc3cccnc3)c2Cl)cc1C(C)C] | Method A: rt = 2.07 min; Obs. Adducts: [M + H]; Obs. Mass: 459.16 Method B: rt = 1.74 min; Obs. Adducts: [M + H]; Obs. Mass: 459.14 | 19 |
| 77 | 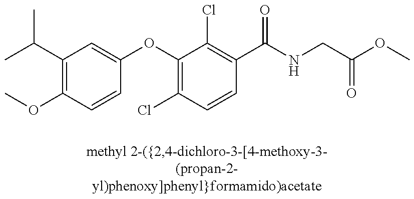 [SMILES: COC(=O)CNC(=O)c1ccc(Cl)c(Oc2ccc(OC)c(C(C)C)c2)c1Cl] | Method A: rt = 2.16 min; Obs. Adducts: [M + H]; Obs. Mass: 425.92 Method B: rt = 2.18 min; Obs. Adducts: [M + H]; Obs. Mass: 425.9 | 19 |
| 78 | 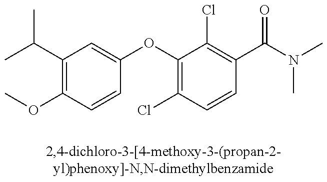 [SMILES: COc1ccc(Oc2c(Cl)ccc(C(=O)N(C)C)c2Cl)cc1C(C)C] | Method A: rt = 2.2 min; Obs. Adducts: [M + H]; Obs. Mass: 381.87 Method B: rt = 2.25 min; Obs. Adducts: [M + H]; Obs. Mass: 382.12 | 19 |
| 79 | 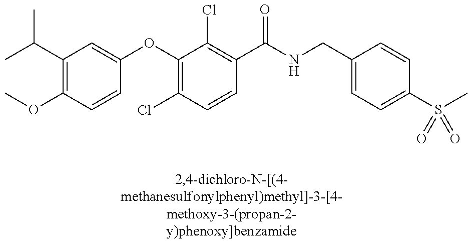 [SMILES: COc1ccc(Oc2c(Cl)ccc(C(=O)NCc3ccc(S(C)(=O)=O)cc3)c2Cl)cc1C(C)C] | Method A: rt = 2.18 min; Obs. Adducts: [M + H]; Obs. Mass: 522.21 Method B: rt = 2.19 min; Obs. Adducts: [M + H]; Obs. Mass: 522.21 | 19 |
| 80 | 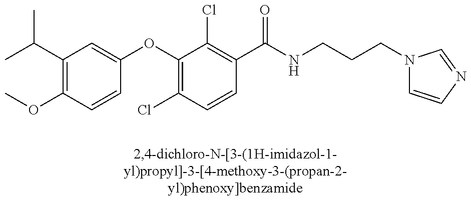 [SMILES: COc1ccc(Oc2c(Cl)ccc(C(=O)NCCCn3ccnc3)c2Cl)cc1C(C)C] | Method A: rt = 2.09 min; Obs. Adducts: [M + H]; Obs. Mass: 462.19 Method B: rt = 1.83 min; Obs. Adducts: [M + H]; Obs. Mass: 461.91 | 19 |
| 81 | 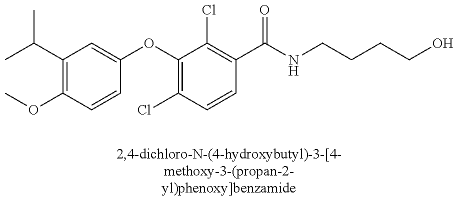 [SMILES: COc1ccc(Oc2c(Cl)ccc(C(=O)NCCCCO)c2Cl)cc1C(C)C] | Method A: rt = 1.93 min; Obs. Adducts: [M + H]; Obs. Mass: 425.98 Method B: rt = 1.97 min; Obs. Adducts: [M + H]; Obs. Mass: 426.22 | 19 |
| 82 | 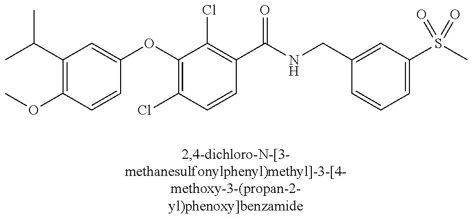 [SMILES: COc1ccc(Oc2c(Cl)ccc(C(=O)NCc3cccc(S(C)(=O)=O)c3)c2Cl)cc1C(C)C] | Method A: rt = 2.18 min; Obs. Adducts: [M + H]; Obs. Mass: 522.2 Method B: rt = 2.19 min; Obs. Adducts: [M + H]; Obs. Mass: 522.19 | 19 |
| 83 | 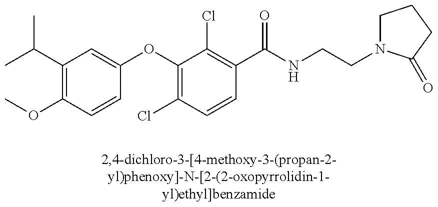 [SMILES: COc1ccc(Oc2c(Cl)ccc(C(=O)NCCN3CCCC3=O)c2Cl)cc1C(C)C] | Method A: rt = 1.98 min; Obs. Adducts: [M + H]; Obs. Mass: 465.07 Method B: rt = 1.94 min; Obs. Adducts: [M + H]; Obs. Mass: 465.13 | 19 |
| 84 | 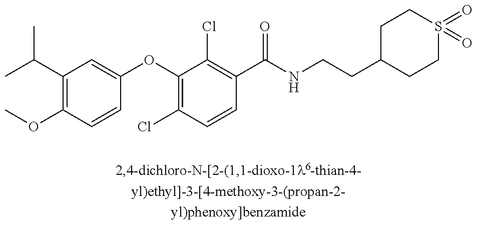 [SMILES: COc1ccc(Oc2c(Cl)ccc(C(=O)NCCC3CCS(=O)(=O)CC3)c2Cl)cc1C(C)C] | Method A: rt = 2.08 min; Obs. Adducts: [M + H]; Obs. Mass: 514.02 Method B: rt = 2.04 min; Obs. Adducts: [M + H]; Obs. Mass: 514.08 | 19 |
| 85 | 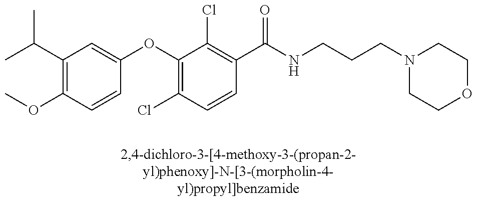 | Method A: rt = 1.97 min; Obs. Adducts: [M + H]; Obs. Mass: 467.27 Method B: rt = 1.71 min; Obs. Adducts: [M + H]; Obs. Mass: 467.13 | 19 |
| 86 | 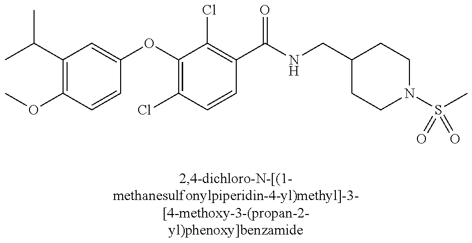 [SMILES: COc1ccc(Oc2c(Cl)ccc(C(=O)NCC3CCN(S(C)(=O)=O)CC3)c2Cl)cc1C(C)C] | Method A: rt = 2.13 min; Obs. Adducts: [M + H]; Obs. Mass: 529.14 Method B: rt = 2.13 min; Obs. Adducts: [M + H]; Obs. Mass: 529.14 | 19 |
| 87 | 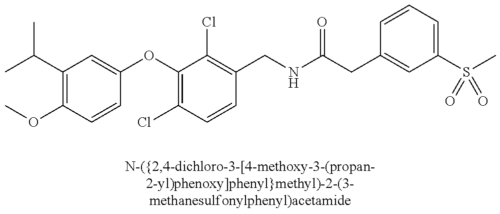 [SMILES: COc1ccc(Oc2c(Cl)ccc(CNC(=O)Cc3cccc(S(C)(=O)=O)c3)c2Cl)cc1C(C)C] | Method A: rt = 2.23 min; Obs. Adducts: [M + H]; Obs. Mass: 536.12 Method B: rt = 2.23 min; Obs. Adducts: [M + H]; Obs. Mass: 536.08 | 20 |
| 88 | 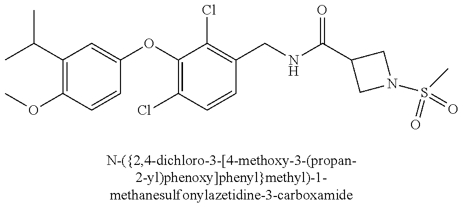 [SMILES: COc1ccc(Oc2c(Cl)ccc(CNC(=O)C3CN(S(C)(=O)=O)C3)c2Cl)cc1C(C)C] | Method A: rt = 2.17 min; Obs. Adducts: [M + H]; Obs. Mass: 501.16 Method B: rt = 2.16 min; Obs. Adducts: [M + H]; Obs. Mass: 501.18 | 20 |
| 89 | 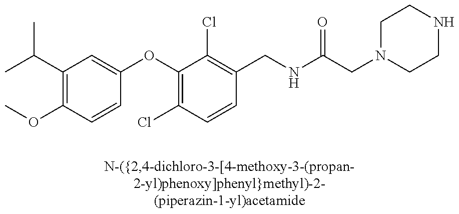 [SMILES: COc1ccc(Oc2c(Cl)ccc(CNC(=O)CN3CCNCC3)c2Cl)cc1C(C)C] | Method A: rt = 1.84 min; Obs. Adducts: [M + H]; Obs. Mass: 466.28 Method B: rt = 1.77 min; Obs. Adducts: [M + H]; Obs. Mass: 465.97 | 20 |
| 90 | 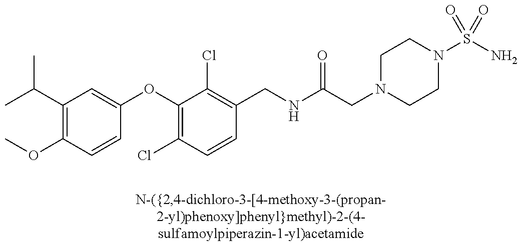 [SMILES: COc1ccc(Oc2c(Cl)ccc(CNC(=O)CN3CCN(S(N)(=O)=O)CC3)c2Cl)cc1C(C)C] | Method A: rt = 2.08 min; Obs. Adducts: [M + H]; Obs. Mass: 545.09 Method B: rt = 1.84 min; Obs. Adducts: [M + H]; Obs. Mass: 545.14 | 20 |
| 91 | 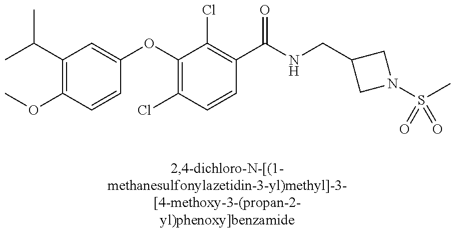 [SMILES: COc1ccc(Oc2c(Cl)ccc(C(=O)NCC3CN(S(C)(=O)=O)C3)c2Cl)cc1C(C)C] | Method A: rt = 2.07 min; Obs. Adducts: [M + H]; Obs. Mass: 500.92 Method B: rt = 2.07 min; Obs. Adducts: [M + H]; Obs. Mass: 501.2 | 19 |
| 92 | 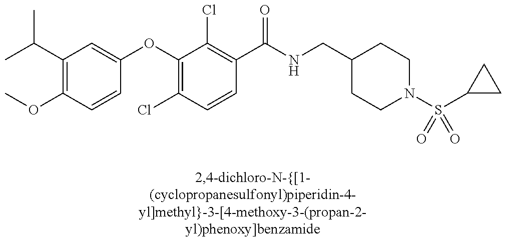 [SMILES: COc1ccc(Oc2c(Cl)ccc(C(=O)NCC3CCN(S(=O)(=O)C4CC4)CC3)c2Cl)cc1C(C)C] | Method A: rt = 2.23 min; Obs. Adducts: [M + H]; Obs. Mass: 555.05 Method B: rt = 2.23 min; Obs. Adducts: [M + H]; Obs. Mass: 555.32 | 19 |
| 93 | 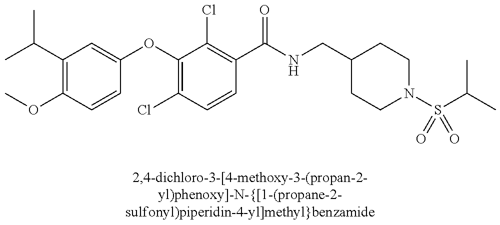 [SMILES: COc1ccc(Oc2c(Cl)ccc(C(=O)NCC3CCN(S(=O)(=O)C(C)C)CC3)c2Cl)cc1C(C)C] | Method A: rt = 2.29 min; Obs. Adducts: [M + H]; Obs. Mass: 557.15 Method B: rt = 2.31 min; Obs. Adducts: [M + H]; Obs. Mass: 557.19 | 19 |
| 94 | 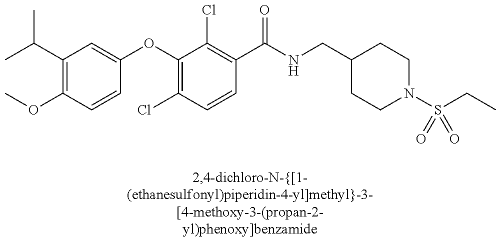 [SMILES: CCS(=O)(=O)N1CCC(CNC(=O)c2ccc(Cl)c(Oc3ccc(OC)c(C(C)C)c3)c2Cl)CC1] | Method A: rt = 2.2 min; Obs. Adducts: [M + H]; Obs. Mass: 543.18 Method B: rt = 2.22 min; Obs. Adducts: [M + H]; Obs. Mass: 543.17 | 19 |
| 95 | 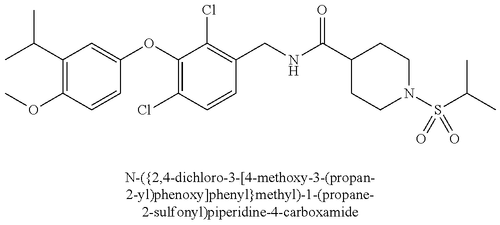 [SMILES: COc1ccc(Oc2c(Cl)ccc(CNC(=O)C3CCN(S(=O)(=O)C(C)C)CC3)c2Cl)cc1C(C)C] | Method A: rt = 2.33 min; Obs. Adducts: [M + H]; Obs. Mass: 557.23 Method B: rt = 2.33 min; Obs. Adducts: [M + H]; Obs. Mass: 556.96 | 20 |
| 96 | 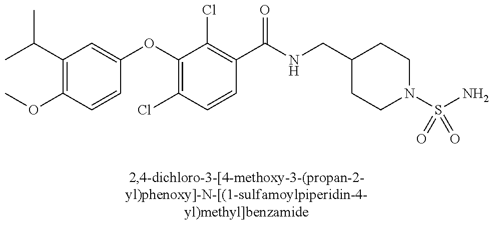 [SMILES: COc1ccc(Oc2c(Cl)ccc(C(=O)NCC3CCN(S(N)(=O)=O)CC3)c2Cl)cc1C(C)C] | Method A: rt = 2.02 min; Obs. Adducts: [M + H]; Obs. Mass: 530.13 Method B: rt = 2.05 min; Obs. Adducts: [M + H]; Obs. Mass: 530.21 | 19 |
| 97 | 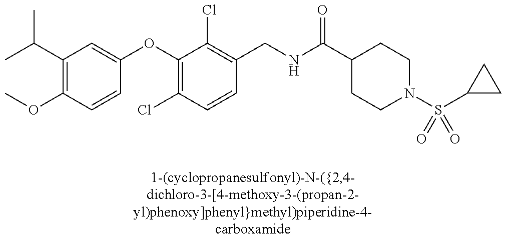 [SMILES: COc1ccc(Oc2c(Cl)ccc(CNC(=O)C3CCN(S(=O)(=O)C4CC4)CC3)c2Cl)cc1C(C)C] | Method B: rt = 2.3 min; Obs. Adducts: [M + H]; Obs. Mass: 555.19 Method A: rt = 2.28 min; Obs. Adducts: [M + H]; Obs. Mass: 555.07 | 20 |
| 98 | 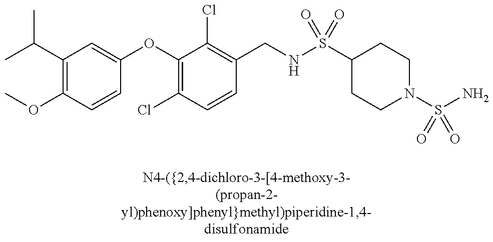 [SMILES: COc1ccc(Oc2c(Cl)ccc(CNS(=O)(=O)C3CCN(S(N)(=O)=O)CC3)c2Cl)cc1C(C)C] | Method A: rt = 2.18 min; Obs. Adducts: [M + H]; Obs. Mass: 565.92 Method B: rt = 2.17 min; Obs. Adducts: [M + H]; Obs. Mass: 566.11 | 20 |
| 99 | 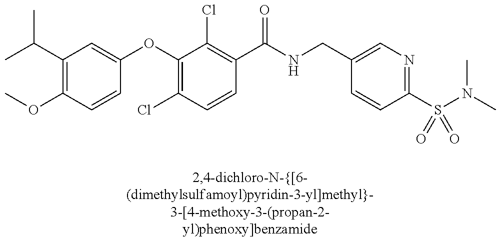 [SMILES: COc1ccc(Oc2c(Cl)ccc(C(=O)NCc3ccc(S(=O)(=O)N(C)C)nc3)c2Cl)cc1C(C)C] | Method A: rt = 2.27 min; Obs. Adducts: [M + H]; Obs. Mass: 552.28 Method B: rt = 2.26 min; Obs. Adducts: [M + H]; Obs. Mass: 552.24 | 19 |
| 100 | 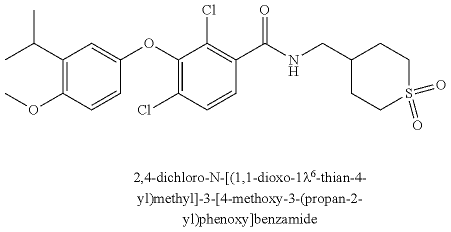 [SMILES: COc1ccc(Oc2c(Cl)ccc(C(=O)NCC3CCS(=O)(=O)CC3)c2Cl)cc1C(C)C] | Method B: rt = 2.11 min; Obs. Adducts: [M + H]; Obs. Mass: 500.26 Method A: rt = 2.05 min; Obs. Adducts: [M + H]; Obs. Mass: 500.28 | 19 |
| 101 | 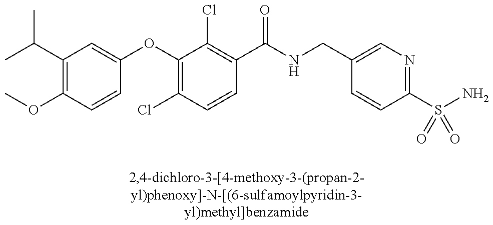 [SMILES: COc1ccc(Oc2c(Cl)ccc(C(=O)NCc3ccc(S(N)(=O)=O)nc3)c2Cl)cc1C(C)C] | Method B: rt = 2.02 min; Obs. Adducts: [M + H]; Obs. Mass: 524.25 Method A: rt = 2.06 min; Obs. Adducts: [M + H]; Obs. Mass: 524.18 | 19 |
| 102 | 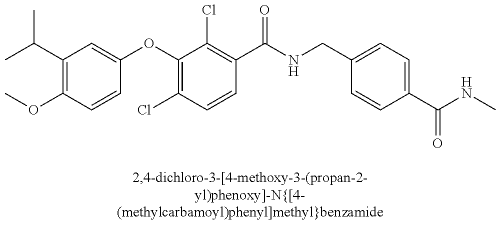 [SMILES: CNC(=O)c1ccc(CNC(=O)c2ccc(Cl)c(Oc3ccc(OC)c(C(C)C)c3)c2Cl)cc1] | Method A: rt = 2.06 min; Obs. Adducts: [M + H]; Obs. Mass: 501.04 Method B: rt = 2.04 min; Obs. Adducts: [M + H]: Obs. Mass: 501.04 | 19 |
| 103 | 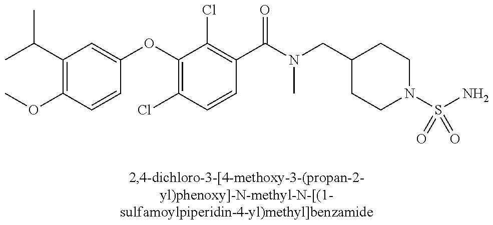 [SMILES: COc1ccc(Oc2c(Cl)ccc(C(=O)N(C)CC3CCN(S(N)(=O)=O)CC3)c2Cl)cc1C(C)C] | Method B: rt = 2.15 min; Obs. Adducts: [M + H]; Obs. Mass: 544.1 Method A: rt = 2.16 min; Obs. Adducts: [M + H]; Obs. Mass: 544.11 | 19 |
| 104 | 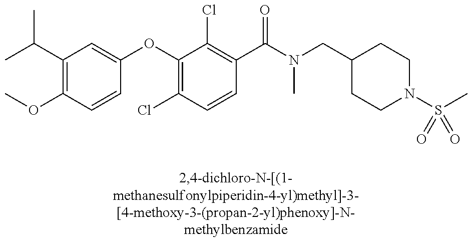 [SMILES: COc1ccc(Oc2c(Cl)ccc(C(=O)N(C)CC3CCN(S(C)(=O)=O)CC3)c2Cl)cc1C(C)C] | Method A: rt = 2.27 min; Obs. Adducts: [M + H]; Obs. Mass: 543.05 Method B: rt = 2.24 min; Obs. Adducts: [M + H]; Obs. Mass: 543.12 | 19 |
| 105 | 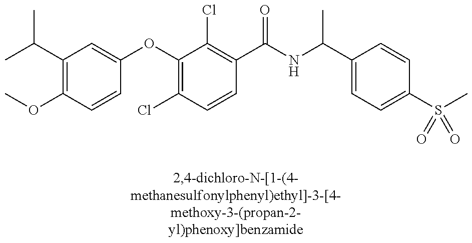 [SMILES: COc1ccc(Oc2c(Cl)ccc(C(=O)NC(C)c3ccc(S(C)(=O)=O)cc3)c2Cl)cc1C(C)C] | Method B: rt = 2.22 min; Obs. Adducts: [M + H]; Obs. Mass: 536.18 Method A: rt = 2.25 min; Obs. Adducts: [M + H]; Obs. Mass: 535.99 | 19 |
| 106 | 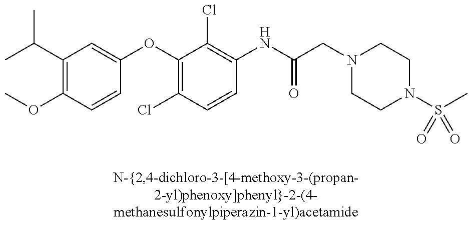 [SMILES: COc1ccc(Oc2c(Cl)ccc(NC(=O)CN3CCN(S(C)(=O)=O)CC3)c2Cl)cc1C(C)C] | Method A: rt = 2.33 min; Obs. Adducts: [M + H]; Obs. Mass: 530.3 Method B: rt = 1.95 min; Obs. Adducts: [M + H]; Obs. Mass: 530.31 | 23 |
| 107 | 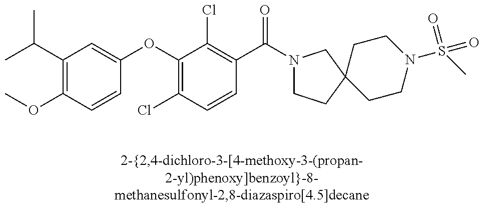 [SMILES: COc1ccc(Oc2c(Cl)ccc(C(=O)N3CCC4(CCN(S(C)(=O)=O)CC4)C3)c2Cl)cc1C(C)C] | Method A: rt = 2.24 min; Obs. Adducts: [M + H]; Obs. Mass: 554.97 Method B: rt = 2.24 min; Obs. Adducts: [M + H]; Obs. Mass: 555.31 | 19 |
| 108 | 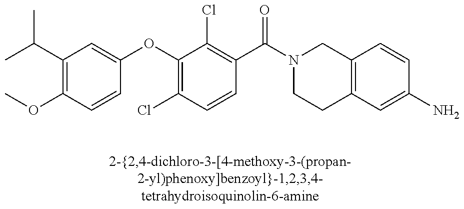 [SMILES: COc1ccc(Oc2c(Cl)ccc(C(=O)N3CCc4cc(N)ccc4C3)c2Cl)cc1C(C)C] | Method A: rt = 2.23 min; Obs. Adducts: [M + H]; Obs. Mass: 485.24 Method B: rt = 1.87 min; Obs. Adducts: [M + H]; Obs. Mass: 485.28 | 19 |
| 109 | 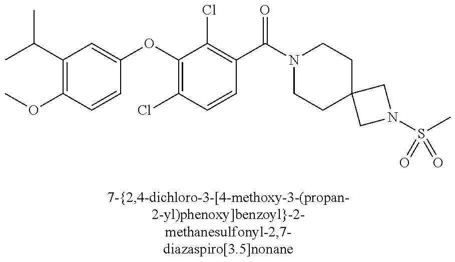 [SMILES: COc1ccc(Oc2c(Cl)ccc(C(=O)N3CCC4(CC3)CN(S(C)(=O)=O)C4)c2Cl)cc1C(C)C] | Method A: rt = 2.18 min; Obs. Adducts: [M + H]; Obs. Mass: 541.16 Method B: rt = 2.19 min; Obs. Adducts: [M + H]; Obs. Mass: 541.14 | 19 |
| 110 | 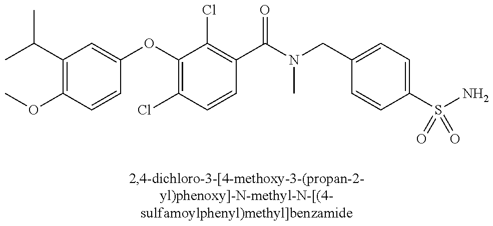 [SMILES: COc1ccc(Oc2c(Cl)ccc(C(=O)N(C)Cc3ccc(S(N)(=O)=O)cc3)c2Cl)cc1C(C)C] | Method B: rt = 2.17 min; Obs. Adducts: [M + H]; Obs. Mass: 537.06 Method A: rt = 2.17 min; Obs. Adducts: [M + H]; Obs. Mass: 537.12 | 19 |
| 111 | 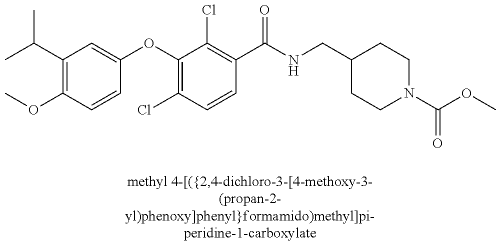 [SMILES: COC(=O)N1CCC(CNC(=O)c2ccc(Cl)c(Oc3ccc(OC)c(C(C)C)c3)c2Cl)CC1] | Method A: rt = 2.22 min; Obs. Adducts: [M + H]; Obs. Mass: 508.92 Method B: rt = 2.24 min; Obs. Adducts: [M + H]; Obs. Mass: 509.06 | 19 |
| 112 | 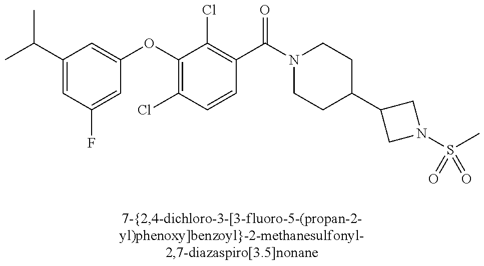 | Method A: rt = 2.2 min; Obs. Adducts: [M + H]; Obs. Mass: 529.02 Method B: rt = 2.15 min; Obs. Adducts: [M + H]; Obs. Mass: 529.34 | 19 |
| 113 | 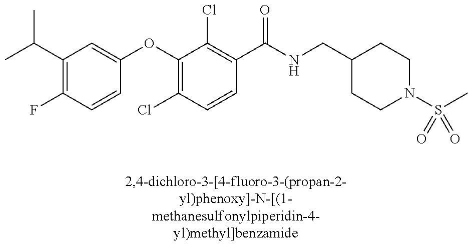 [SMILES: CC(C)c1cc(Oc2c(Cl)ccc(C(=O)NCC3CCN(S(C)(=O)=O)CC3)c2Cl)ccc1F] | Method A: rt = 2.18 min; Obs. Adducts: [M + H]: Obs. Mass: 517.04 Method B: rt = 2.1 min; Obs. Adducts: [M + H]; Obs. Mass: 517.28 | 19 |
| 114 | 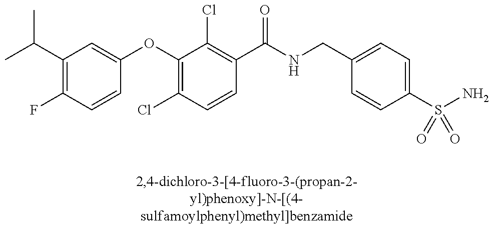 [SMILES: CC(C)c1cc(Oc2c(Cl)ccc(C(=O)NCc3ccc(S(N)(=O)=O)cc3)c2Cl)ccc1F] | Method B: rt = 2.04 min; Obs. Adducts: [M + H]; Obs. Mass: 511.28 Method A: rt = 2.1 min; Obs. Adducts: [M + H]; Obs. Mass: 511.14 | 19 |
| 115 | 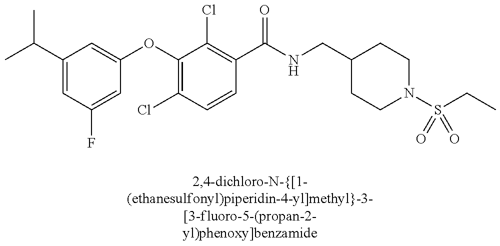 [SMILES: CCS(=O)(=O)N1CCC(CNC(=O)c2ccc(Cl)c(Oc3cc(F)cc(C(C)C)c3)c2Cl)CC1] | Method A: rt = 2.22 min; Obs. Adducts: [M + H]; Obs. Mass: 531.35 Method B: rt = 2.19 min; Obs. Adducts: [M + H]; Obs. Mass: 531.3 | 19 |
| 116 | 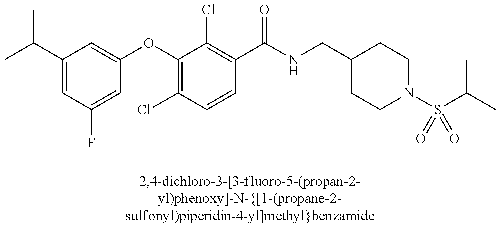 [SMILES: CC(C)c1cc(F)cc(Oc2c(Cl)ccc(C(=O)NCC3CCN(S(=O)(=O)C(C)C)CC3)c2Cl)c1] | Method B: rt = 2.31 min; Obs. Adducts: [M + H]; Obs. Mass: 544.96 Method A: rt = 2.3 min; Obs. Adducts: [M + H]; Obs. Mass: 545.32 | 19 |
| 117 | 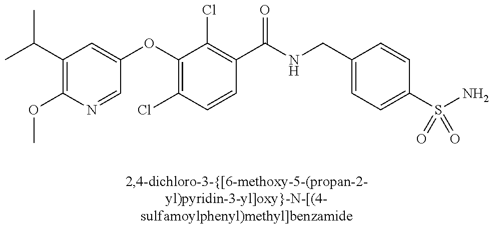 [SMILES: COc1ncc(Oc2c(Cl)ccc(C(=O)NCc3ccc(S(N)(=O)=O)cc3)c2Cl)cc1C(C)C] | Method A: rt = 1.98 min; Obs. Adducts: [M + H]; Obs. Mass: 524.17 Method B: rt = 1.98 min; Obs. Adducts: [M + H]; Obs. Mass: 524.22 | 19 |
| 118 | 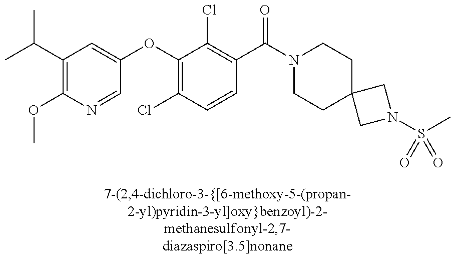 [SMILES: COc1ncc(Oc2c(Cl)ccc(C(=O)N3CCC4(CC3)CN(S(C)(=O)=O)C4)c2Cl)cc1C(C)C] | Method A: rt = 2.11 min; Obs. Adducts: [M + H]; Obs. Mass: 541.96 Method B: rt = 2.11 min; Obs. Adducts: [M + H]; Obs. Mass: 542.38 | 19 |
| 119 | 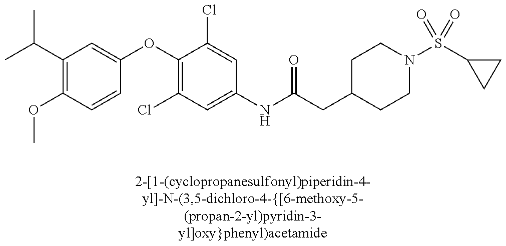 [SMILES: COc1ccc(Oc2c(Cl)cc(NC(=O)CC3CCN(S(=O)(=O)C4CC4)CC3)cc2Cl)cc1C(C)C] | Method A: rt = 2.42 min; Obs. Adducts: [M + H]; Obs. Mass: 556.14 Method B: rt = 2.42 min; Obs. Adducts: [M + H]; Obs. Mass: 555.83 | 15 |
| 120 | 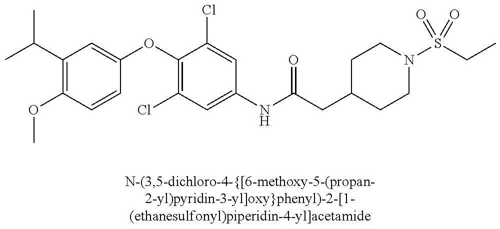 [SMILES: CCS(=O)(=O)N1CCC(CC(=O)Nc2cc(Cl)c(Oc3ccc(OC)c(C(C)C)c3)c(Cl)c2)CC1] | Method A: rt = 2.42 min; Obs. Adducts: [M + H]; Obs. Mass: 543.96 Method B: rt = 2.38 min; Obs. Adducts: [M + H]; Obs. Mass: 543.79 | 15 |
| 121 | 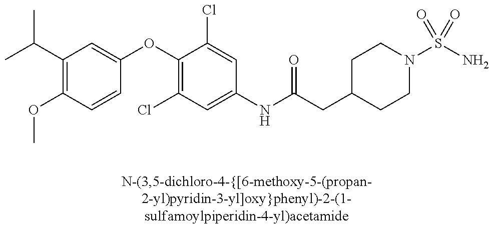 [SMILES: COc1ccc(Oc2c(Cl)cc(NC(=O)CC3CCN(S(N)(=O)=O)CC3)cc2Cl)cc1C(C)C] | Method A: rt = 2.2 min; Obs. Adducts: [M + H]; Obs. Mass: 530.95 Method B: rt = 2.18 min; Obs. Adducts: [M + H]; Obs. Mass: 530.79 | |
| 122 | 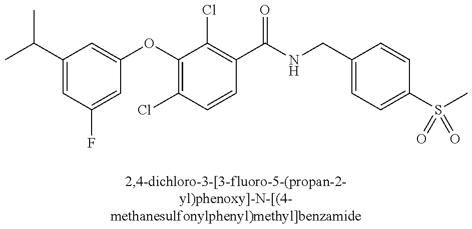 [SMILES: CC(C)c1cc(F)cc(Oc2c(Cl)ccc(C(=O)NCc3ccc(S(C)(=O)=O)cc3)c2Cl)c1] | Method B: rt = 2.18 min; Obs. Adducts: [M + H]; Obs. Mass: 510.16 Method A: rt = 2.18 min; Obs. Adducts: [M + H]; Obs. Mass: 510.25 | 19 |
| 123 | 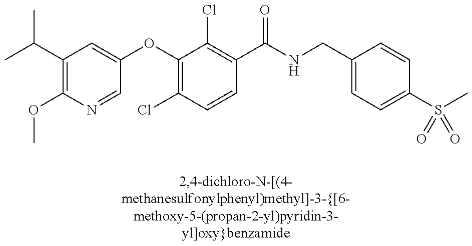 [SMILES: COc1ncc(Oc2c(Cl)ccc(C(=O)NCc3ccc(S(C)(=O)=O)cc3)c2Cl)cc1C(C)C] | Method A: rt = 2.11 min; Obs. Adducts: [M + H]; Obs. Mass: 523.39 Method B: rt = 2.15 min; Obs. Adducts: [M + H]; Obs. Mass: 523.33 | 19 |
| 124 | 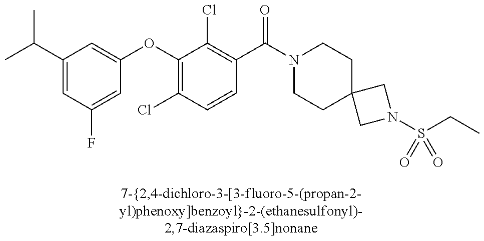 [SMILES: CCS(=O)(=O)N1CC2(CCN(C(=O)c3ccc(Cl)c(Oc4cc(F)cc(C(C)C)c4)c3Cl)CC2)C1] | Method A: rt = 2.29 min; Obs. Adducts: [M + H]; Obs. Mass: 543.08 Method B: rt = 2.3 min; Obs. Adducts: [M + H]; Obs. Mass: 543.35 | 19 |
| 125 | 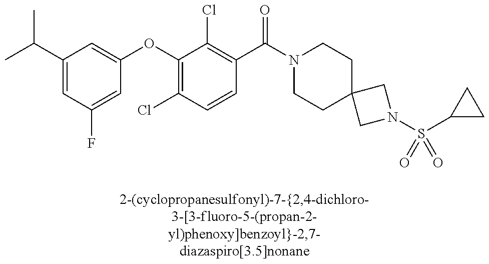 [SMILES: CC(C)c1cc(F)cc(Oc2c(Cl)ccc(C(=O)N3CCC4(CC3)CN(S(=O)(=O)C3CC3)C4)c2Cl)c1] | Method A: rt = 2.31 min; Obs. Adducts: [M + H]; Obs. Mass: 555 Method B: rt = 2.32 min; Obs. Adducts: [M + H]; Obs. Mass: 555.48 | 19 |
| 126 | 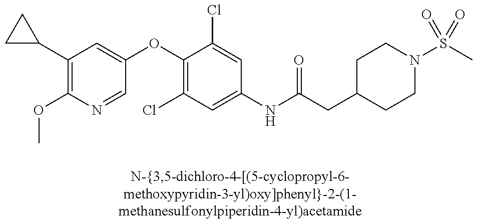 [SMILES: COc1ncc(Oc2c(Cl)cc(NC(=O)CC3CCN(S(C)(=O)=O)CC3)cc2Cl)cc1C1CC1] | Method A: rt = 2.19 min; Obs. Adducts: [M + H]; Obs. Mass: 528.1 Method B: rt = 2.2 min; Obs. Adducts: [M + H]; Obs. Mass: 528.35 | 15 |
| 127 | 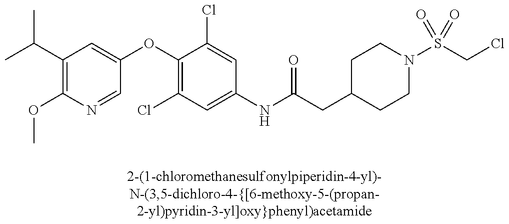 [SMILES: COc1ncc(Oc2c(Cl)cc(NC(=O)CC3CCN(S(=O)(=O)CCl)CC3)cc2Cl)cc1C(C)C] | Method A: rt = 2.46 min; Obs. Adducts: [M + H]; Obs. Mass: 564.36 Method B: rt = 2.46 min; Obs. Adducts: [M + H]; Obs. Mass: 564.35 | 15 |
| 128 | 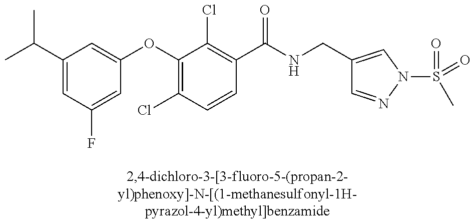 [SMILES: CC(C)c1cc(F)cc(Oc2c(Cl)ccc(C(=O)NCc3cnn(S(C)(=O)=O)c3)c2Cl)c1] | Method A: rt = 2.14 min; Obs. Adducts: [M + H]; Obs. Mass: 499.92 Method B: rt = 2.1 min; Obs. Adducts: [M + H]; Obs. Mass: 499.79 | 19 |
| 129 | 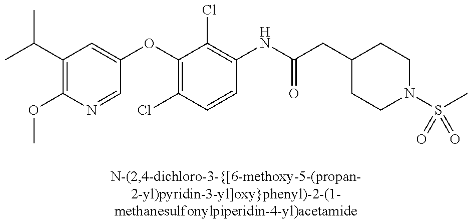 [SMILES: COc1ncc(Oc2c(Cl)ccc(NC(=O)CC3CCN(S(C)(=O)=O)CC3)c2Cl)cc1C(C)C] | Method A: rt = 2.22 min; Obs. Adducts: [M + H]; Obs. Mass: 530.19 Method B: rt = 2.23 min; Obs. Adducts: [M + H]; Obs. Mass: 530.19 | 23 |
| 130 | 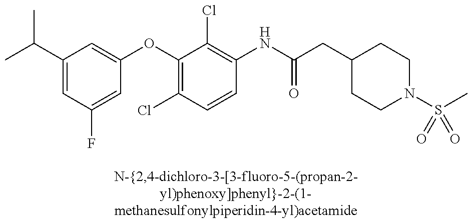 [SMILES: CC(C)c1cc(F)cc(Oc2c(Cl)ccc(NC(=O)CC3CCN(S(C)(=O)=O)CC3)c2Cl)c1] | Method A: rt = 2.34 min; Obs. Adducts: [M + H]; Obs. Mass: 516.98 Method B: rt = 2.22 min; Obs. Adducts: [M + H]; Obs. Mass: 517 | 23 |
| 131 | 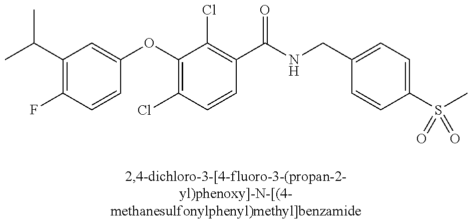 [SMILES: CC(C)c1cc(Oc2c(Cl)ccc(C(=O)NCc3ccc(S(C)(=O)=O)cc3)c2Cl)ccc1F] | Method B: rt = 2.14 min; Obs. Adducts: [M + H]; Obs. Mass: 510.26 Method A: rt = 2.27 min; Obs. Adducts: [M + H]; Obs. Mass: 510.14 | 19 |
| 132 | 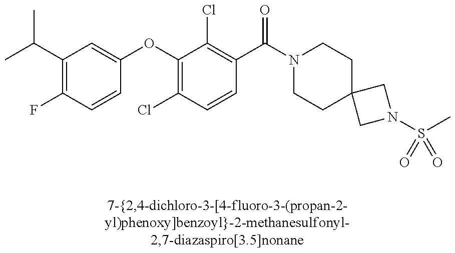 [SMILES: CC(C)c1cc(Oc2c(Cl)ccc(C(=O)N3CCC4(CC3)CN(S(C)(=O)=O)C4)c2Cl)ccc1F] | Method A: rt = 2.16 min; Obs. Adducts: [M + H]; Obs. Mass: 529.2 Method B: rt = 2.15 min; Obs. Adducts: [M + H]; Obs. Mass: 529.01 | 19 |
| 133 | 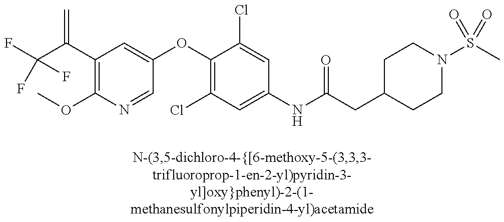 [SMILES: C=C(c1cc(Oc2c(Cl)cc(NC(=O)CC3CCN(S(C)(=O)=O)CC3)cc2Cl)cnc1OC)C(F)(F)F] | Method A: rt = 2.27 min; Obs. Adducts: [M + H]; Obs. Mass: 581.98 Method B: rt = 2.24 min; Obs. Adducts: [M + H]; Obs. Mass: 582.29 | 15 |
| 134 | 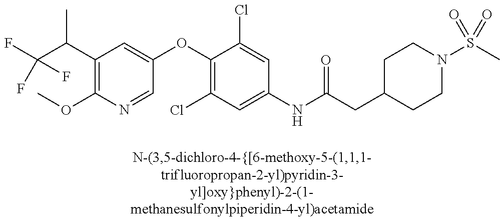 [SMILES: COc1ncc(Oc2c(Cl)cc(NC(=O)CC3CCN(S(C)(=O)=O)CC3)cc2Cl)cc1C(C)C(F)(F)F] | Method A: rt = 2.4 min; Obs. Adducts: [M + H]; Obs. Mass: 584.02 Method B: rt = 2.39 min; Obs. Adducts: [M + H]; Obs. Mass: 584.07 | 15 |
| 135 | 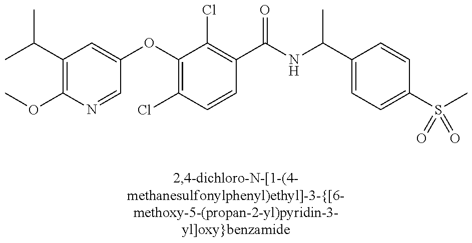 [SMILES: COc1ncc(Oc2c(Cl)ccc(C(=O)NC(C)c3ccc(S(C)(=O)=O)cc3)c2Cl)cc1C(C)C] | Method A: rt = 2.16 min; Obs. Adducts: [M + H]; Obs. Mass: 537.22 Method B: rt = 2.19 min; Obs. Adducts: [M + H]; Obs. Mass: 537.21 | 19 |
| 136 | 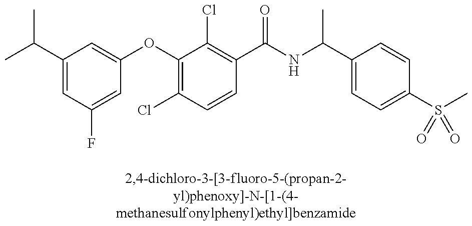 [SMILES: CC(C)c1cc(F)cc(Oc2c(Cl)ccc(C(=O)NC(C)c3ccc(S(C)(=O)=O)cc3)c2Cl)c1] | Method A: rt = 2.22 min; Obs. Adducts: [M + H]; Obs. Mass: 524.1 Method B: rt = 2.25 min; Obs. Adducts: [M + H]; Obs. Mass: 524.2 | 19 |
| 137 | 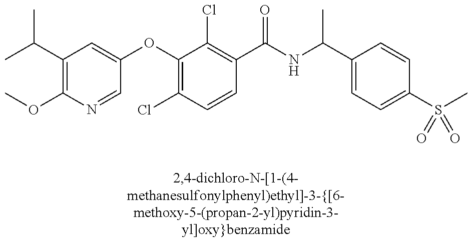 [SMILES: COc1ncc(Oc2c(Cl)ccc(C(=O)NC(C)c3ccc(S(C)(=O)=O)cc3)c2Cl)cc1C(C)C] | Method A: rt = 2.18 min; Obs. Adducts: [M + H]; Obs. Mass: 537.12 Method B: rt = 2.17 min; Obs. Adducts: [M + H]; Obs. Mass: 537.09 | 19 |
| 138 | 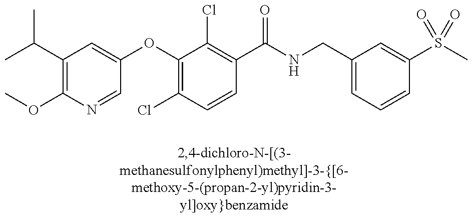 [SMILES: COc1ncc(Oc2c(Cl)ccc(C(=O)NCc3cccc(S(C)(=O)=O)c3)c2Cl)cc1C(C)C] | Method B: rt = 2.11 min; Obs. Adducts: [M + H]; Obs. Mass: 523.14 Method A: rt = 2.09 min; Obs. Adducts: [M + H]; Obs. Mass: 523.29 | 19 |
| 139 | 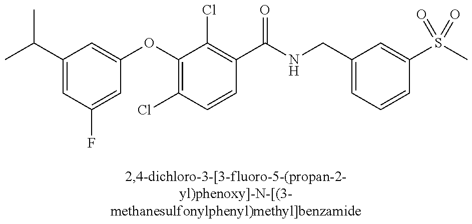 [SMILES: CC(C)c1cc(F)cc(Oc2c(Cl)ccc(C(=O)NCc3cccc(S(C)(=O)=O)c3)c2Cl)c1] | Method A: rt = 2.18 min; Obs. Adducts: [M + H]; Obs. Mass: 510.2 Method B: rt = 2.18 min; Obs. Adducts: [M + H]; Obs. Mass: 510.15 | 19 |
| 140 | 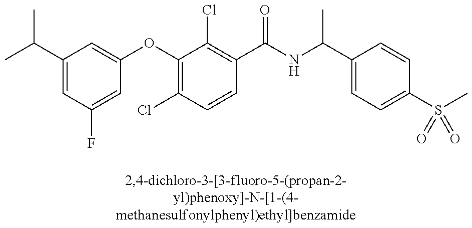 [SMILES: CC(C)c1cc(F)cc(Oc2c(Cl)ccc(C(=O)NC(C)c3ccc(S(C)(=O)=O)cc3)c2Cl)c1] | Method A: rt = 2.24 min; Obs. Adducts: [M + H]; Obs. Mass: 524.1 Method B: rt = 2.23 min; Obs. Adducts: [M + H]; Obs. Mass: 524.28 | 19 |
| 141 | 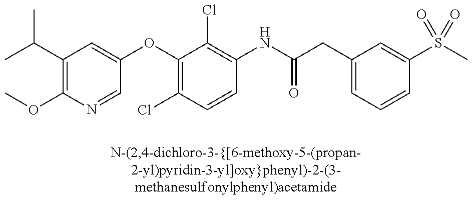 [SMILES: COc1ncc(Oc2c(Cl)ccc(NC(=O)Cc3cccc(S(C)(=O)=O)c3)c2Cl)cc1C(C)C] | Method A: rt = 2.28 min; Obs. Adducts: [M + H]; Obs. Mass: 523.25 Method B: rt = 2.26 min; Obs. Adducts: [M + H]; Obs. Mass: 523.11 | 23 |
| 142 | 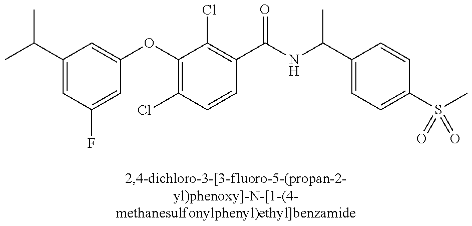 [SMILES: CC(C)c1cc(F)cc(Oc2c(Cl)ccc(C(=O)NC(C)c3ccc(S(C)(=O)=O)cc3)c2Cl)c1] | Method A: rt = 2.24 min; Obs. Adducts: [M + H]; Obs. Mass: 524.24 Method B: rt = 2.23 min; Obs. Adducts: [M + H]: Obs. Mass: 524.24 | 19 |
| 143 | 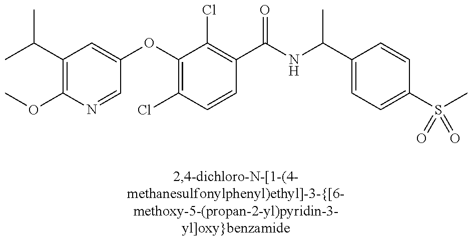 [SMILES: COc1ncc(Oc2c(Cl)ccc(C(=O)NC(C)c3ccc(S(C)(=O)=O)cc3)c2Cl)cc1C(C)C] | Method A: rt = 2.18 min; Obs. Adducts: [M + H]; Obs. Mass: 537.28 Method B: rt = 2.17 min; Obs. Adducts: [M + H]; Obs. Mass: 537.11 | 19 |
| 144 | 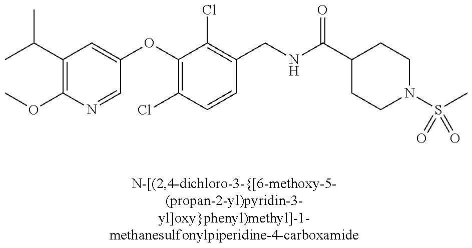 [SMILES: COc1ncc(Oc2c(Cl)ccc(CNC(=O)C3CCN(S(C)(=O)=O)CC3)c2Cl)cc1C(C)C] | Method A: rt = 2.14 min; Obs. Adducts: [M + H]; Obs. Mass: 530.31 Method B: rt = 2.13 min; Obs. Adducts: [M + H]; Obs. Mass: 530.1 | 20 |
| 145 | 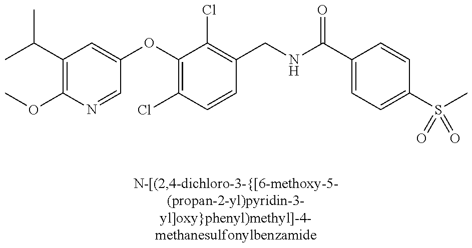 [SMILES: COc1ncc(Oc2c(Cl)ccc(CNC(=O)c3ccc(S(C)(=O)=O)cc3)c2Cl)cc1C(C)C] | Method A: rt = 2.25 min; Obs. Adducts: [M + H]; Obs. Mass: 523.1 Method B: rt = 2.24 min; Obs. Adducts: [M + H]; Obs. Mass: 523.03 | 20 |
| 146 | 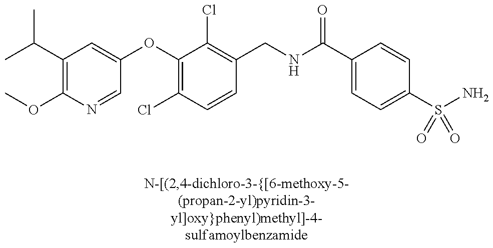 [SMILES: COc1ncc(Oc2c(Cl)ccc(CNC(=O)c3ccc(S(N)(=O)=O)cc3)c2Cl)cc1C(C)C] | Method A: rt = 2.14 min; Obs. Adducts: [M + H]; Obs. Mass: 523.9 Method B: rt = 2.12 min; Obs. Adducts: [M + H]; Obs. Mass: 524.3 | 20 |
| 147 | 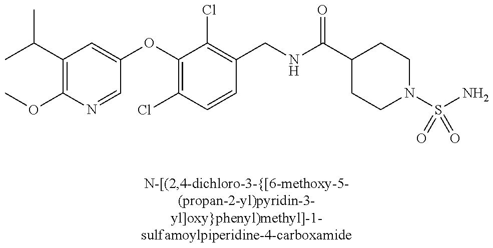 [SMILES: COc1ncc(Oc2c(Cl)ccc(CNC(=O)C3CCN(S(N)(=O)=O)CC3)c2Cl)cc1C(C)C] | Method A: rt = 2.04 min; Obs. Adducts: [M + H]; Obs. Mass: 531.1 Method B: rt = 2.02 min; Obs. Adducts: [M + H]; Obs. Mass: 531.19 | 20 |
| 148 | 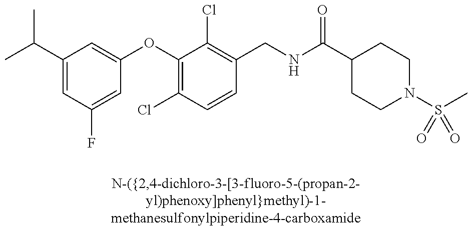 [SMILES: CC(C)c1cc(F)cc(Oc2c(Cl)ccc(CNC(=O)C3CCN(S(C)(=O)=O)CC3)c2Cl)c1] | Method A: rt = 2.23 min; Obs. Adducts: [M + H]; Obs. Mass: 517.34 Method B: rt = 2.23 min; Obs. Adducts: [M + H]; Obs. Mass: 517.15 | 20 |
| 149 | 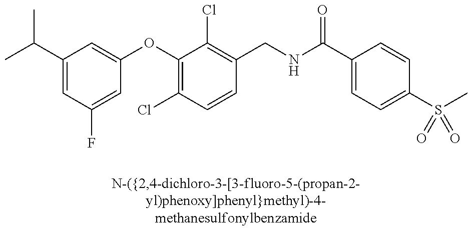 [SMILES: CC(C)c1cc(F)cc(Oc2c(Cl)ccc(CNC(=O)c3ccc(S(C)(=O)=O)cc3)c2Cl)c1] | Method A: rt = 2.33 min; Obs. Adducts: [M + H]; Obs. Mass: 510.21 Method B: rt = 2.33 min; Obs. Adducts: [M + H]; Obs. Mass: 510.06 | 20 |
| 150 | 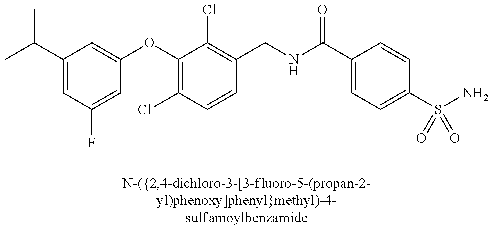 [SMILES: CC(C)c1cc(F)cc(Oc2c(Cl)ccc(CNC(=O)c3ccc(S(N)(=O)=O)cc3)c2Cl)c1] | Method A: rt = 2.16 min; Obs. Adducts: [M + H]; Obs. Mass: 511.12 Method B: rt = 2.14 min; Obs. Adducts: [M + H]; Obs. Mass: 511.08 | 20 |
| 151 | 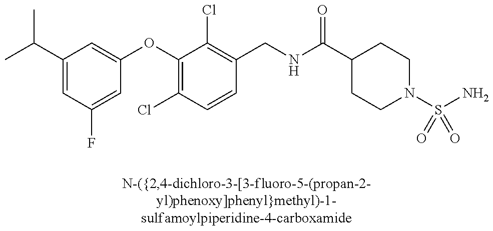 [SMILES: CC(C)c1cc(F)cc(Oc2c(Cl)ccc(CNC(=O)C3CCN(S(N)(=O)=O)CC3)c2Cl)c1] | Method A: rt = 2.1 min; Obs. Adducts: [M + H]; Obs. Mass: 518.09 Method B: rt = 2.09 min; Obs. Adducts: [M + H]; Obs. Mass: 518.13 | 20 |
| 152 | 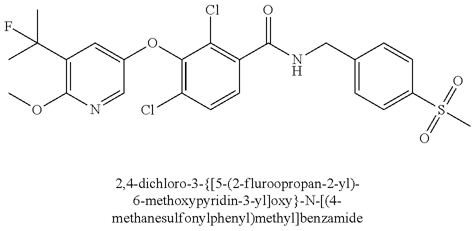 [SMILES: COc1ncc(Oc2c(Cl)ccc(C(=O)NCc3ccc(S(C)(=O)=O)cc3)c2Cl)cc1C(C)(C)F] | Method B: rt = 2.13 min; Obs. Adducts: [M + H]; Obs. Mass: 541.16 Method A: rt = 2.06 min; Obs. Adducts: [M + H]; Obs. Mass: 541.17 | 20 |
| 153 | 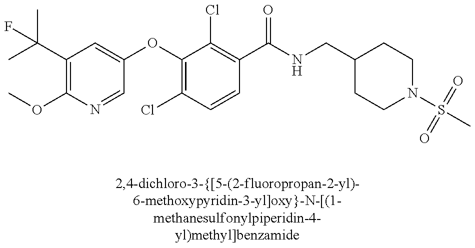 [SMILES: COc1ncc(Oc2c(Cl)ccc(C(=O)NCC3CCN(S(C)(=O)=O)CC3)c2Cl)cc1C(C)(C)F] | Method A: rt = 2.04 min; Obs. Adducts: [M + H]; Obs. Mass: 548.24 Method B: rt = 2.05 min; Obs. Adducts: [M + H]; Obs. Mass: 547.97 | 19 |
| 154 | 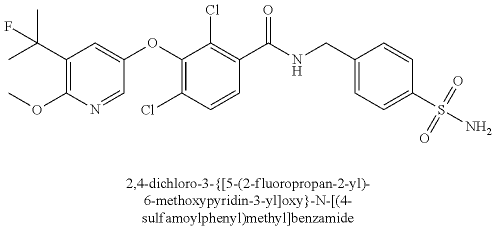 [SMILES: COc1ncc(Oc2c(Cl)ccc(C(=O)NCc3ccc(S(N)(=O)=O)cc3)c2Cl)cc1C(C)(C)F] | Method A: rt = 1.98 min; Obs. Adducts: [M + H]; Obs. Mass: 542.22 Method B: rt = 1.96 min; Obs. Adducts: [M + H]; Obs. Mass: 542.04 | 19 |
| 155 | 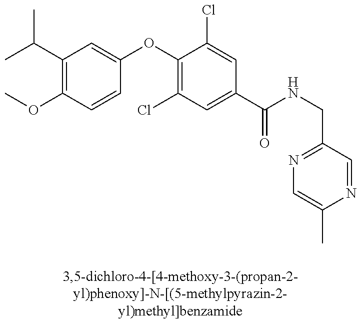 [SMILES: COc1ccc(Oc2c(Cl)cc(C(=O)NCc3cnc(C)cn3)cc2Cl)cc1C(C)C] | Method A: rt = 2.34 min; Obs. Adducts: [M + H]; Obs. Mass: 460.14 Method B: rt = 2.22 min; Obs. Adducts: [M + H]: Obs. Mass: 460.13 | 19 |
| 156 | 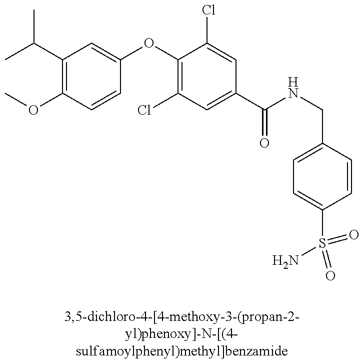 [SMILES: COc1ccc(Oc2c(Cl)cc(C(=O)NCc3ccc(S(N)(=O)=O)cc3)cc2Cl)cc1C(C)C] | Method A: rt = 2.32 min; Obs. Adducts: [M + H]; Obs. Mass: 523.22 Method B: rt = 2.2 min; Obs. Adducts: [M + H]; Obs. Mass: 522.92 | 19 |
| 157 | 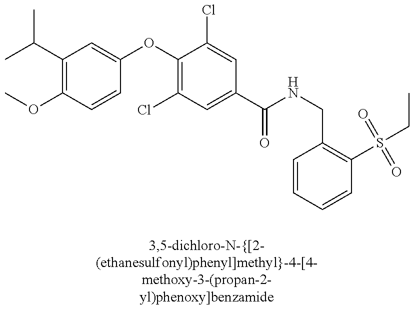 [SMILES: CCS(=O)(=O)c1ccccc1CNC(=O)c1cc(Cl)c(Oc2ccc(OC)c(C(C)C)c2)c(Cl)c1] | Method A: rt = 2.63 min; Obs. Adducts: [M + H]; Obs. Mass: 536.13 Method B: rt = 2.5 min; Obs. Adducts: [M + H]; Obs. Mass: 535.96 | 19 |
| 158 | 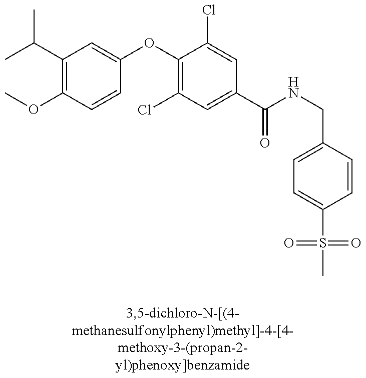 [SMILES: COc1ccc(Oc2c(Cl)cc(C(=O)NCc3ccc(S(C)(=O)=O)cc3)cc2Cl)cc1C(C)C] | Method A: rt = 2.32 min; Obs. Adducts: [M + H]; Obs. Mass: 531.9 Method B: rt = 2.31 min; Obs. Adducts: [M + H]; Obs. Mass: 522.1 | 19 |
| 159 | 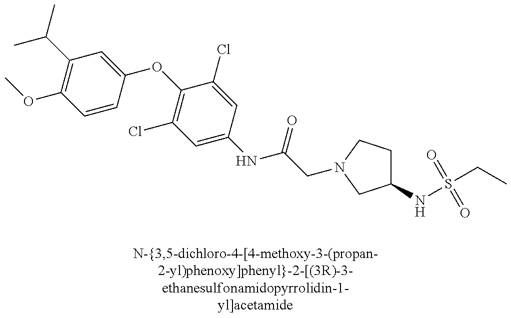 [SMILES: CCS(=O)(=O)N[C@@H]1CCN(CC(=O)Nc2cc(Cl)c(Oc3ccc(OC)c(C(C)C)c3)c(Cl)c2)C1] | Method A: rt = 2.32 min; Obs. Adducts: [M + H]; Obs. Mass: 544.16 Method B: rt = 1.95 min; Obs. Adducts: [M + H]; Obs. Mass: 544.19 | 31 |
| 160 | 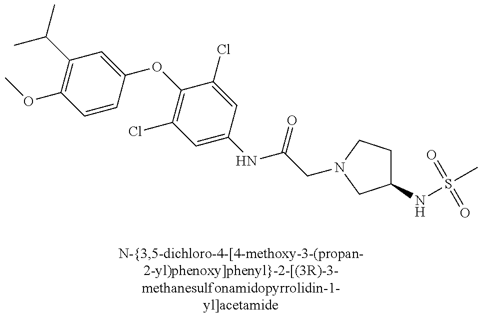 | Method A: rt = 2.24 min; Obs. Adducts: [M + H]; Obs. Mass: 530.12 Method B: rt = 1.82 min; Obs. Adducts: [M + H]; Obs. Mass: 530.09 | 31 |
| 161 | 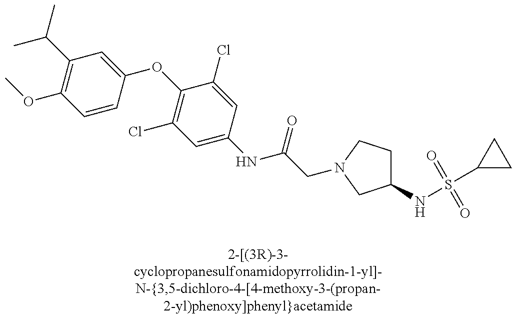 [SMILES: COc1ccc(Oc2c(Cl)cc(NC(=O)CN3CC[C@@H](NS(=O)(=O)C4CC4)C3)cc2Cl)cc1C(C)C] | Method A: rt = 2.35 min; Obs. Adducts: [M + H]; Obs. Mass: 556.18 Method B: rt = 1.97 min; Obs. Adducts: [M + H]; Obs. Mass: 556.23 | 31 |
| 162 | 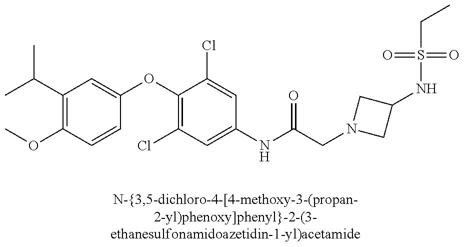 [SMILES: CCS(=O)(=O)NC1CN(CC(=O)Nc2cc(Cl)c(Oc3ccc(OC)c(C(C)C)c3)c(Cl)c2)C1] | Method A: rt = 2.24 min; Obs. Adducts: [M + H]; Obs. Mass: 530.09 Method B: rt = 1.94 min; Obs. Adducts: [M + H]; Obs. Mass: 530.24 | 31 |
| 163 | 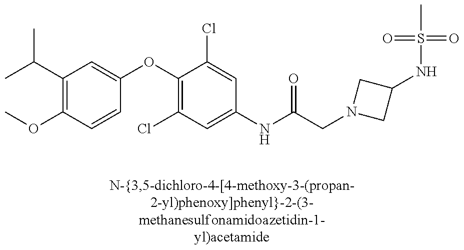 [SMILES: COc1ccc(Oc2c(Cl)cc(NC(=O)CN3CC(NS(C)(=O)=O)C3)cc2Cl)cc1C(C)C] | Method A: rt = 2.18 min; Obs. Adducts: [M + H]; Obs. Mass: 516.07 Method B: rt = 1.9 min; Obs. Adducts: [M + H]; Obs. Mass: 515.98 | 31 |
| 164 | 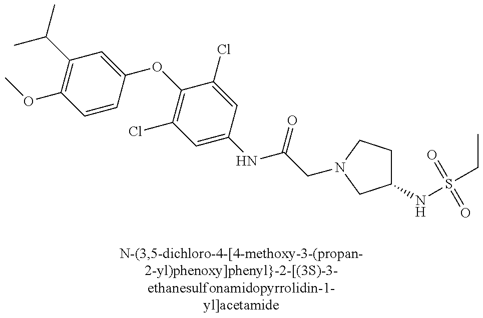 [SMILES: CCS(=O)(=O)N[C@H]1CCN(CC(=O)Nc2cc(Cl)c(Oc3ccc(OC)c(C(C)C)c3)c(Cl)c2)C1] | Method A: rt = 2.35 min; Obs. Adducts: [M + H]; Obs. Mass: 543.98 Method B: rt = 2 min; Obs. Adducts: [M + H]; Obs. Mass: 544.07 | 31 |
| 165 | 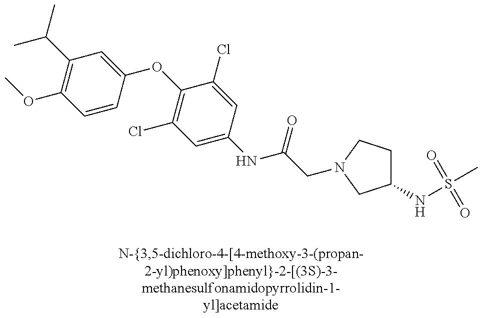 | Method A: rt = 2.25 min; Obs. Adducts: [M + H]; Obs. Mass: 529.93 Method B: rt = 1.9 min; Obs. Adducts: [M + H]; Obs. Mass: 530.18 | 31 |
| 166 | 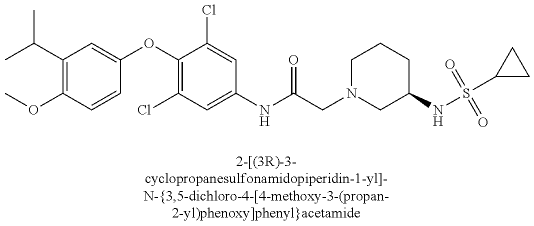 [SMILES: COc1ccc(Oc2c(Cl)cc(NC(=O)CN3CCC[C@@H](NS(=O)(=O)C4CC4)C3)cc2Cl)cc1C(C)C] | Method A: rt = 2.44 min; Obs. Adducts: [M + H]; Obs. Mass: 570.17 Method B: rt = 2.01 min; Obs. Adducts: [M + H]; Obs. Mass: 570.12 | 31 |
| 167 | 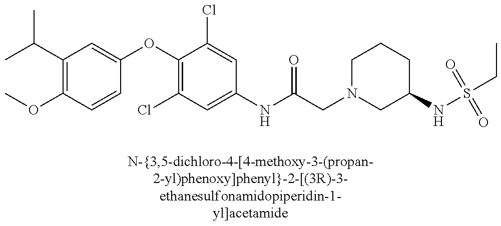 [SMILES: CCS(=O)(=O)N[C@@H]1CCCN(CC(=O)Nc2cc(Cl)c(Oc3ccc(OC)c(C(C)C)c3)c(Cl)c2)C1] | Method A: rt = 2.41 min; Obs. Adducts: [M + H]; Obs. Mass: 558.24 Method B: rt = 1.97 min; Obs. Adducts: [M + H]; Obs. Mass: 557.98 | 31 |
| 168 | 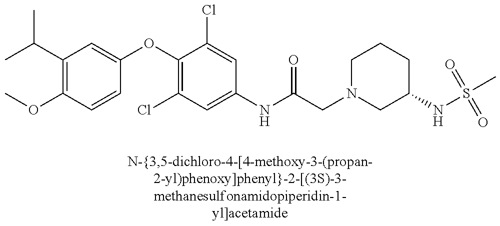 [SMILES: COc1ccc(Oc2c(Cl)cc(NC(=O)CN3CCC[C@H](NS(C)(=O)=O)C3)cc2Cl)cc1C(C)C] | Method A: rt = 2.34 min; Obs. Adducts: [M + H]; Obs. Mass: 543.92 Method B: rt = 1.92 min; Obs. Adducts: [M + H]; Obs. Mass: 543.93 | 31 |
| 169 | 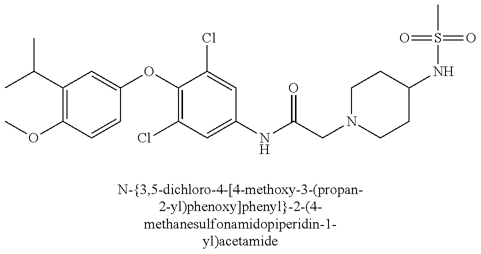 [SMILES: COc1ccc(Oc2c(Cl)cc(NC(=O)CN3CCC(NS(C)(=O)=O)CC3)cc2Cl)cc1C(C)C] | Method A: rt = 2.3 min; Obs. Adducts: [M + H]; Obs. Mass: 543.99 Method B: rt = 1.97 min; Obs. Adducts: [M + H]; Obs. Mass: 544.06 | 31 |
| 170 | 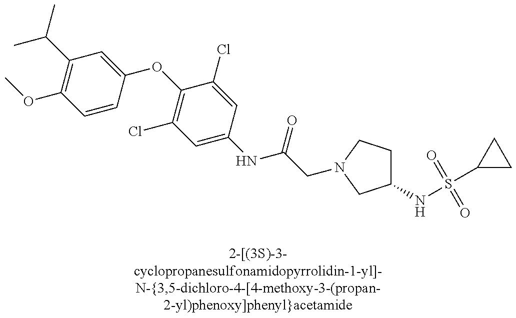 [SMILES: COc1ccc(Oc2c(Cl)cc(NC(=O)CN3CC[C@H](NS(=O)(=O)C4CC4)C3)cc2Cl)cc1C(C)C] | Method A: rt = 2.38 min; Obs. Adducts: [M + H]; Obs. Mass: 555.98 Method B: rt = 2.03 min; Obs. Adducts: [M + H]; Obs. Mass: 556.09 | 31 |
| 171 | 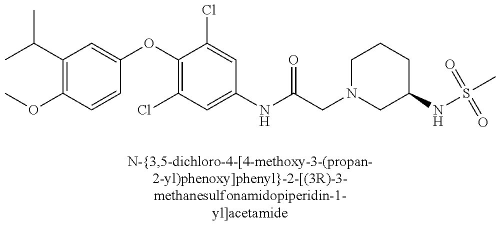 [SMILES: COc1ccc(Oc2c(Cl)cc(NC(=O)CN3CCC[C@@H](NS(C)(=O)=O)C3)cc2Cl)cc1C(C)C] | Method B: rt = 2.04 min; Obs. Adducts: [M + H]; Obs. Mass: 544.06 Method A: rt = 2.36 min; Obs. Adducts: [M + H]; Obs. Mass: 544.12 | 31 |
| 172 | 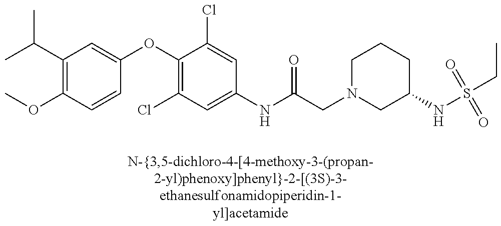 [SMILES: CCS(=O)(=O)N[C@H]1CCCN(CC(=O)Nc2cc(Cl)c(Oc3ccc(OC)c(C(C)C)c3)c(Cl)c2)C1] | Method A: rt = 2.41 min; Obs. Adducts: [M + H]; Obs. Mass: 558.02 Method B: rt = 1.98 min; Obs. Adducts: [M + H]; Obs. Mass: 558.34 | 31 |
| 173 | 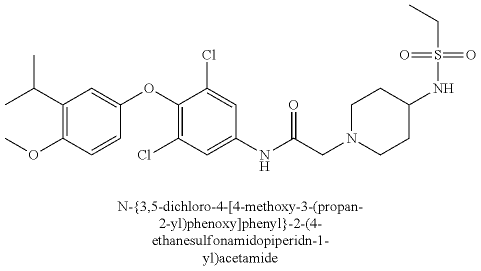 [SMILES: CCS(=O)(=O)NC1CCN(CC(=O)Nc2cc(Cl)c(Oc3ccc(OC)c(C(C)C)c3)c(Cl)c2)CC1] | Method A: rt = 2.34 min; Obs. Adducts: [M + H]; Obs. Mass: 558.07 Method B: rt = 1.95 min; Obs. Adducts: [M + H]; Obs. Mass: 558.24 | 31 |
| 174 | 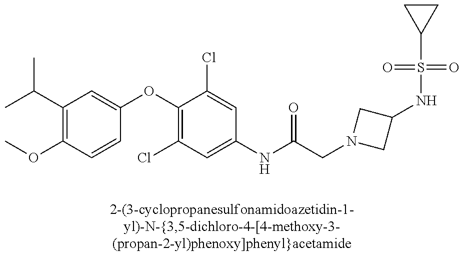 [SMILES: COc1ccc(Oc2c(Cl)cc(NC(=O)CN3CC(NS(=O)(=O)C4CC4)C3)cc2Cl)cc1C(C)C] | Method A: rt = 2.27 min; Obs. Adducts: [M + H]; Obs. Mass: 542.17 | 31 |
| 175 | 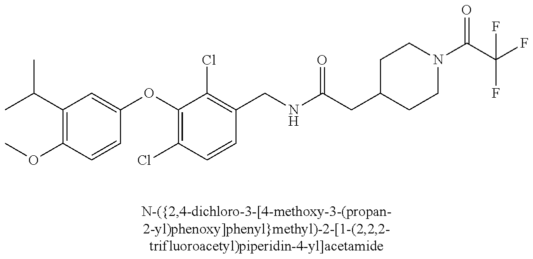 [SMILES: COc1ccc(Oc2c(Cl)ccc(CNC(=O)CC3CCN(C(=O)C(F)(F)F)CC3)c2Cl)cc1C(C)C] | Method A: rt = 2.44 min; Obs. Adducts: [M + H]; Obs. Mass: 560.93 Method B: rt = 2.43 min; Obs. Adducts: [M + H]; Obs. Mass: 560.93 | 20 |
| 176 | 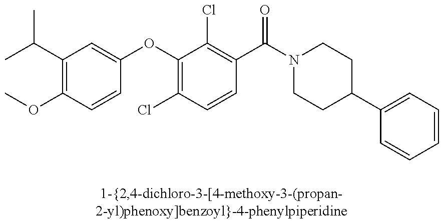 [SMILES: COc1ccc(Oc2c(Cl)ccc(C(=O)N3CCC(c4ccccc4)CC3)c2Cl)cc1C(C)C] | Method A: rt = 2.75 min; Obs. Adducts: [M + H]; Obs. Mass: 498.25 Method B: rt = 2.74 min; Obs. Adducts: [M + H]; Obs. Mass: 497.96 | 19 |
| 177 | 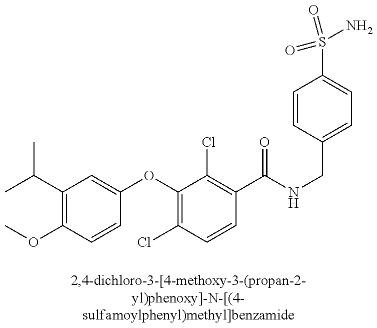 [SMILES: COc1ccc(Oc2c(Cl)ccc(C(=O)NCc3ccc(S(N)(=O)=O)cc3)c2Cl)cc1C(C)C] | Method A: rt = 2.09 min; Obs. Adducts: [M + H]; Obs. Mass: 523.28 Method B: rt = 2.08 min; Obs. Adducts: [M + H]: Obs. Mass: 523.22 | 19 |
| 178 | 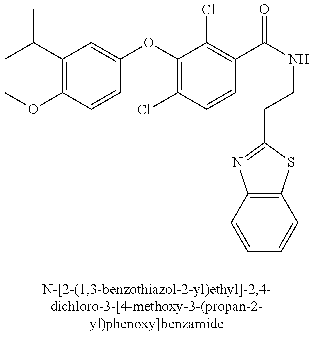 [SMILES: COc1ccc(Oc2c(Cl)ccc(C(=O)NCCc3nc4ccccc4s3)c2Cl)cc1C(C)C] | Method A: rt = 2.52 min; Obs. Adducts: [M + H]; Obs. Mass: 515.02 Method B: rt = 2.51 min; Obs. Adducts: [M + H]; Obs. Mass: 515.16 | 19 |
| 179 | 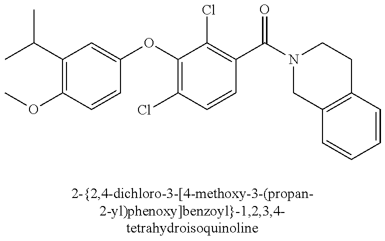 [SMILES: COc1ccc(Oc2c(Cl)ccc(C(=O)N3CCc4ccccc4C3)c2Cl)cc1C(C)C] | Method A: rt = 2.61 min; Obs. Adducts: [M + H]; Obs. Mass: 470.17 Method B: rt = 2.6 min; Obs. Adducts: [M + H]; Obs. Mass: 470.17 | 19 |
| 180 | 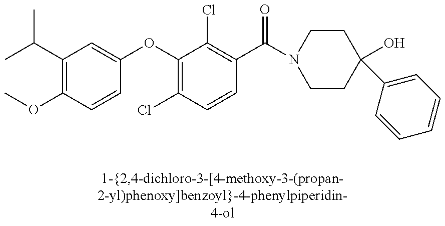 [SMILES: COc1ccc(Oc2c(Cl)ccc(C(=O)N3CCC(O)(c4ccccc4)CC3)c2Cl)cc1C(C)C] | Method A: rt = 2.41 min; Obs. Adducts: [M + H]; Obs. Mass: 514.32 Method B: rt = 2.41 min; Obs. Adducts: [M + H]; Obs. Mass: 514.09 | 19 |
| 181 | 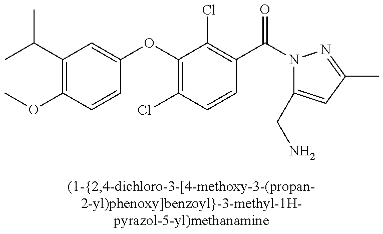 [SMILES: COc1ccc(Oc2c(Cl)ccc(C(=O)n3nc(C)cc3CN)c2Cl)cc1C(C)C] | Method A: rt = 2.06 min; Obs. Adducts: [M + H]; Obs. Mass: 448.23 Method B: rt = 1.96 min; Obs. Adducts: [M + H]; Obs. Mass: 447.98 | 19 |
| 182 | 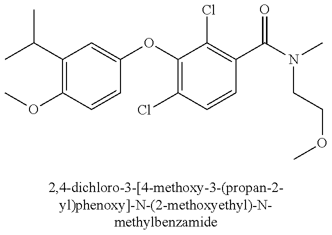 [SMILES: COCCN(C)C(=O)c1ccc(Cl)c(Oc2ccc(OC)c(C(C)C)c2)c1Cl] | Method A: rt = 2.31 min; Obs. Adducts: [M + H]; Obs. Mass: 425.91 Method B: rt = 2.3 min; Obs. Adducts: [M + H]; Obs. Mass: 425.92 | 19 |
| 183 | 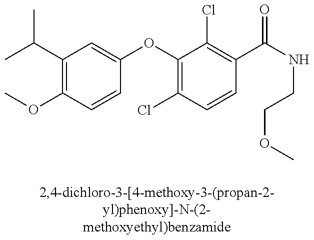 [SMILES: COCCNC(=O)c1ccc(Cl)c(Oc2ccc(OC)c(C(C)C)c2)c1Cl] | Method A: rt = 2.15 min; Obs. Adducts: [M + H]; Obs. Mass: 411.96 Method B: rt = 2.15 min; Obs. Adducts: [M + H]; Obs. Mass: 412.26 | 19 |
| 184 | 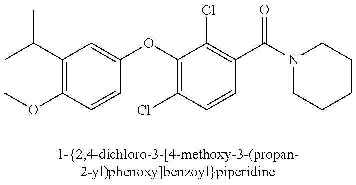 [SMILES: COc1ccc(Oc2c(Cl)ccc(C(=O)N3CCCCC3)c2Cl)cc1C(C)C] | Method A: rt = 2.52 min; Obs. Adducts: [M + H]; Obs. Mass: 422.21 Method B: rt = 2.5 min; Obs. Adducts: [M + H]; Obs. Mass: 421.9 | 19 |
| 185 | 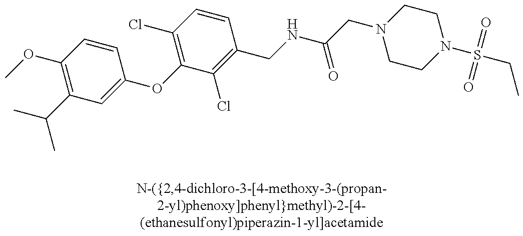 [SMILES: CCS(=O)(=O)N1CCN(CC(=O)NCc2ccc(Cl)c(Oc3ccc(OC)c(C(C)C)c3)c2Cl)CC1] | Method A: rt = 2.28 min; Obs. Adducts: [M + H]; Obs. Mass: 557.97 Method B: rt = 1.96 min; Obs. Adducts: [M + H]; Obs. Mass: 558.01 | 20 |
| 186 | 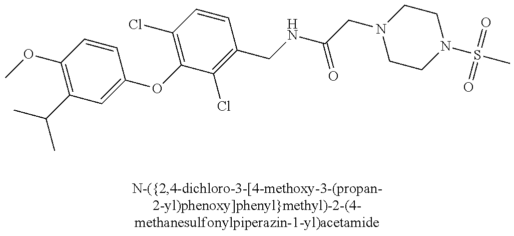 | Method A: rt = 2.2 min; Obs. Adducts: [M + H]; Obs. Mass: 544.15 Method B: rt = 1.9 min; Obs. Adducts: [M + H]; Obs. Mass: 544.09 | 20 |
| 187 | 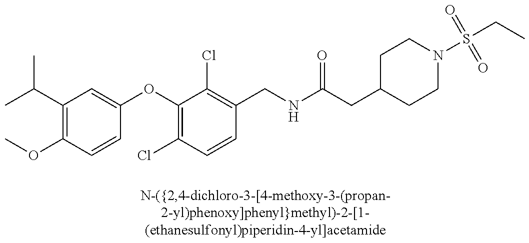 [SMILES: CCS(=O)(=O)N1CCC(CC(=O)NCc2ccc(Cl)c(Oc3ccc(OC)c(C(C)C)c3)c2Cl)CC1] | Method A: rt = 2.27 min; Obs. Adducts: [M + H]; Obs. Mass: 557.24 Method B: rt = 2.26 min; Obs. Adducts: [M + H]; Obs. Mass: 557.22 | 20 |
| 188 | 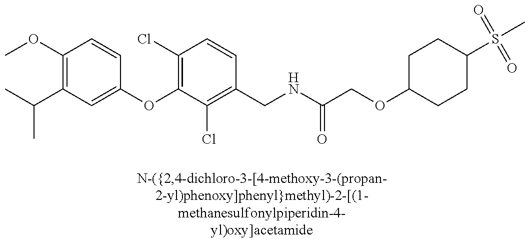 | Method A: rt = 2.25 min; Obs. Adducts: [M + H]; Obs. Mass: 558.9 | 20 |
| 189 | 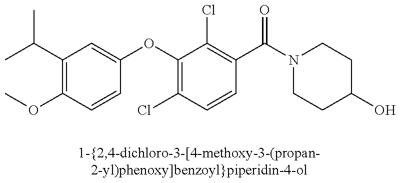 [SMILES: COc1ccc(Oc2c(Cl)ccc(C(=O)N3CCC(O)CC3)c2Cl)cc1C(C)C] | Method A: rt = 1.99 min; Obs. Adducts: [M + H]; Obs. Mass: 438.24 Method B: rt = 1.99 min; Obs. Adducts: [M + H]; Obs. Mass: 438.16 | 19 |
| 190 | 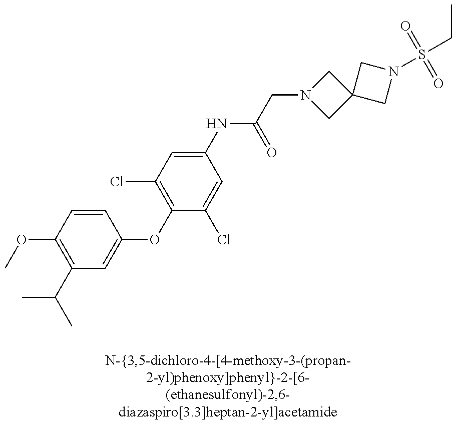 [SMILES: CCS(=O)(=O)N1CC2(CN(CC(=O)Nc3cc(Cl)c(Oc4ccc(OC)c(C(C)C)c4)c(Cl)c3)C2)C1] | Method A: rt = 2.43 min; Obs. Adducts: [M + Na]; Obs. Mass: 580.35 | 31 |
| 191 | 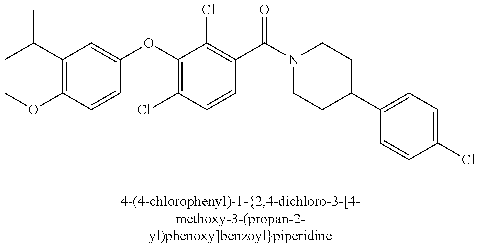 [SMILES: COc1ccc(Oc2c(Cl)ccc(C(=O)N3CCC(c4ccc(Cl)cc4)CC3)c2Cl)cc1C(C)C] | Method A: rt = 2.85 min; Obs. Adducts: [M + H]; Obs. Mass: 532.31 Method B: rt = 2.85 min; Obs. Adducts: [M + H]; Obs. Mass: 532.02 | 19 |
| 192 | 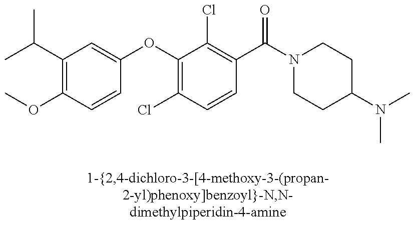 [SMILES: COc1ccc(Oc2c(Cl)ccc(C(=O)N3CCC(N(C)C)CC3)c2Cl)cc1C(C)C] | Method A: rt = 1.87 min; Obs. Adducts: [M + H]; Obs. Mass: 465.32 Method B: rt = 1.78 min; Obs. Adducts: [M + H]; Obs. Mass: 465.34 | 19 |
| 193 | 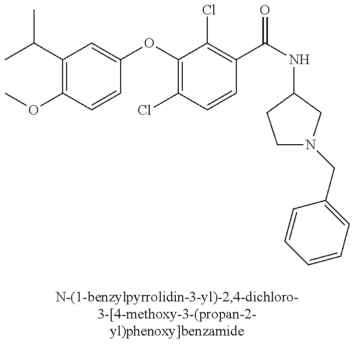 [SMILES: COc1ccc(Oc2c(Cl)ccc(C(=O)NC3CCN(Cc4ccccc4)C3)c2Cl)cc1C(C)C] | Method A: rt = 2.44 min; Obs. Adducts: [M + H]; Obs. Mass: 513.35 Method B: rt = 2 min; Obs. Adducts: [M + H]; Obs. Mass: 513.34 | 19 |
| 194 | 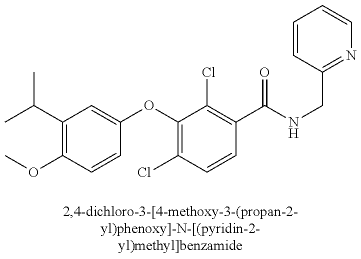 [SMILES: COc1ccc(Oc2c(Cl)ccc(C(=O)NCc3ccccn3)c2Cl)cc1C(C)C] | Method A: rt = 2.19 min; Obs. Adducts: [M + H]; Obs. Mass: 444.89 Method B: rt = 1.85 min; Obs. Adducts: [M + H]; Obs. Mass: 445.18 | 19 |
| 195 | 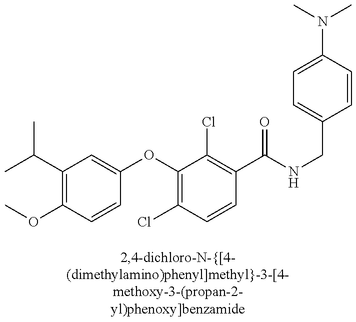 [SMILES: COc1ccc(Oc2c(Cl)ccc(C(=O)NCc3ccc(N(C)C)cc3)c2Cl)cc1C(C)C] | Method A: rt = 2.49 min; Obs. Adducts: [M + H]; Obs. Mass: 487.3 Method B: rt = 1.91 min; Obs. Adducts: [M + H]; Obs. Mass: 487.05 | 19 |
| 196 | 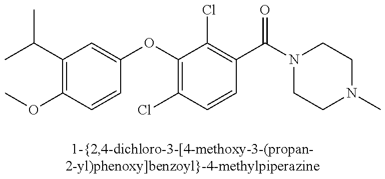 [SMILES: COc1ccc(Oc2c(Cl)ccc(C(=O)N3CCN(C)CC3)c2Cl)cc1C(C)C] | Method A: rt = 2.1 min; Obs. Adducts: [M + H]; Obs. Mass: 437.21 Method B: rt = 1.73 min; Obs. Adducts: [M + H]; Obs. Mass: 437.16 | 19 |
| 197 | 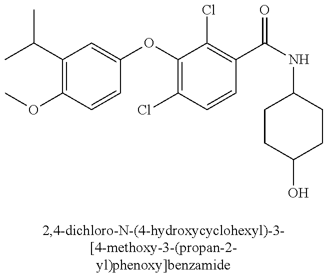 [SMILES: COc1ccc(Oc2c(Cl)ccc(C(=O)NC3CCC(O)CC3)c2Cl)cc1C(C)C] | Method A: rt = 2.04 min; Obs. Adducts: [M + H]; Obs. Mass: 452.3 Method B: rt = 2.05 min; Obs. Adducts: [M + H]; Obs. Mass: 452.04 | 19 |
| 198 | 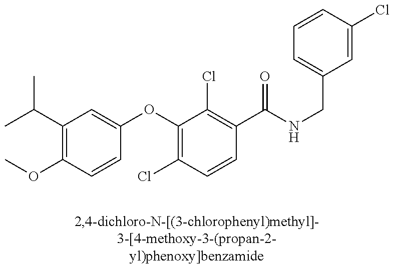 [SMILES: COc1ccc(Oc2c(Cl)ccc(C(=O)NCc3cccc(Cl)c3)c2Cl)cc1C(C)C] | Method A: rt = 2.54 min; Obs. Adducts: [M + H]; Obs. Mass: 478.11 Method B: rt = 2.53 min; Obs. Adducts: [M + H]; Obs. Mass: 478.2 | 19 |
| 199 | 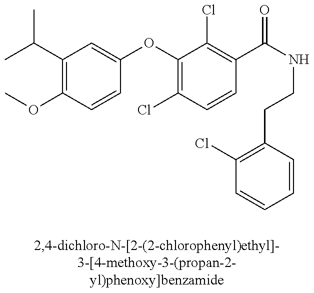 [SMILES: COc1ccc(Oc2c(Cl)ccc(C(=O)NCCc3ccccc3Cl)c2Cl)cc1C(C)C] | Method A: rt = 2.6 min; Obs. Adducts: [M + H]; Obs. Mass: 492.17 Method B: rt = 2.58 min; Obs. Adducts: [M + H]; Obs. Mass: 491.89 | 19 |
| 200 | 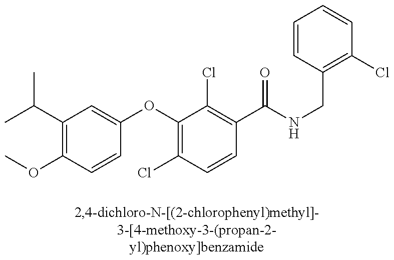 [SMILES: COc1ccc(Oc2c(Cl)ccc(C(=O)NCc3ccccc3Cl)c2Cl)cc1C(C)C] | Method A: rt = 2.53 min; Obs. Adducts: [M + H]; Obs. Mass: 478.15 Method B: rt = 2.52 min; Obs. Adducts: [M + H]; Obs. Mass: 477.96 | 19 |
| 201 | 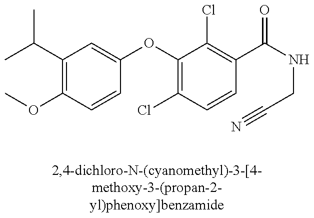 [SMILES: COc1ccc(Oc2c(Cl)ccc(C(=O)NCC#N)c2Cl)cc1C(C)C] | Method A: rt = 2.11 min; Obs. Adducts: [M + H]; Obs. Mass: 393.17 Method B: rt = 2.11 min; Obs. Adducts: [M + H]; Obs. Mass: 393.06 | 19 |
| 202 | 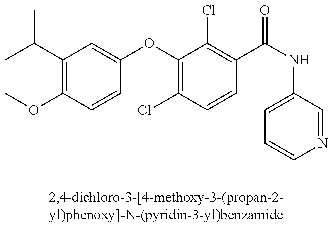 [SMILES: COc1ccc(Oc2c(Cl)ccc(C(=O)Nc3cccnc3)c2Cl)cc1C(C)C] | Method A: rt = 2.17 min; Obs. Adducts: [M + H]; Obs. Mass: 431.09 Method B: rt = 1.87 min; Obs. Adducts: [M + H]; Obs. Mass: 431.11 | 19 |
| 203 | 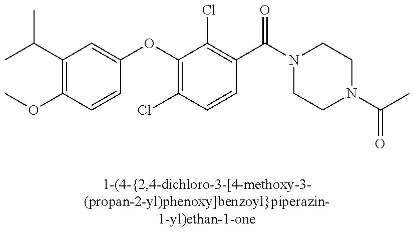 [SMILES: COc1ccc(Oc2c(Cl)ccc(C(=O)N3CCN(C(C)=O)CC3)c2Cl)cc1C(C)C] | Method A: rt = 1.99 min; Obs. Adducts: [M + H]; Obs. Mass: 465.16 Method B: rt = 1.99 min; Obs. Adducts: [M + H]; Obs. Mass: 465.17 | 19 |
| 204 | 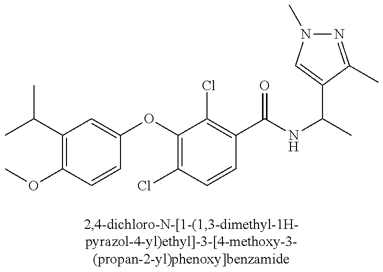 [SMILES: COc1ccc(Oc2c(Cl)ccc(C(=O)NC(C)c3cn(C)nc3C)c2Cl)cc1C(C)C] | Method A: rt = 2.12 min; Obs. Adducts: [M + H]; Obs. Mass: 476.12 Method B: rt = 2.05 min; Obs. Adducts: [M + H]; Obs. Mass: 476.27 | 19 |
| 205 | 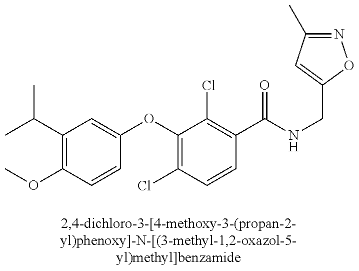 [SMILES: COc1ccc(Oc2c(Cl)ccc(C(=O)NCc3cc(C)no3)c2Cl)cc1C(C)C] | Method A: rt = 2.19 min; Obs. Adducts: [M + H]; Obs. Mass: 449.17 Method B: rt = 2.19 min; Obs. Adducts: [M + H]; Obs. Mass: 449.2 | 19 |
| 206 | 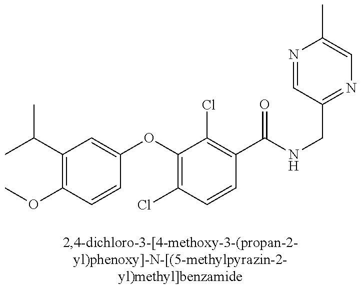 [SMILES: COc1ccc(Oc2c(Cl)ccc(C(=O)NCc3cnc(C)cn3)c2Cl)cc1C(C)C] | Method A: rt = 2.14 min; Obs. Adducts: [M + H]; Obs. Mass: 460.19 Method B: rt = 2.12 min; Obs. Adducts: [M + H]; Obs. Mass: 460.19 | 19 |
| 207 | 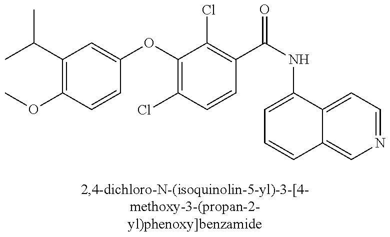 [SMILES: COc1ccc(Oc2c(Cl)ccc(C(=O)Nc3cccc4cnccc34)c2Cl)cc1C(C)C] | Method A: rt = 2.27 min; Obs. Adducts: [M + H]; Obs. Mass: 481.31 Method B: rt = 1.92 min; Obs. Adducts: [M + H]; Obs. Mass: 481.3 | 19 |
| 208 | 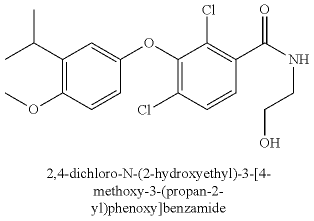 [SMILES: COc1ccc(Oc2c(Cl)ccc(C(=O)NCCO)c2Cl)cc1C(C)C] | Method A: rt = 1.91 min; Obs. Adducts: [M + H]; Obs. Mass: 398.02 Method B: rt = 1.91 min; Obs. Adducts: [M + H]; Obs. Mass: 398.28 | 19 |
| 209 | 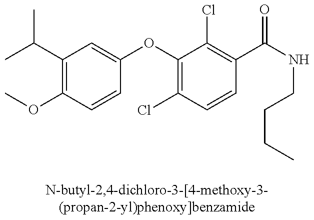 [SMILES: CCCCNC(=O)c1ccc(Cl)c(Oc2ccc(OC)c(C(C)C)c2)c1Cl] | Method A: rt = 2.41 min; Obs. Adducts: [M + H]; Obs. Mass: 410.14 Method B: rt = 2.4 min; Obs. Adducts: [M + H]; Obs. Mass: 410.15 | 19 |
| 210 | 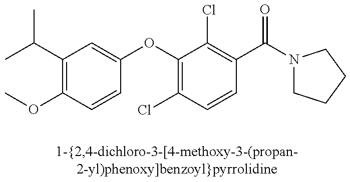 [SMILES: COc1ccc(Oc2c(Cl)ccc(C(=O)N3CCCC3)c2Cl)cc1C(C)C] | Method A: rt = 2.36 min; Obs. Adducts: [M + H]; Obs. Mass: 408.18 Method B: rt = 2.35 min; Obs. Adducts: [M + H]; Obs. Mass: 407.92 | 19 |
| 211 | 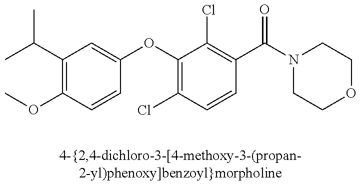 [SMILES: COc1ccc(Oc2c(Cl)ccc(C(=O)N3CCOCC3)c2Cl)cc1C(C)C] | Method A: rt = 2.19 min; Obs. Adducts: [M + H]; Obs. Mass: 424.04 Method B: rt = 2.19 min; Obs. Adducts: [M + H]; Obs. Mass: 424.2 | 19 |
| 212 | 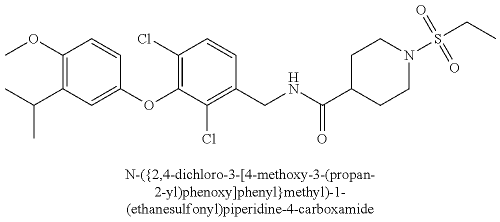 [SMILES: CCS(=O)(=O)N1CCC(C(=O)NCc2ccc(Cl)c(Oc3ccc(OC)c(C(C)C)c3)c2Cl)CC1] | Method A: rt = 2.24 min; Obs. Adducts: [M + H]; Obs. Mass: 543.27 Method B: rt = 2.24 min; Obs. Adducts: [M + H]; Obs. Mass: 543.25 | 20 |
| 213 | 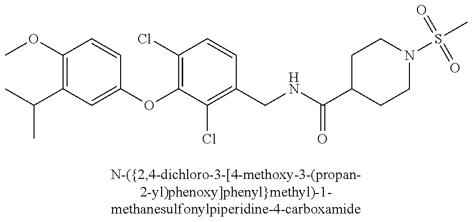 | Method A: rt = 2.18 min; Obs. Adducts: [M + H]; Obs. Mass: 529.05 Method B: rt = 2.17 min; Obs. Adducts: [M + H]; Obs. Mass: 529.29 | 20 |
| 214 | 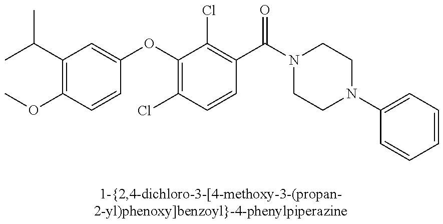 [SMILES: COc1ccc(Oc2c(Cl)ccc(C(=O)N3CCN(c4ccccc4)CC3)c2Cl)cc1C(C)C] | Method A: rt = 2.63 min; Obs. Adducts: [M + H]; Obs. Mass: 499.23 Method B: rt = 2.56 min; Obs. Adducts: [M + H]; Obs. Mass: 499.23 | 19 |
| 215 | 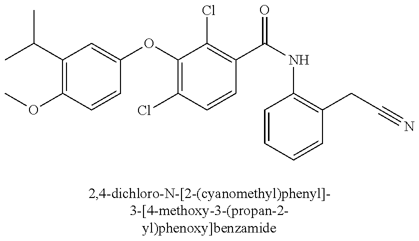 [SMILES: COc1ccc(Oc2c(Cl)ccc(C(=O)Nc3ccccc3CC#N)c2Cl)cc1C(C)C] | Method A: rt = 2.35 min; Obs. Adducts: [M +H]; Obs. Mass: 469.1 Method B: rt = 2.36 min; Obs. Adducts: [M + H]; Obs. Mass: 469.11 | 19 |
| 216 | 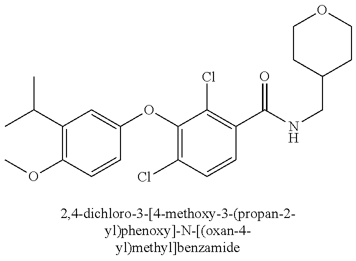 [SMILES: COc1ccc(Oc2c(Cl)ccc(C(=O)NCC3CCOCC3)c2Cl)cc1C(C)C] | Method A: rt = 2.19 min; Obs. Adducts: [M + H]; Obs. Mass: 452.21 Method B: rt = 2.18 min; Obs. Adducts: [M + H]; Obs. Mass: 452.22 | 19 |
| 217 | 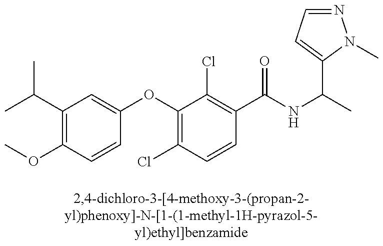 [SMILES: COc1ccc(Oc2c(Cl)ccc(C(=O)NC(C)c3ccnn3C)c2Cl)cc1C(C)C] | Method A: rt = 2.14 min; Obs. Adducts: [M + H]; Obs. Mass: 462.24 Method B: rt = 2.12 min; Obs. Adducts: [M + H]; Obs. Mass; 462.18 | 19 |
| 218 | 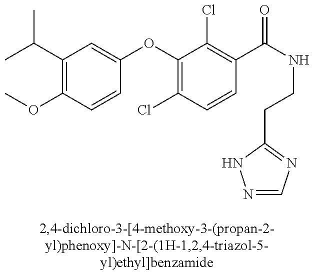 [SMILES: COc1ccc(Oc2c(Cl)ccc(C(=O)NCCc3ncn[nH]3)c2Cl)cc1C(C)C] | Method A: rt = 1.86 min; Obs. Adducts: [M + H]; Obs. Mass: 449.09 Method B: rt = 1.81 min; Obs. Adducts: [M + H]; Obs. Mass: 449.09 | 19 |
| 219 | 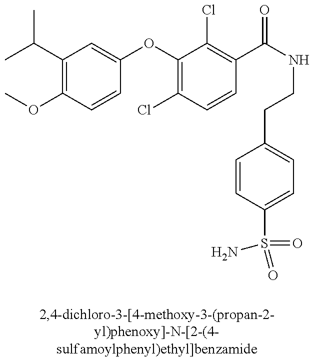 [SMILES: COc1ccc(Oc2c(Cl)ccc(C(=O)NCCc3ccc(S(N)(=O)=O)cc3)c2Cl)cc1C(C)C] | Method A: rt = 2.1 min; Obs. Adducts: [M + H]; Obs. Mass: 537.11 Method B: rt = 2.11 min; Obs. Adducts: [M + H]; Obs. Mass: 537.1 | 19 |
| 220 | 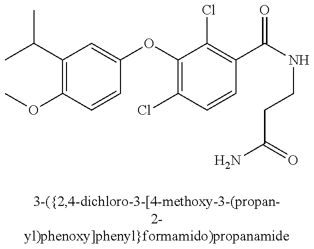 [SMILES: COc1ccc(Oc2c(Cl)ccc(C(=O)NCCC(N)=O)c2Cl)cc1C(C)C] | Method A: rt = 1.8 min; Obs. Adducts: [M + H]; Obs. Mass: 425.11 Method B: rt = 1.81 min; Obs. Adducts: [M + H]; Obs. Mass: 425.11 | 19 |
| 221 | 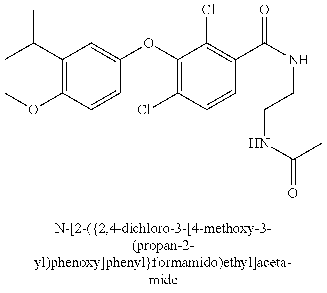 [SMILES: COc1ccc(Oc2c(Cl)ccc(C(=O)NCCNC(C)=O)c2Cl)cc1C(C)C] | Method A: rt = 1.91 min; Obs. Adducts: [M + H]; Obs. Mass: 439.07 Method B: rt = 1.92 min; Obs. Adducts: [M + H]; Obs. Mass: 439.07 | 19 |
| 222 | 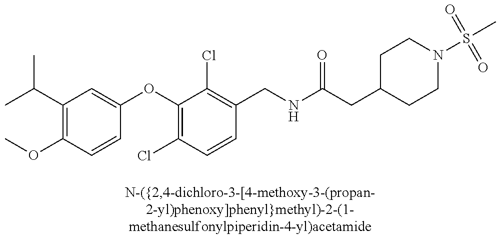 [SMILES: COc1ccc(Oc2c(Cl)ccc(CNC(=O)CC3CCN(S(C)(=O)=O)CC3)c2Cl)cc1C(C)C] | Method A: rt = 2.2 min; Obs. Adducts: [M + H]; Obs. Mass: 543 Method B: rt = 2.19 min; Obs. Adducts: [M + H]; Obs. Mass: 543.23 | 20 |
| 223 | 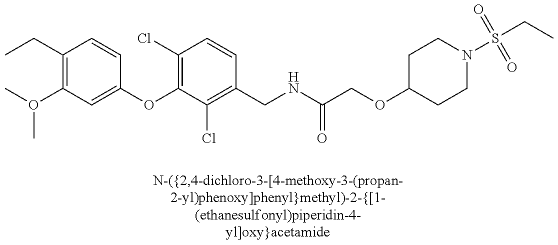 | Method A: rt = 2.29 min; Obs. Adducts: [M + H]; Obs. Mass: 573.05 Method B: rt = 2.29 min; Obs. Adducts: [M + H]; Obs. Mass: 573.27 | 20 |
| 224 | 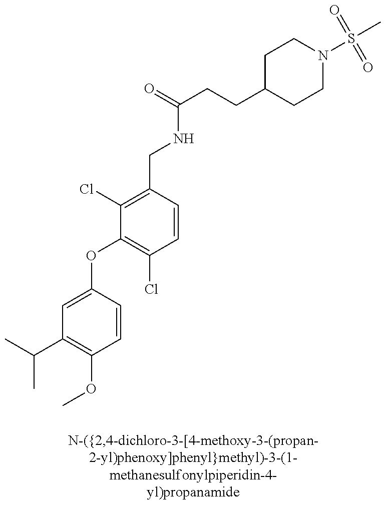 [SMILES: COc1ccc(Oc2c(Cl)ccc(CNC(=O)CCC3CCN(S(C)(=O)=O)CC3)c2Cl)cc1C(C)C] | Method B: rt = 2.24 min; Obs. Adducts: [M + H]; Obs. Mass: 557.22 Method A: rt = 2.24 min; Obs. Adducts: [M + H]; Obs. Mass: 557.12 | 20 |
| 225 | 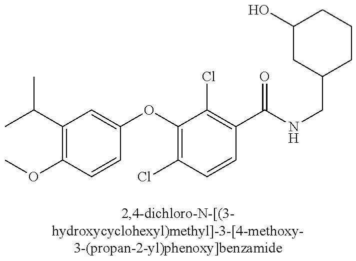 [SMILES: COc1ccc(Oc2c(Cl)ccc(C(=O)NCC3CCCC(O)C3)c2Cl)cc1C(C)C] | Method A: rt = 2.08 min; Obs. Adducts: [M + H]; Obs. Mass: 466.18 Method B: rt = 2.08 min; Obs. Adducts: [M + H]; Obs. Mass: 466.15 | 19 |
| 226 | 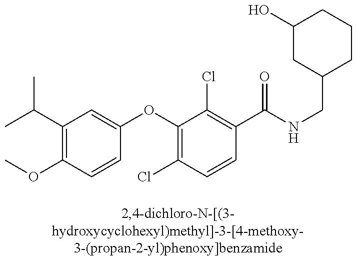 [SMILES: COc1ccc(Oc2c(Cl)ccc(C(=O)NCC3CCCC(O)C3)c2Cl)cc1C(C)C] | Method A: rt = 2.11 min; Obs. Adducts: [M + H]; Obs. Mass: 466.22 Method B: rt = 2.11 min; Obs. Adducts: [M + H]; Obs. Mass: 466.18 | 19 |
| 227 | DEfinition | Method A: rt = 1.91 | 19 |
| | 2,4-dichloro-3-[4-methoxy-3-(propan-2- | min; Obs. Adducts: | |
| | yl)phenoxy]-N-[(6-oxo-1,6- | [M + H]; Obs. Mass: | |
| | dihydropyridin-3-yl)methyl]benzamide | 461.16 | |
| | | Method B: rt = 1.83 | |
| | | min; Obs. Adducts: | |
| | | [M + H]; Obs. Mass: | |
| | | 461.1 | |
| 228 | 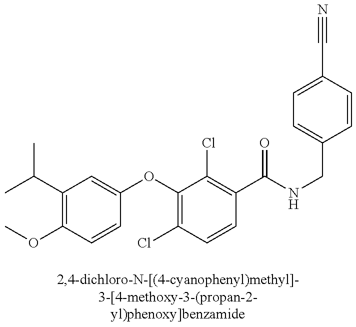 [SMILES: COc1ccc(Oc2c(Cl)ccc(C(=O)NCc3ccc(C#N)cc3)c2Cl)cc1C(C)C] | Method A: rt = 2.43 min; Obs. Adducts: [M + H]; Obs. Mass: 468.88 Method B: rt = 2.28 min; Obs. Adducts: [M + H]; Obs. Mass: 469.12 | 19 |
| 229 | 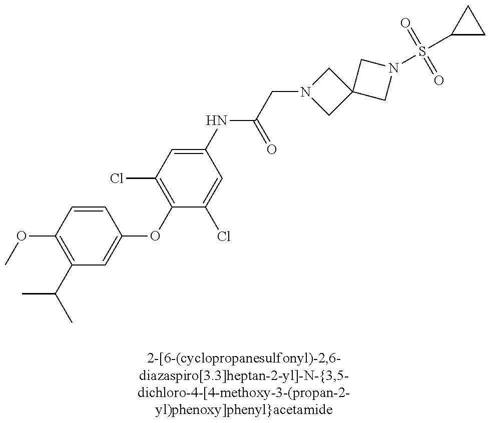 [SMILES: COc1ccc(Oc2c(Cl)cc(NC(=O)CN3CC4(C3)CN(S(=O)(=O)C3CC3)C4)cc2Cl)cc1C(C)C] | Method A: rt = 2.46 min; Obs. Adducts: [M + H]; Obs. Mass: 568.4 Method B: rt = 2.06 min; Obs. Adducts:; Obs. Mass: | 31 |
| 230 | 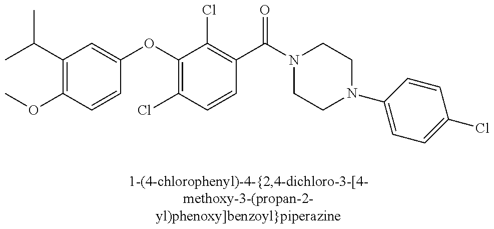 [SMILES: COc1ccc(Oc2c(Cl)ccc(C(=O)N3CCN(c4ccc(Cl)cc4)CC3)c2Cl)cc1C(C)C] | Method B: rt = 2.77 min; Obs. Adducts: [M + H]; Obs. Mass: 533.15 Method A: rt = 2.76 min; Obs. Adducts: [M + H]; Obs. Mass; 533.16 | 19 |
| 231 | 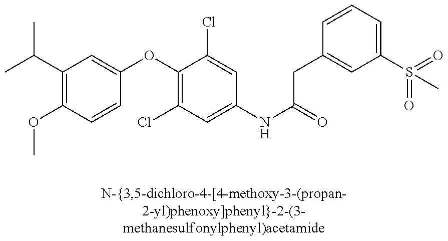 [SMILES: COc1ccc(Oc2c(Cl)cc(NC(=O)Cc3cccc(S(C)(=O)=O)c3)cc2Cl)cc1C(C)C] | Method A: rt = 2.4 min; Obs. Adducts: [M + H]; Obs. Mass: 521.92 Method B: rt = 2.43 min; Obs. Adducts: [M + H]; Obs. Mass: 522.2 | 31 |
| 232 | 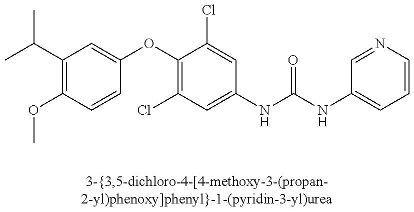 [SMILES: COc1ccc(Oc2c(Cl)cc(NC(=O)Nc3cccnc3)cc2Cl)cc1C(C)C] | Method A: rt = 2.32 min; Obs. Adducts: [M + H]; Obs. Mass: 445.98 Method B: rt = 1.92 min; Obs. Adducts: [M + H]; Obs. Mass: 446.31 | 1 |
| 233 | 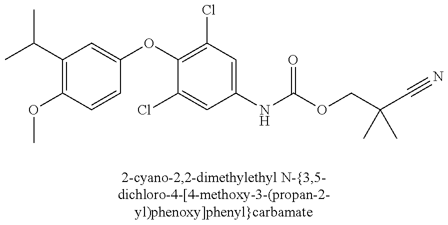 [SMILES: COc1ccc(Oc2c(Cl)cc(NC(=O)OCC(C)(C)C#N)cc2Cl)cc1C(C)C] | Method A: rt = 2.58 min; Obs. Adducts: [M + H]; Obs. Mass: 451.11 Method B: rt = 2.56 min; Obs. Adducts: [M + H]; Obs. Mass: 450.96 | 1 |
| 234 | 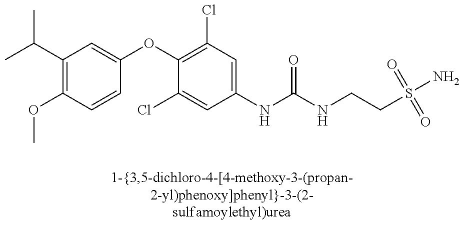 [SMILES: COc1ccc(Oc2c(Cl)cc(NC(=O)NCCS(N)(=O)=O)cc2Cl)cc1C(C)C] | Method A: rt = 2.08 min; Obs. Adducts: [M + H]; Obs. Mass: 476 Method B: rt = 2.08 min; Obs. Adducts: [M + H]; Obs. Mass: 476.04 | 1 |
| 235 | 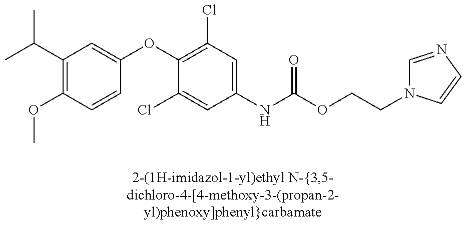 [SMILES: COc1ccc(Oc2c(Cl)cc(NC(=O)OCCn3ccnc3)cc2Cl)cc1C(C)C] | Method A: rt = 2.22 min; Obs. Adducts: [M + H]; Obs. Mass: 464.11 Method B: rt = 1.93 min; Obs. Adducts: [M + H]; Obs. Mass: 464.13 | 1 |
| 236 | 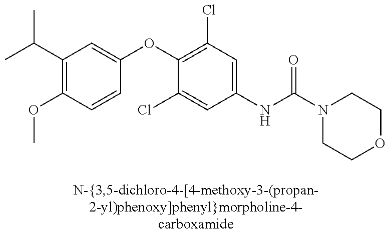 [SMILES: COc1ccc(Oc2c(Cl)cc(NC(=O)N3CCOCC3)cc2Cl)cc1C(C)C] | Method A: rt = 2.27 min; Obs. Adducts: [M + H]; Obs. Mass: 439.11 Method B: rt = 2.25 min; Obs. Adducts: [M + H]; Obs. Mass: 439.1 | 1 |
| 237 | 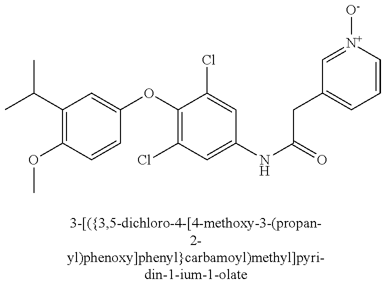 [SMILES: COc1ccc(Oc2c(Cl)cc(NC(=O)Cc3ccc[n+]([O-])c3)cc2Cl)cc1C(C)C] | Method B: rt = 1.94 min; Obs. Adducts: [M + H]; Obs. Mass: 461.1 | 31 |
| 238 | 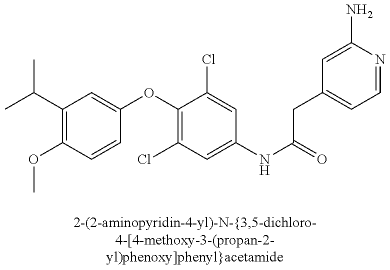 [SMILES: COc1ccc(Oc2c(Cl)cc(NC(=O)Cc3ccnc(N)c3)cc2Cl)cc1C(C)C] | Method A: rt = 2.16 min; Obs. Adducts: [M + H]; Obs. Mass: 460.16 Method B: rt = 1.87 min; Obs. Adducts: [M + H]; Obs. Mass: 460.13 | 2 |
| 239 | 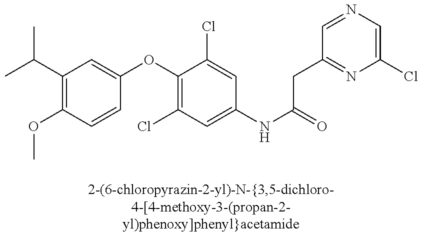 [SMILES: COc1ccc(Oc2c(Cl)cc(NC(=O)Cc3cncc(Cl)n3)cc2Cl)cc1C(C)C] | Method A: rt = 2.49 min; Obs. Adducts: [M + H]; Obs. Mass: 480.15 Method B: rt = 2.43 min; Obs. Adducts: [M + H]; Obs. Mass: 480.11 | 31 |
| 240 | 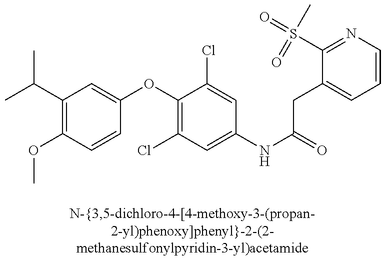 [SMILES: COc1ccc(Oc2c(Cl)cc(NC(=O)Cc3cccnc3S(C)(=O)=O)cc2Cl)cc1C(C)C] | Method B: rt = 2.28 min; Obs. Adducts: [M + H]; Obs. Mass: 523.36 Method A: rt = 2.29 min; Obs. Adducts: [M + H]; Obs. Mass: 523.11 | 3 |
| 241 | 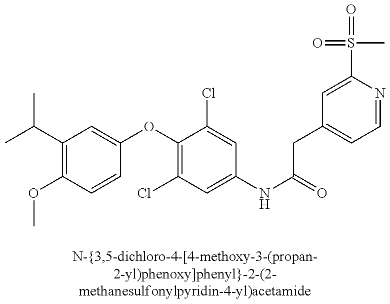 [SMILES: COc1ccc(Oc2c(Cl)cc(NC(=O)Cc3ccnc(S(C)(=O)=O)c3)cc2Cl)cc1C(C)C] | Method A: rt = 2.3 min; Obs. Adducts: [M + H]; Obs. Mass: 523.08 Method B: rt = 2.24 min; Obs. Adducts: [M + H]; Obs. Mass: 523.11 | 3 |
| 242 | 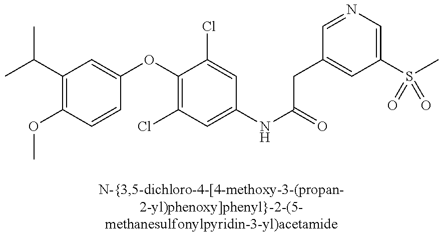 [SMILES: COc1ccc(Oc2c(Cl)cc(NC(=O)Cc3cncc(S(C)(=O)=O)c3)cc2Cl)cc1C(C)C] | Method A: rt = 2.23 min; Obs. Adducts: [M + H]; Obs. Mass: 523.18 Method B: rt = 2.22 min; Obs. Adducts: [M + H]; Obs. Mass: 522.9 | 31 |
| 243 | 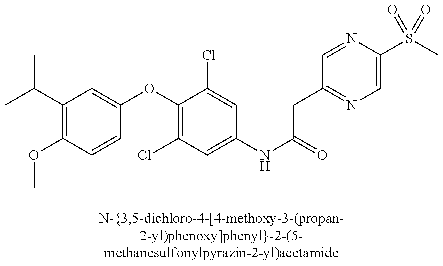 [SMILES: COc1ccc(Oc2c(Cl)cc(NC(=O)Cc3cnc(S(C)(=O)=O)cn3)cc2Cl)cc1C(C)C] | Method A: rt = 2.25 min; Obs. Adducts: [M + H]; Obs. Mass: 524.02 Method B: rt = 2.24 min; Obs. Adducts: [M + H]; Obs. Mass: 524.1 | 3 |
| 244 | 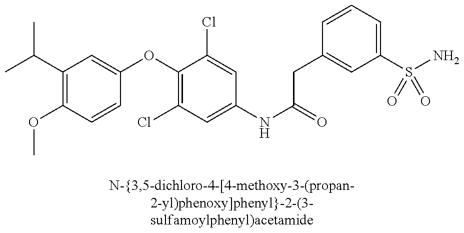 [SMILES: COc1ccc(Oc2c(Cl)cc(NC(=O)Cc3cccc(S(N)(=O)=O)c3)cc2Cl)cc1C(C)C] | Method A: rt = 2.27 min; Obs. Adducts: [M + H]; Obs. Mass: 522.98 Method B: rt = 2.23 min; Obs. Adducts: [M + H]; Obs. Mass: 523.15 | 31 |
| 245 | 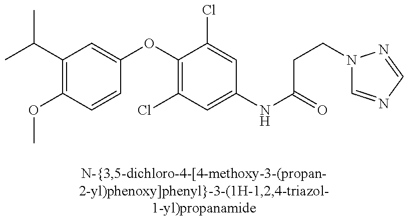 [SMILES: COc1ccc(Oc2c(Cl)cc(NC(=O)CCn3cncn3)cc2Cl)cc1C(C)C] | Method A: rt = 2.12 min; Obs. Adducts: [M + H]; Obs. Mass: 449.13 Method B: rt = 2.08 min; Obs. Adducts: [M + H]; Obs. Mass: 448.88 | 31 |
| 246 | 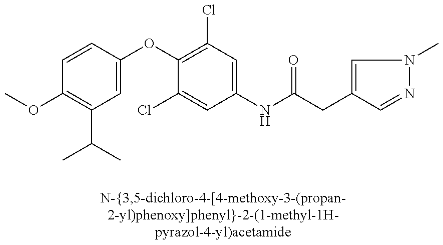 | Method A: rt = 2.2 min; Obs. Adducts: [M + H]; Obs. Mass: 448.12 Method B: rt = 2.24 min; Obs. Adducts: [M + H]; Obs. Mass: 448.17 | 31 |
| 247 | 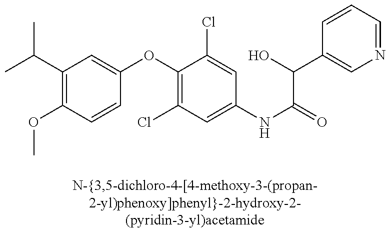 [SMILES: COc1ccc(Oc2c(Cl)cc(NC(=O)C(O)c3cccnc3)cc2Cl)cc1C(C)C] | Method A: rt = 2.18 min; Obs. Adducts: [M + H]; Obs. Mass: 461.12 Method B: rt = 2.03 min; Obs. Adducts: [M + H]; Obs. Mass: 460.89 | 31 |
| 248 | 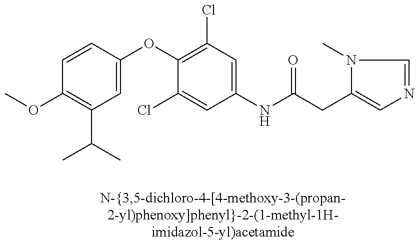 [SMILES: COc1ccc(Oc2c(Cl)cc(NC(=O)Cc3cncn3C)cc2Cl)cc1C(C)C] | Method A: rt = 2.16 min; Obs. Adducts: [M + H]; Obs. Mass: 448.3 Method B: rt = 1.91 min; Obs. Adducts: [M + H]; Obs. Mass: 447.9 | 31 |
| 249 | 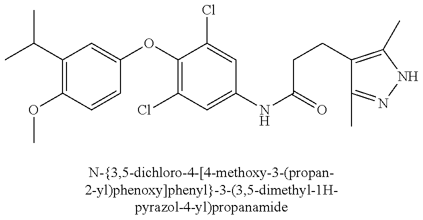 [SMILES: COc1ccc(Oc2c(Cl)cc(NC(=O)CCc3c(C)n[nH]c3C)cc2Cl)cc1C(C)C] | Method A: rt = 2.3 min; Obs. Adducts: [M + H]; Obs. Mass: 476.09 Method B: rt = 2.16 min; Obs. Adducts: [M + H]; Obs. Mass: 476.09 | 31 |
| 250 | 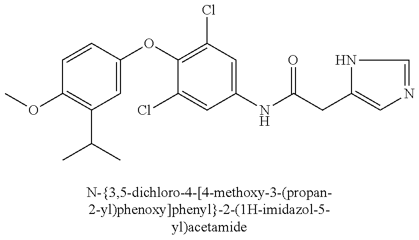 [SMILES: COc1ccc(Oc2c(Cl)cc(NC(=O)Cc3cnc[nH]3)cc2Cl)cc1C(C)C] | Method A: rt = 2.04 min; Obs. Adducts: [M + H]; Obs. Mass: 434.1 Method B: rt = 1.85 min; Obs. Adducts: [M + H]; Obs. Mass: 434.01 | 31 |
| 251 | 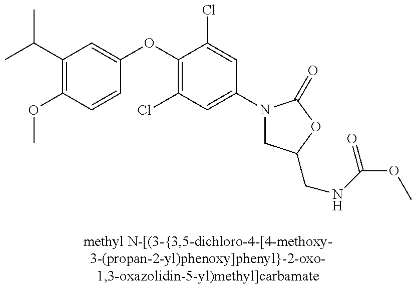 [SMILES: COC(=O)NCC1CN(c2cc(Cl)c(Oc3ccc(OC)c(C(C)C)c3)c(Cl)c2)C(=O)O1] | Method B: rt = 2.28 min; Obs. Adducts: [M + H]; Obs. Mass: 483.09 Method A: rt = 2.38 min; Obs. Adducts: [M + H]; Obs. Mass: 483.16 | 11 |
| 252 | 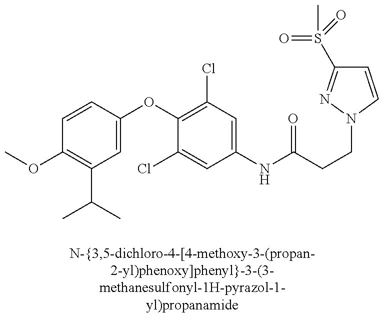 [SMILES: COc1ccc(Oc2c(Cl)cc(NC(=O)CCn3ccc(S(C)(=O)=O)n3)cc2Cl)cc1C(C)C] | Method A: rt = 2.33 min; Obs. Adducts: [M + H]; Obs. Mass; 526.02 Method B: rt = 2.19 min; Obs. Adducts: [M + H]; Obs. Mass: 526.04 | 9 |
| 253 | 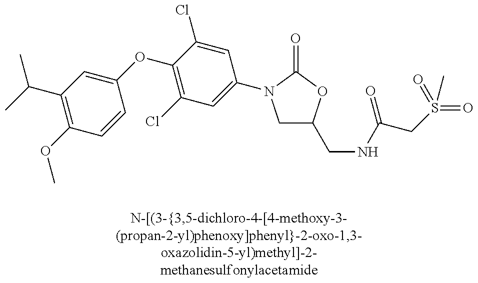 [SMILES: COc1ccc(Oc2c(Cl)cc(N3CC(CNC(=O)CS(C)(=O)=O)OC3=O)cc2Cl)cc1C(C)C] | Method A: rt = 2.12 min; Obs. Adducts: [M + H]; Obs. Mass: 545.25 Method B: rt = 2.14 min; Obs. Adducts: [M + H]; Obs. Mass: 545.11 | 11 |
| 254 — | 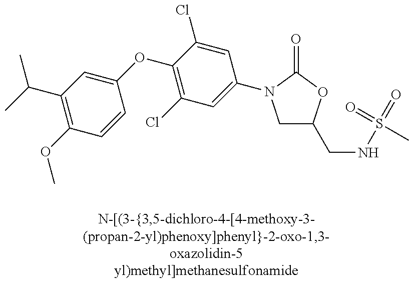 [SMILES: COc1ccc(Oc2c(Cl)cc(N3CC(CNS(C)(=O)=O)OC3=O)cc2Cl)cc1C(C)C] | Method B: rt = 2.22 min; Obs. Adducts: [M + H]; Obs. Mass: 503.17 Method A: rt = 2.22 min; Obs. Adducts: [M + H]; Obs. Mass: 502.93 | 11 |
| 255 | 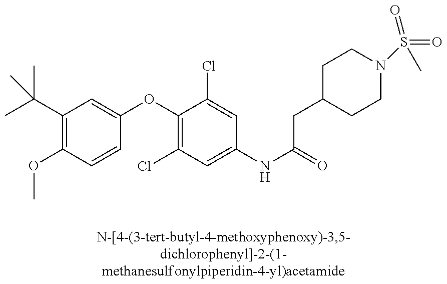 [SMILES: COc1ccc(Oc2c(Cl)cc(NC(=O)CC3CCN(S(C)(=O)=O)CC3)cc2Cl)cc1C(C)(C)C] | Method A: rt = 2.48 min; Obs. Adducts: [M + H]; Obs. Mass: 543.19 Method B: rt = 2.5 min; Obs. Adducts: [M + H]; Obs. Mass: 543.17 | 13 |
| 256 | 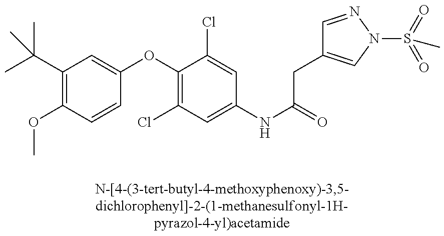 [SMILES: COc1ccc(Oc2c(Cl)cc(NC(=O)Cc3cnn(S(C)(=O)=O)c3)cc2Cl)cc1C(C)(C)C] | Method B: rt = 2.41 min; Obs. Adducts: [M + H]; Obs. Mass: 525.96 Method A: rt = 2.43 min; Obs. Adducts: [M + H]; Obs. Mass: 525.88 | 13 |
| 257 | 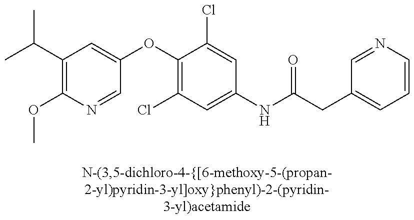 [SMILES: COc1ncc(Oc2c(Cl)cc(NC(=O)Cc3cccnc3)cc2Cl)cc1C(C)C] | Method A: rt = 2.25 min; Obs. Adducts: [M + H]; Obs. Mass: 446.04 Method B: rt = 1.89 min; Obs. Adducts: [M + H]; Obs. Mass: 446.06 | 15 |
| 258 | 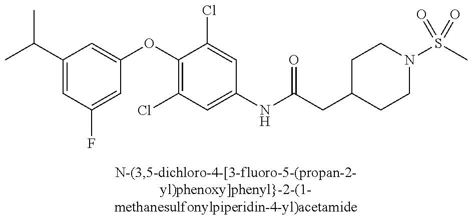 [SMILES: CC(C)c1cc(F)cc(Oc2c(Cl)cc(NC(=O)CC3CCN(S(C)(=O)=O)CC3)cc2Cl)c1] | Method A: rt = 2.47 min; Obs. Adducts: [M + H]; Obs. Mass: 517.05 Method B: rt = 2.44 min; Obs. Adducts: [M + H]; Obs. Mass: 517.02 | 14 |
| 259 | 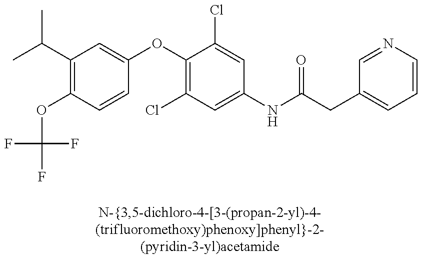 [SMILES: CC(C)c1cc(Oc2c(Cl)cc(NC(=O)Cc3cccnc3)cc2Cl)ccc1OC(F)(F)F] | Method A: rt = 2.53 min; Obs. Adducts: [M + H]; Obs. Mass: 499.28 Method B: rt = 2.18 min; Obs. Adducts: [M + H]; Obs. Mass: 498.95 | 14 |
| 260 | 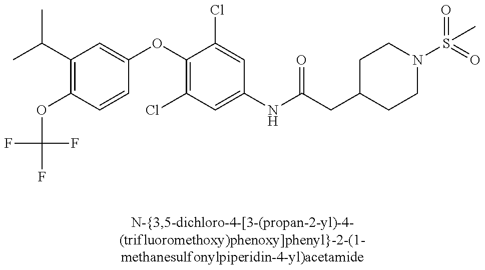 [SMILES: CC(C)c1cc(Oc2c(Cl)cc(NC(=O)CC3CCN(S(C)(=O)=O)CC3)cc2Cl)ccc1OC(F)(F)F] | Method A: rt = 2.62 min; Obs. Adducts: [M + H]; Obs. Mass: 583.08 Method B: rt = 2.59 min; Obs. Adducts: [M + H]; Obs. Mass: 583.02 | 15 |
| 261 | 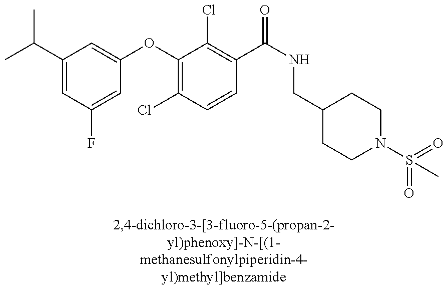 [SMILES: CC(C)c1cc(F)cc(Oc2c(Cl)ccc(C(=O)NCC3CCN(S(C)(=O)=O)CC3)c2Cl)c1] | Method A: rt = 2.22 min; Obs. Adducts: [M + H]; Obs. Mass: 517.07 Method B: rt = 2.19 min; Obs. Adducts: [M + H]; Obs. Mass: 517.1 | 16 |
| 262 | 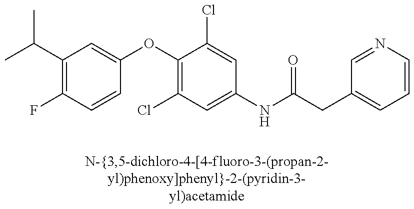 [SMILES: CC(C)c1cc(Oc2c(Cl)cc(NC(=O)Cc3cccnc3)cc2Cl)ccc1F] | Method A: rt = 2.34 min; Obs. Adducts: [M + H]; Obs. Mass: 433.13 Method B: rt = 1.92 min; Obs. Adducts: [M + H]; Obs. Mass: 433.1 | 14 |
| 263 | 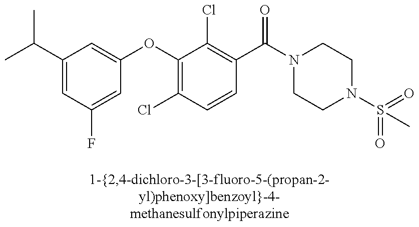 [SMILES: CC(C)c1cc(F)cc(Oc2c(Cl)ccc(C(=O)N3CCN(S(C)(=O)=O)CC3)c2Cl)c1] | Method A: rt = 2.18 min; Obs. Adducts: [M + H]; Obs. Mass: 489.02 Method B: rt = 2.19 min; Obs. Adducts: [M + H]; Obs. Mass: 489.14 | 16 |
| 264 | 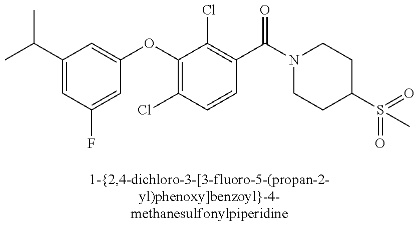 [SMILES: CC(C)c1cc(F)cc(Oc2c(Cl)ccc(C(=O)N3CCC(S(C)(=O)=O)CC3)c2Cl)c1] | Method A: rt = 2.09 min; Obs. Adducts: [M + H]; Obs. Mass: 488.15 Method B: rt = 2.12 min; Obs. Adducts: [M + H]; Obs. Mass: 488.13 | 16 |
| 265 | 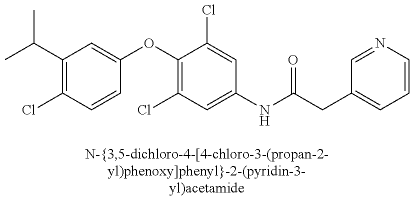 [SMILES: CC(C)c1cc(Oc2c(Cl)cc(NC(=O)Cc3cccnc3)cc2Cl)ccc1Cl] | Method A: rt = 2.43 min; Obs. Adducts: [M + H]; Obs. Mass: 449.19 Method B: rt = 2.07 min; Obs. Adducts: [M + H]; Obs. Mass: 449.11 | 14 |
| 266 | 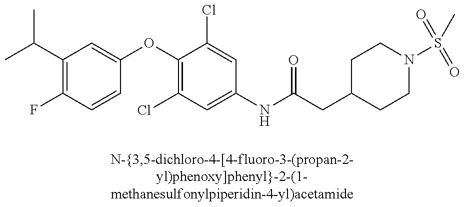 [SMILES: CC(C)c1cc(Oc2c(Cl)cc(NC(=O)CC3CCN(S(C)(=O)=O)CC3)cc2Cl)ccc1F] | Method A: rt = 2.45 min; Obs. Adducts: [M + H]; Obs. Mass: 517.01 Method B: rt = 2.43 min; Obs. Adducts: [M + H]; Obs. Mass: 517.11 | 14 |
| 267 | 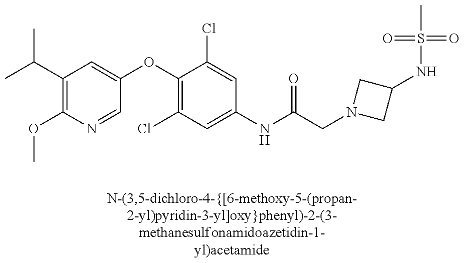 [SMILES: COc1ncc(Oc2c(Cl)cc(NC(=O)CN3CC(NS(C)(=O)=O)C3)cc2Cl)cc1C(C)C] | Method A: rt = 2.11 min; Obs. Adducts: [M + H]; Obs. Mass: 517.24 Method B: rt = 1.75 min; Obs. Adducts: [M + H]; Obs. Mass: 517.24 | 17 |
| 268 | 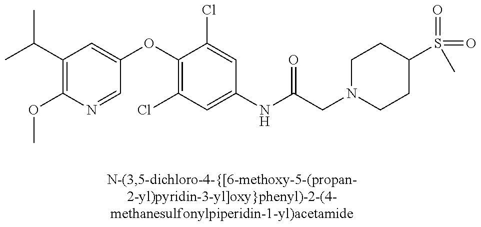 [SMILES: COc1ncc(Oc2c(Cl)cc(NC(=O)CN3CCC(S(C)(=O)=O)CC3)cc2Cl)cc1C(C)C] | Method A: rt = 2.22 min; Obs. Adducts: [M + H]; Obs. Mass: 530.26 Method B: rt = 1.77 min; Obs. Adducts: [M + H]; Obs. Mass: 530.12 | 17 |
| 269 | 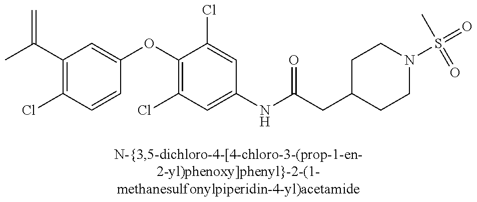 [SMILES: C=C(C)c1cc(Oc2c(Cl)cc(NC(=O)CC3CCN(S(C)(=O)=O)CC3)cc2Cl)ccc1Cl] | Method A: rt = 2.44 min; Obs. Adducts: [M + H]; Obs. Mass: 532.08 Method B: rt = 2.41 min; Obs. Adducts: [M + H]; Obs. Mass: 531.14 | 14 |
| 270 | 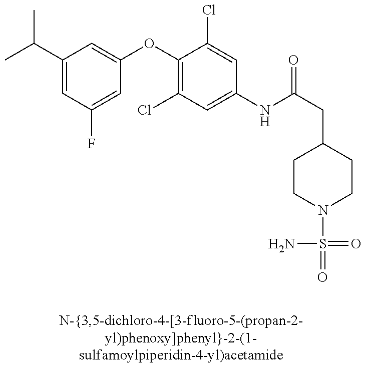 [SMILES: CC(C)c1cc(F)cc(Oc2c(Cl)cc(NC(=O)CC3CCN(S(N)(=O)=O)CC3)cc2Cl)c1] | Method A: rt = 2.36 min; Obs. Adducts: [M + H]; Obs. Mass: 518.13 Method B: rt = 2.32 min; Obs. Adducts: [M + H]; Obs. Mass: 518.07 | 15 |
| 271 | 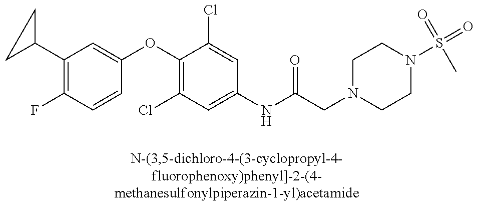 [SMILES: CS(=O)(=O)N1CCN(CC(=O)Nc2cc(Cl)c(Oc3ccc(F)c(C4CC4)c3)c(Cl)c2)CC1] | Method A: rt = 2.22 min; Obs. Adducts: [M + H]; Obs. Mass: 516.27 Method B: rt = 1.79 min; Obs. Adducts: [M + H]; Obs. Mass: 516.28 | 17 |
| 272 | 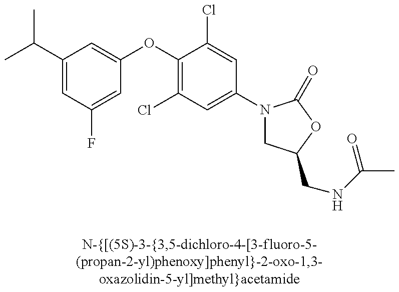 [SMILES: CC(=O)NC[C@H]1CN(c2cc(Cl)c(Oc3cc(F)cc(C(C)C)c3)c(Cl)c2)C(=O)O1] | Method B: rt = 2.16 min; Obs. Adducts: [M + H]; Obs. Mass: 455.41 Method A: rt = 2.98 min; Obs. Adducts: [M + H]; Obs. Mass: 455.24 | 11 |
| 273 | 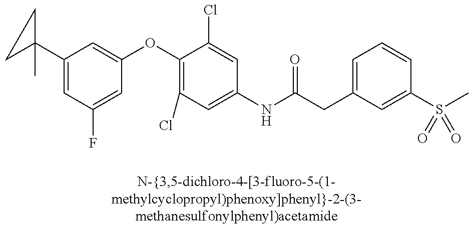 [SMILES: CC1(c2cc(F)cc(Oc3c(Cl)cc(NC(=O)Cc4cccc(S(C)(=O)=O)c4)cc3Cl)c2)CC1] | Method A: rt = 2.41 min; Obs. Adducts: [M + H]; Obs. Mass: 521.93 Method B: rt = 2.42 min; Obs. Adducts: [M + H]; Obs. Mass: 522.14 | 51 |
| 274 | 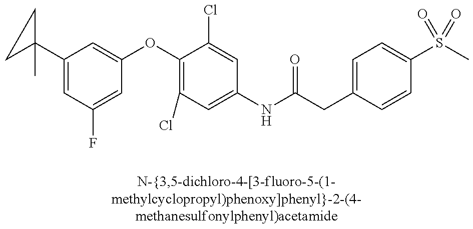 [SMILES: CC1(c2cc(F)cc(Oc3c(Cl)cc(NC(=O)Cc4ccc(S(C)(=O)=O)cc4)cc3Cl)c2)CC1] | Method A: rt = 2.37 min; Obs. Adducts: [M + H]; Obs. Mass: 522.12 Method B: rt = 2.36 min; Obs. Adducts: [M + H]: Obs. Mass: 522.08 | 51 |
| 275 | 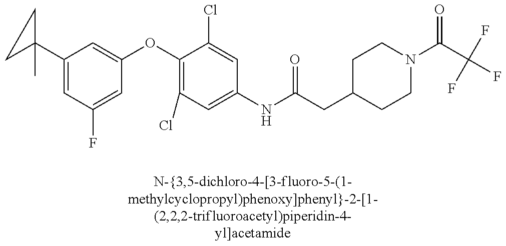 [SMILES: CC1(c2cc(F)cc(Oc3c(Cl)cc(NC(=O)CC4CCN(C(=O)C(F)(F)F)CC4)cc3Cl)c2)CC1] | Method A: rt = 2.7 min; Obs. Adducts: [M + H]; Obs. Mass: 547.16 Method B: rt = 2.69 min; Obs. Adducts: [M + H]; Obs. Mass: 547.14 | 51 |
| 276 | 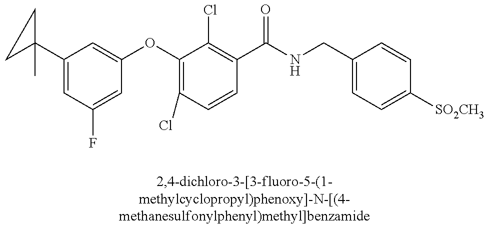 [SMILES: CC1(c2cc(F)cc(Oc3c(Cl)ccc(C(=O)NCc4ccc(S(C)(=O)=O)cc4)c3Cl)c2)CC1] | Method A: rt = 2.16 min; Obs. Adducts: [M + H]; Obs. Mass: 521.95 Method B: rt = 2.17 min; Obs. Adducts: [M + H]; Obs. Mass: 521.94 | 52 |
| 277 | 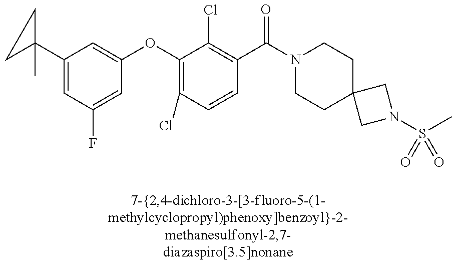 [SMILES: CC1(c2cc(F)cc(Oc3c(Cl)ccc(C(=O)N4CCC5(CC4)CN(S(C)(=O)=O)C5)c3Cl)c2)CC1] | Method A: rt = 2.21 min; Obs. Adducts: [M + H]; Obs. Mass: 541.24 Method B: rt = 2.18 min; Obs. Adducts: [M + H]; Obs. Mass: 541.08 | 52 |
| 278 | 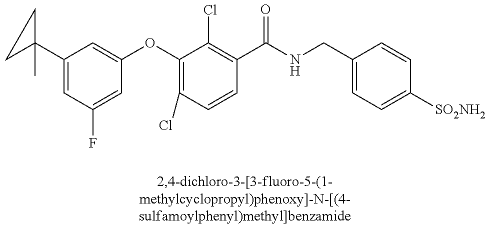 [SMILES: CC1(c2cc(F)cc(Oc3c(Cl)ccc(C(=O)NCc4ccc(S(N)(=O)=O)cc4)c3Cl)c2)CC1] | Method B: rt = 2.15 min; Obs. Adducts: [M + H]; Obs. Mass: 523.23 Method A: rt = 2.1 min; Obs. Adducts: [M + H]; Obs. Mass: 523.17 | 52 |
| 279 | 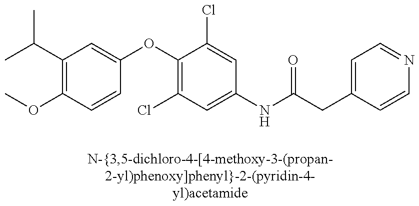 [SMILES: COc1ccc(Oc2c(Cl)cc(NC(=O)Cc3ccncc3)cc2Cl)cc1C(C)C] | Method A: rt = 2.21 min; Obs. Adducts: [M + H]; Obs. Mass: 445.04 Method B: rt = 1.85 min; Obs. Adducts: [M + H]; Obs. Mass: 444.83 | 31 |
| 280 | 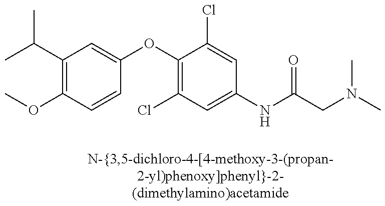 [SMILES: COc1ccc(Oc2c(Cl)cc(NC(=O)CN(C)C)cc2Cl)cc1C(C)C] | Method A: rt = 2.33 min; Obs. Adducts: [M + H]; Obs. Mass: 410.86 Method B: rt = 1.8 min; Obs. Adducts: [M + H]; Obs. Mass: 410.9 | 31 |
| 281 | 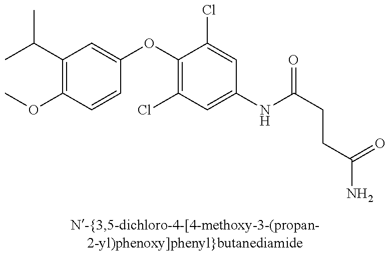 [SMILES: COc1ccc(Oc2c(Cl)cc(NC(=O)CCC(N)=O)cc2Cl)cc1C(C)C] | Method A: rt = 1.97 min; Obs. Adducts: [M + H]; Obs. Mass: 425.04 Method B: rt = 1.96 min; Obs. Adducts: [M + H]; Obs. Mass: 425.06 | 31 |
| 282 | 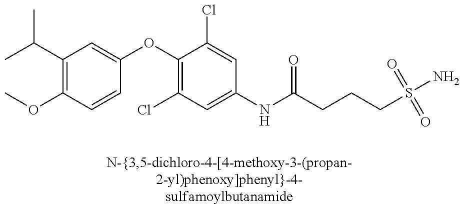 [SMILES: COc1ccc(Oc2c(Cl)cc(NC(=O)CCCS(N)(=O)=O)cc2Cl)cc1C(C)C] | Method A: rt = 2.06 min; Obs. Adducts: [M + H]; Obs. Mass: 474.96 Method B: rt = 2.04 min; Obs. Adducts: [M + H]; Obs. Mass: 475.01 | 31 |
| 283 | 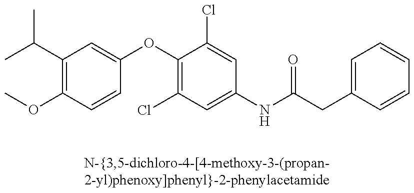 [SMILES: COc1ccc(Oc2c(Cl)cc(NC(=O)Cc3ccccc3)cc2Cl)cc1C(C)C] | Method A: rt = 2.55 min; Obs. Adducts: [M + H]; Obs. Mass: 444.2 Method B: rt = 2.54 min; Obs. Adducts: [M + H]; Obs. Mass: 444.08 | 31 |
| 284 | 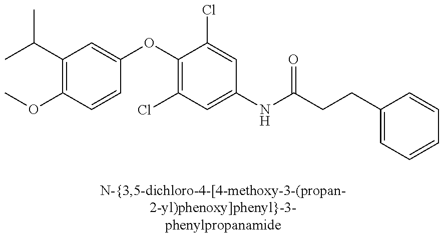 [SMILES: COc1ccc(Oc2c(Cl)cc(NC(=O)CCc3ccccc3)cc2Cl)cc1C(C)C] | Method A: rt = 2.63 min; Obs. Adducts: [M + H]; Obs. Mass: 457.99 Method B: rt = 2.62 min; Obs. Adducts: [M + H]; Obs. Mass: 458.04 | 31 |
| 285 | 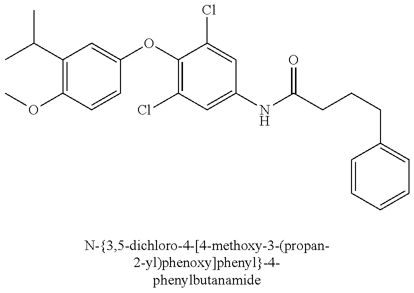 [SMILES: COc1ccc(Oc2c(Cl)cc(NC(=O)CCCc3ccccc3)cc2Cl)cc1C(C)C] | Method A: rt = 2.71 min; Obs. Adducts: [M + H]; Obs. Mass: 472.22 Method B: rt = 2.71 min; Obs. Adducts: [M + H]; Obs. Mass: 471.98 | 31 |
| 286 | 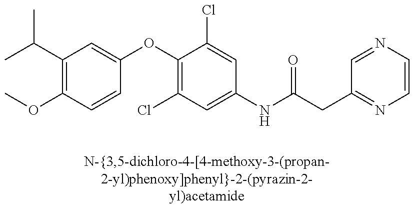 [SMILES: COc1ccc(Oc2c(Cl)cc(NC(=O)Cc3cnccn3)cc2Cl)cc1C(C)C] | Method A: rt = 2.18 min; Obs. Adducts: [M + H]; Obs. Mass: 446.08 Method B: rt = 2.17 min; Obs. Adducts: [M + H]; Obs. Mass: 446.02 | 31 |
| 287 | 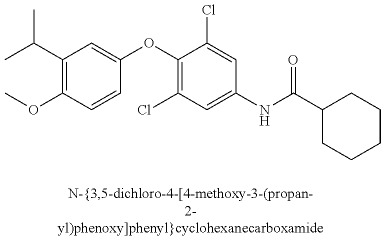 [SMILES: COc1ccc(Oc2c(Cl)cc(NC(=O)C3CCCCC3)cc2Cl)cc1C(C)C] | Method A: rt = 2.71 min; Obs. Adducts: [M + H]; Obs. Mass: 435.88 Method B: rt = 2.7 min; Obs. Adducts: [M + H]; Obs. Mass: 436.15 | 31 |
| 288 | 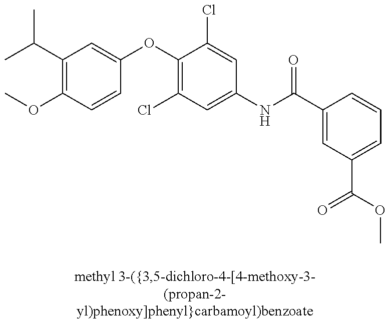 | Method A: rt = 2.61 min; Obs. Adducts: [M + H]; Obs. Mass: 487.96 Method B: rt = 2.6 min; Obs. Adducts: [M + H]; Obs. Mass: 487.74 | 31 |
| 289 | 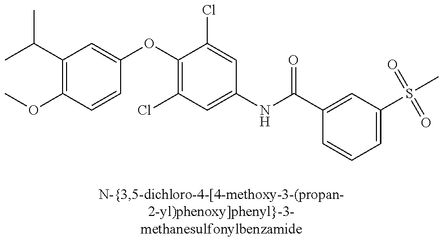 | Method A: rt = 2.38 min; Method B: rt = 2.37 min; Obs. Adducts: [M + H]; Obs. Mass: 508.11 | 31 |
| 290 | 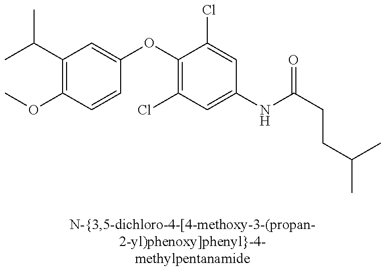 [SMILES: COc1ccc(Oc2c(Cl)cc(NC(=O)CCC(C)C)cc2Cl)cc1C(C)C] | Method A: rt = 2.67 min; Obs. Adducts: [M + H]; Obs. Mass: 424.02 Method B: rt = 2.67 min; Obs. Adducts: [M + H]; Obs. Mass: 424.09 | 31 |
| 291 — | 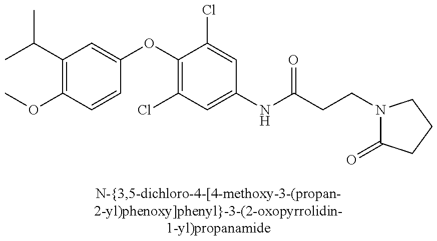 [SMILES: COc1ccc(Oc2c(Cl)cc(NC(=O)CCN3CCCC3=O)cc2Cl)cc1C(C)C] | Method A: rt = 2.15 min; Obs. Adducts: [M + H]; Obs. Mass: 465.04 Method B: rt = 2.14 min; Obs. Adducts: [M + H]; Obs. Mass: 465.08 | 31 |
| 292 | 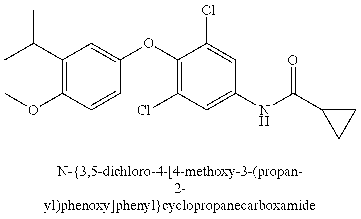 [SMILES: COc1ccc(Oc2c(Cl)cc(NC(=O)C3CC3)cc2Cl)cc1C(C)C] | Method A: rt = 2.42 min; Obs. Adducts: [M + H]; Obs. Mass: 394.04 Method B: rt = 2.41 min; Obs. Adducts: [M + H]; Obs. Mass: 394.05 | 31 |
| 293 | 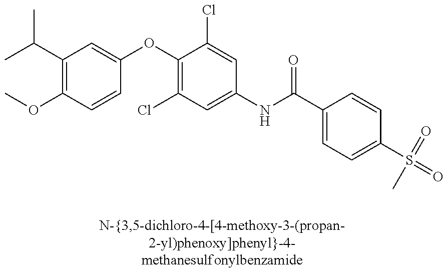 [SMILES: COc1ccc(Oc2c(Cl)cc(NC(=O)c3ccc(S(C)(=O)=O)cc3)cc2Cl)cc1C(C)C] | Method A: rt = 2.37 min Method B: rt = 2.36 min; Obs. Adducts: [M + H]; Obs. Mass: 508.08 | 31 |
| 294 | 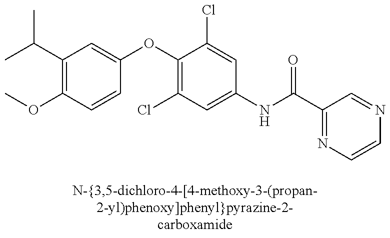 [SMILES: COc1ccc(Oc2c(Cl)cc(NC(=O)c3cnccn3)cc2Cl)cc1C(C)C] | Method A: rt = 2.44 min; Obs. Adducts: [M + H]; Obs. Mass: 432.1 Method B: rt = 2.43 min; Obs. Adducts: [M + H]; Obs. Mass: 432.13 | 31 |
| 295 | 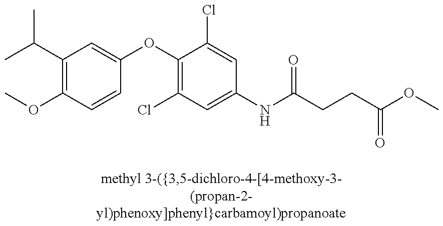 | Method A: rt = 2.31 min; Obs. Adducts: [M + H]; Obs. Mass: 440.12 Method B: rt = 2.3 min; Obs. Adducts: [M + H]; Obs. Mass: 440.04 | 31 |
| 296 | 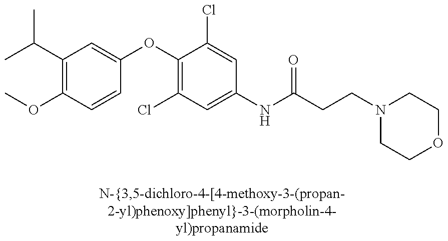 [SMILES: COc1ccc(Oc2c(Cl)cc(NC(=O)CCN3CCOCC3)cc2Cl)cc1C(C)C] | Method B: rt = 2 min; Obs. Adducts: [M + H]; Obs. Mass: 467.24 Method A: rt = 2.35 min; Obs. Adducts: [M + H]; Obs. Mass: 467.21 | 31 |
| 297 | 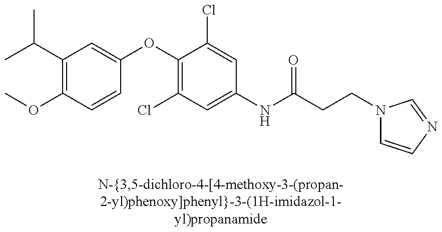 [SMILES: COc1ccc(Oc2c(Cl)cc(NC(=O)CCn3ccnc3)cc2Cl)cc1C(C)C] | Method B: rt = 2 min; Obs. Adducts: [M + H]; Obs. Mass: 448.18 Method A: rt = 2.2 min; Obs. Adducts: [M + H]; Obs. Mass: 448.18 | 31 |
| 298 | 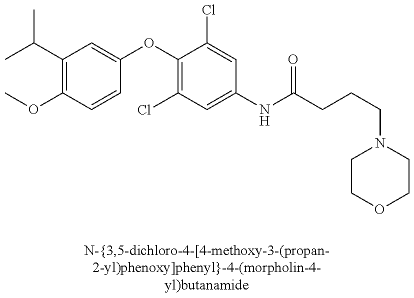 [SMILES: COc1ccc(Oc2c(Cl)cc(NC(=O)CCCN3CCOCC3)cc2Cl)cc1C(C)C] | Method A: rt = 2.15 min; Obs. Adducts: [M + H]; Obs. Mass: 480.91 Method B: rt = 1.86 min; Obs. Adducts: [M + H]; Obs. Mass: 481.04 | 31 |
| 299 | 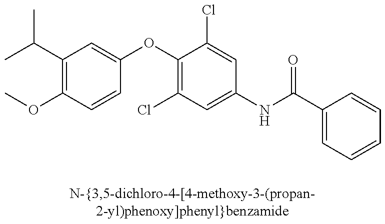 [SMILES: COc1ccc(Oc2c(Cl)cc(NC(=O)c3ccccc3)cc2Cl)cc1C(C)C] | Method B: rt = 2.69 min; Obs. Adducts: [M + H]; Obs. Mass: 430.17 Method A: rt = 2.68 min; Obs. Adducts: [M + H]; Obs. Mass: 430.18 | 31 |
| 300 | 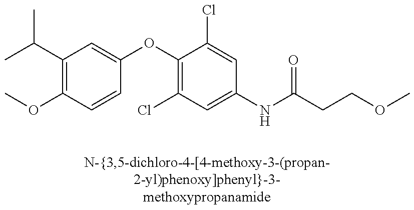 | Method A: rt = 2.28 min; Obs. Adducts: [M + H]; Obs. Mass: 411.96 Method B: rt = 2.27 min; Obs. Adducts: [M + H]; Obs. Mass: 412.07 | 31 |
| 301 | 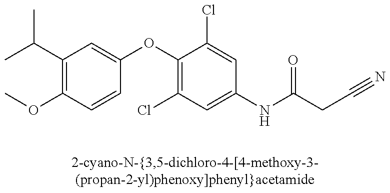 [SMILES: COc1ccc(Oc2c(Cl)cc(NC(=O)CC#N)cc2Cl)cc1C(C)C] | Method B: rt = 2.24 min; Obs. Adducts: [M + H]; Obs. Mass: 392.9 Method A: rt = 2.25 min; Obs. Adducts: [M + H]; Obs. Mass: 393.11 | 31 |
| 302 | 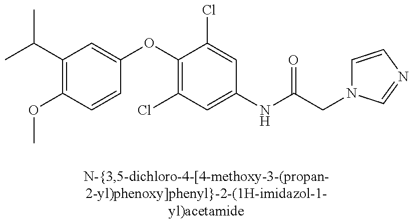 [SMILES: COc1ccc(Oc2c(Cl)cc(NC(=O)Cn3ccnc3)cc2Cl)cc1C(C)C] | Method A: rt = 2.1 min; Obs. Adducts: [M + H]; Obs. Mass: 433.74 Method B: rt = 1.84 min; Obs. Adducts: [M + H]; Obs. Mass: 434.18 | 31 |
| 303 | 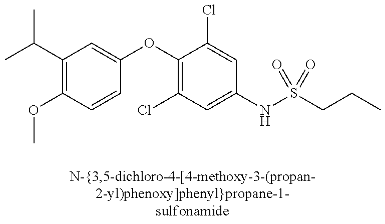 [SMILES: CCCS(=O)(=O)Nc1cc(Cl)c(Oc2ccc(OC)c(C(C)C)c2)c(Cl)c1] | Method B: rt = 2.37 min; Obs. Adducts: [M + H]; Obs. Mass: 432.0 | 33 |
| 304 | 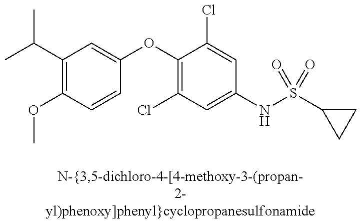 [SMILES: COc1ccc(Oc2c(Cl)cc(NS(=O)(=O)C3CC3)cc2Cl)cc1C(C)C] | Method B: rt = 2.3 min; Obs. Adducts: [M + H]; Obs. Mass: 430 Method A: rt = 2.32 min; Obs. Adducts: [M + NH4]; Obs. Mass: 446.83 | 33 |
| 305 | 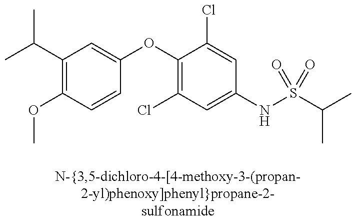 [SMILES: COc1ccc(Oc2c(Cl)cc(NS(=O)(=O)C(C)C)cc2Cl)cc1C(C)C] | Method A: rt = 2.53 min; Obs. Adducts: [M + NH4]; Obs. Mass: 450.8 Method B: rt = 2.54 min | 33 |
| 306 | 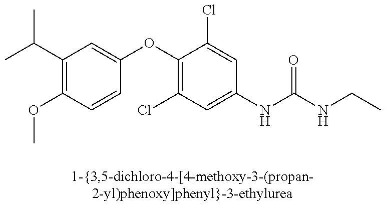 [SMILES: CCNC(=O)Nc1cc(Cl)c(Oc2ccc(OC)c(C(C)C)c2)c(Cl)c1] | Method B: rt = 2.39 min; Obs. Adducts: [M + H]; Obs. Mass: 397.2 | 34 |
| 307 | 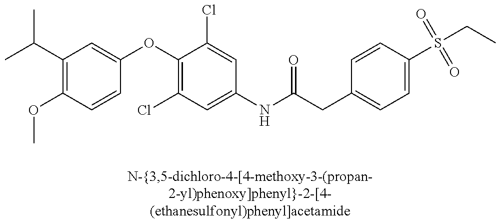 [SMILES: CCS(=O)(=O)c1ccc(CC(=O)Nc2cc(Cl)c(Oc3ccc(OC)c(C(C)C)c3)c(Cl)c2)cc1] | Method A: rt = 2.45 min; Obs. Adducts: [M + H]; Obs. Mass: 536.29 Method B: rt = 2.46 min; Obs. Adducts: [M + H]; Obs. Mass: 536.29 | 31 |
| 308 | 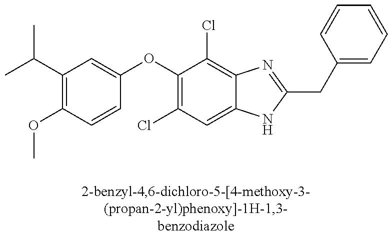 [SMILES: COc1ccc(Oc2c(Cl)cc3[nH]c(Cc4ccccc4)nc3c2Cl)cc1C(C)C] | Method A: rt = 2.42 min; Obs. Adducts: [M + H]; Obs. Mass: 441.07 Method B: rt = 2.19, 2.27 min; Obs. Adducts: [M + H], [M + H]; Obs. Mass: 441.28, 441.28 | 35 |
| 309 | 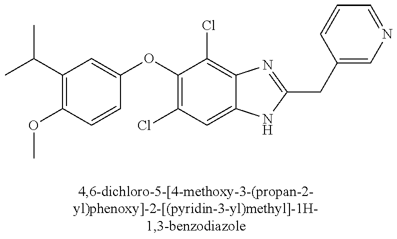 [SMILES: COc1ccc(Oc2c(Cl)cc3[nH]c(Cc4cccnc4)nc3c2Cl)cc1C(C)C] | Method A: rt = 2.11 min; Obs. Adducts: [M + H]; Obs. Mass: 442.03 Method B: rt = 1.83 min; Obs. Adducts: [M + H]; Obs. Mass: 442.26 | 35 |
| 310 | 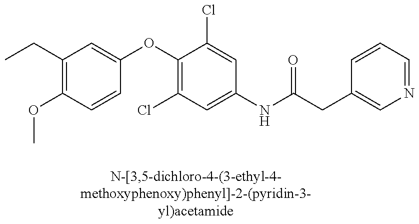 [SMILES: CCc1cc(Oc2c(Cl)cc(NC(=O)Cc3cccnc3)cc2Cl)ccc1OC] | Method B: rt = 1.83 min; Obs. Adducts: [M + H]; Obs. Mass; 431.07 Method A: rt = 2.15 min; Obs. Adducts: [M + H]; Obs. Mass: 431.22 | 37 |
| 311 | 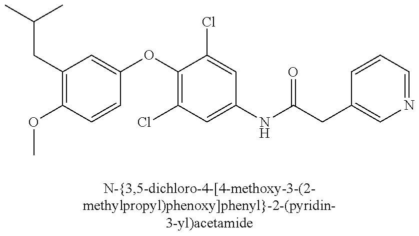 [SMILES: COc1ccc(Oc2c(Cl)cc(NC(=O)Cc3cccnc3)cc2Cl)cc1CC(C)C] | Method A: rt = 2.36 min; Obs. Adducts: [M + H]; Obs. Mass: 459.14 Method B: rt = 1.98 min; Obs. Adducts: [M + H]; Obs. Mass: 459.15 | 37 |
| 312 | 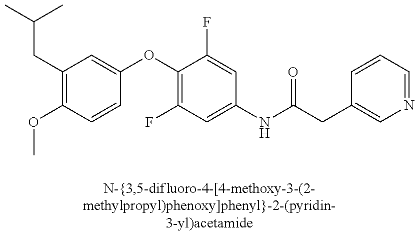 [SMILES: COc1ccc(Oc2c(F)cc(NC(=O)Cc3cccnc3)cc2F)cc1CC(C)C] | Method A: rt = 2.25 min; Obs. Adducts: [M + H]; Obs. Mass: 427.24 Method B: rt = 1.94 min; Obs. Adducts: [M + H]; Obs. Mass: 427.22 | 39 |
| 313 | 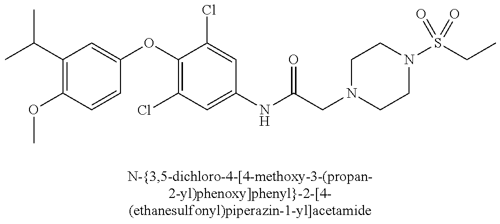 [SMILES: CCS(=O)(=O)N1CCN(CC(=O)Nc2cc(Cl)c(Oc3ccc(OC)c(C(C)C)c3)c(Cl)c2)CC1] | Method A: rt = 2.36 min; Obs. Adducts: [M + H]; Obs. Mass: 544.21 Method B: rt = 1.95 min; Obs. Adducts: [M + H]; Obs. Mass: 544.23 | 47 |
| 314 | 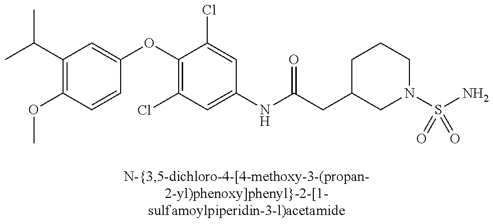 [SMILES: COc1ccc(Oc2c(Cl)cc(NC(=O)CC3CCCN(S(N)(=O)=O)C3)cc2Cl)cc1C(C)C] | Method A: rt = 2.28 min; Obs. Adducts: [M + H]; Obs. Mass: 530.1 Method B: rt = 2.29 min; Obs. Adducts: [M + H]; Obs. Mass: 530.23 | 46 |
| 315 | 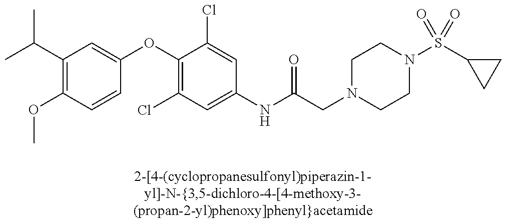 [SMILES: COc1ccc(Oc2c(Cl)cc(NC(=O)CN3CCN(S(=O)(=O)C4CC4)CC3)cc2Cl)cc1C(C)C] | Method A: rt = 2.45 min; Obs. Adducts: [M + H]; Obs. Mass: 556.2 Method B: rt = 1.95 min; Obs. Adducts: [M + H]; Obs. Mass: 556.2 | 47 |
| 316 | 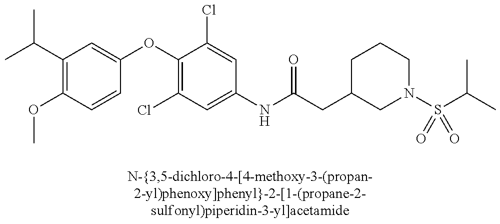 [SMILES: COc1ccc(Oc2c(Cl)cc(NC(=O)CC3CCCN(S(=O)(=O)C(C)C)C3)cc2Cl)cc1C(C)C] | Method A: rt = 2.55 min; Obs. Adducts: [M + H]; Obs. Mass: 557.05 Method B: rt = 2.55 min; Obs. Adducts: [M + H]; Obs. Mass: 557 | 41 |
| 317 | 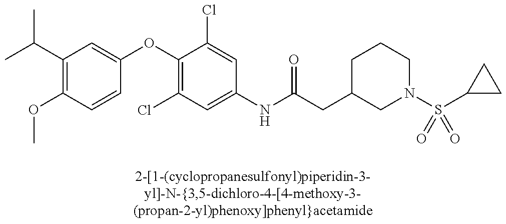 [SMILES: COc1ccc(Oc2c(Cl)cc(NC(=O)CC3CCCN(S(=O)(=O)C4CC4)C3)cc2Cl)cc1C(C)C] | Method A: rt = 2.48 min; Obs. Adducts: [M + H]; Obs. Mass: 555.4 Method B: rt = 2.47 min; Obs. Adducts: [M + H]; Obs. Mass: 555.22 | 41 |
| 318 | 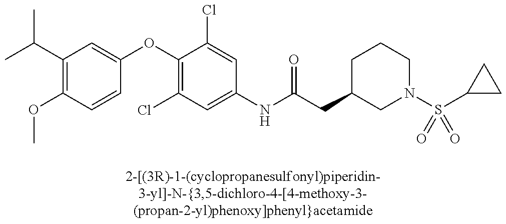 [SMILES: COc1ccc(Oc2c(Cl)cc(NC(=O)C[C@H]3CCCN(S(=O)(=O)C4CC4)C3)cc2Cl)cc1C(C)C] | Method A: rt = 2.51 min; Obs. Adducts: [M + H]; Obs. Mass: 555.05 Method B: rt = 2.47 min; Obs. Adducts: [M + H]; Obs. Mass; 555.2 | 44 |
| 319 | 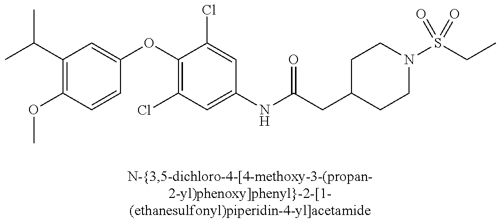 [SMILES: CCS(=O)(=O)N1CCC(CC(=O)Nc2cc(Cl)c(Oc3ccc(OC)c(C(C)C)c3)c(Cl)c2)CC1] | Method A: rt = 2.44 min; Obs. Adducts: [M + H]; Obs. Mass: 542.97 Method B: rt = 2.44 min; Obs. Adducts: [M + H]; Obs. Mass: 543.1 | 45 |
| 320 | 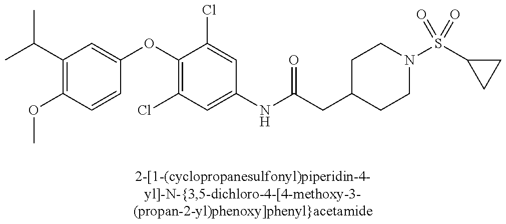 [SMILES: COc1ccc(Oc2c(Cl)cc(NC(=O)CC3CCN(S(=O)(=O)C4CC4)CC3)cc2Cl)cc1C(C)C] | Method A: rt = 2.49 min; Obs. Adducts: [M + H]; Obs. Mass; 555.08 Method B: rt = 2.45 min; Obs. Adducts: [M + H]; Obs. Mass: 555.18 | 45 |
| 321 | 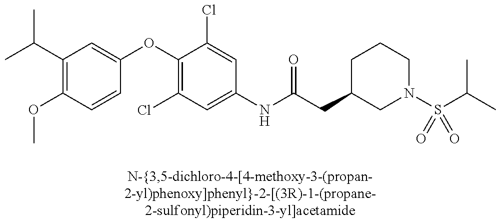 [SMILES: COc1ccc(Oc2c(Cl)cc(NC(=O)C[C@H]3CCCN(S(=O)(=O)C(C)C)C3)cc2Cl)cc1C(C)C] | Method A: rt = 2.57 min; Obs. Adducts: [M + H]; Obs. Mass: 557.02 Method B: rt = 2.52 min; Obs. Adducts: [M + H]; Obs. Mass: 557.08 | 44 |
| 322 | 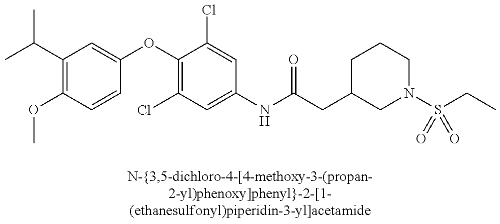 [SMILES: CCS(=O)(=O)N1CCCC(CC(=O)Nc2cc(Cl)c(Oc3ccc(OC)c(C(C)C)c3)c(Cl)c2)C1] | Method A: rt = 2.47 min; Obs. Adducts: [M + H]; Obs. Mass: 543.01 Method B: rt = 2.46 min; Obs. Adducts: [M + H]; Obs. Mass: 542.93 | 41 |
| 323 | 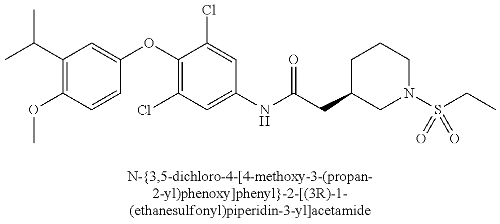 [SMILES: CCS(=O)(=O)N1CCC[C@H](CC(=O)Nc2cc(Cl)c(Oc3ccc(OC)c(C(C)C)c3)c(Cl)c2)C1] | Method A: rt = 2.46 min; Obs. Adducts: [M + H]; Obs. Mass: 543.01 Method B: rt = 2.47 min; Obs. Adducts: [M + H]; Obs. Mass: 543.04 | 44 |
| 324 | 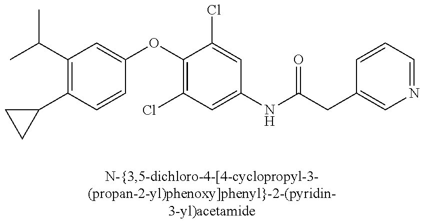 [SMILES: CC(C)c1cc(Oc2c(Cl)cc(NC(=O)Cc3cccnc3)cc2Cl)ccc1C1CC1] | Method A: rt = 2.47 min; Obs. Adducts: [M + H]; Obs. Mass: 454.96 Method B: rt = 2.13 min; Obs. Adducts: [M + H]; Obs. Mass: 455.2 | 49 |
| 325 | 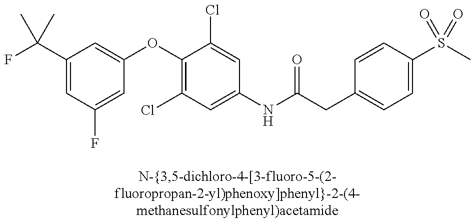 [SMILES: CC(C)(F)c1cc(F)cc(Oc2c(Cl)cc(NC(=O)Cc3ccc(S(C)(=O)=O)cc3)cc2Cl)c1] | Method F: rt = 1.10 min; Obs. Adducts: [M − H]; Obs. Mass: 526.3 | 53 |
| 326 | 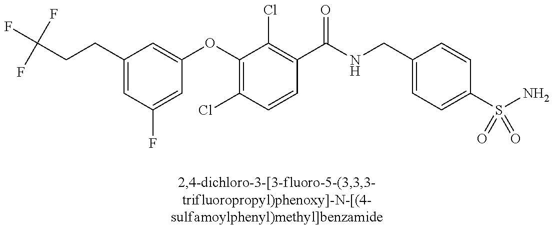 [SMILES: NS(=O)(=O)c1ccc(CNC(=O)c2ccc(Cl)c(Oc3cc(F)cc(CCC(F)(F)F)c3)c2Cl)cc1] | Method C: rt = 0.98 min; Obs. Adducts: [M + H]; Obs. Mass: 565.2 | 53 |
| 327 | 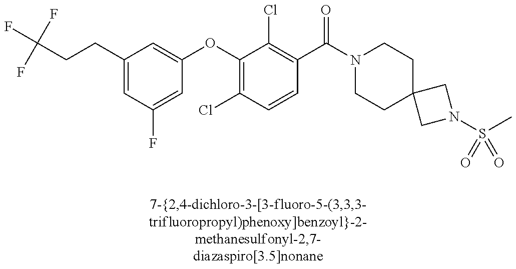 [SMILES: CS(=O)(=O)N1CC2(CCN(C(=O)c3ccc(Cl)c(Oc4cc(F)cc(CCC(F)(F)F)c4)c3Cl)CC2)C1] | Method C: rt = 1.01 min; Obs. Adducts: [M + H]; Obs. Mass: 583.2 | 53 |
| 328 | 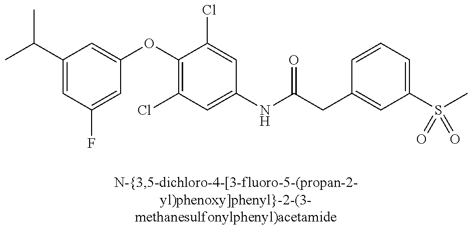 [SMILES: CC(C)c1cc(F)cc(Oc2c(Cl)cc(NC(=O)Cc3cccc(S(C)(=O)=O)c3)cc2Cl)c1] | Method A: rt = 2.41 min; Obs. Adducts: [M + H]; Obs. Mass: 509.76 Method B: rt = 2.35 min; Obs. Adducts: [M + H]; Obs. Mass: 510.35 | 14 |
| 329 | 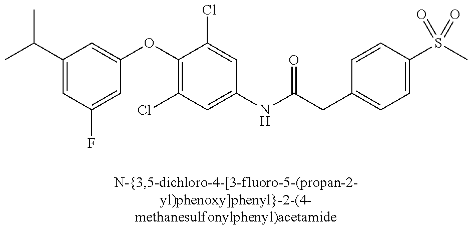 [SMILES: CC(C)c1cc(F)cc(Oc2c(Cl)cc(NC(=O)Cc3ccc(S(C)(=O)=O)cc3)cc2Cl)c1] | Method A: rt = 2.39 min; Obs. Adducts: [M + H]; Obs. Mass: 510.15 Method B: rt = 2.39 min; Obs. Adducts: [M + H]; Obs. Mass: 510.17 | 14 |
| 330 | 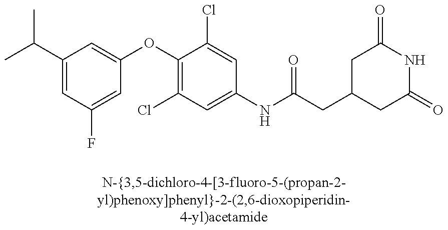 [SMILES: CC(C)c1cc(F)cc(Oc2c(Cl)cc(NC(=O)CC3CC(=O)NC(=O)C3)cc2Cl)c1] | Method A: rt = 2.13 min Method B: rt = 2.13 min; Obs. Adducts: [M + H]; Obs. Mass: 467.35 | 14 |
| 331 | 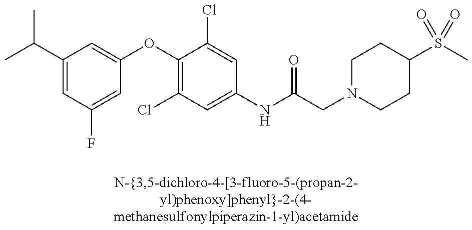 [SMILES: CC(C)c1cc(F)cc(Oc2c(Cl)cc(NC(=O)CN3CCC(S(C)(=O)=O)CC3)cc2Cl)c1] | Method A: rt = 2.32 min; Obs. Adducts: [M + H]; Obs. Mass: 517.33 Method B: rt = 1.82 min; Obs. Adducts: [M + H]; Obs. Mass: 517.33 | 14 |
| 332 | 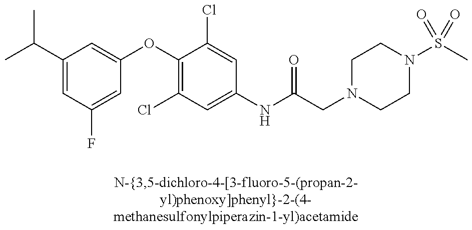 [SMILES: CC(C)c1cc(F)cc(Oc2c(Cl)cc(NC(=O)CN3CCN(S(C)(=O)=O)CC3)cc2Cl)c1] | Method B: rt = 1.88 min; Obs. Adducts: [M + H]; Obs. Mass: 518.22 Method A: rt = 2.37 min; Obs. Adducts: [M + H]; Obs. Mass: 517.9 | 14 |
| 333 | 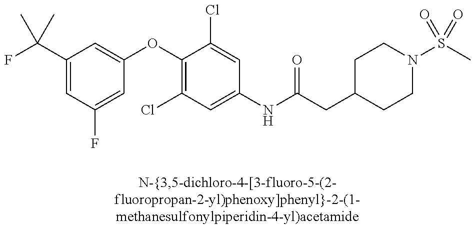 [SMILES: CC(C)(F)c1cc(F)cc(Oc2c(Cl)cc(NC(=O)CC3CCN(S(C)(=O)=O)CC3)cc2Cl)c1] | Method A: rt = 2.27 min; Obs. Adducts: [M + H]; Obs. Mass: 534.81 Method B: rt = 2.25 min; Obs. Adducts: [M + H]; Obs. Mass: 535.32 | 53 |
| 334 | 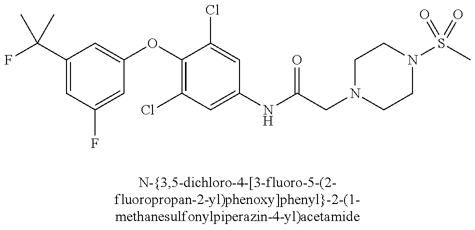 [SMILES: CC(C)(F)c1cc(F)cc(Oc2c(Cl)cc(NC(=O)CN3CCN(S(C)(=O)=O)CC3)cc2Cl)c1] | Method A: rt = 2.2 min; Obs. Adducts: [M + H]; Obs. Mass: 536.12 Method B: rt = 1.81 min; Obs. Adducts: [M + H]; Obs. Mass: 535.96 | 53 |
| 335 | 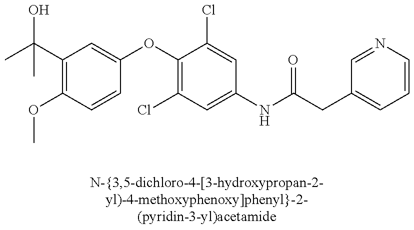 [SMILES: COc1ccc(Oc2c(Cl)cc(NC(=O)Cc3cccnc3)cc2Cl)cc1C(C)(C)O] | Method A: rt = 1.73 min; Obs. Adducts: [M + H]; Obs. Mass: 461.02 Method B: rt = 1.42 min; Obs Adducts: [M + H]; Obs. Mass: 461.1 | 53 |
| 336 | 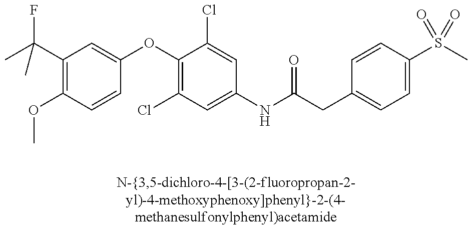 [SMILES: COc1ccc(Oc2c(Cl)cc(NC(=O)Cc3ccc(S(C)(=O)=O)cc3)cc2Cl)cc1C(C)(C)F] | Method A: rt = 2.25 min; Obs. Adducts: [M + NH4]; Obs. Mass: 556.84 Method B: rt = 2.22 min; Obs. Adducts: [M + Na]; Obs. Mass: 562.25 | 53 |
| 337 | 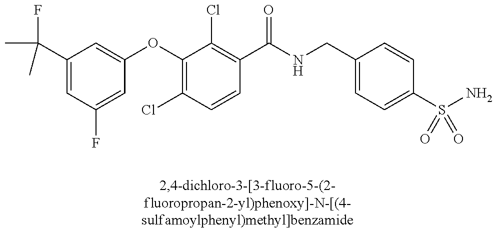 [SMILES: CC(C)(F)c1cc(F)cc(Oc2c(Cl)ccc(C(=O)NCc3ccc(S(N)(=O)=O)cc3)c2Cl)c1] | Method A: rt = 1.94 min; Obs. Adducts:; Obs. Mass: 508.9 Method B: rt = 1.93 min; Obs Adducts:; Obs. Mass: | 53 |
| 338 | 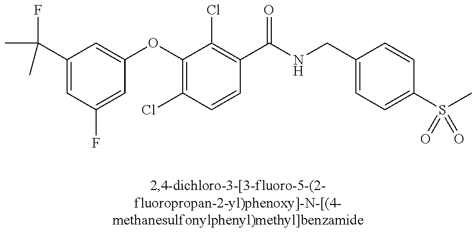 [SMILES: CC(C)(F)c1cc(F)cc(Oc2c(Cl)ccc(C(=O)NCc3ccc(S(C)(=O)=O)cc3)c2Cl)c1] | Method A: rt = 2.06 min; Obs. Adducts: [M + NH4]; Obs. Mass: 546.91 Method B: rt = 1.96 min; Obs. Adducts: [M + Na]; Obs. Mass: 550.04 Method A: rt = 2.1 min; Obs. Adducts: [M + NH4]; Obs. Mass: 544.91 | 53 |
| 339 | 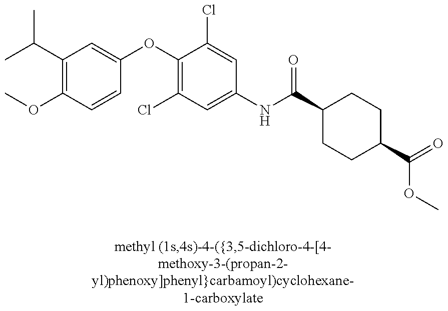 [SMILES: COc1ccc(Oc2c(Cl)cc(NC(=O)[C@H]3CC[C@@H](C(=O)OC)CC3)cc2Cl)cc1C(C)C] | Method A: rt = 2.56 min; Obs. Adducts: [M + H]; Obs. Mass: 494.28 Method B: rt = 2.55 min; Obs. Adducts: [M + H]; Obs. Mass: 494.24 | 31 |
| 340 | 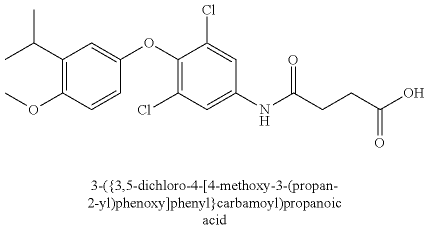 [SMILES: COc1ccc(Oc2c(Cl)cc(NC(=O)CCC(=O)O)cc2Cl)cc1C(C)C] | Method A: rt = 1.76 min; Obs. Adducts: [M + H]; Obs. Mass: 426.03 Method B: rt = 2.18 min; Obs. Adducts: [M + H]; Obs. Mass: 426.3 | 31 |
| 341 | 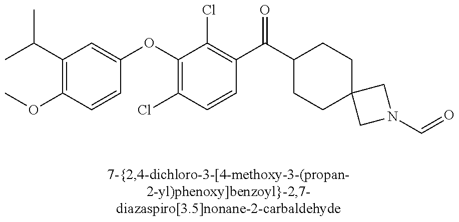 [SMILES: COc1ccc(Oc2c(Cl)ccc(C(=O)C3CCC4(CC3)CN(C=O)C4)c2Cl)cc1C(C)C] | Method C: rt = 0.98 min; Obs. Adducts: [M + H]; Obs. Mass: 490.8 | 19 |
| 342 | 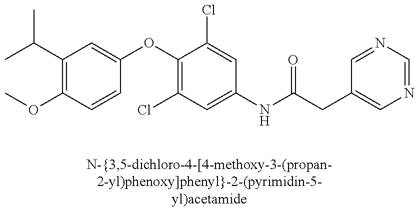 [SMILES: COc1ccc(Oc2c(Cl)cc(NC(=O)Cc3cncnc3)cc2Cl)cc1C(C)C] | Method B: rt = 2.1 min; Obs. Adducts: [M + H]; Obs. Mass: 446.02 Method A: rt = 2.24 min; Obs. Adducts: [M + H]; Obs. Mass: 446.21 | 31 |
| 343 | 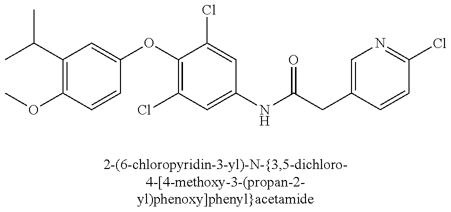 [SMILES: COc1ccc(Oc2c(Cl)cc(NC(=O)Cc3ccc(Cl)nc3)cc2Cl)cc1C(C)C] | Method B: rt = 2.43 min; Obs. Adducts: [M + H]; Obs. Mass: 479.04 Method A: rt = 2.57 min; Obs. Adducts: [M + H]; Obs. Mass: 479.16 | 31 |
| 344 | 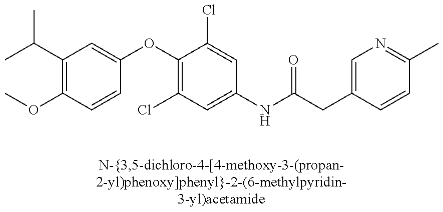 [SMILES: COc1ccc(Oc2c(Cl)cc(NC(=O)Cc3ccc(C)nc3)cc2Cl)cc1C(C)C] | Method B: rt = 1.88 min; Obs. Adducts: [M + H]; Obs. Mass: 459.03 Method B: rt = 2.02 min; Obs. Adducts: [M + H]; Obs. Mass: 459.25 | 31 |
| 345 | 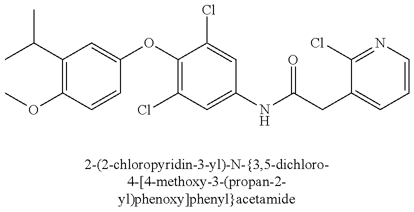 [SMILES: COc1ccc(Oc2c(Cl)cc(NC(=O)Cc3cccnc3Cl)cc2Cl)cc1C(C)C] | Method A: rt = 2.5 min; Obs. Adducts: [M + H]; Obs. Mass: 479.12 Method B: rt = 2.51 min; Obs. Adducts: [M + H]; Obs. Mass; 479.15 | 31 |
| 346 | 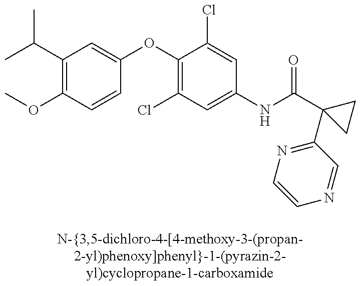 [SMILES: COc1ccc(Oc2c(Cl)cc(NC(=O)C3(c4cnccn4)CC3)cc2Cl)cc1C(C)C] | Method A: rt = 2.48 min; Obs. Adducts: [M + H]; Obs. Mass: 472.11 Method B: rt = 2.58 min; Obs. Adducts: [M + H]; Obs. Mass: 472.29 | 31 |
| 347 | 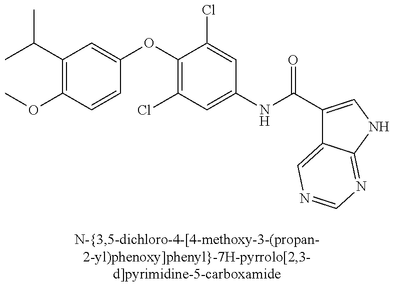 [SMILES: COc1ccc(Oc2c(Cl)cc(NC(=O)c3c[nH]c4ncncc34)cc2Cl)cc1C(C)C] | Method B: rt = 1.9 min; Obs. Adducts: [M + H]; Obs. Mass: 471 Method A: rt = 2.16 min; Obs. Adducts: [M + H]; Obs. Mass: 471.23 | 31 |
| 348 | 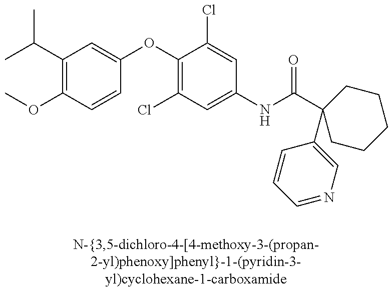 [SMILES: COc1ccc(Oc2c(Cl)cc(NC(=O)C3(c4cccnc4)CCCCC3)cc2Cl)cc1C(C)C] | Method B: rt = 2.25 min; Obs. Adducts: [M + H]; Obs. Mass: 512.97 Method A: rt = 2.71 min; Obs. Adducts: [M + H]; Obs. Mass: 513.42 | 31 |
| 349 | 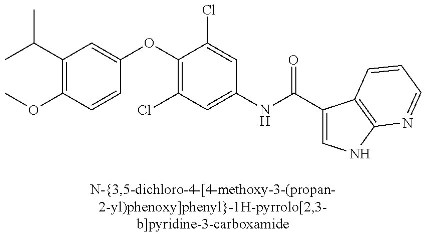 [SMILES: COc1ccc(Oc2c(Cl)cc(NC(=O)c3c[nH]c4ncccc34)cc2Cl)cc1C(C)C] | Method A: rt = 2.39 min; Obs. Adducts: [M + H]; Obs. Mass: 470.15 Method B: rt = 2.26 min; Obs. Adducts: [M + H]; Obs. Mass: 470.17 | 31 |
| 350 | 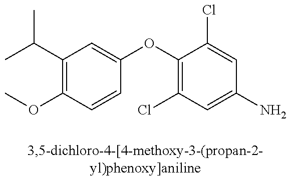 [SMILES: COc1ccc(Oc2c(Cl)cc(N)cc2Cl)cc1C(C)C] | Method C: rt = 1.13 min; Obs. Adducts: [M + H]; Obs. Mass: 326.2 | |
| 351 | 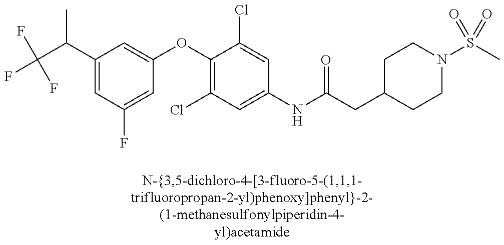 [SMILES: CC(c1cc(F)cc(Oc2c(Cl)cc(NC(=O)CC3CCN(S(C)(=O)=O)CC3)cc2Cl)c1)C(F)(F)F] | Method H: rt = 11.02 min; Obs. Adducts: [M + H]; Obs. Mass: 571.2 | 53 |
| 352 | 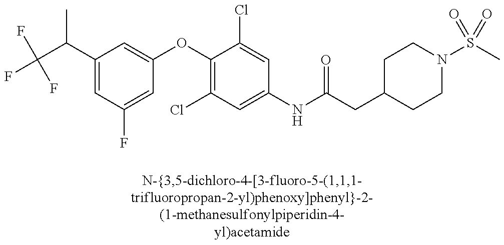 [SMILES: CC(c1cc(F)cc(Oc2c(Cl)cc(NC(=O)CC3CCN(S(C)(=O)=O)CC3)cc2Cl)c1)C(F)(F)F] | Method H: rt = 11.04 min; Obs. Adducts: [M + H]; Obs. Mass: 571.2 | 53 |
| 353 | 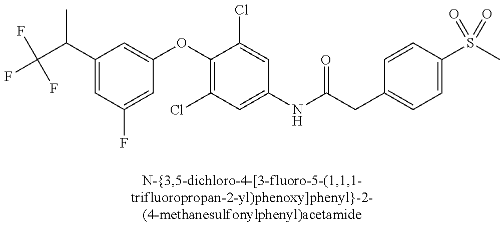 [SMILES: CC(c1cc(F)cc(Oc2c(Cl)cc(NC(=O)Cc3ccc(S(C)(=O)=O)cc3)cc2Cl)c1)C(F)(F)F] | Method C: rt = 1.07 min; Obs. Adducts: [M + H]; Obs. Mass: 564.1 | 53 |
| 354 | 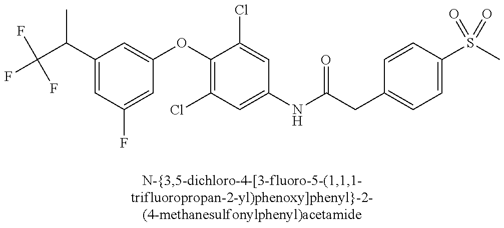 [SMILES: CC(c1cc(F)cc(Oc2c(Cl)cc(NC(=O)Cc3ccc(S(C)(=O)=O)cc3)cc2Cl)c1)C(F)(F)F] | Method C: rt = 1.07 min; Obs. Adducts: [M + H]; Obs. Mass: 564.1 | 53 |
| 355 | 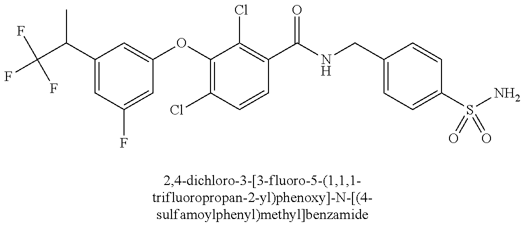 [SMILES: CC(c1cc(F)cc(Oc2c(Cl)ccc(C(=O)NCc3ccc(S(N)(=O)=O)cc3)c2Cl)c1)C(F)(F)F] | Method C: rt = 0.98 min; Obs. Adducts: [M + H]; Obs. Mass: 565.2 | 53 |
| 356 | 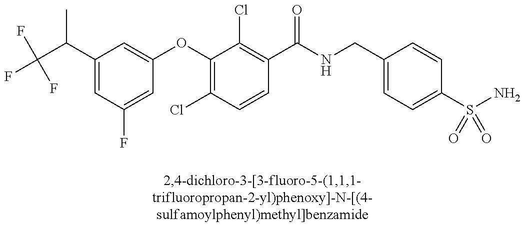 [SMILES: CC(c1cc(F)cc(Oc2c(Cl)ccc(C(=O)NCc3ccc(S(N)(=O)=O)cc3)c2Cl)c1)C(F)(F)F] | Method F: rt = 0.98 min; Obs. Adducts: [M + H]; Obs. Mass: 565.3 | 53 |
| 357 | 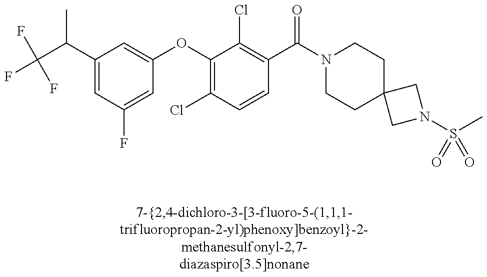 [SMILES: CC(c1cc(F)cc(Oc2c(Cl)ccc(C(=O)N3CCC4(CC3)CN(S(C)(=O)=O)C4)c2Cl)c1)C(F)(F)F] | Method C: rt = 1.01 min; Obs. Adducts: [M + H]; Obs. Mass: 583.2 | 53 |
| 358 | 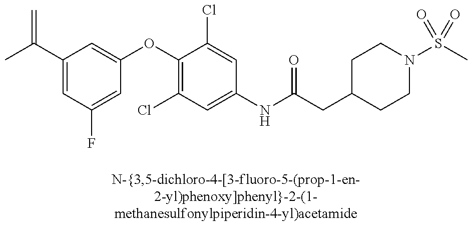 [SMILES: C=C(C)c1cc(F)cc(Oc2c(Cl)cc(NC(=O)CC3CCN(S(C)(=O)=O)CC3)cc2Cl)c1] | Method H: rt = 11.44 min; Obs. Adducts: [M + H]; Obs. Mass: 515.1 | 53 |
| 359 | 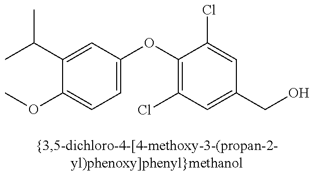 [SMILES: COc1ccc(Oc2c(Cl)cc(CO)cc2Cl)cc1C(C)C] | Method C: rt = 1.12 min; Obs. Adducts: [M − OH]; Obs. Mass: 323.1 | |

### Biological Assay

### RORgT Gal4 Luciferase Reporter Gene Assay

The inhibition potency of each final compound was determined using engineered Jurkat cells overexpressing constitutively active RORgT proteins fused with Gal4 Luc reporter (Jurkat pEx/Gal/hRORγ CLBD/HYG pG5luc/blast). 25 μL of cryopreserved Jurkat cells over expressing ligand binding domain (LBD) of RORgT (aa267-516, NM_005060) and Gal4 Luc, or full length of human RORgT and Gal4 Luc, were plated in 384-well solid white cell culture plates (PerkinElmer 6007899), with a density of 10,000 cells/well in RPMI 1640 cell culture media (Gibco 11875-085). The media contained 0.1% BSA, 10 mM HEPES (Gibco 15360-080), 100 mM Sodium Pyruvate (Gibco 11360-040), 50 mg/mL Hygromycin B (Invitrogen 10687-010), and 10 mg/mL Blasticidin (Invitrogen R210-01).

100 nL of compound at varying concentrations in 3-fold serial dilution, with final concentrations ranging from 40 μM to 0.67 nM, were added to the cells using Labcyte Echo 550. The compound and the cells were incubated for 18 hours at 37° C. in a cell culture incubator. Cells were then lysed with 15 uL of Steady-Glo Luciferase Assay reagent (Promega EZ550), followed by centrifuging the assay plates at 1500 RPM for 1 minute. Subsequently, the plates were read on the Envision (PerkinElmer). The inhibition of constitutive activity of RORgT achieved by graded concentrations of compound was calculated as a percentage of the luminescence signal window reduction over a control compound.

| | RORg_GAL4 |
|---|---|
| Example No. | EC₅₀ (nM) |
| 1 | 829 |
| 2 | 152 |
| 3 | 54 |
| 4 | 121 |
| 5 | 437 |
| 6 | 533 |
| 7 | 173 |
| 8 | 23 |
| 9 | 81 |
| 10 | 2,125 |
| 11 | 77 |
| 12 | 1,097 |
| 13 | 268 |
| 14 | 129 |
| 15 | 14 |
| 16 | 6.1 |
| 17 | 5.2 |
| 18 | 1,867 |
| 19 | 396 |
| 20 | 41 |
| 21 | 650 |
| 22 | 95 |
| 23 | 49 |
| 24 | 45 |
| 25 | 626 |
| 26 | 322 |
| 27 | 30 |
| 28 | 6.9 |
| 29 | 5.8 |
| 30 | 216 |
| 31 | 100 |
| 32 | 296 |
| 33 | 4,281 |
| 34 | 1,064 |
| 35 | 2,054 |
| 36 | 455 |
| 37 | 1,122 |
| 38 | 30 |
| 39 | 3,135 |
| 40 | 2,452 |
| 41 | 356 |
| 42 | 207 |
| 43 | 246 |
| 44 | 120 |
| 45 | 18 |
| 46 | 438 |
| 47 | 15 |
| 48 | 105 |
| 49 | 344 |
| 50 | 75 |
| 51 | 1.7 |
| 52 | 28 |
| 53 | 40 |
| 54 | 34 |
| 55 | 54 |
| 56 | 23 |
| 57 | 16 |
| 58 | 383 |
| 59 | 41 |
| 60 | 139 |
| 61 | 46 |
| 62 | 96 |
| 63 | 31 |
| 64 | 23 |
| 65 | 69 |
| 66 | 268 |
| 67 | 116 |
| 68 | 15 |
| 69 | 481 |
| 70 | 141 |
| 71 | 15 |
| 72 | 33 |
| 73 | 18 |
| 74 | 49 |
| 75 | 33 |
| 76 | 922 |
| 77 | 953 |
| 78 | 1,046 |
| 79 | 18 |
| 80 | 287 |
| 81 | 1,312 |
| 82 | 7,598 |
| 83 | 1,169 |
| 84 | 152 |
| 85 | 304 |
| 86 | 12 |
| 87 | 541 |
| 88 | 24 |
| 89 | 4,082 |
| 90 | 13 |
| 91 | 88 |
| 92 | 24 |
| 93 | 167 |
| 94 | 26 |
| 95 | 24 |
| 96 | 13 |
| 97 | 14 |
| 98 | 20 |
| 99 | 153 |
| 100 | 117 |
| 101 | 14 |
| 102 | 455 |
| 103 | 50 |
| 104 | 15 |
| 105 | 30 |
| 106 | 32 |
| 107 | 35 |
| 108 | 766 |
| 109 | 17 |
| 110 | 39 |
| 111 | 936 |
| 112 | 14 |
| 113 | 27 |
| 114 | 5.2 |
| 115 | 25 |
| 116 | 350 |
| 117 | 6.0 |
| 118 | 61 |
| 119 | 11 |
| 120 | 37 |
| 121 | 13 |
| 122 | 3.6 |
| 123 | 5.4 |
| 124 | 121 |
| 125 | 548 |
| 126 | 3.2 |
| 127 | 35 |
| 128 | 139 |
| 129 | 11 |
| 130 | 5.5 |
| 131 | 2.7 |
| 132 | 6.3 |
| 133 | 4.6 |
| 134 | 3.2 |
| 135 | 11 |
| 136 | 13 |
| 137 | 14 |
| 138 | 225 |
| 139 | 187 |
| 140 | 9.3 |
| 141 | 182 |
| 142 | 71 |
| 143 | 11.5 |
| 144 | 6.6 |
| 145 | 8.5 |
| 146 | 28 |
| 147 | 9.6 |
| 148 | 4.0 |
| 149 | 15 |
| 150 | 8.2 |
| 151 | 9.9 |
| 152 | 2.5 |
| 153 | 195 |
| 154 | 6.6 |
| 155 | 4,748 |
| 156 | 1,926 |
| 157 | 939 |
| 158 | 499 |
| 159 | 377 |
| 160 | 325 |
| 161 | 2,326 |
| 162 | 220 |
| 163 | 340 |
| 164 | 80 |
| 165 | 182 |
| 166 | 424 |
| 167 | 245 |
| 168 | 270 |
| 169 | 93 |
| 170 | 370 |
| 171 | 2,590 |
| 172 | 49 |
| 173 | 265 |
| 174 | 286 |
| 175 | 345 |
| 176 | 83 |
| 177 | 6.7 |
| 178 | 336 |
| 179 | 1,126 |
| 180 | 325 |
| 181 | 214 |
| 182 | 355 |
| 183 | 284 |
| 184 | 246 |
| 185 | 28 |
| 186 | 90 |
| 187 | 522 |
| 188 | 383 |
| 189 | 40 |
| 190 | 989 |
| 191 | 128 |
| 192 | 89 |
| 193 | 866 |
| 194 | 6,464 |
| 195 | 616 |
| 196 | 200 |
| 197 | 860 |
| 198 | 872 |
| 199 | 3,895 |
| 200 | 330 |
| 201 | 511 |
| 202 | 4,491 |
| 203 | 129 |
| 204 | 40 |
| 205 | 564 |
| 206 | 100 |
| 207 | 63 |
| 208 | 508 |
| 209 | 659 |
| 210 | 105 |
| 211 | 46 |
| 212 | 11 |
| 213 | 19 |
| 214 | 434 |
| 215 | 37 |
| 216 | 813 |
| 217 | 45 |
| 218 | 328 |
| 219 | 1,502 |
| 220 | 51 |
| 221 | 560 |
| 222 | 225 |
| 223 | 1,113 |
| 224 | 732 |
| 225 | 920 |
| 226 | 286 |
| 227 | 109 |
| 228 | 102 |
| 229 | 891 |
| 230 | 373 |
| 231 | 26 |
| 232 | 285 |
| 233 | 319 |
| 234 | 635 |
| 235 | 1,275 |
| 236 | 597 |
| 237 | 235 |
| 238 | 752 |
| 239 | 170 |
| 240 | 99 |
| 241 | 64 |
| 242 | 40 |
| 243 | 1,284 |
| 244 | 37 |
| 245 | 1,565 |
| 246 | 123 |
| 247 | 114 |
| 248 | 2,247 |
| 249 | 1,637 |
| 250 | 500 |
| 251 | 433 |
| 252 | 264 |
| 253 | 477 |
| 254 | 655 |
| 255 | 19 |
| 256 | 153 |
| 257 | 70 |
| 258 | 19 |
| 259 | 356 |
| 260 | 102 |
| 261 | 14 |
| 262 | 223 |
| 263 | 105 |
| 264 | 39 |
| 265 | 204 |
| 266 | 11 |
| 267 | 373 |
| 268 | 40 |
| 269 | 185 |
| 270 | 22 |
| 271 | 78 |
| 272 | 260 |
| 273 | 1.1 |
| 274 | 3.9 |
| 275 | 12 |
| 276 | 5.1 |
| 277 | 5.6 |
| 278 | 1.7 |
| 279 | 212 |
| 280 | 656 |
| 281 | 375 |
| 282 | 264 |
| 283 | 224 |
| 284 | 40,000 |
| 285 | 284 |
| 286 | 316 |
| 287 | 132 |
| 288 | 1,266 |
| 289 | 1,480 |
| 290 | 524 |
| 291 | 134 |
| 292 | 940 |
| 293 | 3,272 |
| 294 | 4,521 |
| 295 | 308 |
| 296 | 1,009 |
| 297 | 44 |
| 298 | 727 |
| 299 | 20,000 |
| 300 | 591 |
| 301 | 343 |
| 302 | 185 |
| 303 | 858 |
| 304 | 1,082 |
| 305 | 421 |
| 306 | 1,888 |
| 307 | 16 |
| 308 | 1,278 |
| 309 | 409 |
| 310 | 293 |
| 311 | 153 |
| 312 | 1,439 |
| 313 | 32 |
| 314 | 844 |
| 315 | 88 |
| 316 | 943 |
| 317 | 95 |
| 318 | 76 |
| 319 | 33 |
| 320 | 177 |
| 321 | 149 |
| 322 | 126 |
| 323 | 92 |
| 324 | 214 |
| 325 | 7.2 |
| 326 | 10 |
| 327 | 90 |
| 328 | 19 |
| 329 | 32 |
| 330 | 237 |
| 331 | 116 |
| 332 | 7.6 |
| 333 | 8.1 |
| 334 | 6.6 |
| 335 | 722 |
| 336 | 14 |
| 337 | 15 |
| 338 | 1.8 |
| 339 | 983 |
| 340 | 5,528 |
| 341 | 75 |
| 342 | 136 |
| 343 | 67 |
| 344 | 75 |
| 345 | 124 |
| 346 | 816 |
| 347 | 1,820 |
| 348 | 372 |
| 349 | 1,367 |
| 350 | 2,053 |
| 351 | 5.7 |
| 352 | 15 |
| 353 | 14 |
| 354 | 3.5 |
| 355 | 16 |
| 356 | 17 |
| 357 | 11 |
| 358 | 6.6 |
| 359 | 1,656 |

We claim:

1. A compound of the formula 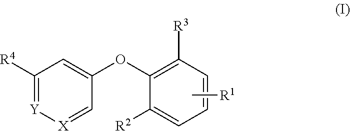
wherein
X is —N— or CR⁵, where R⁵ is hydrogen, C₁₋₃ alkyl, CN or halogen;
Y is CR⁶, where R⁶ is hydrogen, CN, halogen, O—C₁₋₃ alkyl, O—C₁₋₃ haloalkyl or C₃₋₆ cycloalkyl;
R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};
each R^{x} and R^{y} is independently hydrogen or C₁₋₃ alkyl;
R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or aryl, all of said alkyl, heterocyclyl or aryl groups substituted with 0-3 R^{1b};
R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;
R² and R³ are, independently at each occurrence, hydrogen, halogen or C₁₋₃ alkyl;
R⁴ is C1.6 alkyl, C₁₋₆ alkenyl, C₁₋₆ haloalkyl, C₁₋₆ hydroxyalkyl, CO—C₁₋₃ haloalkyl or C₃₋₆ cycloalkyl, each of said groups substituted with 0-2 R^{4a};
R^{4a} is halogen or C₁₋₃ alkyl;
p is 0, 1 or 2;
r is 0, 1, 2, 3 or 4;
or a stereoisomer or pharmaceutically-acceptable salt thereof.

2. The compound according to claim 1 of the formula 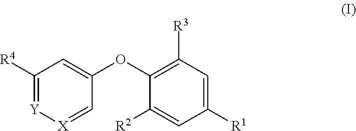
wherein
X is —N— or CR⁵, where R⁵ is hydrogen, C₁₋₃ alkyl, CN or halogen;
Y is CR⁶, where R⁶ is hydrogen, CN, halogen, O—C₁₋₃ alkyl, O—C₁₋₃ haloalkyl or C₃₋₆ cycloalkyl;
R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};
each R^{x} and R^{y} is independently hydrogen or C₁₋₃ alkyl;
R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or aryl, all of said alkyl, heterocyclyl or aryl groups substituted with 0-3 R^{1b};
R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;
R² and R³ are, independently at each occurrence, hydrogen, halogen or C₁₋₃ alkyl;
R⁴ is C₁₋₆ alkyl, C₁₋₆ alkenyl, C₁₋₆ haloalkyl, C₁₋₆ hydroxyalkyl, CO—C₁₋₃ haloalkyl or C₃₋₆ cycloalkyl, each of said groups substituted with 0-2 R^{4a};
R^{4a} is halogen or C₁₋₃ alkyl;
p is 0, 1 or 2;
r is 0, 1, 2, 3 or 4;
or a stereoisomer or pharmaceutically-acceptable salt thereof.

3. The compound according to claim 2 of the formula 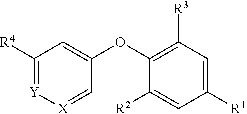
wherein
X is —N— or CR⁵, where R⁵ is hydrogen, C₁₋₃ alkyl, CN or halogen;
Y is CR⁶, where R⁶ is hydrogen, CN, halogen, O—C₁₋₃ alkyl, O—C₁₋₃ haloalkyl or C₃₋₆ cycloalkyl;
R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};
each R^{x} and R^{y} is independently hydrogen or C₁₋₃ alkyl;
R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or aryl, all of said alkyl, heterocyclyl or aryl groups substituted with 0-3 R^{1b};
R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;
R² and R³ are, independently at each occurrence, hydrogen, halogen or C₁₋₃ alkyl;
R⁴ is C₁₋₆ alkyl, C₁₋₆ haloalkyl, C₁₋₆ hydroxyalkyl, CO—C₁₋₃ haloalkyl or C₃₋₆ cycloalkyl, each of said groups substituted with 0-2 R^{4a};
R^{4a} is halogen or C₁₋₃ alkyl;
p is 0 or 1;
r is 0, 1, 2 or 3;
or a stereoisomer or pharmaceutically-acceptable salt thereof.

4. The compound according to claim 3 of the formula 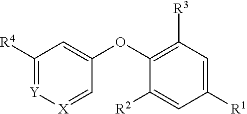
wherein
X is —N— or CR⁵, where R⁵ is hydrogen, C₁₋₃ alkyl, CN or halogen;
Y is CR⁶, where R⁶ is hydrogen, CN, halogen, O—C₁₋₃ alkyl, O—C₁₋₃ haloalkyl or C₃₋₆ cycloalkyl;
R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};
each R^{x} and R^{y} is independently hydrogen or C₁₋₃ alkyl;
R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or phenyl, all of said alkyl, heterocyclyl or phenyl groups substituted with 0-3 R^{1b};
R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;
R² and R³ are, independently at each occurrence, CH₃, Cl or F;
R⁴ is C₁₋₆ alkyl, C₁₋₆ haloalkyl, C₁₋₆ hydroxyalkyl, CO—C₁₋₃ haloalkyl or C₃₋₆ cycloalkyl, each of said groups substituted with 0-2 R^{4a};
R^{4a} is halogen or C₁₋₃ alkyl;
p is 0 or 1;
r is 0, 1, 2 or 3;
or a stereoisomer or pharmaceutically-acceptable salt thereof.

5. The compound according to claim 4 of the formula 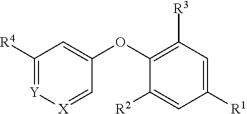
wherein
X is —N— or CR⁵, where R⁵ is hydrogen, C₁₋₃ alkyl, CN or halogen;
Y is CR⁶, where R⁶ is hydrogen, CN, halogen, O—C₁₋₃ alkyl, O—C₁₋₃ haloalkyl or C₃₋₆ cycloalkyl;
R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};
each R^{x} and R^{y} is independently hydrogen or C₁₋₃ alkyl;
R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or phenyl, all of said alkyl, heterocyclyl or phenyl groups substituted with 0-3 R^{1b};
R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;
R² and R³ are, independently at each occurrence, Cl or F;
R⁴ is C₁₋₆ alkyl, C₁₋₆ haloalkyl, C₁₋₆ hydroxyalkyl, CO—C₁₋₃ haloalkyl or C₃₋₆ cycloalkyl, each of said groups substituted with 0-2 R^{4a};
R^{4a} is halogen or C₁₋₃ alkyl;
p is 0 or 1;
r is 0, 1, 2 or 3;
or a stereoisomer or pharmaceutically-acceptable salt thereof.

6. The compound according to claim 5 of the formula 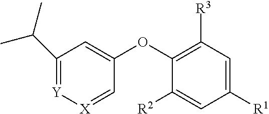
wherein
X is —N— or CR⁵, where R⁵ is hydrogen, C₁₋₃ alkyl, CN or halogen;
Y is CR⁶, where R⁶ is hydrogen, CN, halogen, O—C₁₋₃ alkyl, O—C₁₋₃ haloalkyl or C₃₋₆ cycloalkyl;
R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};
each R^{x} and R^{y} is independently hydrogen or C₁₋₃ alkyl;
R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or phenyl, all of said alkyl, heterocyclyl or phenyl groups substituted with 0-3 R^{1b};
R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;
R² and R³ are, independently at each occurrence, Cl or F;
p is 0 or 1;
r is 0, 1, 2 or 3;
or a stereoisomer or pharmaceutically-acceptable salt thereof.

7. The compound according to claim 6 of the formula 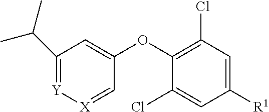
wherein
X is —N— or CR⁵, where R⁵ is hydrogen, C₁₋₃ alkyl, CN or halogen;
Y is CR⁶, where R⁶ is hydrogen, CN, halogen, O—C₁₋₃ alkyl, O—C₁₋₃ haloalkyl or C₃₋₆ cycloalkyl;
R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};
each R^{x} and R^{y} is independently hydrogen or C₁₋₃ alkyl;
R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or phenyl, all of said alkyl, heterocyclyl or phenyl groups substituted with 0-3 R^{1b};
R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;
p is 0 or 1;
r is 0, 1, 2 or 3;
or a stereoisomer or pharmaceutically-acceptable salt thereof.

8. The compound according to claim 1 of the formula 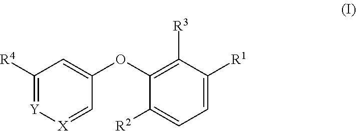
wherein
X is —N— or CR⁵, where R⁵ is hydrogen, C₁₋₃ alkyl, CN or halogen;
Y is CR⁶, where R⁶ is hydrogen, CN, halogen, O—C₁₋₃ alkyl, O—C₁₋₃ haloalkyl or C₃₋₆ cycloalkyl;
R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};
each R^{x} and R^{y} is independently hydrogen or C₁₋₃ alkyl;
R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or aryl, all of said alkyl, heterocyclyl or aryl groups substituted with 0-3 R^{1b};
R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;
R² and R³ are, independently at each occurrence, hydrogen, halogen or C₁₋₃ alkyl;
R⁴ is C₁₋₆ alkyl, C₁₋₆ alkenyl, C₁₋₆ haloalkyl, C₁₋₆ hydroxyalkyl, CO—C₁₋₃ haloalkyl or C₃₋₆ cycloalkyl, each of said groups substituted with 0-2 R^{4a};
R^{4a} is halogen or C₁₋₃ alkyl;
p is 0, 1 or 2;
r is 0, 1, 2, 3 or 4;
or a stereoisomer or pharmaceutically-acceptable salt thereof.

9. The compound according to claim 8 of the formula 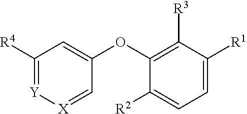
wherein
X is —N— or CR⁵, where R⁵ is hydrogen, C₁₋₃ alkyl, CN or halogen;
Y is CR⁶, where R⁶ is hydrogen, CN, halogen, O—C₁₋₃ alkyl, O—C₁₋₃ haloalkyl or C₃₋₆ cycloalkyl;
R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};
each R^{x} and R^{y} is independently hydrogen or C₁₋₃ alkyl;
R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or aryl, all of said alkyl, heterocyclyl or aryl groups substituted with 0-3 R^{1b};
R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;
R² and R³ are, independently at each occurrence, hydrogen, halogen or C₁₋₃ alkyl;
R⁴ is C₁₋₆ alkyl, C₁₋₆ haloalkyl, C₁₋₆ hydroxyalkyl, CO—C₁₋₃ haloalkyl or C₃₋₆ cycloalkyl, each of said groups substituted with 0-2 R^{4a};
R^{4a} is halogen or C₁₋₃ alkyl;
p is 0 or 1;
r is 0, 1, 2 or 3;
or a stereoisomer or pharmaceutically-acceptable salt thereof.

10. The compound according to claim 9 of the formula 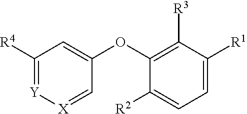
wherein
X is —N— or CR⁵, where R⁵ is hydrogen, C₁₋₃ alkyl, CN or halogen;
Y is CR⁶, where R⁶ is hydrogen, CN, halogen, O—C₁₋₃ alkyl, O—C₁₋₃ haloalkyl or C₃₋₆ cycloalkyl;
R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};
each R^{x} and R^{y} is independently hydrogen or C₁₋₃ alkyl;
R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or phenyl, all of said alkyl, heterocyclyl or phenyl groups substituted with 0-3 R^{1b};
R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;
R² and R³ are, independently at each occurrence, CH₃, Cl or F;
R⁴ is C₁₋₆ alkyl, C₁₋₆ haloalkyl, C₁₋₆ hydroxyalkyl, CO—C₁₋₃ haloalkyl or C₃₋₆ cycloalkyl, each of said groups substituted with 0-2 R^{4a};
R^{4a} is halogen or C₁₋₃ alkyl;
p is 0 or 1;
r is 0, 1, 2 or 3;
or a stereoisomer or pharmaceutically-acceptable salt thereof.

11. The compound according to claim 10 of the formula 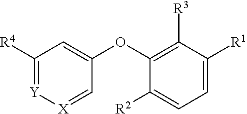
wherein
X is —N— or CR⁵, where R⁵ is hydrogen, C₁₋₃ alkyl, CN or halogen;
Y is CR⁶, where R⁶ is hydrogen, CN, halogen, O—C₁₋₃ alkyl, O—C₁₋₃ haloalkyl or C₃₋₆ cycloalkyl;
R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};
each R^{x} and R^{y} is independently hydrogen or C₁₋₃ alkyl;
R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or phenyl, all of said alkyl, heterocyclyl or phenyl groups substituted with 0-3 R^{1b};
R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;
R² and R³ are, independently at each occurrence, Cl or F;
R⁴ is C₁₋₆ alkyl, C₁₋₆ haloalkyl, C₁₋₆ hydroxyalkyl, CO—C₁₋₃ haloalkyl or C₃₋₆ cycloalkyl, each of said groups substituted with 0-2 R^{4a};
R^{4a} is halogen or C₁₋₃ alkyl;
p is 0 or 1;
r is 0, 1, 2 or 3;
or a stereoisomer or pharmaceutically-acceptable salt thereof.

12. The compound according to claim 11 of the formula 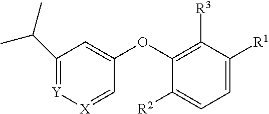
wherein
X is —N— or CR⁵, where R⁵ is hydrogen, C₁₋₃ alkyl, CN or halogen;
Y is CR⁶, where R⁶ is hydrogen, CN, halogen, O—C₁₋₃ alkyl, O—C₁₋₃ haloalkyl or C₃₋₆ cycloalkyl;
R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};
each R^{x} and R^{y} is independently hydrogen or C₁₋₃ alkyl;
R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or phenyl, all of said alkyl, heterocyclyl or phenyl groups substituted with 0-3 R^{1b};
R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;
R² and R³ are, independently at each occurrence, Cl or F;
p is 0 or 1;
r is 0, 1, 2 or 3;
or a stereoisomer or pharmaceutically-acceptable salt thereof.

13. The compound according to claim 12 of the formula 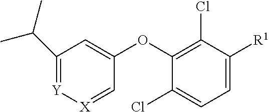
wherein
X is —N— or CR⁵, where R⁵ is hydrogen, C₁₋₃ alkyl, CN or halogen;
Y is CR⁶, where R⁶ is hydrogen, CN, halogen, O—C₁₋₃ alkyl, O—C₁₋₃ haloalkyl or C₃₋₆ cycloalkyl;
R¹ is —(CH₂)ₚ—NHCOO—(CR^{x}R^{y})ᵣ—R^{1a}, —(CH₂)ₚ—NR^{x}CO—(CR^{x}R^{y})ᵣ—R^{1a}, —(CH₂)ₚ—NR^{x}SO₂—(CR^{x}R^{y})ᵣ—R^{1a}, —(CH₂)ₚ—CONR^{x}—(CR^{x}R^{y})ᵣ—R^{1a}, 4-10 membered heterocycle-(CR^{x}R^{y})ᵣ—R^{1a}, —CO-4-10 membered heterocycle-(CR^{x}R^{y})ᵣ—R^{1a};
each R^{x} and R^{y} is independently hydrogen or C₁₋₃ alkyl;
R^{1a} is, independently at each occurrence, hydrogen, CF₃, halogen, CN, OH, COOH, C₁₋₆ alkyl, C₃₋₆ cycloalkyl, C₁₋₃ haloalkyl, C₁₋₃ hydroxyalkyl, CONR^{x}R^{y}, COO—C₁₋₆ alkyl, NHCO—C₁₋₆ alkyl, NH—C₁₋₆ alkyl, NH—SO₂—C₁₋₆ alkyl, NH—SO₂—C₃₋₆ cycloalkyl, SO₂—C₁₋₆ alkyl, 4-10 membered heterocycle or phenyl, all of said alkyl, heterocyclyl or phenyl groups substituted with 0-3 R^{1b};
R^{1b} is, independently at each occurrence, hydrogen, CF₃, halogen, CN, OH, COOH, C₁₋₆ alkyl, CO—NR^{x}R^{y}, CO—C₁₋₃ haloalkyl, COO—C₁₋₆ alkyl, NR^{x}R^{y}, NH—SO₂—C₁₋₆ alkyl, NH—SO₂—C₃₋₆ cycloalkyl, SO₂—C₁₋₆ alkyl, SO₂—C₃₋₆ cycloalkyl, SO₂—NR^{x}R^{y}, or 4-10 membered heterocycle;
p is 0 or 1;
r is 0, 1, 2 or 3;
or a stereoisomer or pharmaceutically-acceptable salt thereof.

14. A compound which is
2,4-dichloro-3-(3-isopropyl-4-methoxyphenoxy)benzonitrile,
4,6-dichloro-5-(3-isopropyl-4-methoxyphenoxy)-2-phenyl-1H-benzo[d]imidazole,
N-({2,4-dichloro-3-[4-methoxy-3-(propan-2-yl)phenoxy]phenyl}methyl)-2-[(1-methanesulfonylpiperidin-4-yl)oxy]acetamide,
N-({2,4-dichloro-3-[4-methoxy-3-(propan-2-yl)phenoxy]phenyl}methyl)-2-{[1-(ethanesulfonyl)piperidin-4-yl]oxy}acetamide,
2-benzyl-4,6-dichloro-5-[4-methoxy-3-(propan-2-yl)phenoxy]-1H-1,3-benzodiazole,
4,6-dichloro-5-[4-methoxy-3-(propan-2-yl)phenoxy]-2-[(pyridin-3-yl)methyl]-1H-1,3-benzodiazole,
3,5-dichloro-4-[4-methoxy-3-(propan-2-yl)phenoxy]aniline, or
{3,5-dichloro-4-[4-methoxy-3-(propan-2-yl)phenoxy]phenyl}methanol
or a pharmaceutically acceptable salt thereof.

15. A pharmaceutical composition comprising one or more compounds according to claim 1 or a pharmaceutically acceptable salt thereof and one or more pharmaceutically acceptable carriers, diluents or excipients.
